(19) United States
(12) Patent Application Publication
(10) Pub. No.: US 2025/0382297 A1
(43) Pub. Date: Dec. 18, 2025

(54) INHIBITORS OF FGFR2 AND FGFR3 AND USES THEREOF

(71) Applicant: Insilico Medicine IP Limited, Hong Kong (HK)

(72) Inventors: Xiao DING, Shanghai (CN); Yingtao LIU, Shanghai (CN); Yazhou WANG, Shanghai (CN); Jianping WU, Shanghai (CN); Jinxin LIU, Shanghai (CN)

(21) Appl. No.: 18/878,618

(22) PCT Filed: Jun. 28, 2023
(86) PCT No.: PCT/CN2023/103171

§ 371 Date: Dec. 23, 2024

(30) Foreign Application Priority Data
Jun. 29, 2022 (WO) PCT/CN2022/102136
Mar. 21, 2023 (WO) PCT/CN2023/082796
Jun. 2, 2023 (WO) PCT/CN2023/097992

(51) Int. Cl. C07D 487/04; A61K 31/4439; A61K 31/4985; A61K 31/5025; A61K 31/506; A61K 31/519; A61K 31/53; C07D 471/04; C07D 513/04

(52) U.S. Cl. CPC C07D 487/04; A61K 31/4439; A61K 31/4985; A61K 31/5025; A61K 31/506; A61K 31/519; A61K 31/53; C07D 471/04; C07D 513/04

(57) ABSTRACT

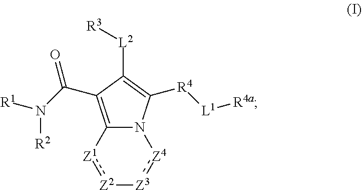

Provided are FGFR2 and FGFR3 inhibitors of Formula (I) and pharmaceutical compositions comprising said inhibitors. The compounds and compositions are useful for the treatment of a disease or disorder associated with FGFR2 and/or FGFR3.

### CROSS-REFERENCE

This patent application claims the benefit of International Application No. PCT/CN2022/102136, filed Jun. 29, 2022, International Application No. PCT/CN2023/082796, filed Mar. 21, 2023, and International Application No. PCT/CN2023/097992, filed Jun. 2, 2023; which are incorporated herein by reference in their entirety.

### BACKGROUND

Fibroblast growth factor receptors (FGFR1, FGFR2, FGFR3, and FGFR4) are a subfamily of receptor tyrosine kinases (RTKs) consisting of an extracellular ligand binding domain and an intracellular kinase domain. Binding of FGF ligands triggers receptor dimerization and subsequent phosphorylation of the substrates such as FGFR substrate 2 (FRS2) and phospholipase Cγ (PLC-γ) to further activate downstream signaling cascades, leading to regulation of key cellular functions, including cell survival, proliferation, differentiation, migration, and angiogenesis (Clin. Cancer Res. 21(12) Jun. 15, 2015). Aberrant activation of FGFR signaling pathway through FGFR fusions, mutations, and/or amplifications can lead to tumor development, progression, and resistance to conventional cancer therapies. Pan-FGFR inhibitors have achieved clear clinical responses in multiple FGFR-altered cancers, however, on-target toxicities are also observed, including FGFR1-mediated dose-limiting toxicities, e.g. hyperphosphatemia and tissue mineralization, and FGFR4-mediated dose-limiting toxicity, e.g. diarrhea. Thus, development of next-generation dual FGFR2/3 inhibitors with higher selectivity, especially against FGFR1, is desired for use in the treatment of cancer and other disorders.

### SUMMARY

Disclosed herein is a compound of Formula (I), or a pharmaceutically acceptable salt or stereoisomer thereof:

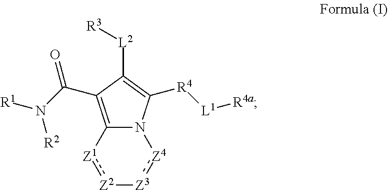

wherein:
   Z¹ is absent, C(R⁵), C(R⁵)(R^{5a}), N, or N(R⁹);
   Z² is C(R⁶), C(R⁶)(R^{6a}), O, S, N, or N(R⁹);
   Z³ is C(R⁷), C(R⁷)(R^{7a}), O, S, N, or N(R⁹);
   Z⁴ is C(R⁸), C(R⁸)(R^{8a}), O, S, N, or N(R⁹);
   wherein when Z¹ is C(R⁵), C(R⁵)(R^{5a}), N, or N(R⁹), then no more than two of Z¹, Z², Z³, and Z⁴ are N or N(R⁹); and when Z¹ is absent, then no more than one of Z², Z³, and Z⁴ is O, S, N or N(R⁹);
   R¹ and R² are independently selected from hydrogen, C₁₋₆alkyl, and C₁₋₆haloalkyl;
   R³ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three groups selected from R^{15a};
   R⁴ is selected from C₃₋₆cycloalkylene, C₂₋₉heterocycloalkylene, C₆₋₁₀arylene, and C₁₋₉heteroarylene, wherein C₃₋₆cycloalkylene, C₂₋₉heterocycloalkylene, C₆₋₁₀arylene, and C₁₋₉heteroarylene are optionally substituted with one, two, or three groups selected from R^{15b};
   L¹ is a bond, —N(R^{9a})—, —N(R^{9a})C(O)—, —C(O)N(R^{9a})—, —N(R^{9a})S(O)₂—, —S(O)₂N(R^{9a})—, —O—, —C(O)—, —OC(O)—, —C(O)O—, —C(S)—, —S—, —S(O)—, —S(O)₂—, —OS(O)—, —OS(O)₂—, C₁₋₆alkylene, C₂₋₆alkenylene, C₂₋₆alkynylene, C₃₋₆cycloalkylene, C₂₋₉heterocycloalkylene, or C₁₋₉heteroarylene, wherein C₁₋₆alkylene, C₂₋₆alkenylene, C₂₋₆alkynylene, C₃₋₆cycloalkylene, C₂₋₉heterocycloalkylene, and C₁₋₉heteroarylene, are optionally substituted with one, two, or three groups selected from R^{15c};
   L² is a bond, C₁₋₆alkylene, C₂₋₆alkenylene, or C₂₋₆alkynylene, wherein C₁₋₆alkylene, C₂₋₆alkenylene, or C₂₋₆alkynylene are optionally substituted with one, two, or three groups selected from R^{15c};
   R^{4a} is selected from halogen, —CN,

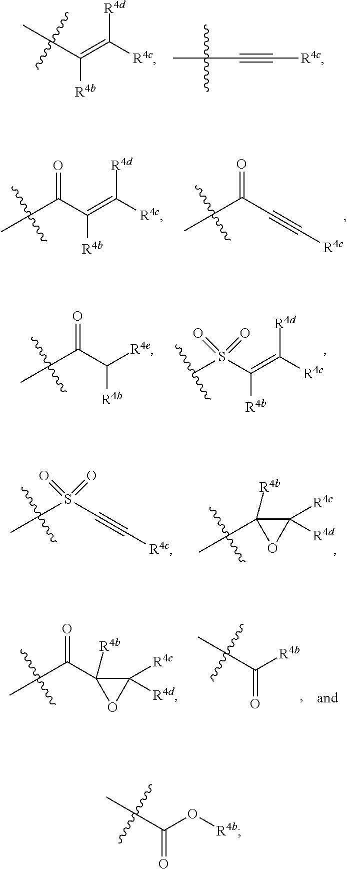

R^{4b}, R^{4c}, and R^{4d} are each independently hydrogen, halogen, —CN, —C(O)R¹³, —C(O)OR¹⁰, —C(O)N(R¹⁰)(R¹¹), —N(R¹⁰)(R¹¹), —C₁₋₆alkyl-N(R¹⁰)(R¹¹), —C(O)N(R¹⁰)OR¹⁰, C₁₋₆alkyl, phenyl, 3 to 7-membered heterocycloalkyl, or 5 or 6-membered heteroaryl, wherein the C₁₋₆alkyl, phenyl, 3 to 7-membered heterocycloalkyl, or 5 or 6-membered heteroaryl is optionally substituted with one, two, or three groups selected from R^{15d}; or
R^{4b} and R^{4c} together with the atoms to which they are attached form a 3 to 14-membered cycloalkyl or 3 to 14-membered heterocycloalkyl; wherein the 3 to 14-membered cycloalkyl and 3 to 14-membered heterocycloalkyl are optionally substituted with 1, 2, 3, or 4 R^{15d}, or R^{4d} and R^{4c} together with the atoms to which they are attached form a 3 to 14-membered cycloalkyl or 3 to 14-membered heterocycloalkyl; wherein the 3 to 14-membered cycloalkyl and 3 to 14-membered heterocycloalkyl are optionally substituted with 1, 2, 3, or 4 R^{15d},
R^{4e} is halogen or —OS(O)₂R¹³;
R⁵, R^{5a}, R⁶, R^{6a}, R⁷, R^{7a}, R⁸, and R^{8a} are independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)(R¹³), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15e};
R⁹ is selected from hydrogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹⁰, —C(O)R¹³, —S(O)R¹³, —C(O)N(R¹⁰)(R¹¹), and —S(O)₂R¹³, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15f};
R^{9a} is selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15g};
each R¹⁰ is independently selected from hydrogen, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, —CH₂—C₆₋₁₀aryl, C₆₋₁₀aryl, C₁₋₉heteroaryl, and —CH₂—C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, —CH₂—C₆₋₁₀aryl, C₆₋₁₀aryl, C₁₋₉heteroaryl, and —CH₂—C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl; or R¹⁰ and R¹¹, together with the nitrogen to which they are attached, form a C₂₋₉heterocycloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R^{15a}, R^{15b}, R^{15c}, R^{15d}, R^{15e}, R^{15f}, and R^{15g} are each independently selected from halogen, oxo, —CN, C₁₋₆alkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)(R¹³), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)(R¹³), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹) and —P(O)(R¹⁰)₂, and
[symbol] indicates a single or double bond such that all valences are satisfied.

In some embodiments, disclosed herein is a compound, or a pharmaceutically acceptable salt or stereoisomer thereof, having the structure of Formula (Ia):

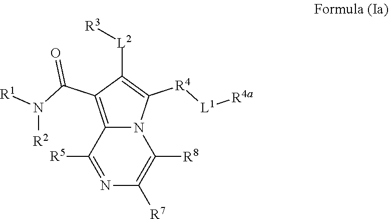

wherein R¹, R², R³, R⁴, R^{4a}, R⁵, R⁷, R⁸, L¹ and L² have the meaning as defined herein.

In some embodiments, disclosed herein is a compound, or a pharmaceutically acceptable salt or stereoisomer thereof, having the structure of Formula (Ib):

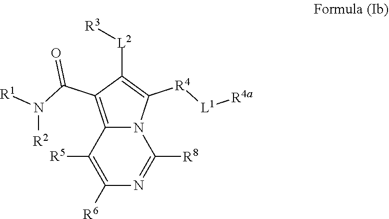

wherein R¹, R², R³, R⁴, R^{4a}, R⁵, R⁶, R⁸, L¹ and L² have the meaning as defined herein.

In some embodiments, disclosed herein is a compound, or a pharmaceutically acceptable salt or stereoisomer thereof, having the structure of Formula (Ic):

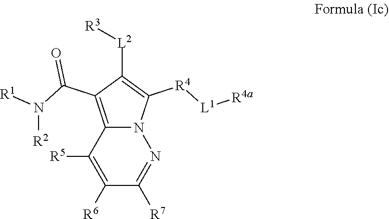

wherein R¹, R², R³, R⁴, R^{4a}, R⁵, R⁶, R⁷, L¹ and L² have the meaning as defined herein.

In some embodiments, disclosed herein is a compound, or a pharmaceutically acceptable salt or stereoisomer thereof, having the structure of Formula (Id):

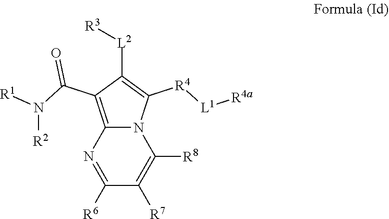

wherein R¹, R², R³, R⁴, R^{4a}, R⁶, R⁷, R⁸, L¹ and L² have the meaning as defined herein.

In some embodiments, disclosed herein is a compound, or a pharmaceutically acceptable salt or stereoisomer thereof, having the structure of Formula (Ie):

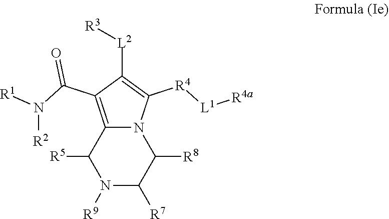

wherein R¹, R², R³, R⁴, R^{4a}, R⁵, R⁷, R⁸, R⁹, L¹ and L² have the meaning as defined herein.

In some embodiments, disclosed herein is a compound, or a pharmaceutically acceptable salt or stereoisomer thereof, having the structure of Formula (Ia′) or Formula (Ia″):

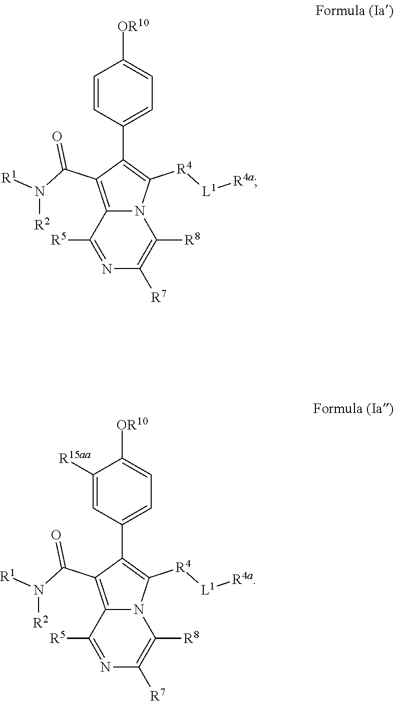

wherein R¹, R², R⁴, R^{4a}, R⁵, R⁷, R⁸, R¹⁰, R^{15aa}, and L¹ have the meaning as defined herein.

In some embodiments, disclosed herein is a compound, or a pharmaceutically acceptable salt or stereoisomer thereof, having the structure of Formula (Ib′) or Formula (Ib″):

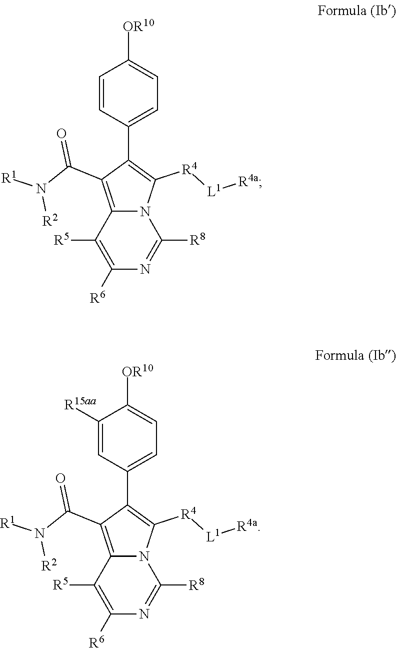

wherein R¹, R², R⁴, R^{4a}, R⁵, R⁶, R⁸, R¹⁰, R^{15aa}, and L¹ have the meaning as defined herein.

In some embodiments, disclosed herein is a compound, or a pharmaceutically acceptable salt or stereoisomer thereof, having the structure of Formula (Ic′) or Formula (Ic″):

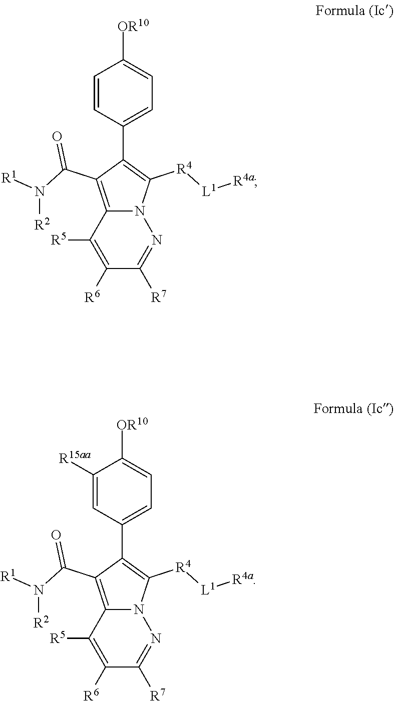

wherein R¹, R², R⁴, R^{4a}, R⁵, R⁶, R⁷, R¹⁰, R^{15aa}, and L¹ have the meaning as defined herein.

In some embodiments is a pharmaceutical composition comprising a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, and at least one pharmaceutically acceptable excipient.

In some embodiments is a method of treating cancer in a mammal in need thereof, comprising administering to the mammal a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof. In some embodiments is a method of treating cancer in a mammal in need thereof, comprising administering to the mammal a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, wherein the cancer is a solid tumor. In some embodiments is a method of treating cancer in a mammal in need thereof, comprising administering to the mammal a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, wherein the cancer is intra-hepatic cholangiocarcinoma, urothelial cancer, gastric cancer, bladder cancer, breast cancer, endometrial cancer, kidney cancer, liver cancer, lung cancer, melanoma, pancreatic cancer, prostate cancer, or thyroid cancer.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

### Definitions

In the following description, certain specific details are set forth in order to provide a thorough understanding of various embodiments. However, one skilled in the art will understand that the disclosed technology may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“oxo” refers to ═O.

“Carboxyl” refers to —COOH.

“Cyano” refers to —CN.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₅alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, COOMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from a hydrocarbon ring system comprising 6 to 30 carbon atoms and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen. As used herein, “arylene” refers to a bivalent aryl radical as described herein. An arylene can be bonded through the aryl at any suitable position. In some embodiments, when an arylene comprises an aryl fused with a cycloalkyl or heterocycloalkyl ring, the arylene is bonded at the aryl and the cycloalkyl, or the aryl and the heterocycloalkyl. In some embodiments, when an arylene comprises an aryl fused with a cycloalkyl or heterocycloalkyl ring, the arylene is bonded only at the aryl.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom), spiro, or bridged ring systems. In some embodiments, the cycloalkyl is fully saturated. Representative cycloalkyls include, but are not limited to, cycloalkyls having from three to fifteen carbon atoms (e.g., C₃-C₁₅ fully saturated cycloalkyl or C₃-C₁₅ cycloalkenyl), from three to ten carbon atoms (e.g., C₃-C₁₀ fully saturated cycloalkyl or C₃-C₁₀ cycloalkenyl), from three to eight carbon atoms (e.g., C₃-C₈ fully saturated cycloalkyl or C₃-C₈ cycloalkenyl), from three to six carbon atoms (e.g., C₃-C₆ fully saturated cycloalkyl or C₃-C₆ cycloalkenyl), from three to five carbon atoms (e.g., C₃-C₅ fully saturated cycloalkyl or C₃-C₅ cycloalkenyl), or three to four carbon atoms (e.g., C₃-C₄ fully saturated cycloalkyl or C₃-C₄ cycloalkenyl). In some embodiments, the cycloalkyl is a 3- to 10-membered fully saturated cycloalkyl or a 3- to 10-membered cycloalkenyl. In some embodiments, the cycloalkyl is a 3- to 6-membered fully saturated cycloalkyl or a 3- to 6-membered cycloalkenyl. In some embodiments, the cycloalkyl is a 5- to 6-membered fully saturated cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen. As used herein, “cycloalkylene” refers to a bivalent cycloalkyl radical as described herein. In some embodiments, when a cycloalkylene comprises a cycloalkyl fused with an aryl or a heteroaryl ring, the cycloalkylene is bonded at the cycloalkyl and the aryl, or the cycloalkyl and the heteroaryl. In some embodiments, when a cycloalkylene comprises a cycloalkyl fused with an aryl or a heteroaryl ring, the cycloalkylene is bonded only at the cycloalkyl.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃) OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom), spiro, or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (e.g., C₂-C₁₅ fully saturated heterocycloalkyl or C₂-C₁₅ heterocycloalkenyl), from two to ten carbon atoms (e.g., C₂-C₁₀ fully saturated heterocycloalkyl or C₂-C₁₀ heterocycloalkenyl), from two to eight carbon atoms (e.g., C₂-C₈ fully saturated heterocycloalkyl or C₂-C₈ heterocycloalkenyl), from two to seven carbon atoms (e.g., C₂-C₇ fully saturated heterocycloalkyl or C₂-C₇ heterocycloalkenyl), from two to six carbon atoms (e.g., C₂-C₆ fully saturated heterocycloalkyl or C₂-C₆ heterocycloalkenyl), from two to five carbon atoms (e.g., C₂-C₅ fully saturated heterocycloalkyl or C₂-C₅ heterocycloalkenyl), or two to four carbon atoms (e.g., C₂-C₄ fully saturated heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered heterocycloalkyl. In some embodiments, the heterocycloalkyl is a 3- to 7-membered heterocycloalkyl. In some embodiments, the heterocycloalkyl is a 3- to 6-membered heterocycloalkyl. In some embodiments, the heterocycloalkyl is a 4- to 6-membered heterocycloalkyl. In some embodiments, the heterocycloalkyl is a 5- to 6-membered heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

As used herein, “heterocycloalkylene” refers to a bivalent heterocycloalkyl radical as described herein. In some embodiments, when a heterocycloalkylene comprises a heterocycloalkyl fused with an aryl or a heteroaryl ring, the heterocycloalkylene is bonded at the heterocycloalkyl and the aryl, or the heterocycloalkyl and the heteroaryl. In some embodiments, when a heterocycloalkylene comprises a heterocycloalkyl fused with an aryl or a heteroaryl ring, the heterocycloalkylene is bonded only at the heterocycloalkyl.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen. As used herein, “heteroarylene” refers to a bivalent heteroaryl radical as described herein. In some embodiments, when a heteroarylene comprises a heteroaryl fused with a cycloalkyl or heterocycloalkyl ring, the heteroarylene is bonded at the heteroaryl and the cycloalkyl, or the heteroaryl and the heterocycloalkyl. In some embodiments, when a heteroarylene comprises a heteroaryl fused with a cycloalkyl or heterocycloalkyl ring, the heteroarylene is bonded only at the heteroaryl.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns that are sterically impractical and/or synthetically non-feasible. Thus, any substituents described should generally be understood as having a maximum molecular weight of about 1,000 daltons, and more typically, up to about 500 daltons.

An “effective amount” or “therapeutically effective amount” refers to an amount of a compound administered to a mammalian subject, either as a single dose or as part of a series of doses, which is effective to produce a desired therapeutic effect.

“Treatment” of an individual (e.g. a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition.

### Compounds

Described herein are compounds of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, useful in the treatment of a disease or disorder associated with FGFR2 and FGFR3 inhibition. In some embodiments, the compounds of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, are useful in the treatment of cancer. In some embodiments, the cancer is selected from intra-hepatic cholangiocarcinoma, urothelial cancer, gastric cancer, bladder cancer, breast cancer, endometrial cancer, kidney cancer, liver cancer, lung cancer, melanoma, pancreatic cancer, prostate cancer, or thyroid cancer.

In some embodiments disclosed herein is a compound of Formula (I), or a pharmaceutically acceptable salt or stereoisomer thereof:

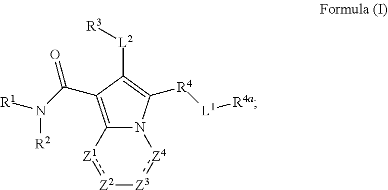

wherein:
   Z¹ is absent, C(R⁵), C(R⁵)(R^{5a}), N, or N(R⁹);
   Z² is C(R⁶), C(R⁶)(R^{6a}), O, S, N, or N(R⁹);
   Z³ is C(R⁷), C(R⁷)(R^{7a}), O, S, N, or N(R⁹);
   Z⁴ is C(R⁸), C(R⁸)(R^{8a}), O, S, N, or N(R⁹);
   wherein when Z¹ is C(R⁵), C(R⁵)(R^{5a}), N, or N(R⁹), then no more than two of Z¹, Z², Z³, and Z⁴ are N or N(R⁹); and when Z¹ is absent, then no more than one of Z², Z³, and Z⁴ is O, S, N or N(R⁹);
   R¹ and R² are independently selected from hydrogen, C₁₋₆alkyl, and C₁₋₆haloalkyl;
   R³ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three groups selected from R^{15a};
   R⁴ is selected from C₃₋₆cycloalkylene, C₂₋₉heterocycloalkylene, C₆₋₁₀arylene, and C₁₋₉heteroarylene, wherein C₃₋₆cycloalkylene, C₂₋₉heterocycloalkylene, C₆₋₁₀arylene, and C₁₋₉heteroarylene are optionally substituted with one, two, or three groups selected from R^{15b};
   L¹ is a bond, —N(R^{9a})—, —N(R^{9a})C(O)—, —C(O)N(R^{9a})—, —N(R^{9a})S(O)₂—, —S(O)₂N(R^{9a})—, —O—, —C(O)—, —OC(O)—, —C(O)O—, —C(S)—, —S—, —S(O)—, —S(O)₂—, —OS(O)—, —OS(O)₂—, C₁₋₆alkylene, C₂₋₆alkenylene, C₂₋₆alkynylene, C₃₋₆cycloalkylene, C₂₋₉heterocycloalkylene, or C₁₋₉heteroarylene, wherein C₁₋₆alkylene, C₂₋₆alkenylene, C₂₋₆alkynylene, C₃₋₆cycloalkylene, C₂₋₉heterocycloalkylene, and C₁₋₉heteroarylene, are optionally substituted with one, two, or three groups selected from R^{15c};
   L² is a bond, C₁₋₆alkylene, C₂₋₆alkenylene, or C₂₋₆alkynylene, wherein C₁₋₆alkylene, C₂₋₆alkenylene, or C₂₋₆alkynylene are optionally substituted with one, two, or three groups selected from R^{15c};
   R^{4a} is selected from halogen, —CN,

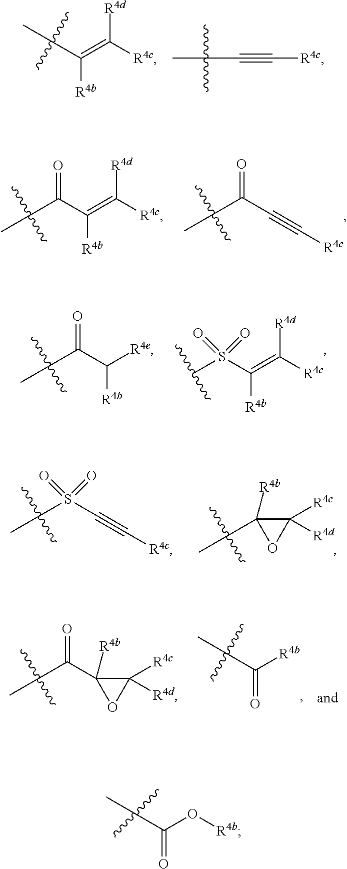

R^{4b}, R^{4c}, and R^{4d} are each independently hydrogen, halogen, —CN, —C(O)R¹³, —C(O)OR¹⁰, —C(O)N(R¹⁰)(R¹¹), —N(R¹⁰)(R¹¹), —C₁₋₆alkyl-N(R¹⁰)(R¹¹), —C(O)N(R¹⁰)OR¹⁰, C₁₋₆alkyl, phenyl, 3 to 7-membered heterocycloalkyl, or 5 or 6-membered heteroaryl; or
R^{4b} and R^{4c} together with the atoms to which they are attached form a 3 to 14-membered cycloalkyl or 3 to 14-membered heterocycloalkyl; wherein the 3 to 14-membered cycloalkyl and 3 to 14-membered heterocycloalkyl are optionally substituted with 1, 2, 3, or 4 R^{15d}, or R^{4d} and R^{4c} together with the atoms to which they are attached form a 3 to 14-membered cycloalkyl or 3 to 14-membered heterocycloalkyl; wherein the 3 to 14-membered cycloalkyl and 3 to 14-membered heterocycloalkyl are optionally substituted with 1, 2, 3, or 4 R^{15d};
R^{4e} is halogen or —OS(O)₂R¹³;
R⁵, R^{5a}, R⁶, R^{6a}, R⁷, R^{7a}, R⁸, and R^{8a} are independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)(R¹³), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15e};
R⁹ is selected from hydrogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹⁰, —C(O)R¹³, —S(O)R¹³, —C(O)N(R¹⁰)(R¹¹), and —S(O)₂R¹³, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15f};
R^{9a} is selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15g};
each R¹⁰ is independently selected from hydrogen, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, —CH₂—C₆₋₁₀aryl, C₆₋₁₀aryl, C₁₋₉heteroaryl, and —CH₂—C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, —CH₂—C₆₋₁₀aryl, C₆₋₁₀aryl, C₁₋₉heteroaryl, and —CH₂—C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl; or R¹⁰ and R¹¹, together with the nitrogen to which they are attached, form a C₂₋₉heterocycloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R^{15a}, R^{15b}, R^{15c}, R^{15d}, R^{15c}, R^{15f}, and R^{15g} are each independently selected from halogen, oxo, —CN, C₁₋₆alkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)(R¹³), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)(R¹³), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹) and —P(O)(R¹⁰)₂, and
[symbol] indicates a single or double bond such that all valences are satisfied.

In some embodiments of a compound of Formula (I), or a pharmaceutically acceptable salt or stereoisomer thereof, Z¹ is C(R⁵), Z² is N, Z³ is C(R⁷), and Z⁴ is C(R⁸). In some embodiments of a compound of Formula (I), or a pharmaceutically acceptable salt or stereoisomer thereof, Z¹ is C(R⁵), Z² is C(R⁶), Z³ is N, and Z⁴ is C(R⁸). In some embodiments of a compound of Formula (I), or a pharmaceutically acceptable salt or stereoisomer thereof, Z¹ is N, Z² is C(R⁶), Z³ is C(R⁷), and Z⁴ is C(R⁸). In some embodiments of a compound of Formula (I), or a pharmaceutically acceptable salt or stereoisomer thereof, Z¹ is C(R⁵), Z² is C(R⁶), Z³ is C(R⁷), and Z⁴ is N. In some embodiments of a compound of Formula (I), or a pharmaceutically acceptable salt or stereoisomer thereof, Z¹ is C(R⁵)(R^{5a}), Z² is N(R⁹), Z³ is C(R⁷)(R^{7a}), and Z⁴ is C(R⁸)(R^{8a}). In some embodiments of a compound of Formula (I), or a pharmaceutically acceptable salt or stereoisomer thereof, Z¹ is absent, Z² is S, Z³ is C(R⁷), and Z⁴ is C(R⁸). In some embodiments of a compound of Formula (I), or a pharmaceutically acceptable salt or stereoisomer thereof, Z¹ is absent, Z² is O, Z³ is C(R⁷), and Z⁴ is C(R⁸). In some embodiments of a compound of Formula (I), or a pharmaceutically acceptable salt or stereoisomer thereof, Z¹ is absent, Z² is N(R⁹), Z³ is C(R⁷), and Z⁴ is C(R⁸).

In some embodiments disclosed herein is a compound of Formula (Ia), or a pharmaceutically acceptable salt or stereoisomer thereof:

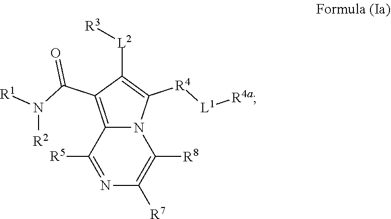

wherein:
   R¹ and R² are independently selected from hydrogen, C₁₋₆alkyl, and C₁₋₆haloalkyl;
   R³ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three groups selected from R^{15a};
   R⁴ is selected from C₃₋₆cycloalkylene, C₂₋₉heterocycloalkylene, C₆₋₁₀arylene, and C₁₋₉heteroarylene, wherein C₃₋₆cycloalkylene, C₂₋₉heterocycloalkylene, C₆₋₁₀arylene, and C₁₋₉heteroarylene are optionally substituted with one, two, or three groups selected from R^{15b};
   L¹ is a bond, —N(R^{9a})—, —N(R^{9a})C(O)—, —C(O)N(R^{9a})—, —N(R^{9a})S(O)₂—, —S(O)₂N(R^{9a})—, —O—, —C(O)—, —OC(O)—, —C(O)O—, —C(S)—, —S—, —S(O)—, —S(O)₂—, —OS(O)—, —OS(O)₂—, C₁₋₆alkylene, C₂₋₆alkenylene, C₂₋₆alkynylene, C₃₋₆cycloalkylene, C₂₋₉heterocycloalkylene, or C₁₋₉heteroarylene, wherein C₁₋₆alkylene, C₂₋₆alkenylene, C₂₋₆alkynylene, C₃₋₆cycloalkylene, C₂₋₉heterocycloalkylene, and C₁₋₉heteroarylene, are optionally substituted with one, two, or three groups selected from R^{15c};
   L² is a bond, C₁₋₆alkylene, C₂₋₆alkenylene, or C₂₋₆alkynylene, wherein C₁₋₆alkylene, C₂₋₆alkenylene, or C₂₋₆alkynylene are optionally substituted with one, two, or three groups selected from R^{15c};
   R^{4a} is selected from halogen, —CN,

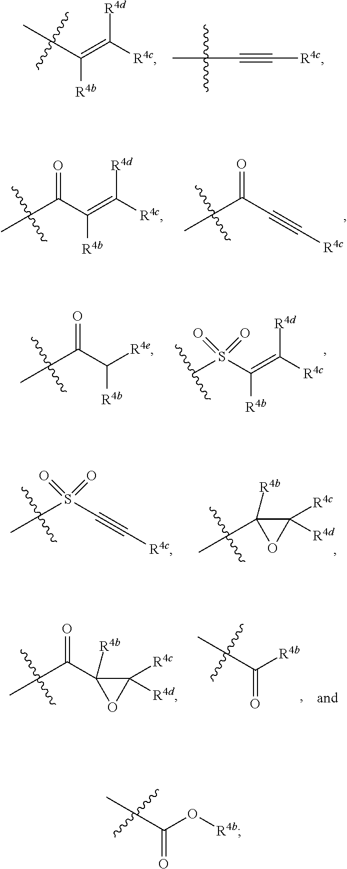

R^{4b}, R^{4c}, and R^{4d} are each independently hydrogen, halogen, —CN, —C(O)R¹³, —C(O)OR¹⁰, —C(O)N(R¹⁰)(R¹¹), —N(R¹⁰)(R¹¹), —C₁₋₆alkyl-N(R¹⁰)(R¹¹), —C(O)N(R¹⁰)OR¹⁰, C₁₋₆alkyl, phenyl, 3 to 7-membered heterocycloalkyl, or 5 or 6-membered heteroaryl, wherein the C₁₋₆alkyl, phenyl, 3 to 7-membered heterocycloalkyl, or 5 or 6-membered heteroaryl is optionally substituted with one, two, or three groups selected from R^{15d}; or
R^{4b} and R⁴ together with the atoms to which they are attached form a 3 to 14-membered cycloalkyl or 3 to 14-membered heterocycloalkyl; wherein the 3 to 14-membered cycloalkyl and 3 to 14-membered heterocycloalkyl are optionally substituted with 1, 2, 3, or 4 R^{15d}, or R^{4d} and R^{4c} together with the atoms to which they are attached form a 3 to 14-membered cycloalkyl or 3 to 14-membered heterocycloalkyl; wherein the 3 to 14-membered cycloalkyl and 3 to 14-membered heterocycloalkyl are optionally substituted with 1, 2, 3, or 4 R^{15d};
R^{4e} is halogen or —OS(O)₂R¹³;
R⁵, R⁷, and R⁸ are independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)(R¹³), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15e};
R^{9a} is selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15g};
each R¹⁰ is independently selected from hydrogen, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, —CH₂—C₆₋₁₀aryl, C₆₋₁₀aryl, C₁₋₉heteroaryl, and —CH₂—C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, —CH₂—C₆₋₁₀aryl, C₆₋₁₀aryl, C₁₋₉heteroaryl, and —CH₂—C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl; or R¹⁰ and R¹¹, together with the nitrogen to which they are attached, form a C₂₋₉heterocycloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and each R^{15a}, R^{15b}, R^{15c}, R^{15d}, R^{15e}, and R^{15g} are each independently selected from halogen, oxo, —CN, C₁₋₆alkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)(R¹³), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₉alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)(R¹³), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹) and —P(O)(R¹⁰)₂.

In some embodiments disclosed herein is a compound of Formula (Ia) having the structure of Formula (Ia′), or a pharmaceutically acceptable salt or stereoisomer thereof:

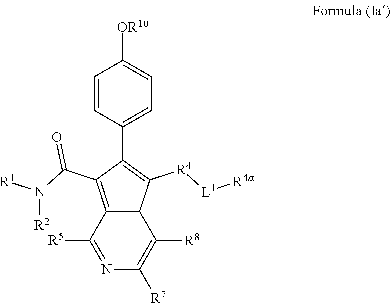

wherein
   R¹, R², R⁴, R^{4a}, R⁵, R⁷, R⁸, and L¹ have the same meaning as those in Formula (Ia), and
   R¹⁰ is C₁₋₉heteroaryl optionally substituted with one, two, or three groups selected from halogen, —CN, hydroxy, C₁₋₆alkyl, C₁₋₆haloalkyl, and C₁₋₆alkoxy.

In some embodiments disclosed herein is a compound of Formula (Ia) having the structure of Formula (Ia″), or a pharmaceutically acceptable salt or stereoisomer thereof:

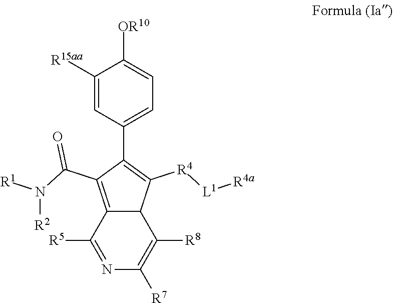

wherein
   R¹, R², R⁴, R^{4a}, R⁵, R⁷, R⁸, and L¹ have the same meaning as those in Formula (Ia), and
   R^{15aa} has the same meaning as R^{15a} in Formula (Ia).

In some embodiments disclosed herein is a compound of Formula (Ib), or a pharmaceutically acceptable salt or stereoisomer thereof:

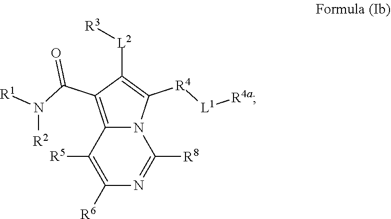

wherein:
   R¹ and R² are independently selected from hydrogen, C₁₋₆alkyl, and C₁₋₆haloalkyl;
   R³ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three groups selected from R^{15a};
   R⁴ is selected from C₃₋₆cycloalkylene, C₂₋₉heterocycloalkylene, C₆₋₁₀arylene, and C₁₋₉heteroarylene, wherein C₃₋₆cycloalkylene, C₂₋₉heterocycloalkylene, C₆₋₁₀arylene, and C₁₋₉heteroarylene are optionally substituted with one, two, or three groups selected from R^{15b};
   L¹ is a bond, —N(R^{9a})—, —N(R^{9a})C(O)—, —C(O)N(R^{9a})—, —N(R^{9a})S(O)₂—, —S(O)₂N(R^{9a})—, —O—, —C(O)—, —OC(O)—, —C(O)O—, —C(S)—, —S—, —S(O)—, —S(O)₂—, —OS(O)—, —OS(O)₂—, C₁₋₆alkylene, C₂₋₆alkenylene, C₂₋₆alkynylene, C₃₋₆cycloalkylene, C₂₋₉heterocycloalkylene, or C₁₋₉heteroarylene, wherein C₁₋₆alkylene, C₂₋₆alkenylene, C₂₋₆alkynylene, C₃₋₆cycloalkylene, C₂₋₉heterocycloalkylene, and C₁₋₉heteroarylene, are optionally substituted with one, two, or three groups selected from R^{15c};
   L² is a bond, C₁₋₆alkylene, C₂₋₆alkenylene, or C₂₋₆alkynylene, wherein C₁₋₆alkylene, C₂₋₆alkenylene, or C₂₋₆alkynylene are optionally substituted with one, two, or three groups selected from R^{15c};
   R^{4a} is selected from halogen, —CN,

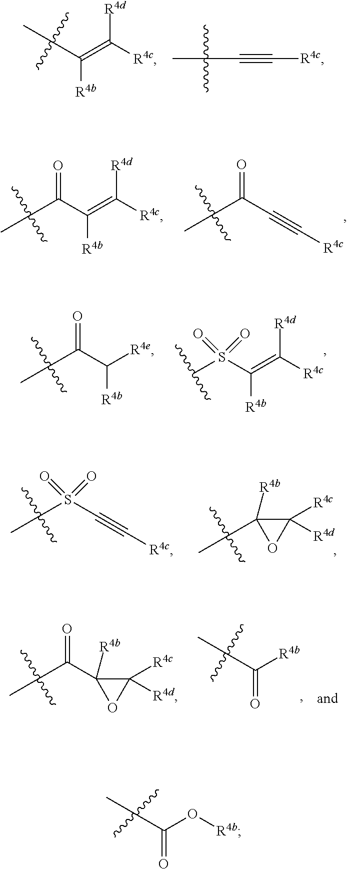

R^{4b}, R^{4c}, and R^{4d} are each independently hydrogen, halogen, —CN, —C(O)R¹³, —C(O)OR¹⁰, —C(O)N(R¹⁰)(R¹¹), —N(R¹⁰)(R¹¹), —C₁₋₆alkyl-N(R¹⁰)(R¹¹), —C(O)N(R¹⁰)OR¹⁰, C₁₋₆alkyl, phenyl, 3 to 7-membered heterocycloalkyl, or 5 or 6-membered heteroaryl, wherein the C₁₋₆alkyl, phenyl, 3 to 7-membered heterocycloalkyl, or 5 or 6-membered heteroaryl is optionally substituted with one, two, or three groups selected from R^{15d}; or
R^{4b} and R^{4c} together with the atoms to which they are attached form a 3 to 14-membered cycloalkyl or 3 to 14-membered heterocycloalkyl; wherein the 3 to 14-membered cycloalkyl and 3 to 14-membered heterocycloalkyl are optionally substituted with 1, 2, 3, or 4 R^{15d}, or R^{4d} and R^{4c} together with the atoms to which they are attached form a 3 to 14-membered cycloalkyl or 3 to 14-membered heterocycloalkyl; wherein the 3 to 14-membered cycloalkyl and 3 to 14-membered heterocycloalkyl are optionally substituted with 1, 2, 3, or 4 R^{15d};
R^{4e} is halogen or —OS(O)₂R¹³;
R⁵, R⁶, and R⁸ are independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)(R¹³), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15e};
R^{9a} is selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15g};
each R¹⁰ is independently selected from hydrogen, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, —CH₂—C₆₋₁₀aryl, C₆₋₁₀aryl, C₁₋₉heteroaryl, and —CH₂—C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, —CH₂—C₆₋₁₀aryl, C₆₋₁₀aryl, C₁₋₉heteroaryl, and —CH₂—C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl; or R¹⁰ and R¹¹, together with the nitrogen to which they are attached, form a C₂₋₉heterocycloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and
each R^{15a}, R^{15b}, R^{15c}, R^{15d}, R^{15e}, and R^{15g} are each independently selected from halogen, oxo, —CN, C₁₋₆alkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)(R¹³), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)(R¹³), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹) and —P(O)(R¹⁰)₂.

In some embodiments disclosed herein is a compound of Formula (Ib) having the structure of Formula (Ib′), or a pharmaceutically acceptable salt or stereoisomer thereof:

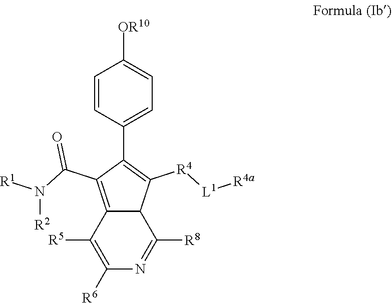

wherein
   R¹, R², R⁴, R^{4a}, R⁵, R⁶, R⁸, and L¹ have the same meaning as those in Formula (Ib), and
   R¹⁰ is C₁₋₉heteroaryl optionally substituted with one, two, or three groups selected from halogen, —CN, hydroxy, C₁₋₆alkyl, C₁₋₆haloalkyl, and C₁₋₆alkoxy.

In some embodiments disclosed herein is a compound of Formula (Ib) having the structure of Formula (Ib″), or a pharmaceutically acceptable salt or stereoisomer thereof:

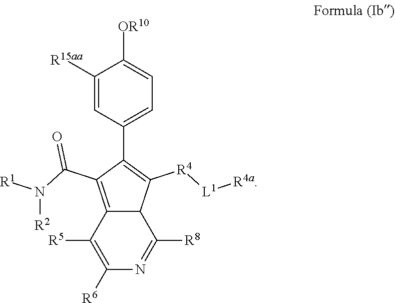

wherein
   R¹, R², R⁴, R^{4a}, R⁵, R⁶, R⁸, R¹⁰, and L¹ have the same meaning as those in Formula (Ib), and
   R^{15aa} has the same meaning as R^{15a} in Formula (Ib).

In some embodiments disclosed herein is a compound of Formula (Ic), or a pharmaceutically acceptable salt or stereoisomer thereof:

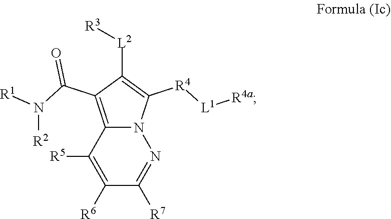

wherein:
   R¹ and R² are independently selected from hydrogen, C₁₋₆alkyl, and C₁₋₆haloalkyl;
   R³ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three groups selected from R^{15a};
   R⁴ is selected from C₃₋₆cycloalkylene, C₂₋₉heterocycloalkylene, C₆₋₁₀arylene, and C₁₋₉heteroarylene, wherein C₃₋₆cycloalkylene, C₂₋₉heterocycloalkylene, C₆₋₁₀arylene, and C₁₋₉heteroarylene are optionally substituted with one, two, or three groups selected from R^{15b};
   L¹ is a bond, —N(R^{9a})—, —N(R^{9a})C(O)—, —C(O)N(R^{9a})—, —N(R^{9a})S(O)₂—, —S(O)₂N(R^{9a})—, —O—, —C(O)—, —OC(O)—, —C(O)O—, —C(S)—, —S—, —S(O)—, —S(O)₂—, —OS(O)—, —OS(O)₂—, C₁₋₆alkylene, C₂₋₆alkenylene, C₂₋₆alkynylene, C₃₋₆cycloalkylene, C₂₋₉heterocycloalkylene, or C₁₋₉heteroarylene, wherein C₁₋₆alkylene, C₂₋₆alkenylene, C₂₋₆alkynylene, C₃₋₆cycloalkylene, C₂₋₉heterocycloalkylene, and C₁₋₉heteroarylene, are optionally substituted with one, two, or three groups selected from R^{15c};
   L² is a bond, C₁₋₆alkylene, C₂₋₆alkenylene, or C₂₋₆alkynylene, wherein C₁₋₆alkylene, C₂₋₆alkenylene, or C₂₋₆alkynylene are optionally substituted with one, two, or three groups selected from R^{15c};
   R^{4a} is selected from halogen, —CN,

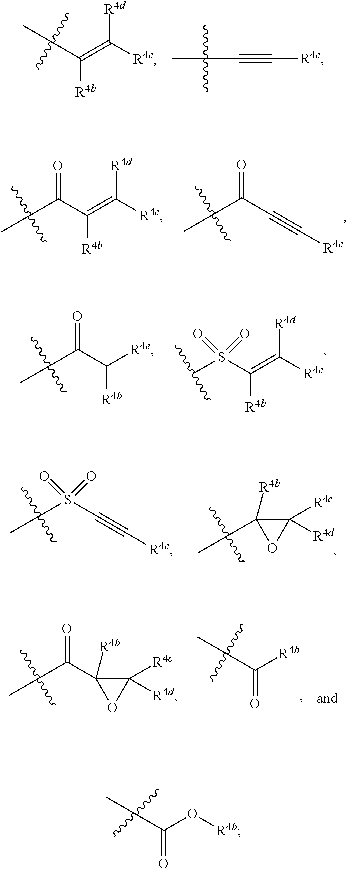

R^{4b}, R^{4e}, and R^{4d} are each independently hydrogen, halogen, —CN, —C(O)R¹³, —C(O)OR¹⁰, —C(O)N(R¹⁰)(R¹¹), —N(R¹⁰)(R¹¹), —C₁₋₆alkyl-N(R¹⁰)(R¹¹), —C(O)N(R¹⁰)OR¹⁰, C₁₋₆alkyl, phenyl, 3 to 7-membered heterocycloalkyl, or 5 or 6-membered heteroaryl, wherein the C₁₋₆alkyl, phenyl, 3 to 7-membered heterocycloalkyl, or 5 or 6-membered heteroaryl is optionally substituted with one, two, or three groups selected from R^{15d}; or
R^{4b} and R^{4c} together with the atoms to which they are attached form a 3 to 14-membered cycloalkyl or 3 to 14-membered heterocycloalkyl; wherein the 3 to 14-membered cycloalkyl and 3 to 14-membered heterocycloalkyl are optionally substituted with 1, 2, 3, or 4 R^{15d}, or R^{4d} and R^{4c} together with the atoms to which they are attached form a 3 to 14-membered cycloalkyl or 3 to 14-membered heterocycloalkyl; wherein the 3 to 14-membered cycloalkyl and 3 to 14-membered heterocycloalkyl are optionally substituted with 1, 2, 3, or 4 R^{15d};
R^{4e} is halogen or —OS(O)₂R¹³;
R⁵, R⁶, and R⁷ are independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)(R¹³), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15c};
R^{9a} is selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15g};
each R¹⁰ is independently selected from hydrogen, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, —CH₂—C₆₋₁₀aryl, C₆₋₁₀aryl, C₁₋₉heteroaryl, and —CH₂—C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, —CH₂—C₆₋₁₀aryl, C₆₋₁₀aryl, C₁₋₉heteroaryl, and —CH₂—C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl; or R¹⁰ and R¹¹, together with the nitrogen to which they are attached, form a C₂₋₉heterocycloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and
each R^{15a}, R^{15b}, R^{15e}, R^{15d}, R^{15e}, and R^{15g} are each independently selected from halogen, oxo, —CN, C₁₋₆alkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)(R¹³), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)(R¹³), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹) and —P(O)(R¹⁰)₂.

In some embodiments disclosed herein is a compound of Formula (Ic) having the structure of Formula (Ic′), or a pharmaceutically acceptable salt or stereoisomer thereof:

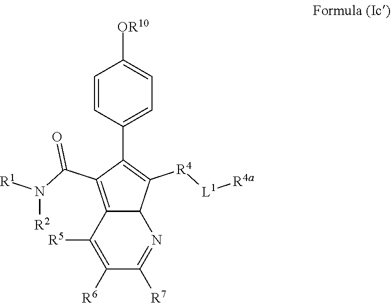

wherein
   R¹, R², R⁴, R^{4a}, R⁵, R⁶, R⁷, and L¹ have the same meaning as those in Formula (Ic), and
   R¹⁰ is C₁₋₉heteroaryl optionally substituted with one, two, or three groups selected from halogen, —CN, hydroxy, C₁₋₆alkyl, C₁₋₆haloalkyl, and C₁₋₆alkoxy.

In some embodiments disclosed herein is a compound of Formula (Ic) having the structure of Formula (Ic″), or a pharmaceutically acceptable salt or stereoisomer thereof:

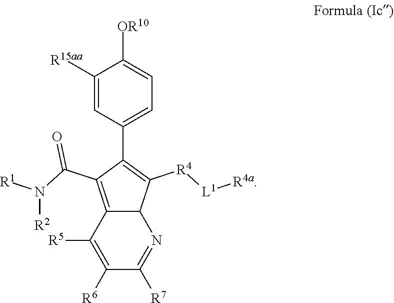

wherein
   R¹, R², R⁴, R^{4a}, R⁵, R⁷, R⁸, and L¹ have the same meaning as those in Formula (Ic), and
   R^{15aa} has the same meaning as R^{15a} in Formula (Ic).

In some embodiments of a compound of Formula (Ia″), (Ib″), or (Ic″), or a pharmaceutically acceptable salt or stereoisomer thereof, wherein R^{15aa} is selected from halogen, —CN, C₁₋₆alkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)(R¹³), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)(R¹³), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹) and —P(O)(R¹⁰)₂. In some embodiments of a compound of Formula (Ia″), (Ib″), or (Ic″), or a pharmaceutically acceptable salt or stereoisomer thereof, wherein R^{15aa} is selected from halogen, —CN, C₁₋₆alkyl, —OR¹⁰, —N(R¹⁰)(R¹¹), —C(O)OR¹⁰, —N(R¹²)S(O)₂R¹³, —C(O)R¹³, —S(O)R¹³, —C(O)N(R¹⁰)(R¹¹), —C(O)C(O)N(R¹⁰)(R¹¹), —N(R¹²)C(O)R¹³, —S(O)₂R¹³, and —S(O)₂N(R¹⁰)(R¹¹)—, wherein C₁₋₆alkyl is optionally substituted with one, two, or three groups independently selected from halogen, —CN, C₁₋₆alkyl, C₁₋₆haloalkyl, C₁₋₆alkoxy, C₁₋₆haloalkoxy, —OR¹⁰, and —N(R¹⁰)(R¹¹). In some embodiments, disclosed herein is a compound of Formula (Ia″), (Ib″), or (Ic″), or a pharmaceutically acceptable salt or stereoisomer thereof, wherein R^{15aa} is selected from halogen, C₁₋₆alkyl, and —OCH₃. In some embodiments, disclosed herein is a compound of Formula (Ia″), (Ib″), or (Ic″), or a pharmaceutically acceptable salt or stereoisomer thereof, wherein R^{15aa} is halogen. In some embodiments, disclosed herein is a compound of Formula (Ia″), (Ib″), or (Ic″), or a pharmaceutically acceptable salt or stereoisomer thereof, wherein R^{15aa} is methyl, or fluorine. In some embodiments, disclosed herein is a compound of Formula (Ia″), (Ib″), or (Ic″), or a pharmaceutically acceptable salt or stereoisomer thereof, wherein R^{15aa} is fluorine.

In some embodiments, disclosed herein is a compound of Formula (Ia′), (Ia″), (Ib′), (Ib″), (Ic′), or (Ic″), or a pharmaceutically acceptable salt or stereoisomer thereof, wherein R¹⁰ is C₁₋₉heteroaryl optionally substituted with one, two, or three groups selected from halogen, —CN, hydroxy, C₁₋₆alkyl, C₁₋₆haloalkyl, and C₁₋₆alkoxy. In some embodiments, disclosed herein is a compound of Formula (Ia′), (Ia″), (Tb′), (Ib″), (Ic′), or (Ic″), or a pharmaceutically acceptable salt or stereoisomer thereof, wherein R¹⁰ is 5 to 6 membered heteroaryl optionally substituted with one, two, or three groups selected from halogen, —CN, hydroxy, C₁₋₆alkyl, C₁₋₆haloalkyl, and C₁₋₆alkoxy. In some embodiments, disclosed herein is a compound of Formula (Ia′), (Ia″), (Ib′), (Ib″), (Ic′), or (Ic″), or a pharmaceutically acceptable salt or stereoisomer thereof, wherein R¹⁰ is 5- to 6-membered heteroaryl optionally substituted with one group selected from C₁₋₆alkyl. In some embodiments, disclosed herein is a compound of Formula (Ia′), (Ia″), (Tb′), (Ib″), (Ic′), or (Ic″), or a pharmaceutically acceptable salt or stereoisomer thereof, wherein R¹⁰ is pyridyl or pyrimidinyl, wherein the pyridyl and pyrimidinyl are optionally substituted with one, two, or three groups selected from halogen, —CN, hydroxy, C₁₋₆alkyl, C₁₋₆haloalkyl, and C₁₋₆alkoxy. In some embodiments, disclosed herein is a compound of Formula (la′), (Ia″), (Ib′), (Ib″), (Ic′), or (Ic″), or a pharmaceutically acceptable salt or stereoisomer thereof, wherein R¹⁰ is pyridyl or pyrimidinyl, wherein the pyridyl and pyrimidinyl are substituted with one, two, or three groups selected from C₁₋₆alkyl. In some embodiments, disclosed herein is a compound of Formula (Ia′), (Ia″), (Ib′), (Ib″), (Ic′), or (Ic″), or a pharmaceutically acceptable salt or stereoisomer thereof, wherein R¹⁰ is optionally substituted with one or more substituents selected from halogen, —CN, —OH, —SF₅, —SH, —S(═O) C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —S(═O)(═NC₁-C₃alkyl) (C₁-C₃alkyl), —NH₂, —NHC₁-C₃alkyl, —N(C₁-C₃alkyl)₂, —N═S(═O) (C₁-C₃alkyl)₂, —O—C₁-C₄alkylene-OH, —O—C₁-C₄alkylene-NH₂, —C(═O) C₁-C₃alkyl, —C(═O)OH, —C(═O)OC₁-C₃alkyl, —C(═O) NH₂, —C(═O)NHC₁-C₃alkyl, —C(═O)N(C₁-C₃alkyl)₂, —P(═O) (C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, C₃-C₆cycloalkyl, and 3- to 6-membered heterocycloalkyl. In some embodiments, disclosed herein is a compound of Formula (Ia′), (Ia″), (Ib′), (Ib″), (Ic′), or (Ic″), or a pharmaceutically acceptable salt or stereoisomer thereof, wherein R¹⁰ is optionally substituted with one or more substituents selected from halogen, —CN, —OH, —SF₅, —SH, —S(═O)₂NH₂, —S(═O)₂NHC₁-C₃alkyl, —S(═O)₂N(C₁-C₃alkyl)₂, —S(═O)(═NC₁-C₃alkyl) (C₁-C₃alkyl), —NH₂, —NHC₁-C₃alkyl, —N(C₁-C₃alkyl)₂, —O—C₁-C₄alkylene-OH, —O—C₁-C₄alkylene-NH₂, —C(═O) C₁-C₃alkyl, —C(═O)OH, —C(═O)OC₁-C₃alkyl, —C(═O) NH₂, —C(═O)NHC₁-C₃alkyl, C₁-C₃alkyl, C₁-C₃alkoxy, C₁-C₃haloalkyl, C₁-C₃haloalkoxy, C₁-C₃hydroxyalkyl, C₁-C₆aminoalkyl, C₁-C₃heteroalkyl, C₃-C₆cycloalkyl, and 3- to 6-membered heterocycloalkyl. In some embodiments, disclosed herein is a compound of Formula (Ia′), (Ia″), (Ib′), (Ib″), (Ic′), or (Ic″), or a pharmaceutically acceptable salt or stereoisomer thereof, wherein R¹⁰ is

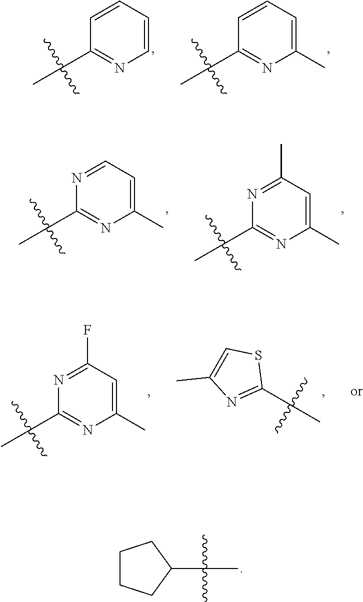

In some embodiments, disclosed herein is a compound of Formula (Ia′), (Ia″), (Ib′), (Ib″), (Ic′), or (Ic″), or a pharmaceutically acceptable salt or stereoisomer thereof, wherein R¹⁰ is

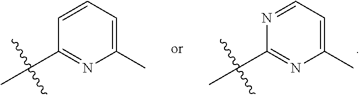

In some embodiments, disclosed herein is a compound of Formula (Ia′), (Ia″), (Ib′), (Ib″), (Ic′), or (Ic″), or a pharmaceutically acceptable salt or stereoisomer thereof, wherein R¹⁰ is

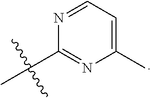 [SMILES: *c1nccc(C)n1]

In some embodiments disclosed herein is a compound of Formula (Id), or a pharmaceutically acceptable salt or stereoisomer thereof:

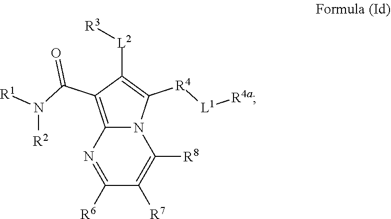

wherein:
   R¹ and R² are independently selected from hydrogen, C₁₋₆alkyl, and C₁₋₉haloalkyl;
   R³ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three groups selected from R^{15a};
   R⁴ is selected from C₃₋₆cycloalkylene, C₂₋₉heterocycloalkylene, C₆₋₁₀arylene, and C₁₋₉heteroarylene, wherein C₃₋₆cycloalkylene, C₂₋₉heterocycloalkylene, C₆₋₁₀arylene, and C₁₋₉heteroarylene are optionally substituted with one, two, or three groups selected from R^{15b};
   L¹ is a bond, —N(R^{9a})—, —N(R^{9a})C(O)—, —C(O)N(R^{9a})—, —N(R^{9a})S(O)₂—, —S(O)₂N(R^{9a})—, —O—, —C(O)—, —OC(O)—, —C(O)O—, —C(S)—, —S—, —S(O)—, —S(O)₂—, —OS(O)—, —OS(O)₂—, C₁₋₆alkylene, C₂₋₆alkenylene, C₂₋₆alkynylene, C₃₋₆cycloalkylene, C₂₋₉heterocycloalkylene, or C₁₋₉heteroarylene, wherein C₁₋₆alkylene, C₂₋₆alkenylene, C₂₋₆alkynylene, C₃₋₆cycloalkylene, C₂₋₉heterocycloalkylene, and C₁₋₉heteroarylene, are optionally substituted with one, two, or three groups selected from R^{15c};
   L² is a bond, C₁₋₆alkylene, C₂₋₆alkenylene, or C₂₋₆alkynylene, wherein C₁₋₆alkylene, C₂₋₆alkenylene, or C₂₋₆alkynylene are optionally substituted with one, two, or three groups selected from R^{15c};
   R^{4a} is selected from halogen, —CN,

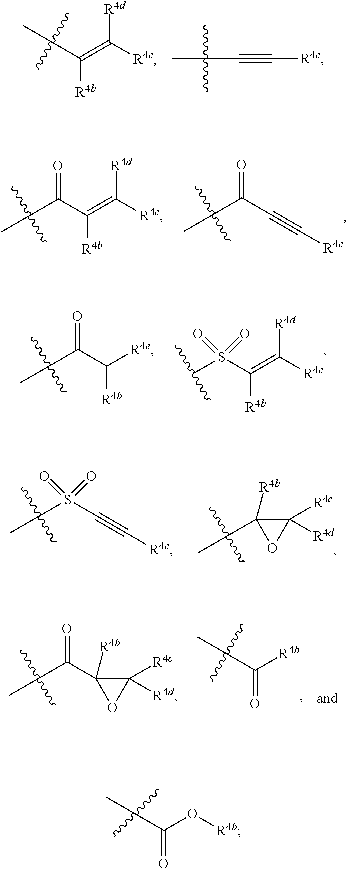

R^{4b}, R^{4c}, and R^{4d} are each independently hydrogen, halogen, —CN, —C(O)R¹³, —C(O)OR¹⁰, —C(O)N(R¹⁰)(R¹¹), —N(R¹⁰)(R¹¹), —C₁₋₆alkyl-N(R¹⁰)(R¹¹), —C(O)N(R¹⁰)OR¹⁰, C₁₋₆alkyl, phenyl, 3 to 7-membered heterocycloalkyl, or 5 or 6-membered heteroaryl, wherein the C₁₋₆alkyl, phenyl, 3 to 7-membered heterocycloalkyl, or 5 or 6-membered heteroaryl is optionally substituted with one, two, or three groups selected from R^{15d}; or
R^{4b} and R^{4c} together with the atoms to which they are attached form a 3 to 14-membered cycloalkyl or 3 to 14-membered heterocycloalkyl; wherein the 3 to 14-membered cycloalkyl and 3 to 14-membered heterocycloalkyl are optionally substituted with 1, 2, 3, or 4 R^{15d}, or R^{4d} and R^{4c} together with the atoms to which they are attached form a 3 to 14-membered cycloalkyl or 3 to 14-membered heterocycloalkyl; wherein the 3 to 14-membered cycloalkyl and 3 to 14-membered heterocycloalkyl are optionally substituted with 1, 2, 3, or 4 R^{15d};
R^{4e} is halogen or —OS(O)₂R¹³;
R⁶, R⁷, and R⁸ are independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)(R¹³), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15e};
R^{9a} is selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15g};
each R¹⁰ is independently selected from hydrogen, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, —CH₂—C₆₋₁₀aryl, C₆₋₁₀aryl, C₁₋₉heteroaryl, and —CH₂—C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, —CH₂—C₆₋₁₀aryl, C₆₋₁₀aryl, C₁₋₉heteroaryl, and —CH₂—C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl; or R¹⁰ and R¹¹, together with the nitrogen to which they are attached, form a C₂₋₉heterocycloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and
each R^{15a}, R^{15b}, R^{15c}, R^{15d}, R^{15c}, and R^{15g} are each independently selected from halogen, oxo, —CN, C₁-(alkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)(R¹³), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)(R¹³), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹) and —P(O)(R¹⁰)₂.

In some embodiments disclosed herein is a compound of Formula (Ie), or a pharmaceutically acceptable salt or stereoisomer thereof:

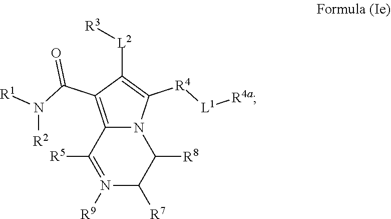

wherein:
   R¹ and R² are independently selected from hydrogen, C₁₋₆alkyl, and C₁₋₆haloalkyl;
   R³ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three groups selected from R^{15a};
   R⁴ is selected from C₃₋₆cycloalkylene, C₂₋₉heterocycloalkylene, C₆₋₁₀arylene, and C₁₋₉heteroarylene, wherein C₃₋₆cycloalkylene, C₂₋₉heterocycloalkylene, C₆₋₁₀arylene, and C₁₋₉heteroarylene are optionally substituted with one, two, or three groups selected from R^{15b};
   L¹ is a bond, —N(R^{9a})—, —N(R^{9a})C(O)—, —C(O)N(R^{9a})—, —N(R^{9a})S(O)₂—, —S(O)₂N(R^{9a})—, —O—, —C(O)—, —OC(O)—, —C(O)O—, —C(S)—, —S—, —S(O)—, —S(O)₂—, —OS(O)—, —OS(O)₂—, C₁₋₆alkylene, C₂₋₆alkenylene, C₂₋₆alkynylene, C₃₋₆cycloalkylene, C₂₋₉heterocycloalkylene, or C₁₋₉heteroarylene, wherein C₁₋₆alkylene, C₂₋₆alkenylene, C₂₋₆alkynylene, C₃₋₆cycloalkylene, C₂₋₉heterocycloalkylene, and C₁₋₉heteroarylene, are optionally substituted with one, two, or three groups selected from R^{15c};
   L² is a bond, C₁₋₆alkylene, C₂₋₆alkenylene, or C₂₋₆alkynylene, wherein C₁₋₆alkylene, C₂₋₆alkenylene, or C₂₋₆alkynylene are optionally substituted with one, two, or three groups selected from R^{15c};
   R^{4a} is selected from halogen, —CN,

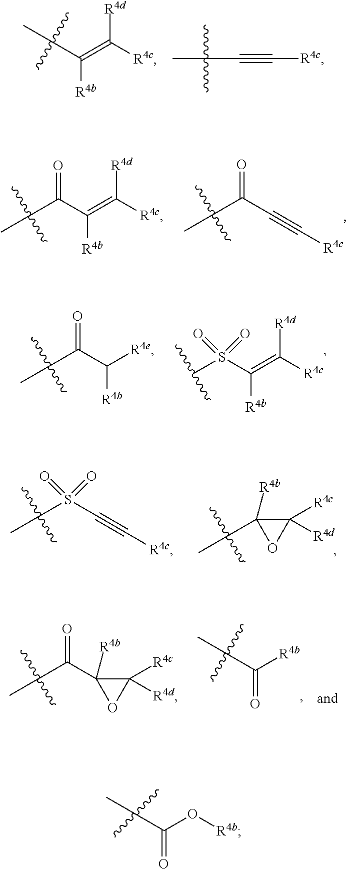

R^{4b}, R^{4c}, and R^{4d} are each independently hydrogen, halogen, —CN, —C(O)R¹³, —C(O)OR¹⁰, —C(O)N(R¹⁰)(R¹¹), —N(R¹⁰)(R¹¹), —C₁₋₆alkyl-N(R¹⁰)(R¹¹), —C(O)N(R¹⁰)OR¹⁰, C₁₋₆alkyl, phenyl, 3 to 7-membered heterocycloalkyl, or 5 or 6-membered heteroaryl, wherein the C₁₋₆alkyl, phenyl, 3 to 7-membered heterocycloalkyl, or 5 or 6-membered heteroaryl is optionally substituted with one, two, or three groups selected from R^{15d}; or
R^{4b} and R^{4c} together with the atoms to which they are attached form a 3 to 14-membered cycloalkyl or 3 to 14-membered heterocycloalkyl; wherein the 3 to 14-membered cycloalkyl and 3 to 14-membered heterocycloalkyl are optionally substituted with 1, 2, 3, or 4 R^{15d}, or R^{4d} and R^{4c} together with the atoms to which they are attached form a 3 to 14-membered cycloalkyl or 3 to 14-membered heterocycloalkyl; wherein the 3 to 14-membered cycloalkyl and 3 to 14-membered heterocycloalkyl are optionally substituted with 1, 2, 3, or 4 R^{15d};
R^{4e} is halogen or —OS(O)₂R¹³;
R⁵, R⁷, and R⁸ are independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)(R¹³), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15c};
R⁹ is selected from hydrogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹⁰, —C(O)R¹³, —S(O)R¹³, —C(O)N(R¹⁰)(R¹¹), and —S(O)₂R¹³, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15f};
R^{9a} is selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15g};
each R¹⁰ is independently selected from hydrogen, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, —CH₂—C₆₋₁₀aryl, C₆₋₁₀aryl, C₁₋₉heteroaryl, and —CH₂—C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, —CH₂—C₆₋₁₀aryl, C₆₋₁₀aryl, C₁₋₉heteroaryl, and —CH₂—C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl; or R¹⁰ and R¹¹, together with the nitrogen to which they are attached, form a C₂₋₉heterocycloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and
each R^{15a}, R^{15b}, R^{15e}, R^{15d}, R^{15e}, R^{15f}, and R^{15g} are each independently selected from halogen, oxo, —CN, C₁₋₆alkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)(R¹³), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)(R¹³), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹) and —P(O)(R¹⁰)₂.

In some embodiments of a compound of Formula (Ie), or a pharmaceutically acceptable salt or stereoisomer thereof, R⁹ is selected from hydrogen and C₁₋₆alkyl optionally substituted with one, two, or three groups selected from R^{15f}; In some embodiments of a compound of Formula (Ie), or a pharmaceutically acceptable salt or stereoisomer thereof, R⁹ is selected from hydrogen and unsubstituted C₁₋₆alkyl. In some embodiments of a compound of Formula (Ie), or a pharmaceutically acceptable salt or stereoisomer thereof, R⁹ is hydrogen. In some embodiments of a compound of Formula (Ie), or a pharmaceutically acceptable salt or stereoisomer thereof, R⁹ is C₁₋₆alkyl optionally substituted with one, two, or three groups selected from R^{15f}; In some embodiments of a compound of Formula (Ie), or a pharmaceutically acceptable salt or stereoisomer thereof, R⁹ is unsubstituted C₁₋₆alkyl.

In some embodiments of a compound of Formula (I), (Ia), (Ia′), (Ia″), (Ib), (Ib′), (Ib″), (Ic), (Ic′), (Ic″), or (Ie), or a pharmaceutically acceptable salt or stereoisomer thereof, R⁵ is selected from hydrogen, halogen, —OR¹⁰, and C₁₋₆alkyl, wherein C₁₋₆alkyl is optionally substituted with one, two, or three groups selected from R^{15e}. In some embodiments of a compound of Formula (I), (Ia), (Ia′), (Ia″), (Ib), (Ib′), (Ib″), (Ic), (Ic′), (Ic″), or (Ie), or a pharmaceutically acceptable salt or stereoisomer thereof, R⁵ is selected from hydrogen and C₁₋₆alkyl optionally substituted with one, two, or three groups selected from R^{15e}. In some embodiments of a compound of Formula (I), (Ia), (Ia′), (Ia″), (Ib), (Ib′), (Ib″), (Ic), (Ic′), (Ic″), or (Ie), or a pharmaceutically acceptable salt or stereoisomer thereof, R⁵ is selected from hydrogen and unsubstituted C₁₋₆alkyl. In some embodiments of a compound of Formula (I), (Ia), (Ia′), (Ia″), (Ib), (Ib′), (Ib″), (Ic), (Ic′), (Ic″), or (Ie), or a pharmaceutically acceptable salt or stereoisomer thereof, R⁵ is hydrogen. In some embodiments of a compound of Formula (I), (Ia), (Ia′), (Ia″), (Ib), (Ib′), (Ib″), (Ic), (Ic′), (Ic″), or (Ie), or a pharmaceutically acceptable salt or stereoisomer thereof, R⁵ is C₁₋₆alkyl optionally substituted with one, two, or three groups selected from R^{15e}. In some embodiments of a compound of Formula (I), (Ia), (Ia′), (Ia″), (Ib), (Ib′), (Ib″), (Ic), (Ic′), (Ic″), or (Ie), or a pharmaceutically acceptable salt or stereoisomer thereof, R⁵ is unsubstituted C₁₋₆alkyl. In some embodiments of a compound of Formula (I), (Ia), (Ia′), (Ia″), (Ib), (Ib′), (Ib″), (Ic), (Ic′), (Ic″), or (Ie), or a pharmaceutically acceptable salt or stereoisomer thereof, R⁵ is halogen. In some embodiments of a compound of Formula (I), (Ia), (Ia′), (Ia″), (Ib), (Ib′), (Ib″), (Ic), (Ic′), (Ic″), or (Ie), or a pharmaceutically acceptable salt or stereoisomer thereof, R⁵ is —OR¹⁰. In some embodiments of a compound of Formula (I), (Ia), (Ia′), (Ia″), (Ib), (Ib′), (Ib″), (Ic), (Ic′), (Ic″), or (Ie), or a pharmaceutically acceptable salt or stereoisomer thereof, R⁵ is —OR¹⁰ and R¹⁰ is selected from hydrogen and C₁₋₆alkyl.

In some embodiments of a compound of Formula (I), (Ib), (Ib′), (Ib″), (Ic), (Ic′), (Ic″), or (Id), or a pharmaceutically acceptable salt or stereoisomer thereof, R⁶ is selected from hydrogen, halogen, —OR¹⁰, and C₁₋₆alkyl, wherein C₁₋₆alkyl is optionally substituted with one, two, or three groups selected from R^{15c}. In some embodiments of a compound of Formula (I), (Ib), (Ib′), (Ib″), (Ic), (Ic′), (Ic″), or (Id), or a pharmaceutically acceptable salt or stereoisomer thereof, R⁶ is selected from hydrogen and C₁₋₆alkyl optionally substituted with one, two, or three groups selected from R^{15c}. In some embodiments of a compound of Formula (I), (Ib), (Ib′), (Ib″), (Ic), (Ic′), (Ic″), or (Id), or a pharmaceutically acceptable salt or stereoisomer thereof, R⁶ is selected from hydrogen and unsubstituted C₁₋₆alkyl. In some embodiments of a compound of Formula (I), (Ib), (Ib′), (Ib″), (Ic), (Ic′), (Ic″), or (Id), or a pharmaceutically acceptable salt or stereoisomer thereof, R⁶ is hydrogen. In some embodiments of a compound of Formula (I), (Ib), (Ib′), (Ib″), (Ic), (Ic′), (Ic″), or (Id), or a pharmaceutically acceptable salt or stereoisomer thereof, R⁶ is C₁₋₆alkyl optionally substituted with one, two, or three groups selected from R^{15e}. In some embodiments of a compound of Formula (I), (Ib), (Ib′), (Ib″), (Ic), (Ic′), (Ic″), or (Id), or a pharmaceutically acceptable salt or stereoisomer thereof, R⁶ is unsubstituted C₁₋₆alkyl. In some embodiments of a compound of Formula (I), (Ib), (Ib′), (Ib″), (Ic), (Ic′), (Ic″), or (Id), or a pharmaceutically acceptable salt or stereoisomer thereof, R⁶ is unsubstituted C₁₋₃alkyl. In some embodiments of a compound of Formula (I), (Ib), (Ib′), (Ib″), (Ic), (Ic′), (Ic″), or (Id), or a pharmaceutically acceptable salt or stereoisomer thereof, R⁶ is methyl. In some embodiments of a compound of Formula (I), or a pharmaceutically acceptable salt or stereoisomer thereof, R⁶ is halogen. In some embodiments of a compound of Formula (I), (Ib), (Ib′), (Ib″), (Ic), (Ic′), (Ic″), or (Id), or a pharmaceutically acceptable salt or stereoisomer thereof, R⁶ is —OR¹⁰. In some embodiments of a compound of Formula (I), (Ib), (Ib′), (Ib″), (Ic), (Ic′), (Ic″), or (Id), or a pharmaceutically acceptable salt or stereoisomer thereof, R⁶ is —OR¹⁰ and R¹⁰ is selected from hydrogen and C₁₋₆alkyl.

In some embodiments of a compound of Formula (I), (Ia), (Ia′), (Ia″), (Ic), (Ic′), (Ic″), (Id), or (Ie), or a pharmaceutically acceptable salt or stereoisomer thereof, R⁷ is selected from hydrogen, halogen, —OR¹⁰, and C₁₋₆alkyl, wherein C₁₋₆alkyl is optionally substituted with one, two, or three groups selected from R^{15e}. In some embodiments of a compound of Formula (I), (Ia), (Ia′), (Ia″), (Ic), (Ic′), (Ic″), (Id), or (Ie), or a pharmaceutically acceptable salt or stereoisomer thereof, R⁷ is selected from hydrogen and C₁₋₆alkyl optionally substituted with one, two, or three groups selected from R^{15e}. In some embodiments of a compound of Formula (I), (Ia), (Ia′), (Ia″), (Ic), (Ic′), (Ic″), (Id), or (Ie), or a pharmaceutically acceptable salt or stereoisomer thereof, R⁷ is selected from hydrogen and unsubstituted C₁₋₆alkyl. In some embodiments of a compound of Formula (I), (Ia), (Ia′), (Ia″), (Ic), (Ic′), (Ic″), (Id), or (Ie), or a pharmaceutically acceptable salt or stereoisomer thereof, R⁷ is hydrogen. In some embodiments of a compound of Formula (I), (Ia), (Ia′), (Ia″), (Ic), (Ic′), (Ic″), (Id), or (Ie), or a pharmaceutically acceptable salt or stereoisomer thereof, R⁷ is C₁₋₆alkyl optionally substituted with one, two, or three groups selected from R^{15e}. In some embodiments of a compound of Formula (I), (Ia), (Ia′), (Ia″), (Ic), (Ic′), (Ic″), (Id), or (Ie), or a pharmaceutically acceptable salt or stereoisomer thereof, R⁷ is unsubstituted C₁₋₆alkyl. In some embodiments of a compound of Formula (I), (Ia), (Ia′), (Ia″), (Ic), (Ic′), (Ic″), (Id), or (Ie), or a pharmaceutically acceptable salt or stereoisomer thereof, R⁷ is halogen. In some embodiments of a compound of Formula (I), (Ia), (Ia′), (Ia″), (Ic), (Ic′), (Ic″), (Id), or (Ie), or a pharmaceutically acceptable salt or stereoisomer thereof, R⁷ is —OR¹⁰. In some embodiments of a compound of Formula (I), (Ia), (Ia′), (Ia″), (Ic), (Ic′), (Ic″), (Id), or (Ie), or a pharmaceutically acceptable salt or stereoisomer thereof, R⁷ is —OR¹⁰ and R¹⁰ is selected from hydrogen and C₁₋₆alkyl.

In some embodiments of a compound of Formula (I), (Ia), (Ia′), (Ia″), (Ib), (Ib′), (Ib″), (Id), or (Ie), or a pharmaceutically acceptable salt or stereoisomer thereof, R⁸ is selected from hydrogen, halogen, —OR¹⁰, and C₁₋₆alkyl, wherein C₁₋₆alkyl is optionally substituted with one, two, or three groups selected from R^{15c}. In some embodiments of a compound of Formula (I), (Ia), (Ia′), (Ia″), (Ib), (Ib′), (Ib″), (Id), or (Ie), or a pharmaceutically acceptable salt or stereoisomer thereof, R⁸ is selected from hydrogen and C₁₋₆alkyl optionally substituted with one, two, or three groups selected from R^{15c}. In some embodiments of a compound of Formula (I), (Ia), (Ia′), (Ia″), (Ib), (Ib′), (Ib″), (Id), or (Ie), or a pharmaceutically acceptable salt or stereoisomer thereof, R⁸ is selected from hydrogen and unsubstituted C₁₋₆alkyl. In some embodiments of a compound of Formula (I), (Ia), (Ia′), (Ia″), (Ib), (Ib′), (Ib″), (Id), or (Ie), or a pharmaceutically acceptable salt or stereoisomer thereof, R⁸ is hydrogen. In some embodiments of a compound of Formula (I), (Ia), (Ia′), (Ia″), (Ib), (Ib′), (Ib″), (Id), or (Ie), or a pharmaceutically acceptable salt or stereoisomer thereof, R⁸ is C₁₋₆alkyl optionally substituted with one, two, or three groups selected from R^{15e}. In some embodiments of a compound of Formula (I), (Ia), (Ia′), (Ia″), (Ib), (Ib′), (Ib″), (Id), or (Ie), or a pharmaceutically acceptable salt or stereoisomer thereof, R⁸ is unsubstituted C₁₋₆alkyl. In some embodiments of a compound of Formula (I), (Ia), (Ia′), (Ia″), (Ib), (Ib′), (Ib″), (Id), or (Ie), or a pharmaceutically acceptable salt or stereoisomer thereof, R⁸ is halogen. In some embodiments of a compound of Formula (I), (Ia), (Ia′), (Ia″), (Ib), (Ib′), (Ib″), (Id), or (Ie), or a pharmaceutically acceptable salt or stereoisomer thereof, R⁸ is —OR¹⁰. In some embodiments of a compound of Formula (I), (Ia), (Ia′), (Ia″), (Ib), (Ib′), (Ib″), (Id), or (Ie), or a pharmaceutically acceptable salt or stereoisomer thereof, R⁸ is —OR¹⁰ and R¹⁰ is selected from hydrogen and C₁₋₆alkyl.

In some embodiments of a compound of Formula (I), (Ia), (Ib), (Ic), (Id), or (Ie), or a pharmaceutically acceptable salt or stereoisomer thereof, L² is a bond or C₁₋₆alkylene optionally substituted with one, two, or three groups selected from R^{15c}. In some embodiments of a compound of Formula (I), (Ia), (Ib), (Ic), (Id), or (Ie), or a pharmaceutically acceptable salt or stereoisomer thereof, L² is a bond. In some embodiments of a compound of Formula (I), (Ia), (Ib), (Ic), (Id), or (Ie), or a pharmaceutically acceptable salt or stereoisomer thereof, L² is C₁₋₆alkylene optionally substituted with one, two, or three groups selected from R^{15c}. In some embodiments of a compound of Formula (I), (Ia), (Tb), (Ic), (Id), or (Ie), or a pharmaceutically acceptable salt or stereoisomer thereof, L² is unsubstituted C₁₋₆alkylene.

In some embodiments of a compound of Formula (I), (Ia), (Ib), (Ic), (Id), or (Ie), or a pharmaceutically acceptable salt or stereoisomer thereof, R³ is selected from C₆₋₁₀aryl and C₁₋₉heteroaryl, wherein C₆₋₁₀aryl and C₁₋₉heteroaryl are optionally substituted with one, two, or three groups selected from R^{15a}. In some embodiments of a compound of Formula (I), (Ia), (Ib), (Ic), (Id), or (Ie), or a pharmaceutically acceptable salt or stereoisomer thereof, R³ is C₆₋₁₀aryl optionally substituted with one, two, or three groups selected from R^{15a}. In some embodiments of a compound of Formula (I), (Ia), (Ib), (Ic), (Id), or (Ie), or a pharmaceutically acceptable salt or stereoisomer thereof, R³ is phenyl optionally substituted with one, two, or three groups selected from R^{15a}. In some embodiments of a compound of Formula (I), (Ia), (Ib), (Ic), (Id), or (Ie), or a pharmaceutically acceptable salt or stereoisomer thereof, R³ is phenyl substituted with one, two, or three groups selected from R^{15a}. In some embodiments of a compound of Formula (I), (Ia), (Ib), (Ic), (Id), or (Ie), or a pharmaceutically acceptable salt or stereoisomer thereof, R³ is C₁₋₉heteroaryl optionally substituted with one, two, or three groups selected from R^{15a}. In some embodiments of a compound of Formula (I), (Ia), (Ib), (Ic), (Id), or (Ie), or a pharmaceutically acceptable salt or stereoisomer thereof, R³ is C₁₋₉heteroaryl substituted with one, two, or three groups selected from R^{15a}.

In some embodiments of a compound of Formula (I), (Ia), (Ib), (Ic), (Id), or (Ie), or a pharmaceutically acceptable salt or stereoisomer thereof, R³ is substituted with R^{15a} and R^{15a} is selected from —OCH₃,

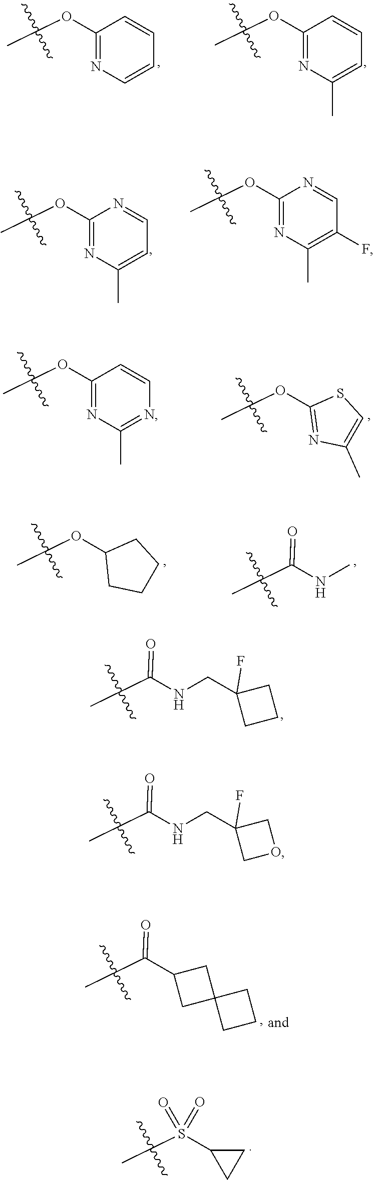

In some embodiments of a compound of Formula (I), (Ia), (Ib), (Ic), (Id), or (Ie), or a pharmaceutically acceptable salt or stereoisomer thereof, R³ is substituted with R^{15a} and R^{15a} is

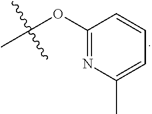 [SMILES: *Oc1cccc(C)n1]

In some embodiments of a compound of Formula (I), (Ia), (Ib), (Ic), (Id), or (Ie), or a pharmaceutically acceptable salt or stereoisomer thereof, R³ is substituted with R^{15a} and R^{15a} is

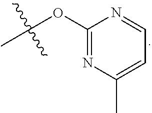 [SMILES: *Oc1nccc(C)n1]

In some embodiments of a compound of Formula (I), (Ia), (Ib), (Ic), (Id), or (Ie), or a pharmaceutically acceptable salt or stereoisomer thereof, R³ is substituted with R^{15a} and R^{15a} is

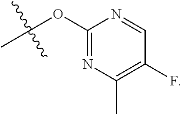 [SMILES: *Oc1ncc(F)c(C)n1]

In some embodiments of a compound of Formula (I), (Ia), (Ib), (Ic), (Id), or (Ie), or a pharmaceutically acceptable salt or stereoisomer thereof, R³ is substituted with R^{15a} and R^{15a} is

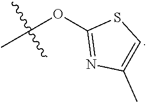 [SMILES: *Oc1nc(C)cs1]

In some embodiments of a compound of Formula (I), (Ia), (Ib), (Ic), (Id), or (Ie), or a pharmaceutically acceptable salt or stereoisomer thereof, R³ is substituted with R^{15a} and R^{15a} is

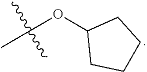 [SMILES: *OC1CCCC1]

In some embodiments of a compound of Formula (I), (Ia), (Ib), (Ic), (Id), or (Ie), or a pharmaceutically acceptable salt or stereoisomer thereof, R³ is substituted with R^{15a} and R^{15a} is

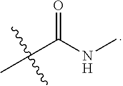 [SMILES: *C(=O)NC]

In some embodiments of a compound of Formula (I), (Ia), (Ib), (Ic), (Id), or (Ie), or a pharmaceutically acceptable salt or stereoisomer thereof, R³ is substituted with R^{15a} and R^{15a} is

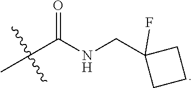 [SMILES: *C(=O)NCC1(F)CCC1]

In some embodiments of a compound of Formula (I), (Ia), (Ib), (Ic), (Id), or (Ie), or a pharmaceutically acceptable salt or stereoisomer thereof, R³ is substituted with R^{15a} and R^{15a} is

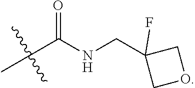 [SMILES: *C(=O)NCC1(F)COC1]

In some embodiments of a compound of Formula (I), (Ia), (Ib), (Ic), (Id), or (Ie), or a pharmaceutically acceptable salt or stereoisomer thereof, R³ is substituted with R^{15a} and R^{15a} is

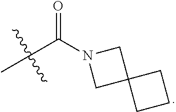 [SMILES: *C(=O)N1CC2(CCC2)C1]

In some embodiments of a compound of Formula (I), (Ia), (Ib), (Ic), (Id), or (Ie), or a pharmaceutically acceptable salt or stereoisomer thereof, R³ is substituted with R^{15a} and R^{15a} is

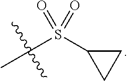 [SMILES: *S(=O)(=O)C1CC1]

In some embodiments of a compound of Formula (I), (Ia), (Ib), (Ic), (Id), or (Ie), or a pharmaceutically acceptable salt or stereoisomer thereof, R³ is substituted with R^{15a} and R^{15a} is

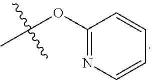 [SMILES: *Oc1ccccn1]

In some embodiments of a compound of Formula (I), (Ia), (Ib), (Ic), (Id), or (Ie), or a pharmaceutically acceptable salt or stereoisomer thereof, R³ is substituted with R^{15a} and R^{15a} is-OCH₃. In some embodiments of a compound of Formula (I), (Ia), (Ib), (Ic), (Id), or (Ie), or a pharmaceutically acceptable salt or stereoisomer thereof, R³ is substituted with a second R^{15a} wherein the second R^{15a} is halogen. In some embodiments of a compound of Formula (I), (Ia), (Ib), (Ic), (Id), or (Ie), or a pharmaceutically acceptable salt or stereoisomer thereof, R³ is substituted with a second R^{15a} wherein the second R^{15a} is-F. In some embodiments of a compound of Formula (I), (Ia), (Ib), (Ic), (Id), or (Ie), or a pharmaceutically acceptable salt or stereoisomer thereof, R³ is substituted with a second R^{15a} wherein the second R^{15a} is C₁₋₆alkoxy. In some embodiments of a compound of Formula (I), (Ia), (Ib), (Ic), (Id), or (Ie), or a pharmaceutically acceptable salt or stereoisomer thereof, R³ is substituted with a second R^{15a} wherein the second R^{15a} is —OCH₃. In some embodiments of a compound of Formula (I), (Ia), (Ib), (Ic), (Id), or (Ie), or a pharmaceutically acceptable salt or stereoisomer thereof, R³ is substituted with a second R^{15a} wherein the second R^{15a} is C₁₋₆alkyl. In some embodiments of a compound of Formula (I), (Ia), (Ib), (Ic), (Id), or (Ie), or a pharmaceutically acceptable salt or stereoisomer thereof, R³ is substituted with a second R^{15a} wherein the second R^{15a} is —CH₃.

In some embodiments of a compound of Formula (I), (Ia), (Ib), (Ic), (Id), or (Ie), or a pharmaceutically acceptable salt or stereoisomer thereof, R³ is

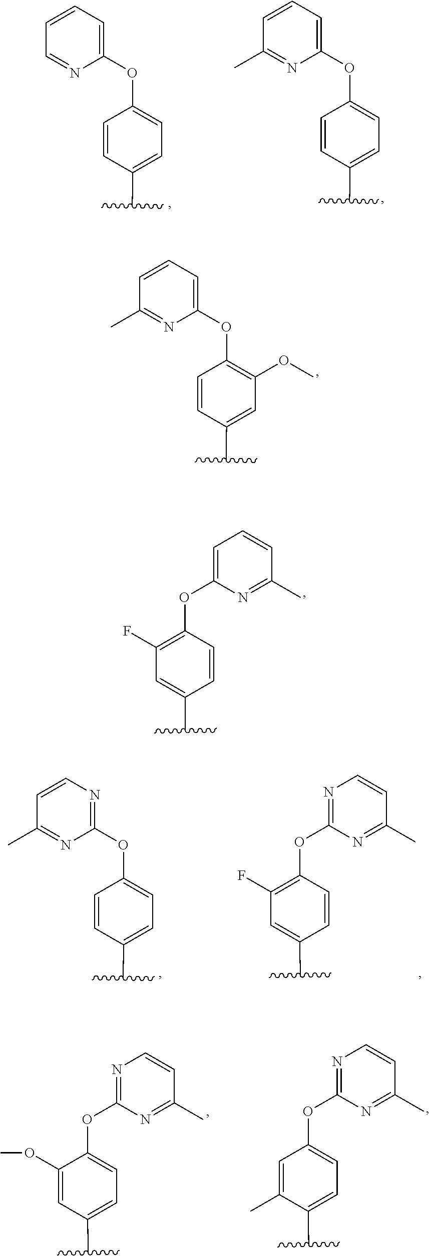

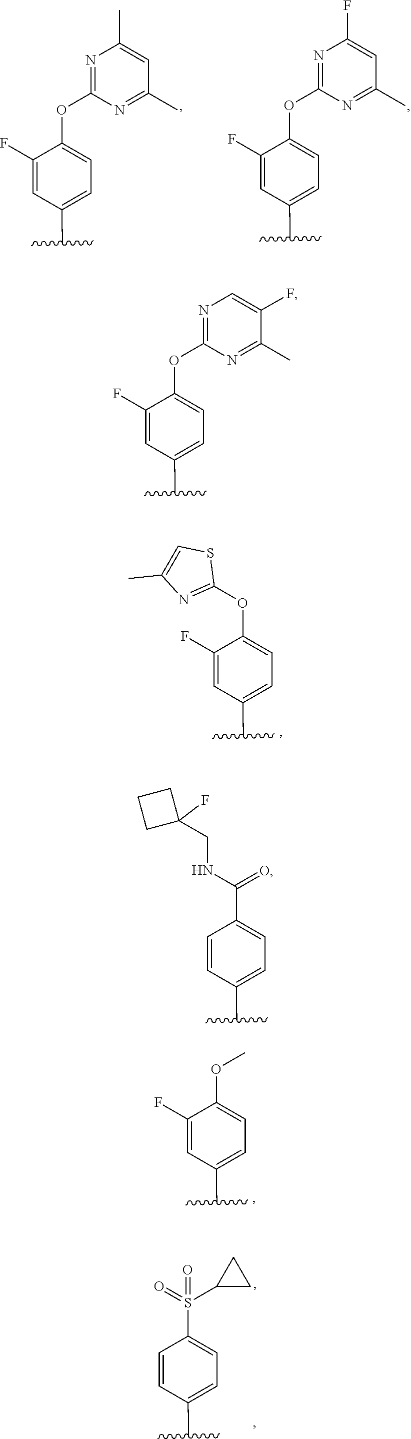

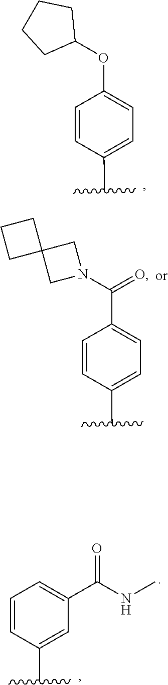 [SMILES: *c1ccc(OC2CCCC2)cc1 ; *c1ccc(C(=O)N2CC3(CCC3)C2)cc1 ; *c1cccc(C(=O)NC)c1]

In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R⁴ is selected from C₆₋₁₀arylene and C₁₋₉heteroarylene, wherein C₆₋₁₀arylene and C₁₋₉heteroarylene are optionally substituted with one, two, or three groups selected from R^{15b}. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R⁴ is selected from C₂₋₉heterocycloalkylene, C₆₋₁₀arylene, and C₁₋₉heteroarylene, wherein the C₂₋₉heterocycloalkylene, C₆₋₁₀arylene and C₁₋₉heteroarylene are optionally substituted with one, two, or three groups selected from R^{15b}. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R⁴ is C₆₋₁₀arylene optionally substituted with one, two, or three groups selected from R^{15b}. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R⁴ is phenylene optionally substituted with one, two, or three groups selected from R^{15b}. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R⁴ is phenylene substituted with one, two, or three groups selected from R^{15b}. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R⁴ is unsubstituted phenylene. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R⁴ is

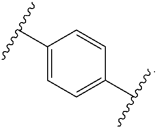 [SMILES: *c1ccc(*)cc1]

In some embodiments of a compound of Formula (I), (Ia), (Ia′), (Ia″), (Ib), (Tb′), (Ib″), (Ic), (Ic′), (Ic″), (Id), or (Ie), or a pharmaceutically acceptable salt or stereoisomer thereof, R⁴ is an C₆₋₁₀arylene comprising a phenyl fused with C₂₋₉ heterocycloalkyl, wherein the C₆₋₁₀arylene is optionally substituted with one, two, or three groups selected from R^{15b}. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R⁴ is an C₆₋₁₀arylene comprising a phenyl fused with 5-6 membered heterocycloalkyl, wherein the C₆₋₁₀arylene is optionally substituted with one, two, or three groups selected from R^{15b}. In some embodiments, the fused C₂₋₉ heterocycloalkyl comprises 1, 2, or 3 heteroatoms selected from the group consisting of O, S, N, or any combination thereof. In some embodiments, the fused C₂₋₉heterocycloalkylene comprises 1, 2, or 3 nitrogens. In some embodiments, the fused C₂₋₉heterocycloalkylene comprises 1 nitrogen. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R⁴ is

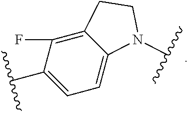 [SMILES: *c1ccc2c(c1F)CCN2*]

In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R⁴ is C₁₋₉heteroarylene optionally substituted with one, two, or three groups selected from R^{15b}. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R⁴ is C₁₋₉heteroarylene substituted with one, two, or three groups selected from R^{15b}. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R⁴ is pyridyl optionally substituted with one, two, or three groups selected from R^{15b}. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R⁴ is pyridyl substituted with one, two, or three groups selected from R^{15b}. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R⁴ is C₂₋₉heterocycloalkylene optionally substituted with one, two, or three groups selected from R^{15b}. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R⁴ is C₂₋₉heterocycloalkylene substituted with one, two, or three groups selected from R^{15b}. In some embodiments, the C₂₋₉heterocycloalkylene comprises 1, 2, 3, 4, 5, or 6 heteroatoms selected from the group consisting of O, S, N, or any combination thereof. In some embodiments, the C₂₋₉heterocycloalkylene comprises 1, 2, or 3 heteroatoms selected from the group consisting of O, S, N, or any combination thereof. In some embodiments, the C₂₋₉heterocycloalkylene comprises 1, 2, or 3 nitrogens. In some embodiments, the C₂₋₉heterocycloalkylene comprises 1 nitrogen. In some embodiments, the C₂₋₉heterocycloalkylene is monocyclic. In some embodiments, the C₂₋₉heterocycloalkylene is bicyclic. In some embodiments, the C₂₋₉heterocycloalkylene is a spiro ring system. In some embodiments, the C₂₋₉heterocycloalkylene is

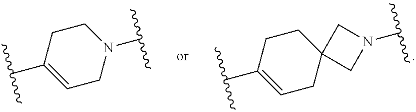 [SMILES: *C1=CCN(*)CC1 ; *C1=CCC2(CC1)CN(*)C2]

In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R⁴ is C₃₋₆cycloalkylene optionally substituted with one, two, or three groups selected from R^{15b}. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R⁴ is C₃₋₆cycloalkylene optionally substituted with one, two, or three groups selected from R^{15b}. In some embodiments, the C₃₋₆cycloalkylene is saturated. In some embodiments, the C₃₋₆cycloalkylene is unsaturated. In some embodiments, the C₃₋₆cycloalkylene is partially saturated. In some embodiments, the C₃₋₆cycloalkylene comprises one double bond. In some embodiments, the C₃₋₆cycloalkylene comprises two double bonds. In some embodiment, the C₃₋₆cycloalkylene is

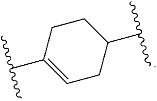 [SMILES: *C1=CCC(*)CC1]

In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R⁴ is

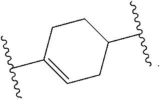 [SMILES: *C1=CCC(*)CC1]

In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R⁴ is C₃₋₆cycloalkylene optionally substituted with one, two, or three groups selected from R^{15b}.

In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R⁴ is C₃₋₁₀cycloalkylene optionally substituted with one, two, or three groups selected from R^{15b}. In some embodiments, the C₃₋₁₀cycloalkylene is a bicyclic ring. In some embodiments, the C₃₋₁₀cycloalkylene comprises a cycloalkyl fused with an aryl or heteroaryl. In some embodiments, the C₃₋₁₀cycloalkylene comprises a cycloalkyl fused with an aryl or heteroaryl, wherein the cycloalkyl is attached to the carbon marked with

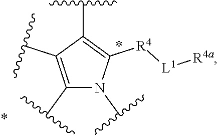

and the aryl or heteroaryl is attached to L¹.

In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R⁴ is C₂₋₉heterocycloalkylene optionally substituted with one, two, or three groups selected from R^{15b}. In some embodiments, the C₂₋₉heterocycloalkylene is a monocyclic ring. In some embodiments, R⁴ is

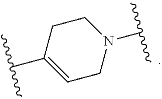 [SMILES: *C1=CCN(*)CC1]

In some embodiments, the C₂₋₉heterocycloalkylene is a bicyclic ring. In some embodiments, R⁴ is

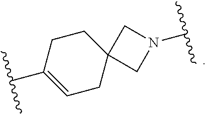 [SMILES: *C1=CCC2(CC1)CN(*)C2]

In some embodiments, the C₂₋₉heterocycloalkylene comprises a heterocycloalkyl fused with an aryl or heteroaryl. In some embodiments, the C₂₋₉heterocycloalkylene comprises a heterocycloalkyl fused with an aryl or heteroaryl, wherein the heterocycloalkyl is attached to the carbon marked with

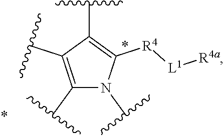

and the aryl or heteroaryl is attached to L¹.

In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R⁴ is C₆₋₁₀arylene optionally substituted with one, two, or three groups selected from R^{15b}. In some embodiments, the C₆₋₁₀arylene is a monocyclic ring. In some embodiments, R⁴ is an optionally substituted phenylene. In some embodiments, R⁴ is

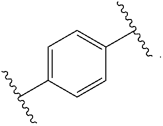 [SMILES: *c1ccc(*)cc1]

In some embodiments, R⁴ is

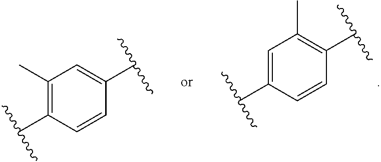

In some embodiments, R⁴ is

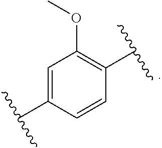 [SMILES: *c1ccc(*)c(OC)c1]

In some embodiments, R⁴ is

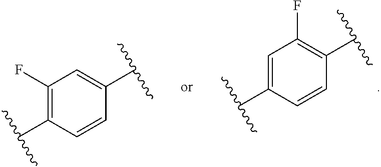 [SMILES: *c1ccc(*)c(F)c1 ; *c1ccc(*)c(F)c1]

In some embodiments, R⁴ is

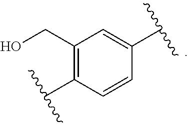 [SMILES: *c1ccc(*)c(CO)c1]

In some embodiments, the C₆₋₁₀arylene is a bicyclic ring. In some embodiments, the C₆₋₁₀arylene comprises a phenyl fused with a cycloalkyl or heterocycloalkyl. In some embodiments, the C₆₋₁₀arylene comprises a phenyl fused with a 5-6 membered cycloalkyl or 5-6 membered heterocycloalkyl. In some embodiments, the C₆₋₁₀arylene comprises a phenyl fused with a cycloalkyl or heterocycloalkyl, wherein the phenyl is attached to the carbon marked with *

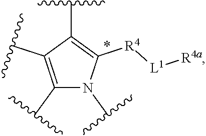

and the cycloalkyl or heterocycloalkyl is attached to L¹. In some embodiments, R⁴ is

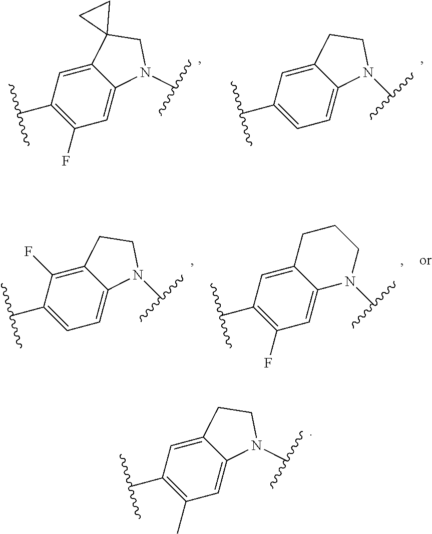 [SMILES: *c1cc2c(cc1F)N(*)CC21CC1 ; *c1ccc2c(c1)CCN2* ; *c1ccc2c(c1F)CCN2* ; *c1cc2c(cc1F)N(*)CCC2 ; *c1cc2c(cc1C)N(*)CC2]

In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R⁴ is C₁₋₉heteroarylene optionally substituted with one, two, or three groups selected from R^{15b}. In some embodiments, the C₁₋₉heteroarylene is a monocyclic ring. In some embodiments, R⁴ is

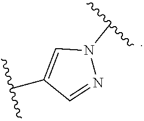 [SMILES: *c1cnn(*)c1]

In some embodiments, R⁴ is

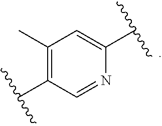 [SMILES: *c1cc(C)c(*)cn1]

In some embodiments, R⁴ is

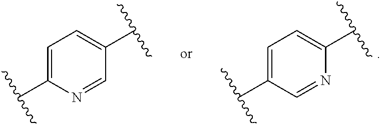 [SMILES: *c1ccc(*)nc1 ; *c1ccc(*)nc1]

In some embodiments, the C₁₋₉heteroarylene is a bicyclic ring. In some embodiments, the C₁₋₉heteroarylene comprises a heteroaryl fused with a cycloalkyl or heterocycloalkyl. In some embodiments, the C₁₋₉heteroarylene comprises a heteroaryl fused with a 5-6 membered cycloalkyl or 5-6 membered heterocycloalkyl. In some embodiments, the C₁₋₉heteroarylene comprises a heteroaryl fused with a cycloalkyl or heterocycloalkyl, wherein the heteoaryl is attached to the carbon marked with *

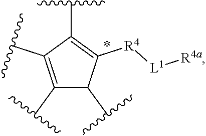

and the cycloalkyl or heterocycloalkyl is attached to L¹. In some embodiments, R⁴ is

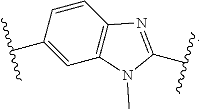 [SMILES: *c1ccc2nc(*)n(C)c2c1]

In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, L¹ is a bond, —N(R^{9a})—, —N(R^{9a})C(O)—, —C(O)N(R^{9a})—, —N(R^{9a})S(O)₂—, —S(O)₂N(R^{9a})—, —O—, —C(O)—, —OC(O)—, —C(O)O—, —S—, —S(O)—, —S(O)₂—, or C₁₋₆alkylene optionally substituted with one, two, or three groups selected from R^{15c}. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, L¹ is a bond, —N(R^{9a})—, —N(R^{9a})C(O)—, —C(O)N(R^{9a})—, or —C(O)—. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, L¹ is —N(R^{9a})—. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, L¹ is —N(H)—. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, L¹ is —N(R^{9a})C(O)—. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, L¹ is —N(H)C(O)—. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, L¹ is —C(O)N(R^{9a})—. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, L¹ is —C(O)N(H)—. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, L¹ is —C(O)—. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, L¹ is a bond. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, L¹ is C₂₋₉heterocycloalkylene optionally substituted with one, two, or three groups selected from R^{15c}. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, L¹ is 4-6 membered heterocycloalkylene optionally substituted with one, two, or three groups selected from R^{15c}. n some embodiments of a compound of Formula (I), (Ia), (la′), (Ia″), (Ib), (Ib′), (Ib″), (Ic), (Ic′), (Ic″), (Id), or (Ie), or a pharmaceutically acceptable salt or stereoisomer thereof, L¹ is

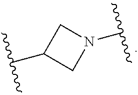 [SMILES: *C1CN(*)C1]

In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4a} is selected from

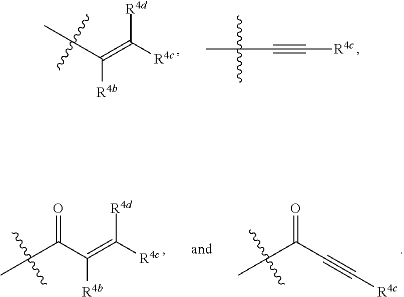

In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4a} is

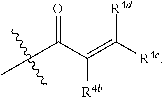

In some embodiments, disclosed herein is a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, wherein R^{4a} is

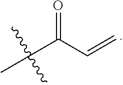 [SMILES: *C(=O)C=C]

In some embodiments, disclosed herein is a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, wherein R^{4a} is

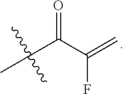 [SMILES: *C(=O)C(=C)F]

In some embodiments, disclosed herein is a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, wherein R^{4a} is

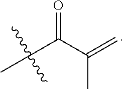 [SMILES: *C(=O)C(=C)C]

In some embodiments, disclosed herein is a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, wherein R^{4a} is

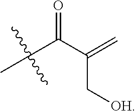 [SMILES: *C(=O)C(=C)CO]

In some embodiments, disclosed herein is a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, wherein R^{4a} is

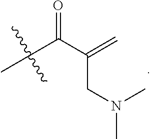 [SMILES: *C(=O)C(=C)CN(C)C]

In some embodiments, disclosed herein is a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, wherein R^{4a} is

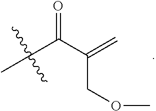 [SMILES: *C(=O)C(=C)COC]

In some embodiments, disclosed herein is a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, wherein R^{4a} is

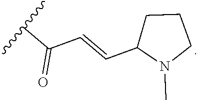 [SMILES: *C(=O)/C=C/C1CCCN1C]

In some embodiments, disclosed herein is a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, wherein R^{4a} is

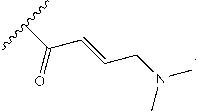 [SMILES: *C(=O)/C=C/CN(C)C]

In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4a} is

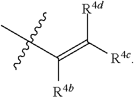

In some embodiments, disclosed herein is a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, wherein R^{4a} is

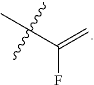 [SMILES: *C(=C)F]

In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4a} is

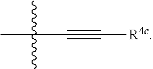

In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4a} is

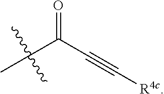

In some embodiments, disclosed herein is a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, wherein R^{4a} is

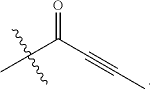 [SMILES: *C(=O)C#CC]

In some embodiments, disclosed herein is a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, wherein R^{4a} is

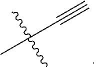 [SMILES: *C#C]

In some embodiments, disclosed herein is a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, wherein R^{4a} is

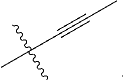 [SMILES: *C#CC]

In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4b}, R^{4c}, and R^{4d} are each independently hydrogen, halogen, —CN, —C(O)R¹³, —C(O)OR¹⁰, —C(O)N(R¹⁰)(R¹¹), —N(R¹⁰)(R¹¹), —C₁₋₆alkyl-N(R¹⁰)(R¹¹), —C(O)N(R¹⁰)OR¹⁰, C₁₋₆alkyl, phenyl, 3 to 7-membered heterocycloalkyl, or 5 or 6-membered heteroaryl. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4b}, R^{4c}, and R^{4d} are each independently hydrogen, C₁₋₆alkyl, or —C₁₋₆alkyl-N(R¹⁰)(R¹¹). In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4b}, R^{4c}, and R^{4d} are each independently hydrogen, halogen, C₁₋₆alkyl, or —C₁₋₆alkyl-N(R¹⁰)(R¹¹), wherein the alkyl is optionally substituted with one, two, or three R^{15d}. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4b} is hydrogen. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4b} is hydrogen, halogen, or C₁₋₆alkyl, wherein the alkyl is optionally substituted with one, two, or three R^{15d}. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4b} is hydrogen. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4b} is halogen. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4b} is F. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4b} is C₁₋₆alkyl. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4b} is C₁₋₆alkyl optionally substituted with one, two, or three R^{15d}. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4b} is C₁₋₃alkyl. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R⁴⁵ is CH₃. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4b} is C₁₋₆alkyl substituted with one, two, or three R^{15d}. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4b} is C₁₋₆alky-OR¹⁰. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4b} is C₁₋₃alky-OR¹⁰. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4b} is —CH₂OH or CH₂OCH₃. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4c} is hydrogen. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R⁴ is C₁₋₆alkyl. In some embodiments of a compound of Formula (I), (Ta), (Ia′), (Ia″), (Ib), (Ib′), (Ib″), (Ic), (Ic′), (Ic″), (Id), or (Ie), or a pharmaceutically acceptable salt or stereoisomer thereof, R^{4c} is —C₁₋₆alkyl-N(R¹⁰)(R¹¹). In some embodiments of a compound of Formula (I), (Ia), (la′), (Ia″), (Ib), (Ib′), (Ib″), (Ic), (Ic′), (Ic″), (Id), or (Ie), or a pharmaceutically acceptable salt or stereoisomer thereof, R^{4d} is —CH₂—N(CH₃)₂. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4d} is hydrogen. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4d} is C₁₋₆alkyl. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4d} is —C₁₋₆alkyl-N(R¹⁰)(R¹¹). In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4d} is —CH₂—N(CH₃)₂. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4e} and R^{4d} are each independently hydrogen or —C₁₋₆alkyl-N(R¹⁰)(R¹¹). In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4c} and R^{4d} are each independently hydrogen or —CH₂—N(CH₃)₂. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4c} and R^{4d} are each independently hydrogen. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R^{4c} is —CH₂—N(CH₃)₂ and R^{4d} is hydrogen. In some embodiments, R^{4b}, R^{4c}, and/or R^{4d} are hydrogen that comprise deuterium at a percentage higher than the natural abundance of deuterium. In some embodiments, R^{4b}, R^{4c}, and/or R^{4d} are hydrogen wherein one or more protiums are replaced with one or more deuteriums.

In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof,

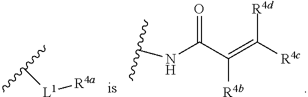

In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof,

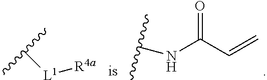

In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof,

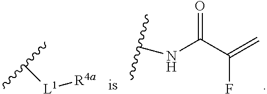

In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof,

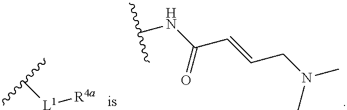

In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R¹ and R² are independently selected from hydrogen and C₁₋₆alkyl. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R¹ and R² are hydrogen. In some embodiments of a compound of Formula (I), or a pharmaceutically acceptable salt or stereoisomer thereof, R¹ is hydrogen R² is C₁₋₆alkyl.

In some embodiments, disclosed herein is a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, wherein R¹⁰ is optionally substituted with one or more substituents selected from halogen, —CN, —OH, —SF₅, —SH, —S(═O) C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —S(═O)(═NC₁-C₃alkyl) (C₁-C₃alkyl), —NH₂, —NHC₁-C₃alkyl, —N(C₁-C₃alkyl)₂, —N═S(═O) (C₁-C₃alkyl)₂, —O—C₁-C₄alkylene-OH, —O—C₁-C₄alkylene-NH₂, —C(═O) C₁-C₃alkyl, —C(═O)OH, —C(═O)OC₁-C₃alkyl, —C(═O) NH₂, —C(═O)NHC₁-C₃alkyl, —C(═O)N(C₁-C₃alkyl)₂, —P(═O) (C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, C₃-C₆cycloalkyl, and 3- to 6-membered heterocycloalkyl. In some embodiments, disclosed herein is a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, wherein R¹⁰ is optionally substituted with one or more substituents selected from halogen, —CN, —OH, —SF₅, —SH, —S(═O)₂NH₂, —S(═O)₂NHC₁-C₃alkyl, —S(═O)₂N(C₁-C₃alkyl)₂, —S(═O)(═NC₁-C₃alkyl) (C₁-C₃alkyl), —NH₂, —NHC₁-C₃alkyl, —N(C₁-C₃alkyl)₂, —O—C₁-C₄alkylene-OH, —O—C₁-C₄alkylene-NH₂, —C(═O) C₁-C₃alkyl, —C(═O)OH, —C(═O)OC₁-C₃alkyl, —C(═O) NH₂, —C(═O)NHC₁-C₃alkyl, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, C₃-C₆cycloalkyl, and 3- to 6-membered heterocycloalkyl.

In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, each R¹¹ is independently selected from hydrogen, C₁₋₆alkyl, and C₁₋₆haloalkyl.

In some embodiments of a compound of Formula (I), (Ia), (la′), (Ia″), (Ib), (Ib′), (Ib″), (Ic), (Ic′), (Ic″), (Id), or (Ie), or a pharmaceutically acceptable salt or stereoisomer thereof, R¹⁰ and R¹¹ together with the nitrogen to which they are attached, form a C₂₋₉heterocycloalkyl. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, R¹⁰ and R¹¹ together with the nitrogen to which they are attached, form

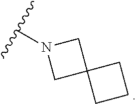 [SMILES: *N1CC2(CCC2)C1]

In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, each R¹³ is independently selected C₁₋₆alkyl, C₃₋₆cycloalkyl, C₂₋₉heterocycloalkyl, wherein C₁₋₆alkyl, C₃₋₆cycloalkyl, C₂₋₉heterocycloalkyl are optionally substituted with one, two, or three groups selected from halogen, C₁₋₆alkyl, C₁₋₆haloalkyl. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, each R¹³ is

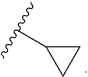 [SMILES: *C1CC1]

In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, each R^{15b} is independently selected from halogen, oxo, —CN, C₁₋₆alkyl, C₃₋₁₀cycloalkyl, —CH₂—C₃₋₆cycloalkyl, C₂₋₉heterocycloalkyl, —CH₂—C₂₋₉heterocycloalkyl, —OR¹⁰, —SR¹⁰, —SF₅, —N(R¹⁰)(R¹¹), —C(O)OR¹⁰, wherein C₁₋₆alkyl, C₃₋₁₀cycloalkyl, —CH₂—C₃₋₆cycloalkyl, C₂₋₉heterocycloalkyl, and —CH₂—C₂₋₉heterocycloalkyl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OH, —SH, and amino. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, each R^{15b} is independently selected from halogen, C₁₋₆alkyl, —OR¹⁰, wherein C₁₋₆alkyl is optionally substituted with one, two, or three groups independently selected from halogen, C₁₋₆alkyl, C₁₋₆haloalkyl, C₁₋₆alkoxy, C₁₋₆haloalkoxy, —OH, —SH, and amino. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, each R^{15b} is halogen. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, each R^{15b} is C₁₋₆alkyl, wherein C₁₋₆alkyl is optionally substituted with one, two, or three groups independently selected from halogen, C₁₋₆alkyl, C₁₋₆haloalkyl, C₁₋₆alkoxy, C₁₋₆haloalkoxy, —OH, —SH, and amino. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, each R^{15b} is halogen. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, each R^{15b} is C₁₋₆alkyl, wherein C₁₋₆alkyl is optionally substituted with one, two, or three groups independently selected from —OH. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, each R^{15b} is —OR¹⁰.

In some embodiments of a compound of Formula (I), (Ia), (la′), (Ia″), (Ib), (Ib′), (Ib″), (Ic), (Ic′), (Ic″), (Id), or (Ie), or a pharmaceutically acceptable salt or stereoisomer thereof, each R^{15c} is independently selected from halogen, oxo, —CN, C₁₋₆alkyl, C₃₋₁₀cycloalkyl, —CH₂—C₃₋₆cycloalkyl, C₂₋₉heterocycloalkyl, —CH₂—C₂₋₉heterocycloalkyl, —OR¹⁰, —SR¹⁰, —SF₅, —N(R¹⁰)(R¹¹), —C(O)OR¹⁰, wherein the C₁₋₆alkyl, C₃₋₁₀cycloalkyl, —CH₂—C₃₋₆cycloalkyl, C₂₋₉heterocycloalkyl, and —CH₂—C₂₋₉heterocycloalkyl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OH, —SH, and amino. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, each R^{15c} is independently selected from halogen, oxo, —OH, —CN, C₁₋₆alkyl, C₁₋₆alkoxyl, and amino, wherein the C₁₋₆alkyl is optionally substituted with one, two, or three groups independently selected from halogen, oxo, —CN, —OH, —SH, and amino. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, each R^{15c} is independently selected from halogen, —OH, C₁₋₆alkoxyl, and C₁₋₆alkyl, wherein the C₁₋₆alkyl is optionally substituted with one, two, or three halogen.

In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, each R^{15d} is independently selected from halogen, oxo, —CN, C₁₋₆alkyl, C₃₋₁₀cycloalkyl, —CH₂—C₃₋₆cycloalkyl, C₂₋₉heterocycloalkyl, —CH₂—C₂₋₉heterocycloalkyl, —OR¹⁰, —SR¹⁰, —SF₅, —N(R¹⁰)(R¹¹), —C(O)OR¹⁰, wherein the C₁₋₆alkyl, C₃₋₁₀cycloalkyl, —CH₂—C₃₋₆cycloalkyl, C₂₋₉heterocycloalkyl, and —CH₂—C₂₋₉heterocycloalkyl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OH, —SH, and amino. In some embodiments of a compound of Formula ((I), (Ia), (Ia′), (Ia″), (Ib), (Ib′), (Ib″), (Ic), (Ic′), (Ic″), (Id), or (Ie), or a pharmaceutically acceptable salt or stereoisomer thereof, each R^{15d} is independently selected from halogen, oxo, —OH, —CN, C₁₋₆alkyl, C₁₋₆alkoxyl, and amino, wherein the C₁₋₆alkyl is optionally substituted with one, two, or three groups independently selected from halogen, oxo, —CN, —OH, —SH, and amino. In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, each R^{15d} is independently selected from halogen, —OH, and C₁₋₆alkyl, wherein the C₁₋₆alkyl is optionally substituted with one, two, or three halogen.

In some embodiments of a compound of Formula (I), or a pharmaceutically acceptable salt or stereoisomer thereof, each R^{15e} is independently selected from hydrogen, C₁₋₆alkyl, and C₁₋₆haloalkyl.

In some embodiments of a compound of Formula (I), or a pharmaceutically acceptable salt or stereoisomer thereof, each R^{15f} is independently selected from hydrogen, C₁₋₆alkyl, and C₁₋₆haloalkyl.

In some embodiments of a compound of Formula (I), (Ia), (Ia′), (Ia″), (Ib), (Ib′), (Ib″), (Ic), (Ic′), (Ic″), (Id), or (Ie), or a pharmaceutically acceptable salt or stereoisomer thereof, each R^{15g} is independently selected from hydrogen, C₁₋₆alkyl, and C₁₋₆haloalkyl.

Any combination of the groups described above for the various variables is contemplated herein. Throughout the specification, groups and substituents thereof are chosen by one skilled in the field to provide stable moieties and compounds.

In some embodiments, disclosed herein is a compound, or a pharmaceutically acceptable salt or stereoisomer thereof, selected from:

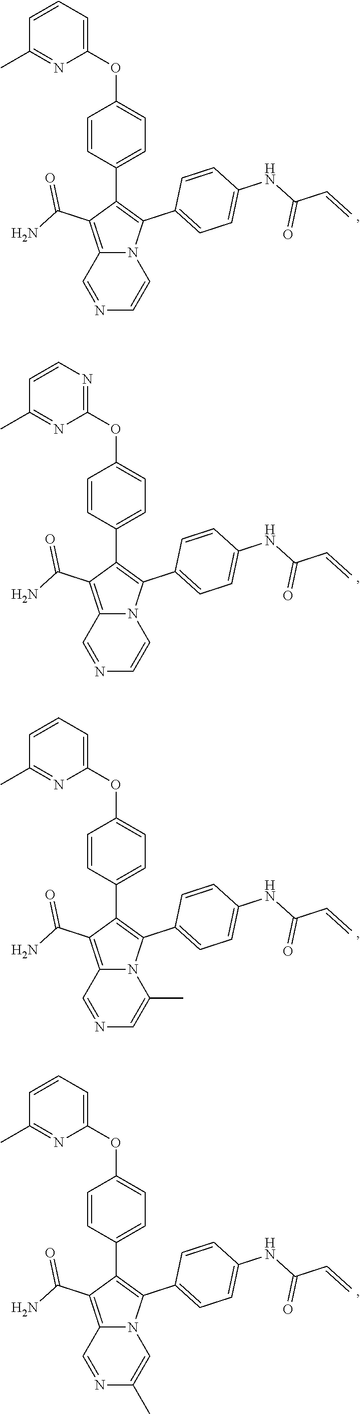

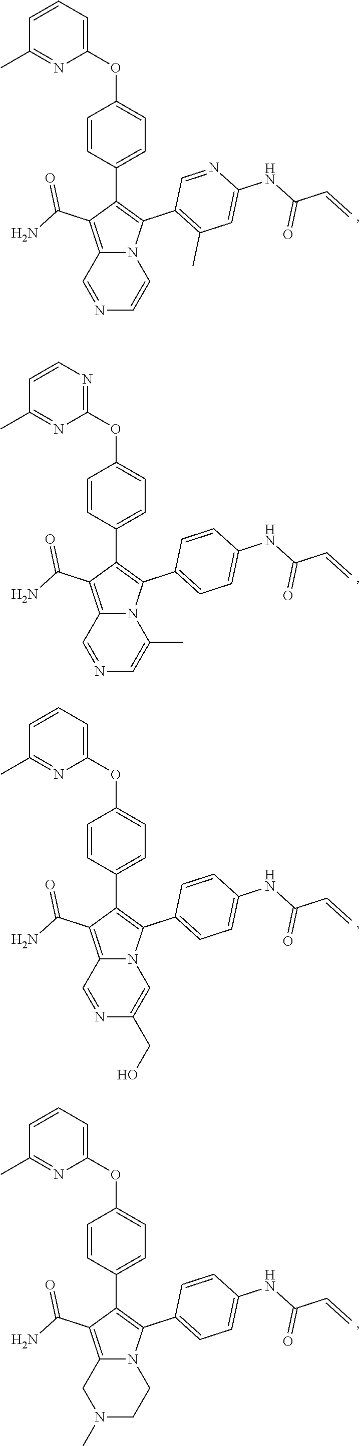

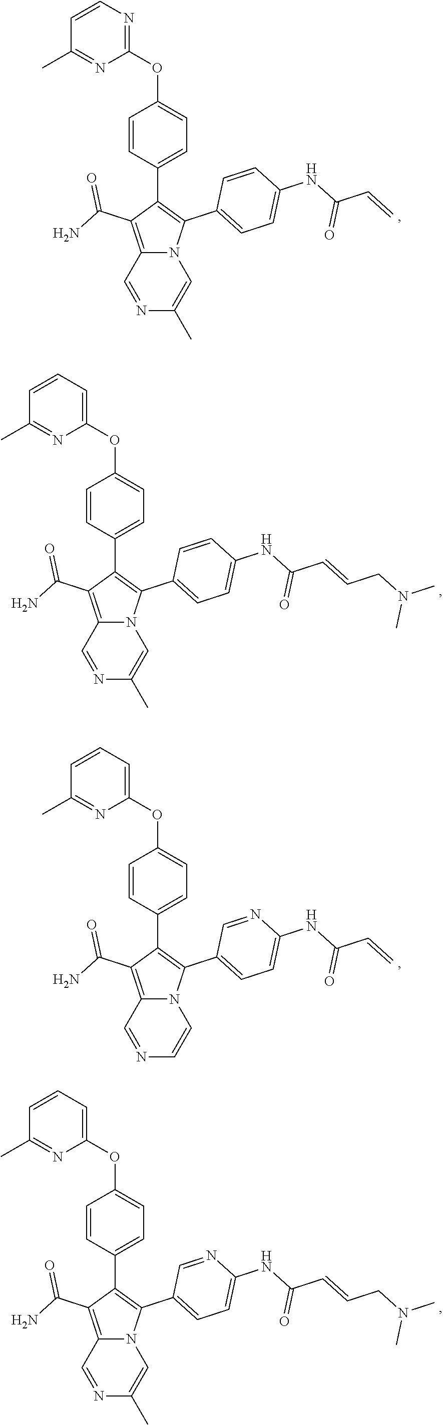

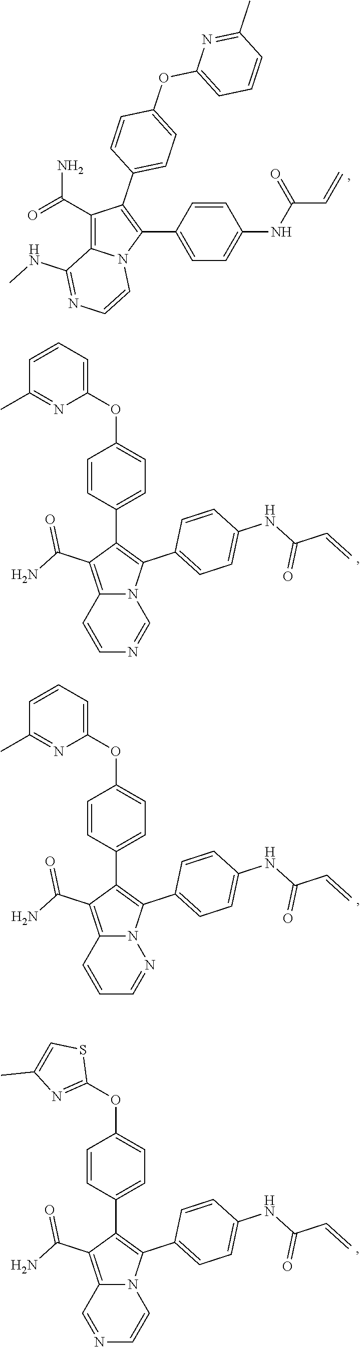

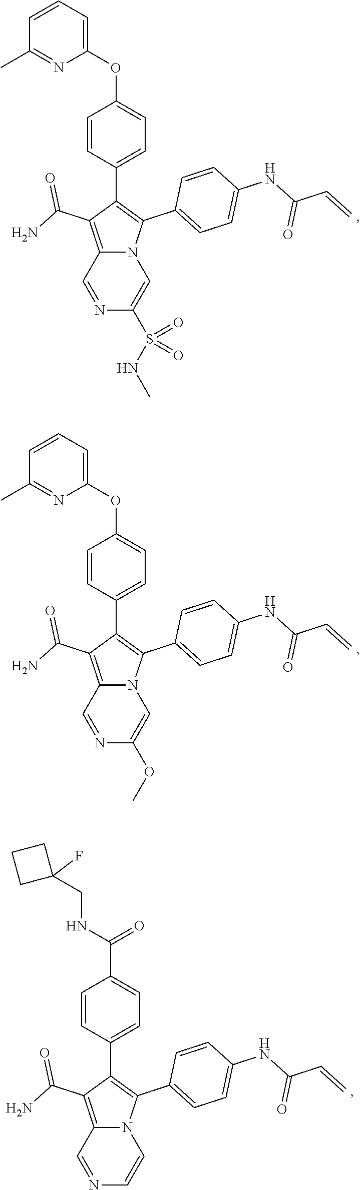

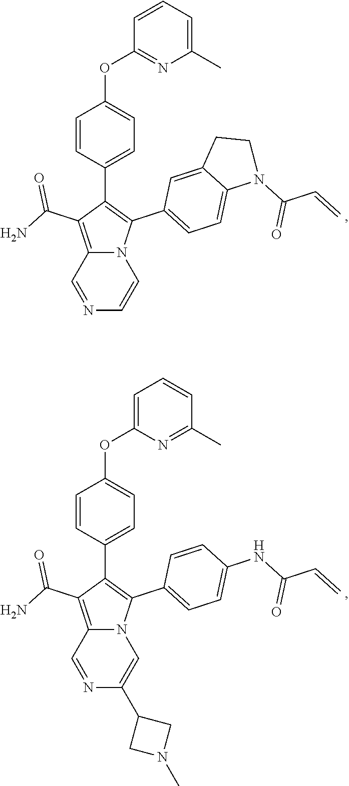 [SMILES: C=CC(=O)N1CCc2cc(-c3c(-c4ccc(Oc5cccc(C)n5)cc4)c(C(N)=O)c4cnccn34)ccc21 ; C=CC(=O)Nc1ccc(-c2c(-c3ccc(Oc4cccc(C)n4)cc3)c(C(N)=O)c3cnc(C4CN(C)C4)cn23)cc1]

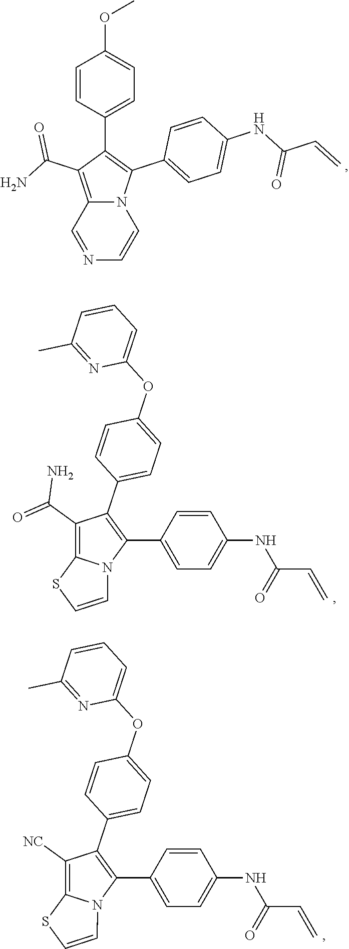

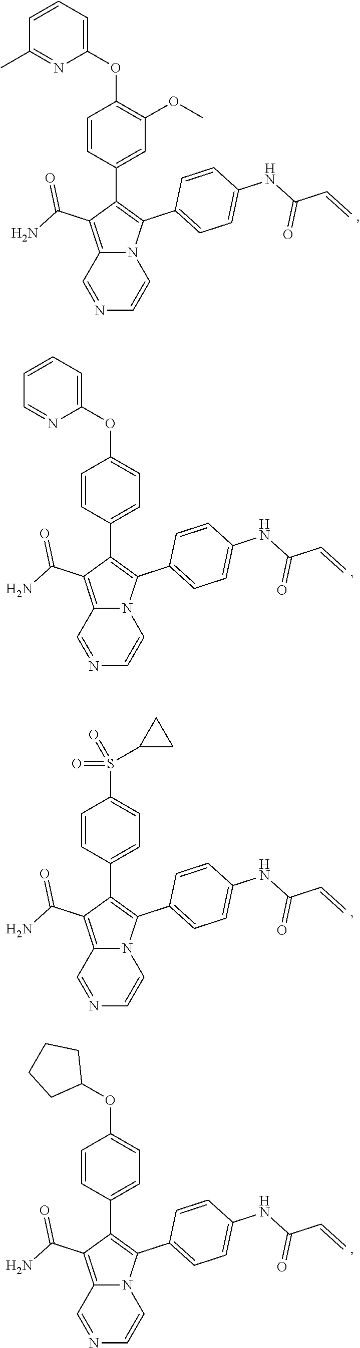 [SMILES: C=CC(=O)Nc1ccc(-c2c(-c3ccc(Oc4cccc(C)n4)c(OC)c3)c(C(N)=O)c3cnccn23)cc1 ; C=CC(=O)Nc1ccc(-c2c(-c3ccc(Oc4ccccn4)cc3)c(C(N)=O)c3cnccn23)cc1 ; C=CC(=O)Nc1ccc(-c2c(-c3ccc(S(=O)(=O)C4CC4)cc3)c(C(N)=O)c3cnccn23)cc1 ; C=CC(=O)Nc1ccc(-c2c(-c3ccc(OC4CCCC4)cc3)c(C(N)=O)c3cnccn23)cc1]

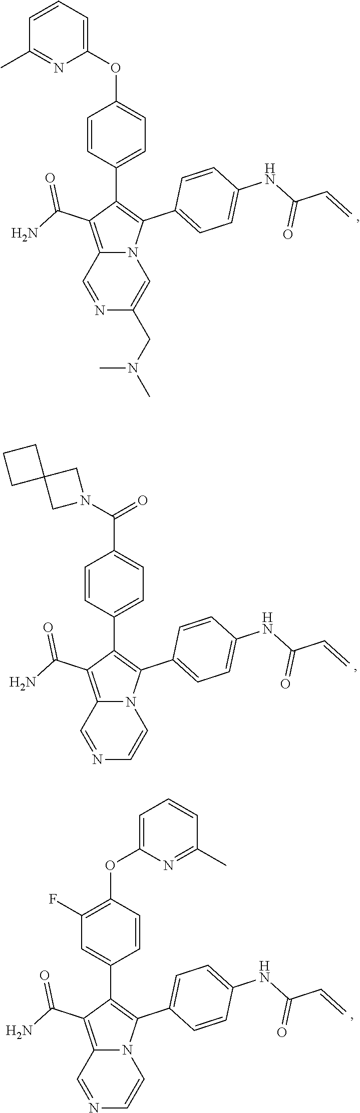 [SMILES: C=CC(=O)Nc1ccc(-c2c(-c3ccc(Oc4cccc(C)n4)cc3)c(C(N)=O)c3cnc(CN(C)C)cn23)cc1 ; C=CC(=O)Nc1ccc(-c2c(-c3ccc(C(=O)N4CC5(CCC5)C4)cc3)c(C(N)=O)c3cnccn23)cc1 ; C=CC(=O)Nc1ccc(-c2c(-c3ccc(Oc4cccc(C)n4)c(F)c3)c(C(N)=O)c3cnccn23)cc1]

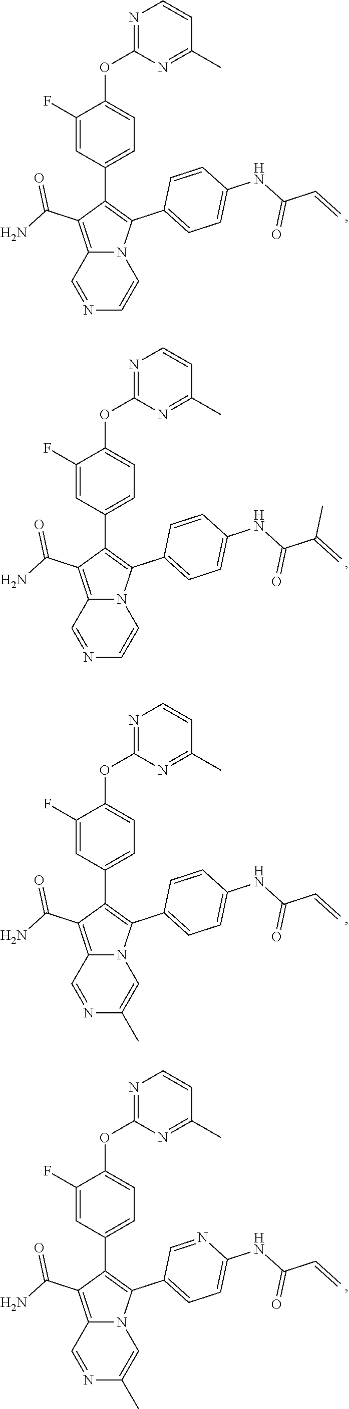

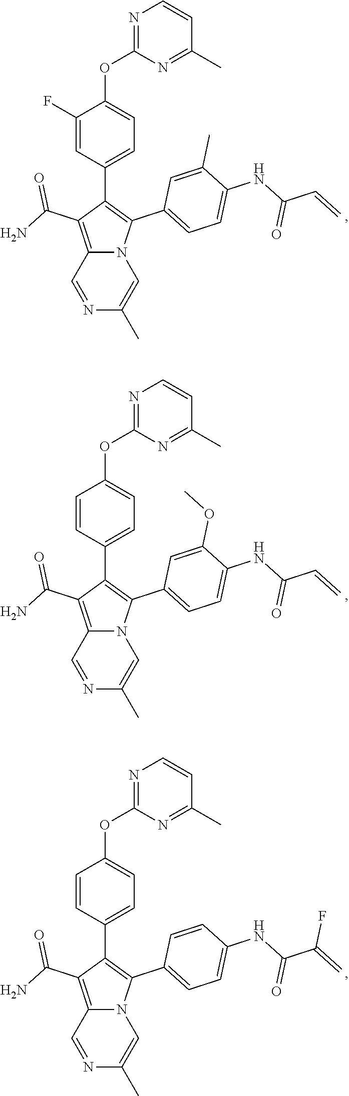 [SMILES: C=CC(=O)Nc1ccc(-c2c(-c3ccc(Oc4nccc(C)n4)c(F)c3)c(C(N)=O)c3cnc(C)cn23)cc1C ; C=CC(=O)Nc1ccc(-c2c(-c3ccc(Oc4nccc(C)n4)cc3)c(C(N)=O)c3cnc(C)cn23)cc1OC ; C=C(F)C(=O)Nc1ccc(-c2c(-c3ccc(Oc4nccc(C)n4)cc3)c(C(N)=O)c3cnc(C)cn23)cc1]

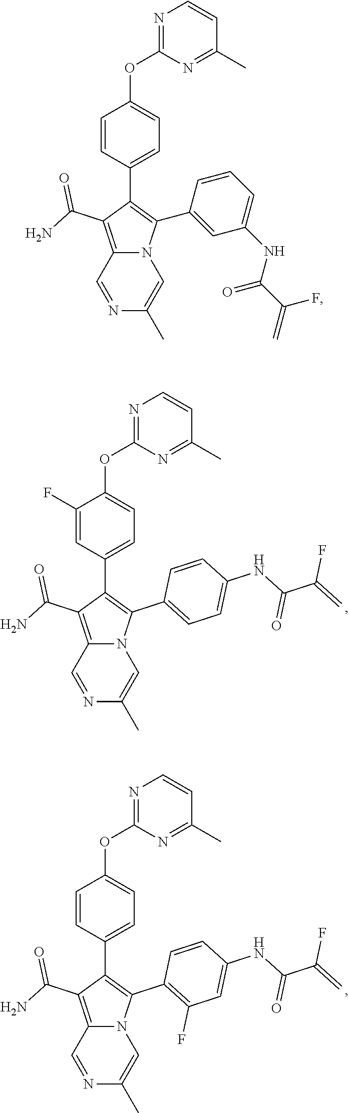 [SMILES: C=C(F)C(=O)Nc1cccc(-c2c(-c3ccc(Oc4nccc(C)n4)cc3)c(C(N)=O)c3cnc(C)cn23)c1 ; C=C(F)C(=O)Nc1ccc(-c2c(-c3ccc(Oc4nccc(C)n4)c(F)c3)c(C(N)=O)c3cnc(C)cn23)cc1 ; C=C(F)C(=O)Nc1ccc(-c2c(-c3ccc(Oc4nccc(C)n4)cc3)c(C(N)=O)c3cnc(C)cn23)c(F)c1]

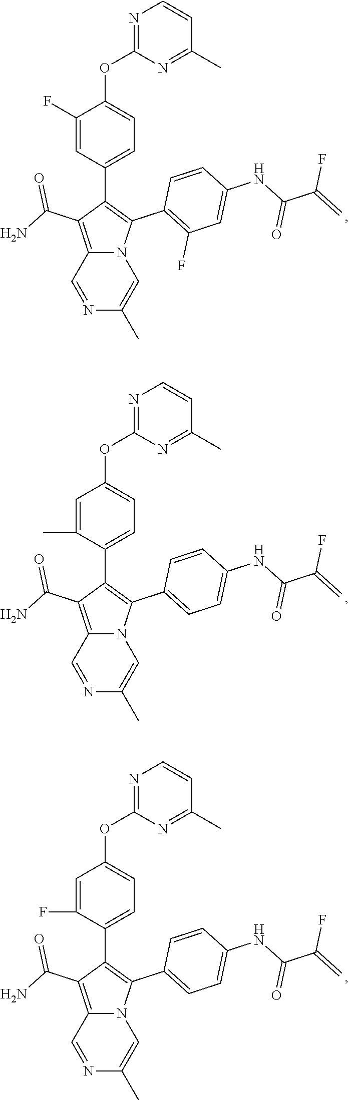 [SMILES: C=C(F)C(=O)Nc1ccc(-c2c(-c3ccc(Oc4nccc(C)n4)c(F)c3)c(C(N)=O)c3cnc(C)cn23)c(F)c1 ; C=C(F)C(=O)Nc1ccc(-c2c(-c3ccc(Oc4nccc(C)n4)cc3C)c(C(N)=O)c3cnc(C)cn23)cc1 ; C=C(F)C(=O)Nc1ccc(-c2c(-c3ccc(Oc4nccc(C)n4)cc3F)c(C(N)=O)c3cnc(C)cn23)cc1]

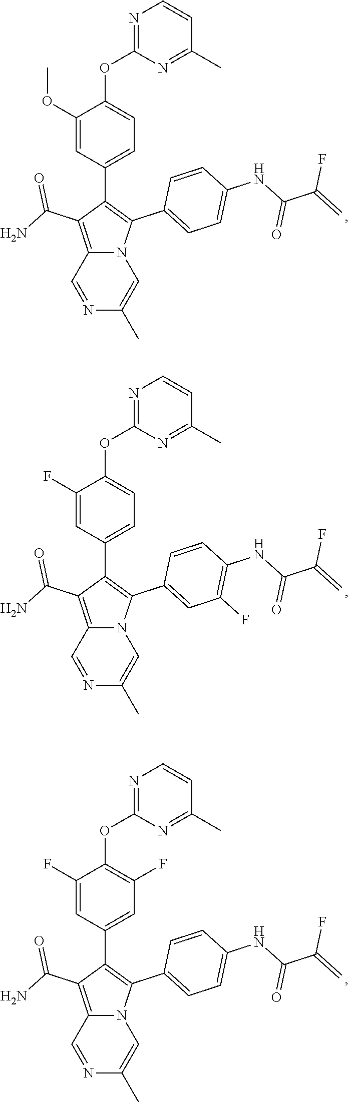

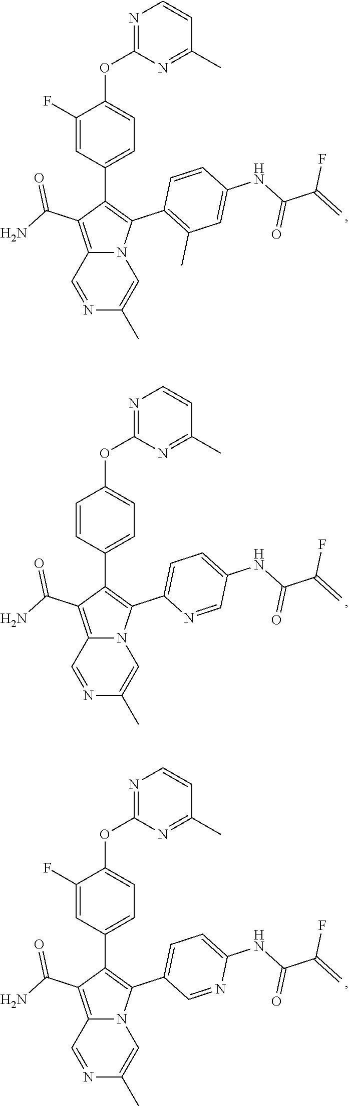 [SMILES: C=C(F)C(=O)Nc1ccc(-c2c(-c3ccc(Oc4nccc(C)n4)c(F)c3)c(C(N)=O)c3cnc(C)cn23)c(C)c1 ; C=C(F)C(=O)Nc1ccc(-c2c(-c3ccc(Oc4nccc(C)n4)cc3)c(C(N)=O)c3cnc(C)cn23)nc1 ; C=C(F)C(=O)Nc1ccc(-c2c(-c3ccc(Oc4nccc(C)n4)c(F)c3)c(C(N)=O)c3cnc(C)cn23)cn1]

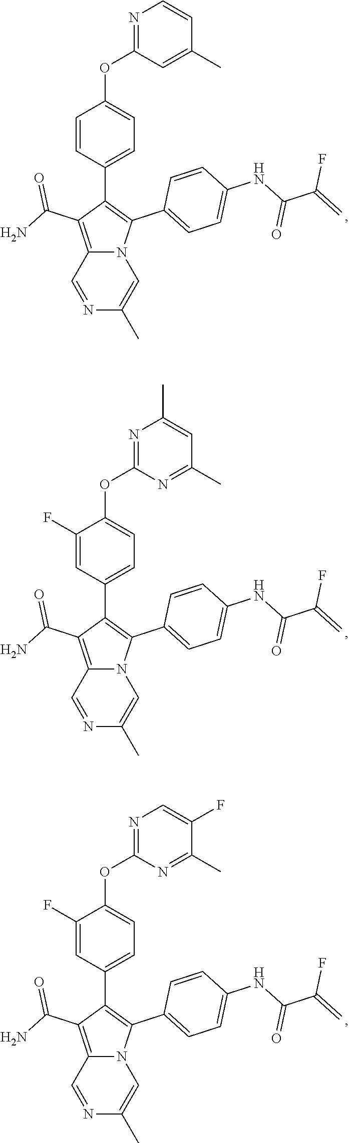 [SMILES: C=C(F)C(=O)Nc1ccc(-c2c(-c3ccc(Oc4cc(C)ccn4)cc3)c(C(N)=O)c3cnc(C)cn23)cc1 ; C=C(F)C(=O)Nc1ccc(-c2c(-c3ccc(Oc4nc(C)cc(C)n4)c(F)c3)c(C(N)=O)c3cnc(C)cn23)cc1 ; C=C(F)C(=O)Nc1ccc(-c2c(-c3ccc(Oc4ncc(F)c(C)n4)c(F)c3)c(C(N)=O)c3cnc(C)cn23)cc1]

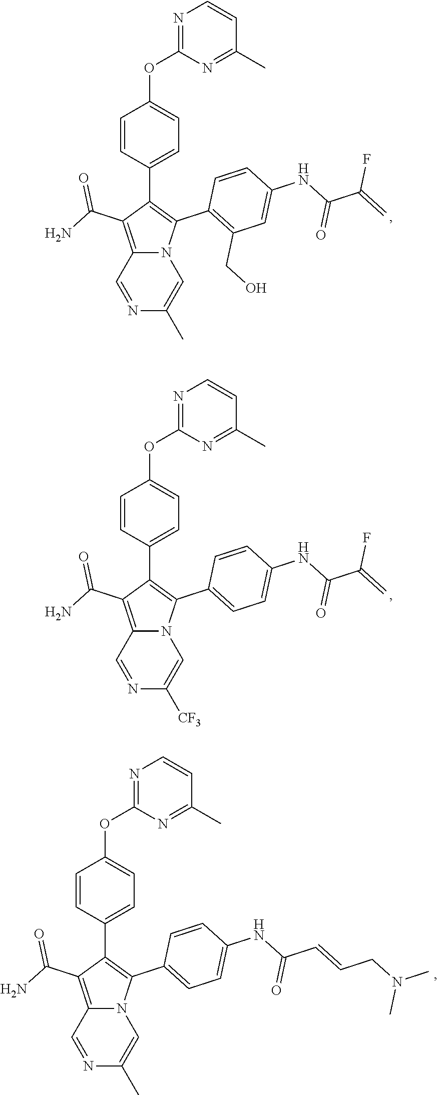

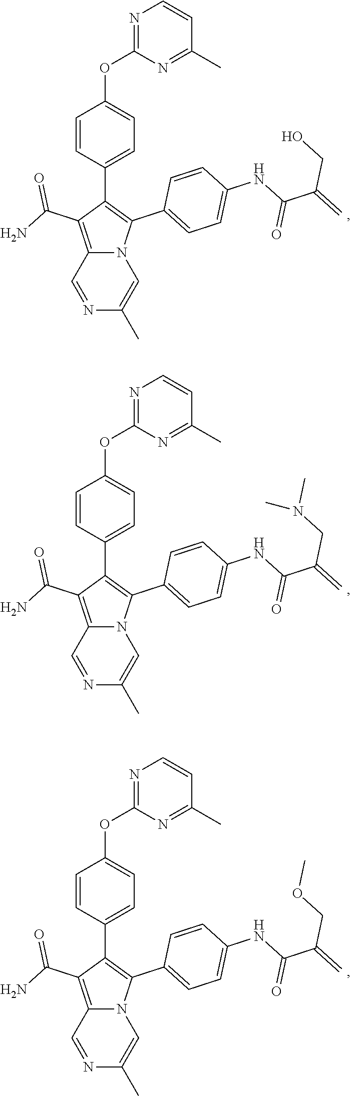

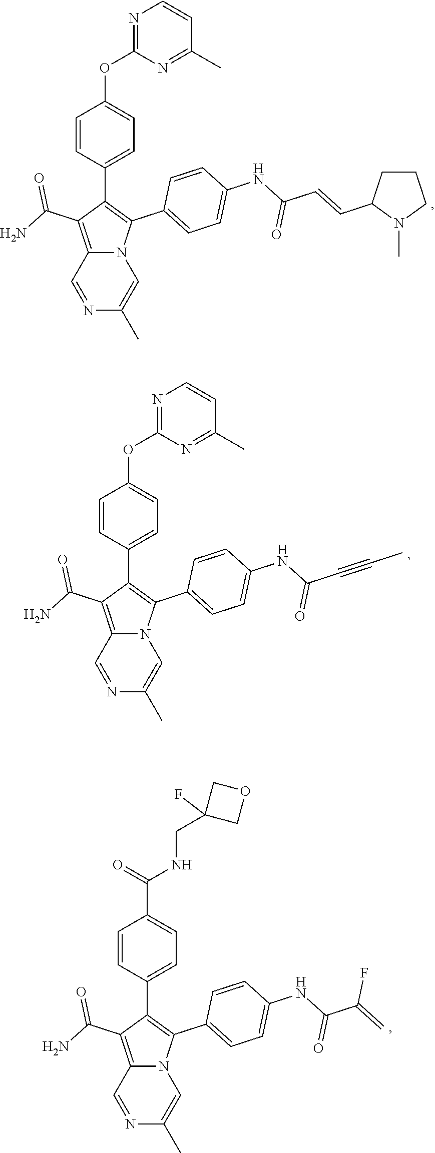 [SMILES: Cc1cn2c(-c3ccc(NC(=O)/C=C/C4CCCN4C)cc3)c(-c3ccc(Oc4nccc(C)n4)cc3)c(C(N)=O)c2cn1 ; CC#CC(=O)Nc1ccc(-c2c(-c3ccc(Oc4nccc(C)n4)cc3)c(C(N)=O)c3cnc(C)cn23)cc1 ; C=C(F)C(=O)Nc1ccc(-c2c(-c3ccc(C(=O)NCC4(F)COC4)cc3)c(C(N)=O)c3cnc(C)cn23)cc1]

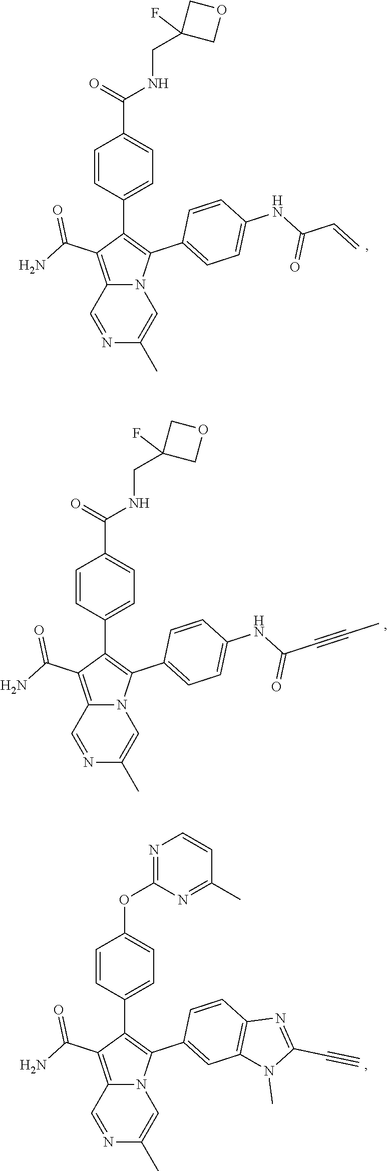 [SMILES: C=CC(=O)Nc1ccc(-c2c(-c3ccc(C(=O)NCC4(F)COC4)cc3)c(C(N)=O)c3cnc(C)cn23)cc1 ; CC#CC(=O)Nc1ccc(-c2c(-c3ccc(C(=O)NCC4(F)COC4)cc3)c(C(N)=O)c3cnc(C)cn23)cc1 ; C#Cc1nc2ccc(-c3c(-c4ccc(Oc5nccc(C)n5)cc4)c(C(N)=O)c4cnc(C)cn34)cc2n1C]

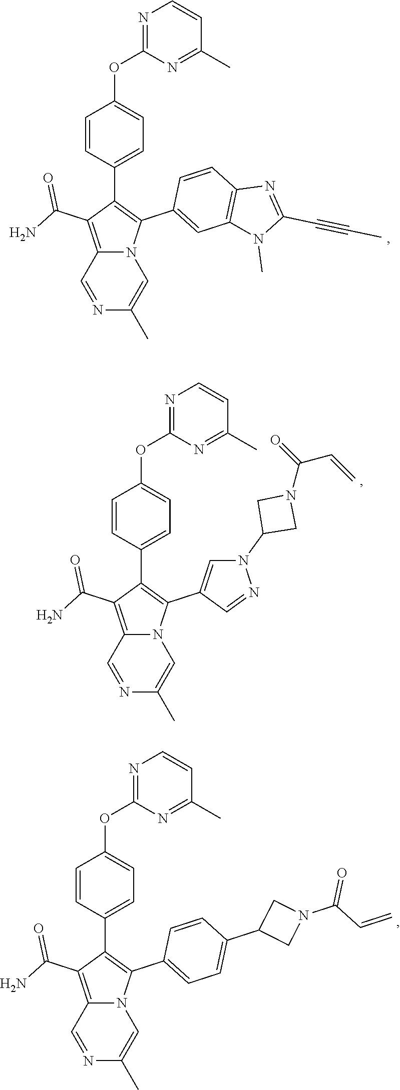

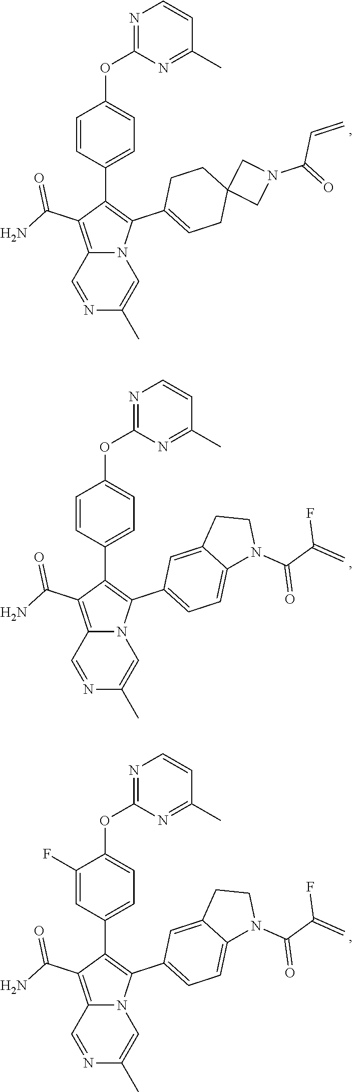

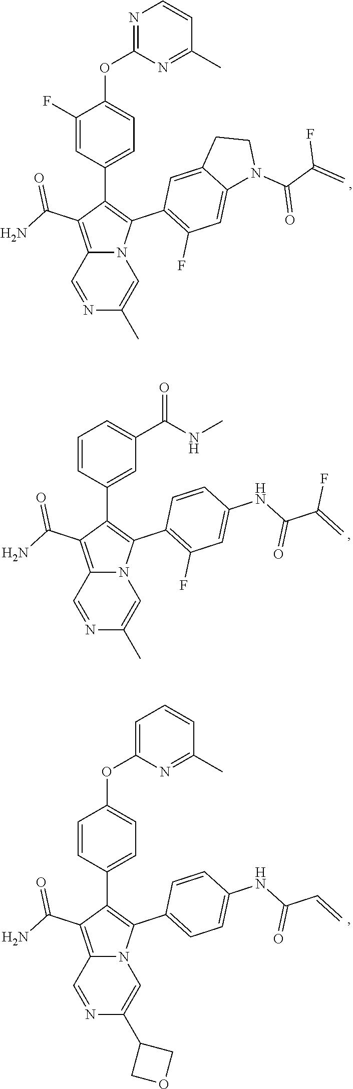 [SMILES: C=C(F)C(=O)N1CCc2cc(-c3c(-c4ccc(Oc5nccc(C)n5)c(F)c4)c(C(N)=O)c4cnc(C)cn34)c(F)cc21 ; C=C(F)C(=O)Nc1ccc(-c2c(-c3cccc(C(=O)NC)c3)c(C(N)=O)c3cnc(C)cn23)c(F)c1 ; C=CC(=O)Nc1ccc(-c2c(-c3ccc(Oc4cccc(C)n4)cc3)c(C(N)=O)c3cnc(C4COC4)cn23)cc1]

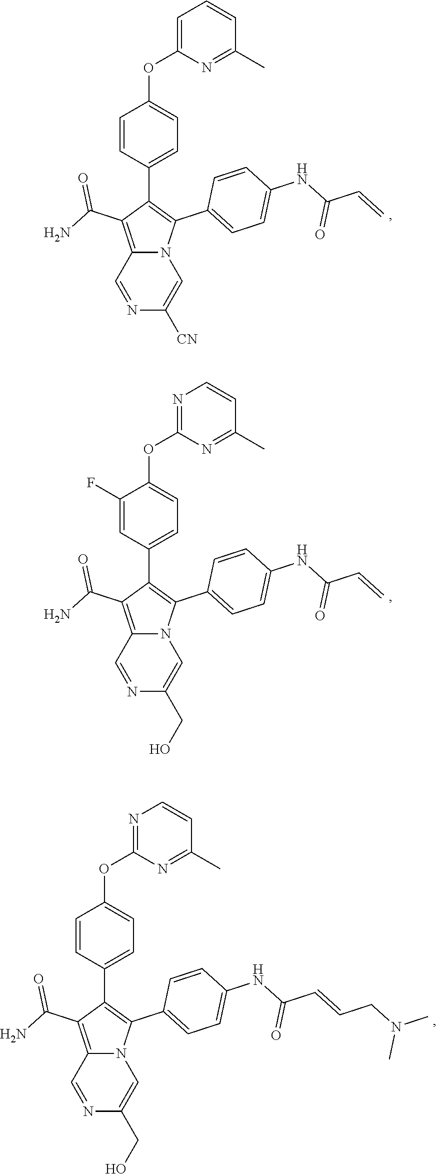 [SMILES: C=CC(=O)Nc1ccc(-c2c(-c3ccc(Oc4cccc(C)n4)cc3)c(C(N)=O)c3cnc(C#N)cn23)cc1 ; C=CC(=O)Nc1ccc(-c2c(-c3ccc(Oc4nccc(C)n4)c(F)c3)c(C(N)=O)c3cnc(CO)cn23)cc1 ; Cc1ccnc(Oc2ccc(-c3c(C(N)=O)c4cnc(CO)cn4c3-c3ccc(NC(=O)/C=C/CN(C)C)cc3)cc2)n1]

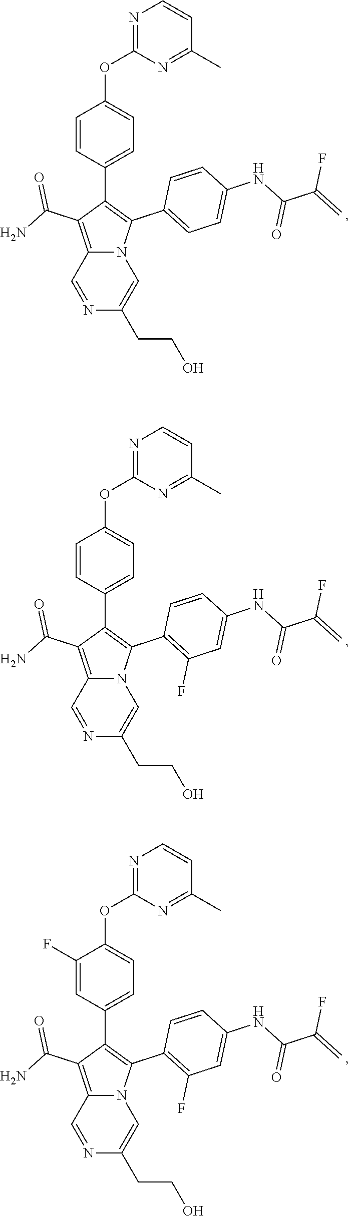 [SMILES: C=C(F)C(=O)Nc1ccc(-c2c(-c3ccc(Oc4nccc(C)n4)cc3)c(C(N)=O)c3cnc(CCO)cn23)cc1 ; C=C(F)C(=O)Nc1ccc(-c2c(-c3ccc(Oc4nccc(C)n4)cc3)c(C(N)=O)c3cnc(CCO)cn23)c(F)c1 ; C=C(F)C(=O)Nc1ccc(-c2c(-c3ccc(Oc4nccc(C)n4)c(F)c3)c(C(N)=O)c3cnc(CCO)cn23)c(F)c1]

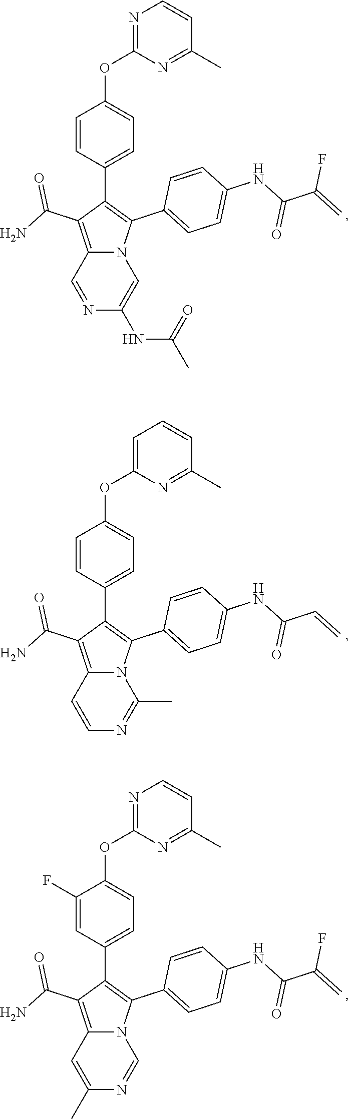 [SMILES: C=C(F)C(=O)Nc1ccc(-c2c(-c3ccc(Oc4nccc(C)n4)cc3)c(C(N)=O)c3cnc(NC(C)=O)cn23)cc1 ; C=CC(=O)Nc1ccc(-c2c(-c3ccc(Oc4cccc(C)n4)cc3)c(C(N)=O)c3ccnc(C)n23)cc1 ; C=C(F)C(=O)Nc1ccc(-c2c(-c3ccc(Oc4nccc(C)n4)c(F)c3)c(C(N)=O)c3cc(C)ncn23)cc1]

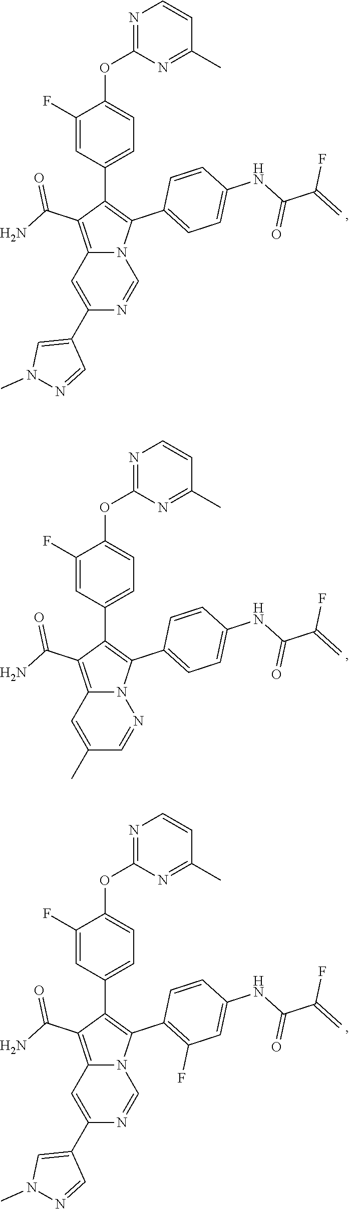

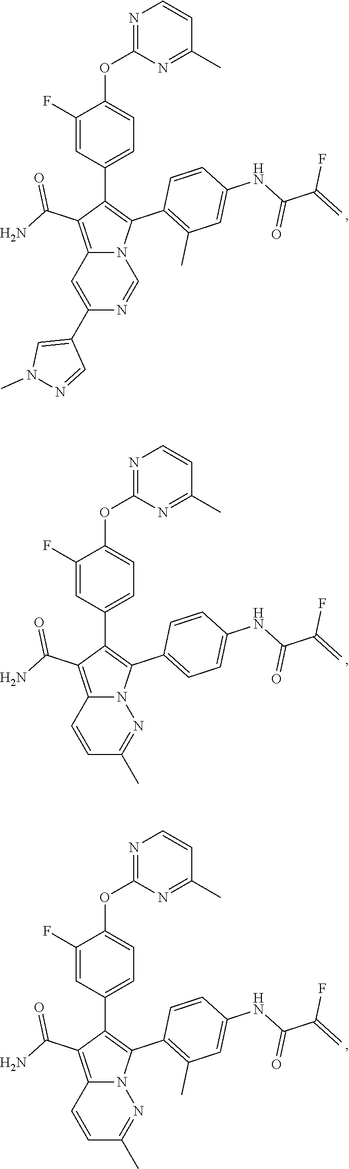 [SMILES: C=C(F)C(=O)Nc1ccc(-c2c(-c3ccc(Oc4nccc(C)n4)c(F)c3)c(C(N)=O)c3cc(-c4cnn(C)c4)ncn23)c(C)c1 ; C=C(F)C(=O)Nc1ccc(-c2c(-c3ccc(Oc4nccc(C)n4)c(F)c3)c(C(N)=O)c3ccc(C)nn23)cc1 ; C=C(F)C(=O)Nc1ccc(-c2c(-c3ccc(Oc4nccc(C)n4)c(F)c3)c(C(N)=O)c3ccc(C)nn23)c(C)c1]

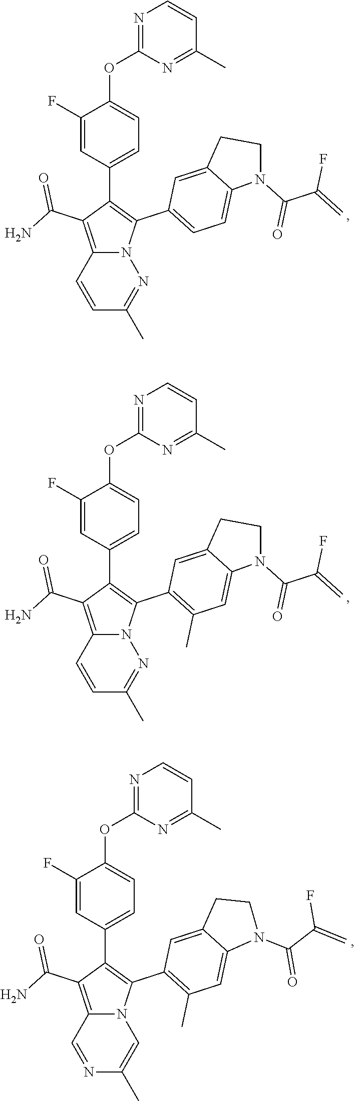

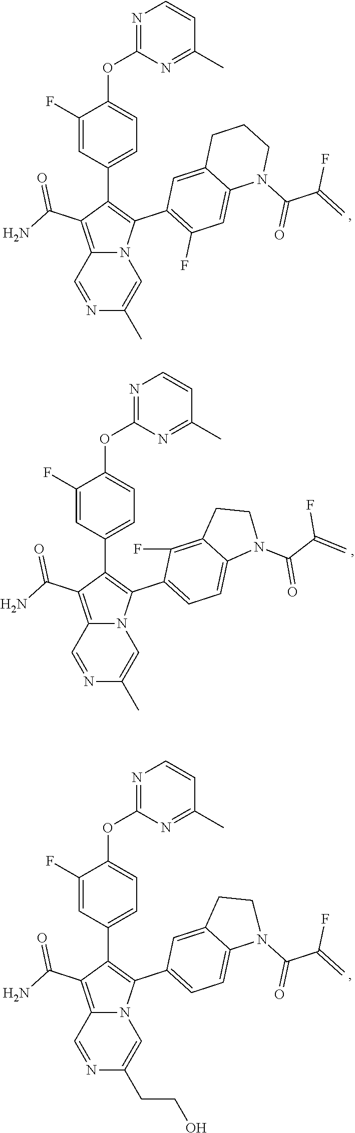

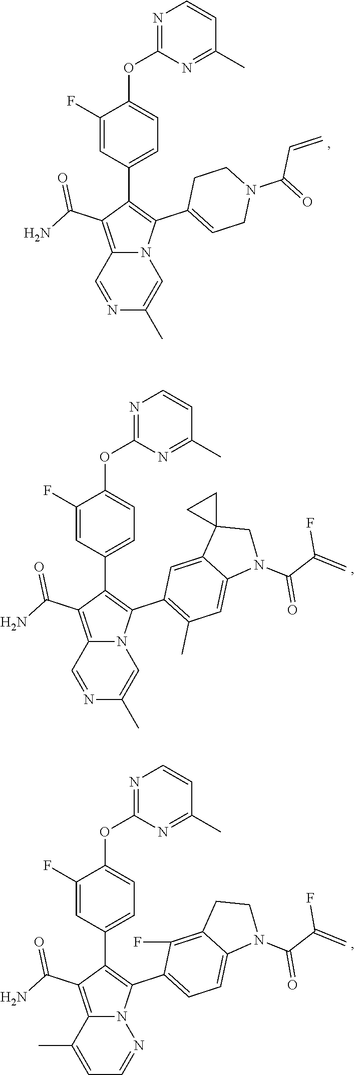

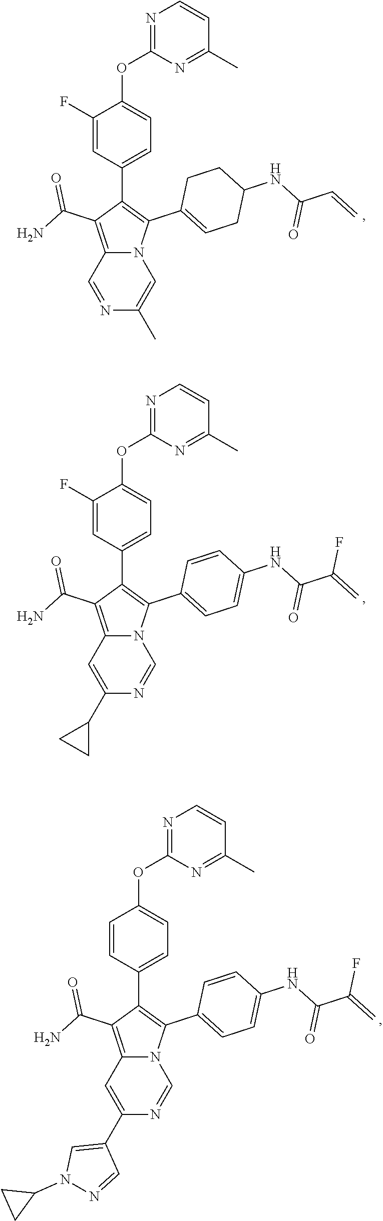 [SMILES: C=CC(=O)NC1CC=C(c2c(-c3ccc(Oc4nccc(C)n4)c(F)c3)c(C(N)=O)c3cnc(C)cn23)CC1 ; C=C(F)C(=O)Nc1ccc(-c2c(-c3ccc(Oc4nccc(C)n4)c(F)c3)c(C(N)=O)c3cc(C4CC4)ncn23)cc1 ; C=C(F)C(=O)Nc1ccc(-c2c(-c3ccc(Oc4nccc(C)n4)cc3)c(C(N)=O)c3cc(-c4cnn(C5CC5)c4)ncn23)cc1]

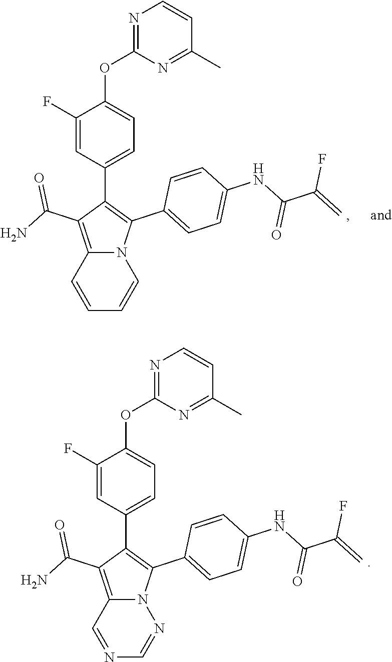 [SMILES: C=C(F)C(=O)Nc1ccc(-c2c(-c3ccc(Oc4nccc(C)n4)c(F)c3)c(C(N)=O)c3ccccn23)cc1 ; C=C(F)C(=O)Nc1ccc(-c2c(-c3ccc(Oc4nccc(C)n4)c(F)c3)c(C(N)=O)c3cncnn23)cc1]

In some embodiments, disclosed herein is a compound, or a pharmaceutically acceptable salt or stereoisomer thereof, selected from:

[chemical structure]

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine, and chloride, such as ²H (D), ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts or stereoisomers thereof which contain the aforementioned isotopes and/or other isotopes of other atoms are within the scope of this disclosure. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability.

In some embodiments, the abundance of deuterium in each of the substituents disclosed herein is independently at least 1%, at least 10%, at least 20%, at least 30%, at least 40%, at least 50%, at least 60%, at least 70%, at least 80%, at least 90%, or 100% by molar. In some embodiments, one or more of the substituents disclosed herein comprise deuterium at a percentage higher than the natural abundance of deuterium. In some embodiments, one or more 1H are replaced with one or more deuteriums in one or more of the substituents disclosed herein.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or stereoisomer thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undecanoate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein or stereoisomer thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Method of Treatment

Disclosed herein is a method of treating a disease in which inhibition of FGFR2 and/or FGFR3 is beneficial, the method comprising administering a compound of Formula (I), (Ia), (Ia′), (Ia″), (Ib), (Ib′), (Ib″), (Ic), (Ic′), (Ic″), (Id), or (Ie) disclosed herein, or a pharmaceutically acceptable salt or stereoisomer thereof. In some embodiments disclosed herein is a method of treating a disease in which inhibition of FGFR2 is beneficial, the method comprising administering a compound of Formula (I), (Ia), (Ia′), (Ia″), (Ib), (Ib′), (Ib″), (Ic), (Ic′), (Ic″), (Id), or (Ie) disclosed herein, or a pharmaceutically acceptable salt or stereoisomer thereof. In some embodiments disclosed herein is a method of treating a disease in which inhibition of FGFR3 is beneficial, the method comprising administering a compound of Formula (I), (Ia), (Ia′), (Ia″), (Ib), (Ib′), (Ib″), (Ic), (Ic′), (Ic″), (Id), or (Ie) disclosed herein, or a pharmaceutically acceptable salt or stereoisomer thereof. In some embodiments disclosed herein is a method of treating a disease in which inhibition of FGFR2 and FGFR3 is beneficial, the method comprising administering a compound of Formula (I), (Ia), (Ia′), (Ia″), (Ib), (Ib′), (Ib″), (Ic), (Ic′), (Ic″), (Id), or (Ie) disclosed herein, or a pharmaceutically acceptable salt or stereoisomer thereof.

Disclosed herein is a method of treating a disease or disorder associated with FGFR2 and/or FGFR3, the method comprising administering to the subject a compound of Formula (I), (Ia), (Ia′), (Ia″), (Ib), (Ib′), (Ib″), (Ic), (Ic′), (Ic″), (Id), or (Ie) disclosed herein, or a pharmaceutically acceptable salt or stereoisomer thereof. In some embodiments disclosed herein is a method of treating a disease or disorder associated with FGFR2, the method comprising administering to the subject a compound of Formula (I), (Ia), (Ia′), (Ia″), (Ib), (Ib′), (Ib″), (Ic), (Ic′), (Ic″), (Id), or (Ie) disclosed herein, or a pharmaceutically acceptable salt or stereoisomer thereof. In some embodiments disclosed herein is a method of treating a disease or disorder associated with FGFR3, the method comprising administering to the subject a compound of Formula (I), (Ia), (Ia′), (Ia″), (Ib), (Ib′), (Ib″), (Ic), (Ic′), (Ic″), (Id), or (Ie) disclosed herein, or a pharmaceutically acceptable salt or stereoisomer thereof. In some embodiments disclosed herein is a method of treating a disease or disorder associated with FGFR2 and FGFR3, the method comprising administering to the subject a compound of Formula (I), (Ia), (Ia′), (Ia″), (Ib), (Ib′), (Ib″), (Ic), (Ic′), (Ic″), (Id), or (Ie) disclosed herein, or a pharmaceutically acceptable salt or stereoisomer thereof.

Disclosed herein is a method of treating cancer in a subject, the method comprising administering to the subject a compound of Formula (I), (Ia), (Ia′), (Ia″), (Ib), (Ib′), (Ib″), (Ic), (Ic′), (Ic″), (Id), or (Ie) disclosed herein, or a pharmaceutically acceptable salt or stereoisomer thereof. In some embodiments is a method of treating cancer in a subject, the method comprising administering to the subject a compound of Formula (I), (Ia), (Ia′), (Ia″), (Ib), (Ib′), (Ib″), (Ic), (Ic′), (Ic″), (Id), or (Ie) disclosed herein, or a pharmaceutically acceptable salt or stereoisomer thereof, wherein the cancer is selected from intra-hepatic cholangiocarcinoma, urothelial cancer, gastric cancer, bladder cancer, breast cancer, endometrial cancer, kidney cancer, liver cancer, lung cancer, melanoma, pancreatic cancer, prostate cancer, or thyroid cancer. In some embodiments is a method of treating intra-hepatic cholangiocarcinoma in a subject, the method comprising administering to the subject a compound of Formula (I), (Ia), (Ia′), (Ia″), (Ib), (Ib′), (Ib″), (Ic), (Ic′), (Ic″), (Id), or (Ie) disclosed herein, or a pharmaceutically acceptable salt or stereoisomer thereof. In some embodiments is a method of treating urothelial cancer in a subject, the method comprising administering to the subject a compound of Formula (I), (Ia), (Ia′), (Ia″), (Ib), (Ib′), (Ib″), (Ic), (Ic′), (Ic″), (Id), or (Ie) disclosed herein, or a pharmaceutically acceptable salt or stereoisomer thereof. In some embodiments is a method of treating gastric cancer in a subject, the method comprising administering to the subject a compound of Formula (I), (Ia), (Ia′), (Ia″), (Ib), (Tb′), (Ib″), (Ic), (Ic′), (Ic″), (Id), or (Ie) disclosed herein, or a pharmaceutically acceptable salt or stereoisomer thereof. In some embodiments is a method of treating bladder cancer in a subject, the method comprising administering to the subject a compound of Formula (I), (Ia), (Ia′), (Ia″), (Ib), (Ib′), (Ib″), (Ic), (Ic′), (Ic″), (Id), or (Ie) disclosed herein, or a pharmaceutically acceptable salt or stereoisomer thereof. In some embodiments is a method of treating breast cancer in a subject, the method comprising administering to the subject a compound of Formula (I), (Ia), (Ia′), (Ia″), (Ib), (Ib′), (Ib″), (Ic), (Ic′), (Ic″), (Id), or (Ie) disclosed herein, or a pharmaceutically acceptable salt or stereoisomer thereof. In some embodiments is a method of treating endometrial cancer in a subject, the method comprising administering to the subject a compound of Formula (I), (Ia), (Ia′), (Ia″), (Ib), (Ib′), (Ib″), (Ic), (Ic′), (Ic″), (Id), or (Ie) disclosed herein, or a pharmaceutically acceptable salt or stereoisomer thereof. In some embodiments is a method of treating kidney cancer in a subject, the method comprising administering to the subject a compound of Formula (I), (Ia), (Ia′), (Ia″), (Ib), (Ib′), (Ib″), (Ic), (Ic′), (Ic″), (Id), or (Ie) disclosed herein, or a pharmaceutically acceptable salt or stereoisomer thereof. In some embodiments is a method of treating liver cancer in a subject, the method comprising administering to the subject a compound of Formula (I), (Ia), (Ia′), (Ia″), (Ib), (Ib′), (Ib″), (Ic), (Ic′), (Ic″), (Id), or (Ie) disclosed herein, or a pharmaceutically acceptable salt or stereoisomer thereof. In some embodiments is a method of treating lung cancer in a subject, the method comprising administering to the subject a compound of Formula (I), (Ia), (Ia′), (Ia″), (Ib), (Ib′), (Ib″), (Ic), (Ic′), (Ic″), (Id), or (Ie) disclosed herein, or a pharmaceutically acceptable salt or stereoisomer thereof. In some embodiments is a method of treating melanoma in a subject, the method comprising administering to the subject a compound of Formula (I), (Ia), (Ia′), (Ia″), (Ib), (Ib′), (Ib″), (Ic), (Ic′), (Ic″), (Id), or (Ie) disclosed herein, or a pharmaceutically acceptable salt or stereoisomer thereof. In some embodiments is a method of treating pancreatic cancer in a subject, the method comprising administering to the subject a compound of Formula (I), (Ia), (Ia′), (Ia″), (Ib), (Ib′), (Ib″), (Ic), (Ic′), (Ic″), (Id), or (Ie) disclosed herein, or a pharmaceutically acceptable salt or stereoisomer thereof. In some embodiments is a method of treating prostate cancer in a subject, the method comprising administering to the subject a compound of Formula (I), (Ia), (Ia′), (Ia″), (Ib), (Ib′), (Ib″), (Ic), (Ic′), (Ic″), (Id), or (Ie) disclosed herein, or a pharmaceutically acceptable salt or stereoisomer thereof. In some embodiments is a method of treating thyroid cancer in a subject, the method comprising administering to the subject a compound of Formula (I), (Ia), (Ia′), (Ia″), (Ib), (Ib′), (Ib″), (Ic), (Ic′), (Ic″), (Id), or (Ie) disclosed herein, or a pharmaceutically acceptable salt or stereoisomer thereof.

### Dosing

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder, or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of or risk factor for the disease being treated and is currently in remission, a pharmaceutical composition comprising a compound described herein, or a pharmaceutically acceptable salt thereof, in order to prevent a return of the symptoms of the disease or condition.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage, or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₁₀ and the ED₉₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose or multiple doses; (ii) the time between multiple administrations is every 6-12 hours; (iii) the compound is administered to the mammal every 1 to 2 days; or (iv) the compound is administered to the subject every week or every month.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

### Pharmaceutical Compositions/Formulations

The compounds described herein are administered to a subject in need thereof, either alone or in combination with pharmaceutically acceptable carriers, excipients, or diluents, in a pharmaceutical composition, according to standard pharmaceutical practice. In some embodiments, the compounds of this disclosure may be administered to animals. The compounds can be administered orally or parenterally, including the intravenous, intramuscular, intraperitoneal, subcutaneous, rectal, and topical routes of administration.

In another aspect, provided herein are pharmaceutical compositions comprising a compound of Formula (I), (Ia), (Ia′), (Ia″), (Ib), (Ib′), (Ib″), (Ic), (Ic′), (Ic″), (Id), or (Ie) described herein, or a pharmaceutically acceptable salt or stereoisomer thereof, and at least one pharmaceutically acceptable excipient. In some embodiments is a pharmaceutical compositions comprising a compound of Formula (I) described herein, or a pharmaceutically acceptable salt or stereoisomer thereof, and at least one pharmaceutically acceptable excipient. In some embodiments is a pharmaceutical compositions comprising a compound of Formula (Ia) described herein, or a pharmaceutically acceptable salt or stereoisomer thereof, and at least one pharmaceutically acceptable excipient. In some embodiments is a pharmaceutical compositions comprising a compound of Formula (Ib) described herein, or a pharmaceutically acceptable salt or stereoisomer thereof, and at least one pharmaceutically acceptable excipient. In some embodiments is a pharmaceutical compositions comprising a compound of Formula (Ic) described herein, or a pharmaceutically acceptable salt or stereoisomer thereof, and at least one pharmaceutically acceptable excipient. In some embodiments is a pharmaceutical compositions comprising a compound of Formula (Id) described herein, or a pharmaceutically acceptable salt or stereoisomer thereof, and at least one pharmaceutically acceptable excipient. In some embodiments is a pharmaceutical compositions comprising a compound of Formula (Ie) described herein, or a pharmaceutically acceptable salt or stereoisomer thereof, and at least one pharmaceutically acceptable excipient.

Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The pharmaceutical compositions described herein are administered to a subject by appropriate administration routes, including, but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

Pharmaceutical compositions including compounds described herein, or a pharmaceutically acceptable salt or stereoisomer thereof are manufactured in a conventional manner, such as, by way of example only, by means of conventional mixing, dissolving, granulating, dragee-making, levigating, emulsifying, encapsulating, entrapping, or compression processes.

Pharmaceutical compositions for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active compound doses.

Pharmaceutical compositions that are administered orally include push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as tale or magnesium stearate and, optionally, stabilizers. In soft capsules, the active compounds are dissolved or suspended in suitable liquids, such as fatty oils, liquid paraffin, or liquid polyethylene glycols. In some embodiments, stabilizers are added.

Pharmaceutical compositions for parental use are formulated as infusions or injections. In some embodiments, the pharmaceutical composition suitable for injection or infusion includes sterile aqueous solutions, or dispersions, or sterile powders comprising a compound described herein, or a pharmaceutically acceptable salt or stereoisomer thereof. In some embodiments, the pharmaceutical composition comprises a liquid carrier. In some embodiments, the liquid carrier is a solvent or liquid dispersion medium comprising, for example, water, saline, ethanol, a polyol (for example, glycerol, propylene glycol, liquid polyethylene glycols, and the like), vegetable oils, nontoxic glyceryl esters, and any combinations thereof. In some embodiments, the pharmaceutical compositions further comprise a preservative to prevent growth of microorganisms.

### EXAMPLES

The following examples are provided for illustrative purposes only and not to limit the scope of the claims provided herein.

As used above, and throughout the description of the disclosure, the following abbreviations, unless otherwise indicated, shall be understood to have the following meanings:
ACN or MeCN acetonitrile
AcOH acetic acid
Ac acetyl
Bn benzyl
BOC or Boc tert-butyl carbamate
i-Bu iso-butyl
t-Bu tert-butyl
CDI 1,1-carbonyldiimidazole
DBU 1,8-diazabicyclo[5.4.0]undec-7-ene
DCE dichloroethane (ClCH₂CH₂Cl)
DCM dichloromethane (CH₂Cl₂)
DIBAL-H diisobutylaluminum hydride
DIPEA or DIEA diisopropylethylamine
DMAP 4-(N,N-dimethylamino)pyridine
DME 1,2-dimethoxyethane
DMF N,N-dimethylformamide
DMA N,N-dimethylacetamide
DMPU N,N′-dimethylpropyleneurea
DMSO dimethylsulfoxide
DPPA diphenyl phosphoryl azide
Dppf or dppf 1,1′-bis(diphenylphosphino) ferrocene
EDC or EDCI N-(3-dimethylaminopropyl)-N′-ethylcarbodiimide hydrochloride
eq equivalent(s)
Et ethyl
Et₂O diethyl ether
EtOH ethanol
EA or EtOAc ethyl acetate
HATU 1-[bis(dimethylamino)methylene]-1H-1,2,3-triazolo[4,5-b]pyridinium 3-oxid hexafluorophosphate
HOBt 1-hydroxybenzotriazole
HPLC high performance liquid chromatography
KOAc potassium acetate
KOtBu potassium tert-butoxide
KHMDS potassium bis(trimethylsilyl)amide
NaHMDS sodium bis(trimethylsilyl)amide
LiHMDS lithium bis(trimethylsilyl)amide
LAH/LiAlH₄ lithium aluminum anhydride
LCMS liquid chromatography mass spectrometry
Me methyl
MeOH methanol
MS mass spectroscopy
MTBE methyl tert-butyl ether
NBS N-bromosuccinimide
NMP N-methyl-pyrrolidin-2-one
NMR nuclear magnetic resonance
PE petroleum ether
Ph phenyl
iPr/i-Pr iso-propyl
PyAOP 7-Azabenzotriazol-1-yloxy)tripyrrolidinophosphonium hexafluorophosphate
RP-HPLC reverse-phase high-pressure liquid chromatography
rt room temperature
SEM 2-(trimethylsilyl) ethoxymethyl
tert-butyldimethylsilyl TBS
TEA triethylamine
TFA trifluoroacetic acid
THF tetrahydrofuran
TLC thin layer chromatography
TMS trimethylsilyl

### Example 1:6-(4-acrylamidophenyl)-7-(4-((6-methylpyridin-2-yl)oxy)phenyl)pyrrolo[1,2-a]pyrazine-8-carboxamide

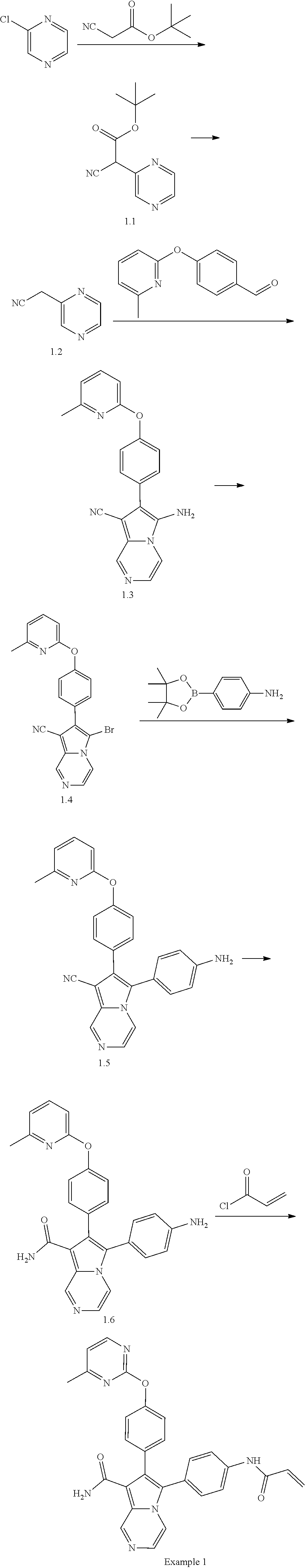

### Step 1. tert-butyl 2-cyano-2-(pyrazin-2-yl)acetate

To a solution of 2-chloropyrazine (8.0 g, 69.85 mmol) in THF (250 mL) were added tert-butyl cyanoacetate (19.72 g, 139.70 mmol) and t-BuOK (19.59 g, 174.62 mmol). The reaction mixture was stirred at 80° C. for 48 h. After cooling to rt, the mixture was poured into water (300 mL) and extracted with EtOAc (3×300 mL) to remove the impurity. The water layer was adjusted to pH˜3 with 1N HCl solution and extracted with DCM (3×300 mL). The combined organic layers were washed with brine, dried over Na₂SO₄, filtered, and concentrated under reduced pressure to afford the title compound (9.5 g, 62%). LC-MS (ESI+): m/z 164.0 [M+H−56]⁺.

### Step 2. 2-(pyrazin-2-yl) acetonitrile

To a solution of tert-butyl 2-cyano-2-(pyrazin-2-yl)acetate (4.5 g, 20.53 mmol) in DCM (20 mL) was added TFA (20 mL). The reaction mixture was stirred at rt for 5 h. Then the mixture was poured into saturated NaHCO₃ solution slowly (300 mL) and extracted with EtOAc (3×100 mL). The combined organic layers were washed with brine, dried over Na₂SO₄, filtered, and concentrated under reduced pressure to give a residue which was purified by flash column chromatography on silica gel (eluting with PE:EtOAc=1:1) to afford the title compound (1.1 g, 45%) as a yellow oil. ¹H NMR (400 MHZ, DMSO-d₆) δ 8.78-8.53 (m, 3H), 4.33 (s, 2H).

### Step 3. 6-amino-7-(4-((6-methylpyridin-2-yl)oxy)phenyl)pyrrolo[1,2-a]pyrazine-8-carbonitrile

To a solution of 2-(pyrazin-2-yl) acetonitrile (500 mg, 4.20 mmol) in MeOH (15 mL) were added 4-((6-methylpyridin-2-yl)oxy)benzaldehyde (895 mg, 4.20 mmol), DBU (1.9 g, 12.60 mmol) and acetyl cyanide (290 mg, 4.20 mmol). The reaction mixture was stirred at 120° C. for 20 min under microwave irradiation. After cooling to rt, the mixture was quenched by water (100 mL) and extracted with DCM (3×100 mL). The combined organic layers were washed with brine, dried over Na₂SO₄, filtered, and concentrated under reduced pressure to give a residue which was purified by flash column chromatography on silica gel (eluting with MeOH:DCM=10:1) to afford the title compound (0.9 g, 63%). LC-MS (ESI+): m/z 342.2 [M+H]⁺.

### Step 4. 6-bromo-7-(4-((6-methylpyridin-2-yl)oxy)phenyl)pyrrolo[1,2-a]pyrazine-8-carbonitrile

To a solution of 6-amino-7-(4-((6-methylpyridin-2-yl)oxy)phenyl)pyrrolo[1,2-a]pyrazine-8-carbonitrile (1.3 g, 3.81 mmol) in MeCN (40 mL) were added tert-butyl nitrite (1.18 g, 11.42 mmol) and CuBr₂ (1.28 g, 5.71 mmol). The reaction mixture was stirred at rt for 0.5 h. Then the mixture was concentrated under reduced pressure to give a residue which was purified by prep-HPLC (eluted with 5% to 70% MeCN in H₂O containing 0.1% NH₄OH) to afford the title compound (170 mg, 11%). LC-MS (ESI+): m/z 405.0 [M+H]⁺.

### Step 5. 6-(4-aminophenyl)-7-(4-((6-methylpyridin-2-yl)oxy)phenyl)pyrrolo[1,2-a]pyrazine-8-carbonitrile

To a solution of 6-bromo-7-(4-((6-methylpyridin-2-yl)oxy)phenyl)pyrrolo[1,2-a]pyrazine-8-carbonitrile (170 mg, 0.42 mmol) in 1,4-dixane (3 mL) and water (0.3 mL) were added 4-aminophenylboronic acid pinacol ester (137.86 mg, 0.63 mmol), K₂CO₃ (116 mg, 0.84 mmol) and Pd(dppf)Cl₂ (31 mg, 0.04 mmol). The mixture was stirred at 100° C. for 1 h under microwave irradiation and N₂. After cooling to rt, the mixture was quenched by water (30 mL) and extracted with EtOAc (3×30 mL). The combined organic layers were washed with brine, dried over Na₂SO₄, filtered, and concentrated under reduced pressure to give a residue which was purified by flash column chromatography on silica gel (eluting with MeOH:DCM=20:1) to afford the title compound (140 mg, 81%). LC-MS (ESI+): m/z 418.2 [M+H]⁺.

### Step 6. 6-(4-aminophenyl)-7-(4-((6-methylpyridin-2-yl)oxy)phenyl)pyrrolo[1,2-a]pyrazine-8-carboxamide

A mixture of 6-(4-aminophenyl)-7-(4-((6-methylpyridin-2-yl)oxy)phenyl)pyrrolo[1,2-a]pyrazine-8-carbonitrile (130 mg, 0.31 mmol) and concentrated H₂SO₄ (6 mL) was stirred at 30° C. for 8 h. The mixture was poured into water (100 mL) slowly, adjusted to pH˜8 with 6N NaOH solution and extracted with DCM (3×100 mL). The combined organic layers were washed with brine, dried over Na₂SO₄, filtered, and concentrated under reduced pressure to give a crude product (130 mg, 97%). LC-MS (ESI+): m/z 436.2 [M+H]⁺.

### Step 7. 6-(4-acrylamidophenyl)-7-(4-((6-methylpyridin-2-yl)oxy)phenyl)pyrrolo[1,2-a]pyrazine-8-carboxamide

To a solution of 6-(4-aminophenyl)-7-(4-((6-methylpyridin-2-yl)oxy)phenyl)pyrrolo[1,2-a]pyrazine-8-carboxamide (130 mg, 0.30 mmol) in acetone (5 mL) were added K₂CO₃ solution (1.65 mL, 0.5 mol/L in water) and acryloyl chloride (0.55 mL, 0.5 mol/L in acetone) at 0° C. The mixture was stirred at 0° C. for 1 h. The mixture was quenched by water (50 mL) and extracted with DCM (3×50 mL). The combined organic layers were washed with brine, dried over Na₂SO₄, filtered, and concentrated under reduced pressure to give a residue which was purified by prep-HPLC (eluted with 26% to 36% MeCN in H₂O containing 0.1% FA) to afford the title compound (16 mg, 12%). LC-MS (ESI+): m/z 490.2 [M+H]⁺. ¹H NMR (400 MHZ, DMSO-d₆) δ 10.31 (s, 1H), 9.33 (s, 1H), 8.05 (m, 1H), 7.84-7.71 (m, 3H), 7.67 (m, 1H), 7.41 (bs, 1H), 7.36-7.25 (m, 4H), 7.07 (m, 2H), 7.02 (m, 1H), 6.76 (m, 1H), 6.65 (bs, 1H), 6.45 (m, 1H), 6.28 (m, 1H), 5.78 (m, 1H), 2.34 (s, 3H).

### Example 2:6-(4-acrylamidophenyl)-7-(4-((4-methylpyrimidin-2-yl)oxy)phenyl)pyrrolo[1,2-a]pyrazine-8-carboxamide

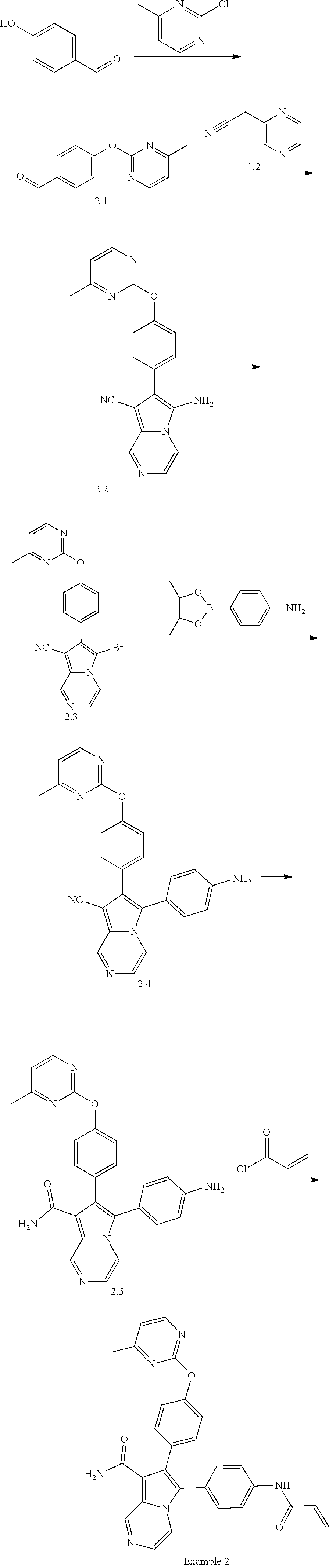

### Step 1. 4-((4-methylpyrimidin-2-yl)oxy)benzaldehyde

To a solution of 4-hydroxybenzaldehyde (500 mg, 4.09 mmol) and 2-chloro-4-methylpyrimidine (632 mg, 4.91 mmol) in DMF (10 mL) was added CsF (1.87 g, 12.28 mmol). The reaction mixture was stirred at 120° C. for 12 hrs. After cooling to rt, the reaction mixture was concentrated under reduced pressure to give a residue which was purified by flash column chromatography on silica gel (eluting with 0˜50% ethyl acetate in petroleum ether) to afford the title compound (380 mg, 43%). LC-MS (ESI+): m/z 215.4 [M+H]⁺.

### Step 2. 6-amino-7-(4-((4-methylpyrimidin-2-yl)oxy)phenyl)pyrrolo[1,2-a]pyrazine-8-carbonitrile

To a mixture of 4-((4-methylpyrimidin-2-yl)oxy)benzaldehyde (576 mg, 2.69 mmol) and 2-(pyrazin-2-yl) acetonitrile (320 mg, 2.69 mmol) in MeOH (10 mL) were added DBU (1.23 g, 8.06 mmol) and acetyl cyanide (186 mg, 2.69 mmol). The reaction mixture was stirred at 100° C. under microwave irradiation for 20 mins under N₂. After cooling to rt, the reaction mixture was quenched by water 20 mL and extracted with ethyl acetate (30 mL*3). The combined organic layers were washed with brine, dried over Na₂SO₄, filtered, and concentrated under reduced pressure to give a residue which was purified by flash column chromatography on silica gel (eluting with 0˜10% methanol in dichloromethane) to afford the title compound (450 mg, 49%). LC-MS (ESI+): m/z 343.0 [M+H]⁺.

### Step 3. 6-bromo-7-(4-((4-methylpyrimidin-2-yl)oxy)phenyl)pyrrolo[1,2-a]pyrazine-8-carbonitrile

To a solution of 6-amino-7-(4-((4-methylpyrimidin-2-yl)oxy)phenyl)pyrrolo[1,2-a]pyrazine-8-carbonitrile (450 mg, 1.31 mmol) in HBr (10 mL, 33% in AcOH) was added CuBr₂ (382 mg, 1.71 mmol) at 0° C. Then a solution of NaNO₂ (454 mg, 6.57 mmol) in H₂O (4 mL) was added to the mixture. The reaction mixture was stirred at 0° C. for 0.5 hr. Then the pH of the solution was adjusted to 7˜8 by Na₂CO₃ (aq.). The mixture was extracted with DCM (20 ml*3). The combined organic layers were washed with brine, dried over Na₂SO₄, filtered, and concentrated under reduced pressure to give a residue which was purified by flash column chromatography on silica gel (eluting with 0˜10% methanol in dichloromethane) to afford the title compound (280 mg, 53%). LC-MS (ESI+): m/z 405.8 [M+H]⁺.

### Step 4. 6-(4-aminophenyl)-7-(4-((4-methylpyrimidin-2-yl)oxy)phenyl)pyrrolo[1,2-a]pyrazine-8-carbonitrile

A mixture of 6-bromo-7-(4-((4-methylpyrimidin-2-yl)oxy)phenyl)pyrrolo[1,2-a]pyrazine-8-carbonitrile (280 mg, 689 μmol), 4-(4,4,5,5-tetramethyl-1,3,2-dioxaborolan-2-yl) aniline (302 mg, 1.38 mmol), Na₂CO₃ (219 mg, 2.07 mmol) and Pd-118 (45 mg, 68.9 μmol) in dioxane (10 mL) and H₂O (1 mL) was degassed and purged with N₂ for 3 times. The mixture was stirred at 90° C. for 2 hrs under N₂ atmosphere. After cooling to rt, the reaction mixture was concentrated under reduced pressure to give a residue which was purified by flash column chromatography on silica gel (eluting with 0˜30% ethyl acetate in petroleum ether) to afford the title compound (180 mg, 62%). LC-MS (ESI+): m/z 419.1 [M+H]⁺.

### Step 5. 6-(4-aminophenyl)-7-(4-((4-methylpyrimidin-2-yl)oxy)phenyl)pyrrolo[1,2-a]pyrazine-8-carboxamide

A mixture of 6-(4-aminophenyl)-7-(4-((4-methylpyrimidin-2-yl)oxy)phenyl)pyrrolo[1,2-a]pyrazine-8-carbonitrile (180 mg, 430 μmol) in concentrated H₂SO₄ (3 mL) was stirred at 30° C. for 8 hrs. Then the mixture was poured into water (20 mL) slowly, adjusted to pH˜7 with Na₂CO₃ and extracted with EtOAc (3×20 mL). The combined organic layers were washed with brine, dried over Na₂SO₄, filtered, and concentrated under reduced pressure to afford the title compound (160 mg, 85%). LC-MS (ESI+): m/z 437.2 [M+H]⁺.

### Step 6. 6-(4-acrylamidophenyl)-7-(4-((4-methylpyrimidin-2-yl)oxy)phenyl)pyrrolo[1,2-a]pyrazine-8-carboxamide

To a solution of 6-(4-aminophenyl)-7-(4-((4-methylpyrimidin-2-yl)oxy)phenyl)pyrrolo[1,2-a]pyrazine-8-carboxamide (160 mg, 367 μmol) in Acetone (2 mL) and H₂O (0.4 mL) was added K₂CO₃ (102 mg, 734.8 μmol). Then prop-2-enoyl chloride (40 mg, 440.9 μmol) was added to the solution at 0° C. The reaction mixture was stirred at 25° C. for 1 hr. Then 10 mL of water was added to the mixture. After extraction with ethyl acetate (10 mL*3), the combined organic layers were washed with brine, dried over Na₂SO₄, filtered, and concentrated under reduced pressure to give a residue which was purified by flash column chromatography on silica gel (eluting with 0˜10% MeOH in DCM) to afford the title compound (26 mg, 15%). LC-MS (ESI+): m/z 491.2 [M+H]⁺. ¹H NMR (400 MHZ, DMSO-d₆) δ 10.33 (s, 1H), 9.28 (d, J=1.5 Hz, 1H), 8.47 (d, J=5.0 Hz, 1H), 7.99 (dd, J=1.4, 4.9 Hz, 1H), 7.77 (d, J=8.8 Hz, 2H), 7.66 (d, J=4.8 Hz, 1H), 7.44 (br s, 1H), 7.32 (m, 4H), 7.20-7.11 (m, 3H), 6.75 (br s, 1H), 6.51-6.39 (m, 1H), 6.33-6.20 (m, 1H), 5.83-5.75 (m, 1H), 2.41 (s, 3H).

### Example 3:6-(6-acrylamidopyridin-3-yl)-7-(4-((6-methylpyridin-2-yl)oxy)phenyl)pyrrolo[1,2-a]pyrazine-8-carboxamide

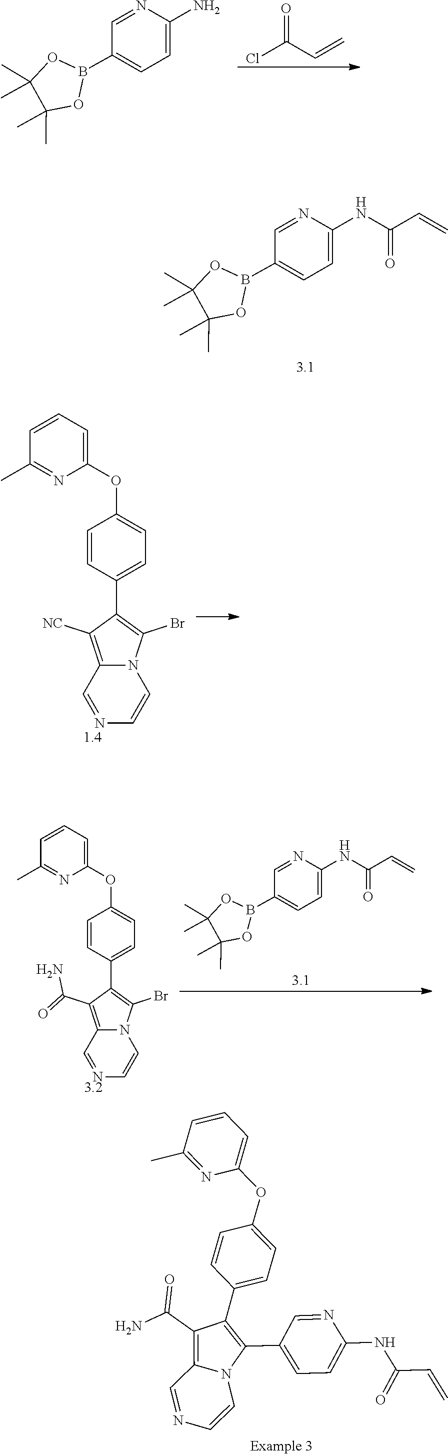

### Step 1. N-(5-(4,4,5,5-tetramethyl-1,3,2-dioxaborolan-2-yl) pyridin-2-yl) acrylamide

To a solution of 5-(4,4,5,5-tetramethyl-1,3,2-dioxaborolan-2-yl) pyridin-2-amine (200 mg, 908.8 μmol) and TEA (138 mg, 1.36 mmol, 189.7 μL) in DCM (3 mL) was added prop-2-enoyl chloride (82 mg, 908.8 μmol, 74.1 μL) at 0° C. The reaction mixture was stirred at 0° C. for 0.5 hr. Then the mixture was concentrated under reduced pressure to give a residue which was purified by flash column chromatography on silica gel (eluting with 0˜30% ethyl acetate in petroleum ether) to afford the title compound (100 mg, 40%) as a colorless oil. ¹H NMR (400 MHZ, DMSO-d₆) δ 10.92 (s, 1H), 8.55 (d, J=0.8 Hz, 1H), 8.22 (d, J=8.3 Hz, 1H), 8.01 (dd, J=1.8, 8.3 Hz, 1H), 6.68-6.54 (m, 1H), 6.33 (dd, J=1.8, 17.1 Hz, 1H), 5.84-5.72 (m, 1H), 1.31 (s, 12H).

### Step 2. 6-bromo-7-(4-((6-methylpyridin-2-yl)oxy)phenyl)pyrrolo[1,2-a]pyrazine-8-carboxamide

A mixture of 6-bromo-7-(4-((6-methylpyridin-2-yl)oxy)phenyl)pyrrolo[1,2-a]pyrazine-8-carbonitrile (100 mg, 246.8 μmol) in H₂SO₄ (2 mL) was stirred at 30° C. for 12 hrs. Then the mixture was poured into water (20 mL) slowly, adjusted to pH˜7 with Na₂CO₃ and extracted with EtOAc (3×20 mL). The combined organic layers were washed with brine, dried over Na₂SO₄, filtered, and concentrated under reduced pressure to give the title compound (100 mg). LC-MS (ESI+): m/z 423.0 [M+H]⁺.

### Step 3. 6-(6-acrylamidopyridin-3-yl)-7-(4-((6-methylpyridin-2-yl)oxy)phenyl)pyrrolo[1,2-a]pyrazine-8-carboxamide

A mixture of 6-bromo-7-(4-((6-methylpyridin-2-yl)oxy)phenyl)pyrrolo[1,2-a]pyrazine-8-carboxamide (100 mg, 236.3 μmol), N-(5-(4,4,5,5-tetramethyl-1,3,2-dioxaborolan-2-yl) pyridin-2-yl) acrylamide (65 mg, 236.3 μmol), K₂CO₃ (82 mg, 590.7 μmol) and Pd-118 (15 mg, 23.6 μmol) in dioxane (4 mL) and H₂O (1 mL) was degassed and purged with N₂ for 3 times. The mixture was stirred at 80° C. for 2 hrs. After cooling to rt, the mixture was diluted with water 10 mL and extracted with ethyl acetate (10 mL*3). The combined organic layers were washed with brine, dried over Na₂SO₄, filtered, and concentrated under reduced pressure to give a residue which was purified by prep-HPLC (column: Boston Prime C₁₈ 150*30 mm*5 um; mobile phase: [water (NH₃H₂O+NH₄HCO₃)-ACN]; B %: 30%-50%, 9 min) to afford the title compound (18 mg, 15%). LC-MS (ESI+): m/z 491.1 [M+H]⁺. ¹H NMR (400 MHz, DMSO-d₆) δ 10.89 (s, 1H), 9.35 (d, J=1.3 Hz, 1H), 8.28 (d, J=8.5 Hz, 1H), 8.24 (d, J=1.8 Hz, 1H), 8.11 (m, 1H), 7.87 (dd, J=2.3, 8.5 Hz, 1H), 7.79-7.67 (m, 2H), 7.43 (br s, 1H), 7.36-7.28 (m, 2H), 7.09 (d, J=8.8 Hz, 2H), 7.03 (d, J=7.5 Hz, 1H), 6.78 (d, J=8.3 Hz, 1H), 6.69-6.56 (m, 2H), 6.34 (m, 1H), 5.86-5.77 (m, 1H), 2.34 (s, 3H).

### Example 4:6-(6-acrylamido-4-methylpyridin-3-yl)-7-(4-((6-methylpyridin-2-yl)oxy)phenyl)pyrrolo[1,2-a]pyrazine-8-carboxamide

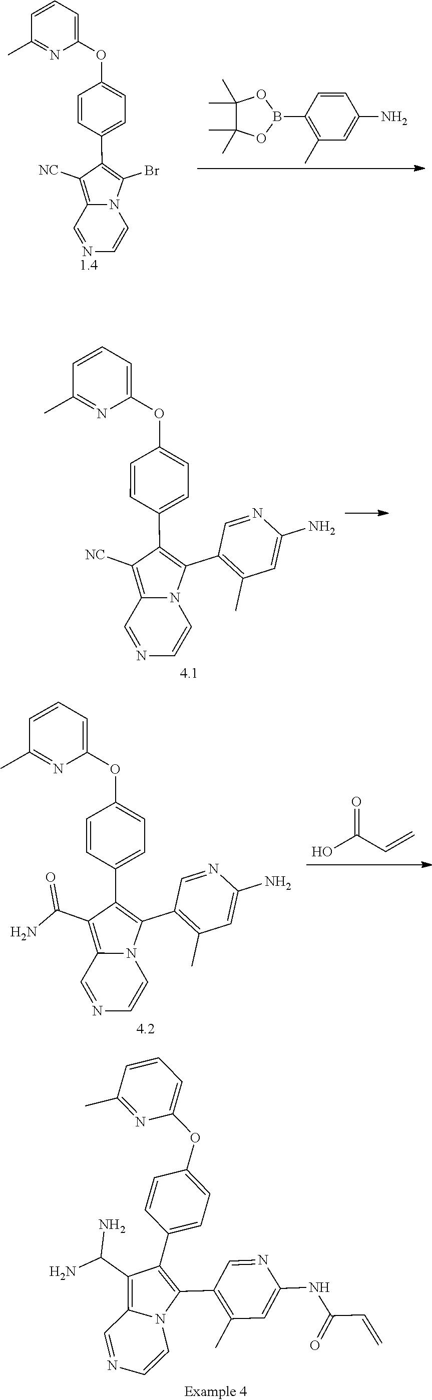

### Step 1. 6-(6-amino-4-methylpyridin-3-yl)-7-(4-((6-methylpyridin-2-yl)oxy)phenyl)pyrrolo[1,2-a]pyrazine-8-carbonitrile

To a solution of 6-bromo-7-(4-((6-methylpyridin-2-yl)oxy)phenyl)pyrrolo[1,2-a]pyrazine-8-carbonitrile (100 mg, 0.24 mmol) in 1,4-dioxane (10 mL) and water (0.1 mL) were added 4-methyl-5-(4,4,5,5-tetramethyl-1,3,2-dioxaborolan-2-yl) pyridin-2-amine (300 mg, 1.28 mmol), K₂CO₃ (102 mg, 0.74 mmol) and XPhos Pd G2 (19 mg, 0.03 mmol). The mixture was stirred at 100° C. for 1 h under microwave irradiation and N₂. After cooling to rt, the mixture was quenched by water (30 mL) and extracted with EtOAc (3×30 mL). The combined organic layers were washed with brine, dried over Na₂SO₄, filtered, and concentrated under reduced pressure to give a residue which was purified by flash column chromatography on silica gel (eluting with DCM:MeOH=20:1) to afford the title compound (65 mg, 63%). LC-MS (ESI+): m/z 433.2 [M+H]⁺.

### Step 2. 6-(6-amino-4-methylpyridin-3-yl)-7-(4-((6-methylpyridin-2-yl)oxy)phenyl)pyrrolo[1,2-a]pyrazine-8-carboxamide

A mixture of 6-(6-amino-4-methylpyridin-3-yl)-7-(4-((6-methylpyridin-2-yl)oxy)phenyl)pyrrolo[1,2-a]pyrazine-8-carbonitrile (65 mg, 0.15 mmol) and concentrated H₂SO₄ (3 mL) was stirred at 30° C. for 8 h. Then the mixture was poured into water (20 mL) slowly, adjusted to pH˜8 with 6N NaOH solution and extracted with DCM (3×30 mL). The combined organic layers were dried over Na₂SO₄, filtered, and evaporated to afford the title compound (30 mg, 45%). LC-MS (ESI+): m/z 451.2 [M+H]⁺.

### Step 3. 6-(6-acrylamido-4-methylpyridin-3-yl)-7-(4-((6-methylpyridin-2-yl)oxy)phenyl)pyrrolo[1,2-a]pyrazine-8-carboxamide

To a solution of 6-(6-amino-4-methylpyridin-3-yl)-7-(4-((6-methylpyridin-2-yl)oxy)phenyl)pyrrolo[1,2-a]pyrazine-8-carboxamide (20 mg, 0.044 mmol) in DCM (2 mL) were added TEA (0.1 mL, 0.67 mmol), acrylic acid (3.1 mL, 0.1 M in DCM, 0.31 mmol) and T₃P (200 mg, 50% in EtOAc, 0.31 mmol)) at 0° C. The mixture was stirred at 25° C. for 1 h. Then the mixture was quenched by water (15 mL) and extracted with DCM (3×10 mL). The combined organic layers were washed with brine, dried over Na₂SO₄, filtered, and concentrated under reduced pressure to give a residue which was purified by prep-HPLC (eluted with 30% to 46% MeCN in H₂O containing 0.1% FA) to afford the title compound (0.59 mg, 2%). LC-MS (ESI+): m/z 505.3 [M+H]⁺. ¹H NMR (400 MHZ, CDCl₃) δ 10.02 (s, 1H), 8.85 (s, 1H), 8.39 (s, 1H), 8.05 (s, 1H), 7.81 (s, 1H), 7.63 (t, J=8.0 Hz, 1H), 7.52 (s, 1H), 7.25 (m, 2H), 7.14 (d, J=8.0 Hz, 2H), 6.95 (d, J=8.0 Hz, 1H), 6.73 (d, J=8.0 Hz, 1H), 6.53 (d, J=16.0 Hz, 1H), 6.33 (dd, J=16.0, 8.0 Hz, 1H), 5.90 (d, J=8.0 Hz, 1H), 5.78-5.63 (m, 1H), 2.45 (s, 3H), 2.06 (s, 3H).

### Example 5:6-(4-acrylamidophenyl)-4-methyl-7-(4-((6-methylpyridin-2-yl)oxy)phenyl)pyrrolo[1,2-a]pyrazine-8-carboxamide

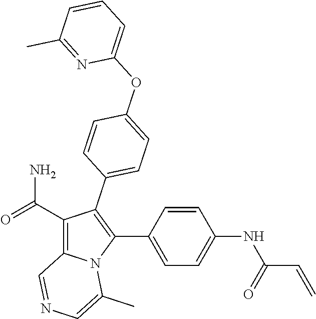 [SMILES: C=CC(=O)Nc1ccc(-c2c(-c3ccc(Oc4cccc(C)n4)cc3)c(C(N)=O)c3cncc(C)n23)cc1]

### Example 5

### Preparation of tert-butyl 2-cyano-2-(6-methylpyrazin-2-yl)acetate (Intermediate 5.1)

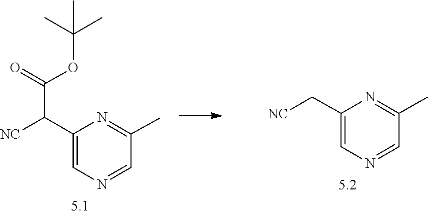

In a similar fashion according to the procedure for intermediate 1.1, intermediate 5.1 was synthesized by replacing 2-chloropyrazine with 2-chloro-6-methylpyrazine.

### Preparation of 2-(6-methylpyrazin-2-yl) acetonitrile (Intermediate 5.2)

To a solution of tert-butyl 2-cyano-2-(6-methylpyrazin-2-yl)acetate (7.0 g, 30.01 mmol) in DMSO (70 mL) and H₂O (70 mL) was added LiBr (3.92 g, 45.01 mmol). The mixture was stirred at 130° C. for 1 h. After cooling to rt, the mixture was poured into saturated NaHCO₃ solution slowly (300 mL) and extracted with EtOAc (3×100 mL). The combined organic layers were washed with brine (200 mL), dried over Na₂SO₄, filtered, and evaporated to afford the title compound (3.5 g, 88%) as a yellow oil. LC-MS (ESI+): m/z 134.2 [M+H]⁺.

### Preparation of 6-(4-acrylamidophenyl)-4-methyl-7-(4-((6-methylpyridin-2-yl)oxy)phenyl)pyrrolo[1,2-a]pyrazine-8-carboxamide (Example 5)

In a similar fashion according to the procedure for Example 1, Example 5 was synthesized by replacing 2-(pyrazin-2-yl) acetonitrile with 2-(6-methylpyrazin-2-yl) acetonitrile (intermediate 5.2). The residue was purified by prep-HPLC (eluted with 55% to 60% MeCN in H₂O containing 0.1% FA) to afford the title compound (17 mg, 19%). LC-MS (ESI+): m/z 504.2 [M+H]⁺0.1H NMR (400 MHZ, DMSO-d₆) δ 10.30 (s, 1H), 9.29 (s, 1H), 7.71 (t, J=8.0 Hz, 1H), 7.63 (d, J=8.0 Hz, 2H), 7.44 (s, 1H), 7.35 (d, J=8.0 Hz, 3H), 7.24 (d, J=8.0 Hz, 2H), 7.00 (d, J=8.0 Hz, 3H), 6.71 (d, J=8.0 Hz, 1H), 6.44 (dd, J₁=16.8, J₂=10.0 Hz, 2H), 6.27 (dd, J₁=16.0, J₂=2.0 Hz, 1H), 5.77 (dd, J₁=10.0, J₂=2.0 Hz, 1H), 2.31 (s, 3H), 1.98 (s, 3H).

### Example 6:6-(4-acrylamidophenyl)-4-methyl-7-(4-((4-methylpyrimidin-2-yl)oxy)phenyl)pyrrolo[1,2-a]pyrazine-8-carboxamide

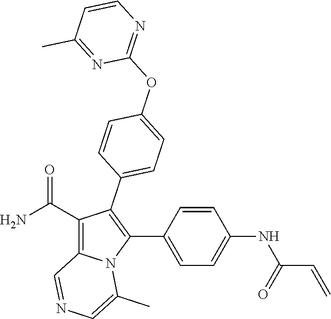 [SMILES: C=CC(=O)Nc1ccc(-c2c(-c3ccc(Oc4nccc(C)n4)cc3)c(C(N)=O)c3cncc(C)n23)cc1]

### Example 6

### Preparation of Intermediate 6.2

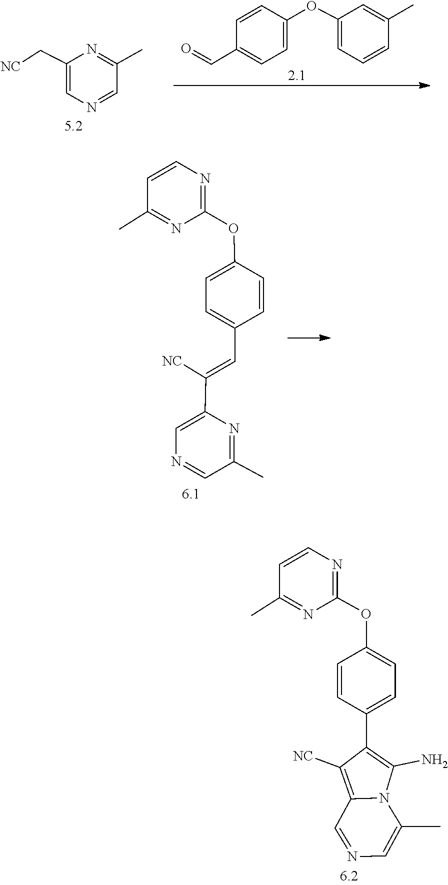

### Step 1. 2-(6-methylpyrazin-2-yl)-3-(4-((4-methylpyrimidin-2-yl)oxy)phenyl) acrylonitrile

To a solution of 2-(6-methylpyrazin-2-yl) acetonitrile (2 g, 15.02 mmol) in MeOH (200 ml) were added 4-((4-methylpyrimidin-2-yl)oxy)benzaldehyde (intermediate 2.1) (3.86 g, 18.02 mmol) and NH₄OAc (8.34 g, 108.15 mmol). The mixture was stirred at 70° C. for 3 h. After cooling to rt, the precipitate was filtered. The solid was washed with water (20 ml) and dried in vacuum to afford the title compound (3.5 g, 71%). LC-MS (ESI+): m/z 330.3 [M+H]⁺.

### Step 2. 6-amino-4-methyl-7-(4-((4-methylpyrimidin-2-yl)oxy)phenyl)pyrrolo[1,2-a]pyrazine-8-carbonitrile

To a solution of 2-(6-methylpyrazin-2-yl)-3-(4-((4-methylpyrimidin-2-yl)oxy)phenyl) acrylonitrile (1.0 g, 3.04 mmol) in MeOH (20 mL) were added DBU (1.39 g, 9.11 mmol) and acetyl cyanide (0.21 g, 3.04 mmol). The mixture was stirred at 120° C. for 20 min under microwave irradiation and N₂. After cooling to rt, the mixture was quenched by water (100 mL) and extracted with DCM (3×100 mL). The combined organic layers were washed with brine, dried over Na₂SO₄, filtered, and concentrated under reduced pressure to give a residue which was purified by flash column chromatography on silica gel (eluting with MeOH:DCM=20:1) to afford the title compound (1 g, 92%). LC-MS (ESI+): m/z 357.2 [M+H]⁺.

In a similar fashion according to the procedure for Example 2, Example 6 was synthesized by replacing 6-amino-7-(4-((4-methylpyrimidin-2-yl)oxy)phenyl)pyrrolo[1,2-a]pyrazine-8-carbonitrile with 6-amino-4-methyl-7-(4-((4-methylpyrimidin-2-yl)oxy)phenyl)pyrrolo[1,2-a]pyrazine-8-carbonitrile. The residue was purified by prep-HPLC (eluted with 55% to 60% MeCN in H₂O containing 0.1% FA) to afford the title compound (6 mg, yield for two steps 8%). LC-MS (ESI+): m/z 505.1 [M+H]⁺. ¹H NMR (400 MHZ, DMSO-d₆) δ 10.29 (s, 1H), 9.25 (s, 1H), 8.44 (d, J=8.0 Hz, 1H), 8.37 (s, 1H), 7.64 (d, J=8.0 Hz, 2H), 7.43 (s, 1H), 7.37 (d, J=8.0 Hz, 2H), 7.27 (d, J=8.0 Hz, 2H), 7.13 (d, J=8.0 Hz, 1H), 7.09 (d, J=8.0 Hz, 2H), 6.53 (s, 1H), 6.44 (dd, J₁=16.0, J₂=12.0 Hz, 1H), 6.30-6.22 (m, 1H), 5.79-5.74 (m, 1H), 2.38 (s, 3H), 1.97 (s, 3H).

### Example 7:6-(4-acrylamidophenyl)-3-methyl-7-(4-((6-methylpyridin-2-yl)oxy)phenyl)pyrrolo[1,2-a]pyrazine-8-carboxamide

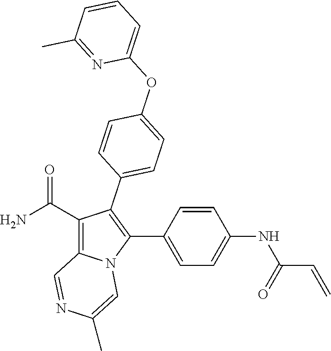 [SMILES: C=CC(=O)Nc1ccc(-c2c(-c3ccc(Oc4cccc(C)n4)cc3)c(C(N)=O)c3cnc(C)cn23)cc1]

### Example 7

### Preparation of Intermediate 7.1

### Step 1. 2-(5-methylpyrazin-2-yl) acetonitrile

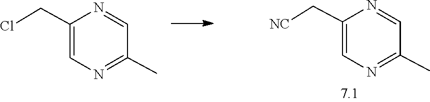

A mixture of 2-(chloromethyl)-5-methylpyrazine hydrochloride (4.00 g, 22.3 mmol), KI (1.11 g, 6.70 mmol) and KCN (2.18 g, 33.51 mmol) in EtOH (200 mL) and H₂O (40 mL) was degassed and purged with N₂ for 3 times. The mixture was stirred at 80° C. for 5 hrs under N₂ atmosphere. After cooling to rt, 200 mL of Na₂CO₃ (aq) was added to the solution. The solution was diluted with 200 mL water and extracted with ethyl acetate (250 mL*2). The combined organic layers were washed with brine, dried over Na₂SO₄, filtered, and concentrated under reduced pressure to give a residue which was purified by flash column chromatography on silica gel (eluting with 0˜30% ethyl acetate in petroleum ether) to afford the title compound (2.10 g, 71%). ¹H NMR: (400 MHZ, CDCl₃) δ 8.59 (s, 1H), 8.51-8.40 (m, 1H), 3.94 (s, 2H), 2.67-2.57 (m, 3H).

In a similar fashion according to the procedure for Example 2, Example 7 was synthesized by replacing 2-(pyrazin-2-yl) acetonitrile with 2-(5-methylpyrazin-2-yl) acetonitrile and replacing 4-((4-methylpyrimidin-2-yl)oxy)benzaldehyde with 4-((6-methylpyridin-2-yl)oxy)benzaldehyde. The residue was purified by prep-HPLC (FA condition; column: Boston Prime C₁₈ 150*30 mm*5 um; mobile phase: [water (FA)-ACN]; B %: 20%-40%, 2 min) to afford the title compound (26 mg, 13%). LC-MS (ESI+): m/z 504.2 [M+H]⁺. ¹H NMR (400 MHz, DMSO-d₆) δ 10.31 (s, 1H), 9.27 (s, 1H), 7.85 (s, 1H), 7.79-7.70 (m, 3H), 7.42-7.24 (m, 5H), 7.12-6.98 (m, 3H), 6.76 (d, J=8.0 Hz, 1H), 6.45 (dd, J=10.0, 17.1 Hz, 2H), 6.33-6.23 (m, 1H), 5.79 (d, J=10.5 Hz, 1H), 2.35 (s, 3H), 2.34 (s, 3H).

### Example 8:6-(4-acrylamidophenyl)-3-methyl-7-(4-((4-methylpyrimidin-2-yl)oxy)phenyl)pyrrolo[1,2-a]pyrazine-8-carboxamide

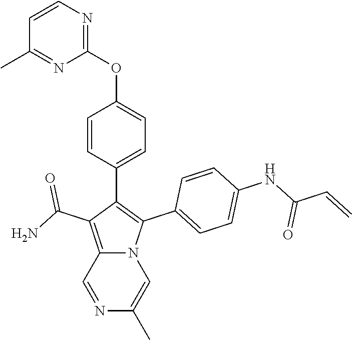 [SMILES: C=CC(=O)Nc1ccc(-c2c(-c3ccc(Oc4nccc(C)n4)cc3)c(C(N)=O)c3cnc(C)cn23)cc1]

### Example 8

In a similar fashion according to the procedure for Example 2, Example 8 was synthesized by replacing 2-(pyrazin-2-yl) acetonitrile with 2-(5-methylpyrazin-2-yl) acetonitrile. The residue was purified by prep-HPLC (eluted with 20% to 36% MeCN in H₂O containing 0.1% FA) to afford the title compound (40 mg, 38%). LC-MS (ESI+): m/z 505.2 [M+H]⁺. ¹H NMR (400 MHZ, DMSO-d₆) δ 10.31 (s, 1H), 9.24 (s, 1H), 8.46 (d, J=8.0 Hz, 1H), 7.81 (s, 1H), 7.76 (d, J=8.0 Hz, 2H), 7.37-7.28 (m, 5H), 7.16 (s, 2H), 7.15 (d, J=8.0 Hz, 1H), 6.57 (br s, 1H), 6.48-6.39 (m, 1H), 6.28 (dd, J₁=16.0, J₂=2.0 Hz, 1H), 5.81-5.75 (m, 1H), 2.41 (s, 3H), 2.34 (s, 3H).

### Example 9: (E)-6-(4-(4-(dimethylamino) but-2-enamido)phenyl)-3-methyl-7-(4-((6-methylpyridin-2-yl)oxy)phenyl)pyrrolo[1,2-a]pyrazine-8-carboxamide

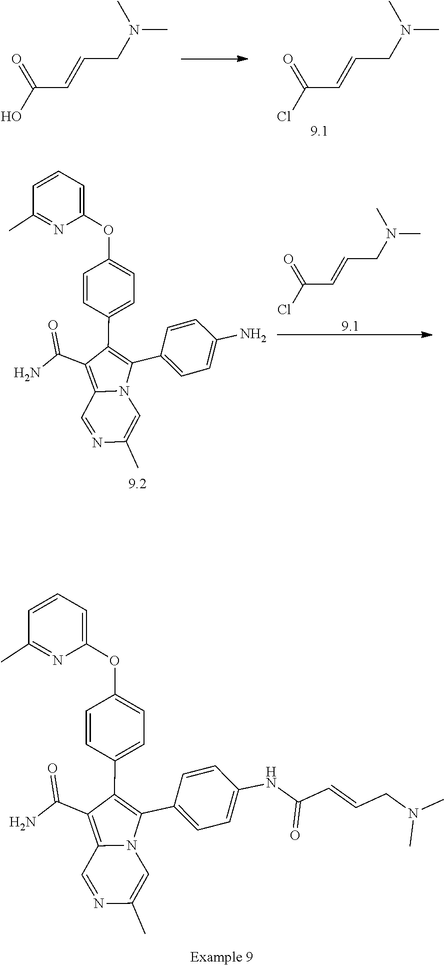

### Step 1. (E)-4-(dimethylamino) but-2-enoyl chloride hydrochloride

To a solution (E)-4-(dimethylamino) but-2-enoic acid hydrochloride (200 mg, 1.212 mmol) in THF (2 mL) were added oxalyl chloride (0.1 mL, 1.212 mmol) and DMF (0.001 mL, 0.012 mmol) at 0° C. The mixture was stirred at 30° C. for 1.5 h. The mixture was used for next step directly.

### Step 2. (E)-6-(4-(4-(dimethylamino) but-2-enamido)phenyl)-3-methyl-7-(4-((6-methylpyridin-2-yl)oxy)phenyl)pyrrolo[1,2-a]pyrazine-8-carboxamide

In a similar fashion according to the procedure for intermediate 2.5, intermediate 9.2 was synthesized by replacing 2-(pyrazin-2-yl) acetonitrile with 2-(5-methylpyrazin-2-yl) acetonitrile and replacing 4-((4-methylpyrimidin-2-yl)oxy)benzaldehyde with 4-((6-methylpyridin-2-yl)oxy)benzaldehyde.

To a solution of 6-(4-aminophenyl)-3-methyl-7-(4-((6-methylpyridin-2-yl)oxy)phenyl)pyrrolo[1,2-a]pyrazine-8-carboxamide (80 mg, 0.18 mmol) in DCM (3 mL) were added TEA (0.074 mL, 0.534 mmol) and (E)-4-(dimethylamino) but-2-enoyl chloride hydrochloride (0.44 mL from step 1, 0.26 mmol) at 0° C. The mixture was stirred at 0° C. for 3 h. Then the mixture was quenched by water (50 mL) and extracted with DCM (3×30 mL). The combined organic layers were washed with brine, dried over Na₂SO₄, filtered, and concentrated under reduced pressure to give a residue which was purified by prep-HPLC (eluted with 25% to 37% MeCN in H₂O containing 0.1% FA) to afford the title compound (6.5 mg, 7%). LC-MS (ESI+): m/z 561.4 [M+H]⁺. ¹H NMR (400 MHZ, DMSO-d₆) δ 10.20 (s, 1H), 9.27 (s, 1H), 7.83 (s, 1H), 7.77-7.69 (m, 3H), 7.33-7.22 (m, 4H), 7.06 (d, J=8.0 Hz, 2H), 7.01 (d, J=8.0 Hz, 1H), 6.80-6.70 (m, 2H), 6.46 (br s, 1H), 6.28 (d, J=16.0 Hz, 1H), 3.06 (d, J=8.0 Hz, 2H), 2.34 (s, 6H), 2.18 (s, 6H).

### Example 10: (E)-6-(6-(4-(dimethylamino) but-2-enamido)pyridin-3-yl)-3-methyl-7-(4-((6-methylpyridin-2-yl)oxy)phenyl)pyrrolo[1,2-a]pyrazine-8-carboxamide

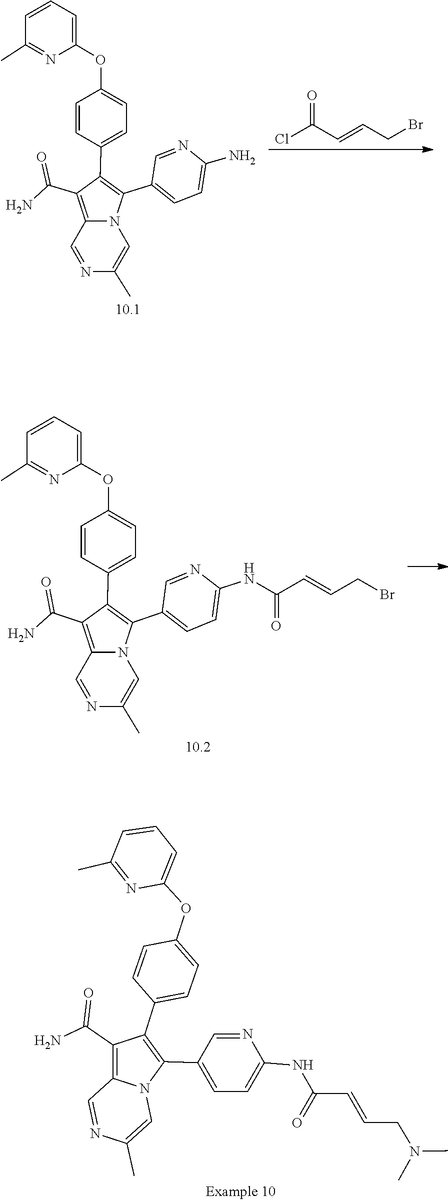 [SMILES: Cc1cn2c(-c3ccc(N)nc3)c(-c3ccc(Oc4cccc(C)n4)cc3)c(C(N)=O)c2cn1 ; O=C(Cl)/C=C/CBr ; Cc1cn2c(-c3ccc(NC(=O)/C=C/CBr)nc3)c(-c3ccc(Oc4cccc(C)n4)cc3)c(C(N)=O)c2cn1 ; Cc1cn2c(-c3ccc(NC(=O)/C=C/CN(C)C)nc3)c(-c3ccc(Oc4cccc(C)n4)cc3)c(C(N)=O)c2cn1]

### Step 1. (E)-6-(6-(4-bromobut-2-enamido)pyridin-3-yl)-3-methyl-7-(4-((6-methylpyridin-2-yl)oxy)phenyl)pyrrolo[1,2-a]pyrazine-8-carboxamide

In a similar fashion according to the procedure for intermediate 2.5, intermediate 10.1 was synthesized by replacing 2-(pyrazin-2-yl) acetonitrile with 2-(5-methylpyrazin-2-yl) acetonitrile, replacing 4-((4-methylpyrimidin-2-yl)oxy)benzaldehyde with 4-((6-methylpyridin-2-yl)oxy)benzaldehyde and replacing 4-(4,4,5,5-tetramethyl-1,3,2-dioxaborolan-2-yl) aniline with 5-(4,4,5,5-tetramethyl-1,3,2-dioxaborolan-2-yl) pyridin-2-amine.

To a solution of 6-(6-aminopyridin-3-yl)-3-methyl-7-(4-((6-methylpyridin-2-yl)oxy)phenyl)pyrrolo[1,2-a]pyrazine-8-carboxamide (80 mg, 0.18 mmol) in DCM (2 mL) were added TEA (54 mg, 0.36 mmol) and (E)-4-bromobut-2-enoyl chloride (65 mg, 0.36 mmol) at 0° C. The mixture was stirred at rt for 18 h. The mixture was quenched by water (10 mL) and extracted with DCM (3×10 mL). The combined organic layers were washed with brine, dried over Na₂SO₄, filtered, and concentrated under reduced pressure to give a residue which was purified by prep-TLC (DCM:MeOH=20:1) to afford the title compound (12 mg, 0.02 mmol, 11%) as a yellow oil. LC-MS (ESI+): m/z 597.1 [M+H]⁺.

### Step 2. (E)-6-(6-(4-(dimethylamino) but-2-enamido)pyridin-3-yl)-3-methyl-7-(4-((6-methylpyridin-2-yl)oxy)phenyl)pyrrolo[1,2-a]pyrazine-8-carboxamide

To a solution of (E)-6-(6-(4-bromobut-2-enamido)pyridin-3-yl)-3-methyl-7-(4-((6-methylpyridin-2-yl)oxy)phenyl)pyrrolo[1,2-a]pyrazine-8-carboxamide (12 mg, 0.02 mmol) in MeCN (2 mL) were added K₂CO₃ (3 mg, 0.02 mmol) and dimethylamine hydrochloride (0.1 mL, 0.2 M in MeCN). The mixture was stirred at 60° C. for 2 h. After cooling to rt, the mixture was quenched by water (10 mL) and extracted with DCM (3×10 mL). The combined organic layers were washed with brine, dried over Na₂SO₄, filtered, and concentrated under reduced pressure to give a residue which was purified by prep-HPLC (eluted with 22% to 35% MeCN in H₂O containing 0.1% FA) to afford the title compound (0.6 mg, 5%). LC-MS (ESI+): m/z 562.2 [M+H]⁺. ¹H NMR (400 MHZ, DMSO-d₆) δ 10.74 (s, 1H), 9.30 (s, 1H), 8.43-8.22 (m, 3H), 8.19 (s, 1H), 7.94 (s, 1H), 7.82 (dd, J=8.0, 2.0 Hz, 1H), 7.74 (t, J=8.0 Hz, 1H), 7.30 (d, J=8.0 Hz, 2H), 7.08 (d, J=8.0 Hz, 2H), 7.02 (d, J=8.0 Hz, 1H), 6.90-6.65 (m, 2H), 6.45 (d, J=16.0 Hz, 1H), 3.05 (d, J=8.0 Hz, 2H), 2.36 (s, 3H), 2.33 (s, 3H), 2.17 (s, 6H).

### Example 11:6-(4-acrylamidophenyl)-3-(hydroxymethyl)-7-(4-((6-methylpyridin-2-yl)oxy)phenyl)pyrrolo[1,2-a]pyrazine-8-carboxamide

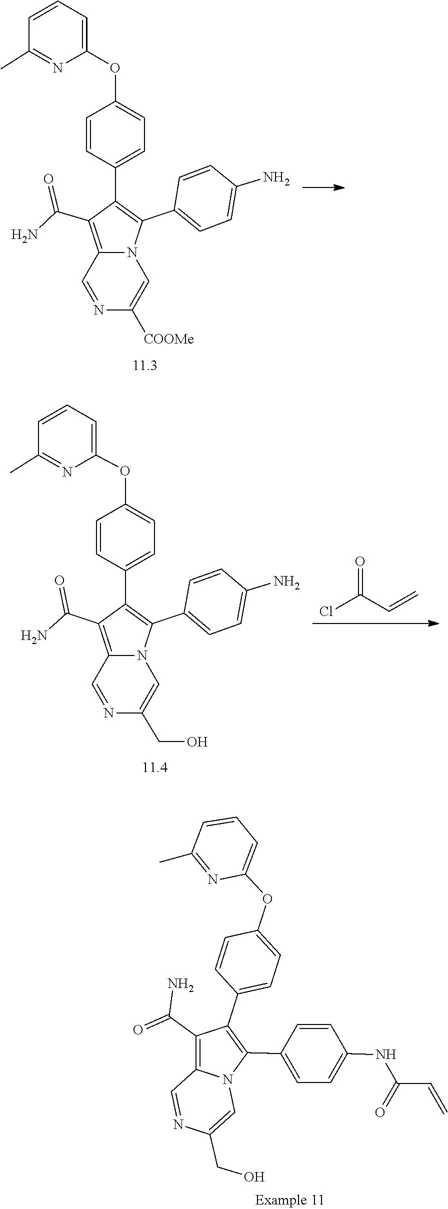

### Preparation of methyl 5-(cyanomethyl) pyrazine-2-carboxylate (intermediate 11.2)

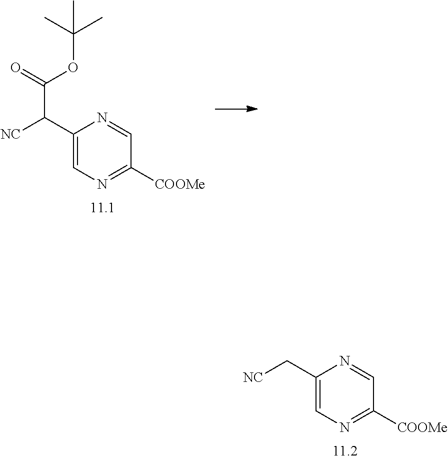 [SMILES: COC(=O)c1cnc(C(C#N)C(=O)OC(C)(C)C)cn1 ; COC(=O)c1cnc(CC#N)cn1]

In a similar fashion according to the procedure for intermediate 1.1, intermediate 11.1 was synthesized by replacing 2-chloropyrazine with methyl 5-chloropyrazine-2-carboxylate.

To a mixture of methyl 5-(2-(tert-butoxy)-1-cyano-2-oxoethyl) pyrazine-2-carboxylate (9.2 g, 33.18 mmol) in toluene (200 mL) was added p-TsOH (5.71 g, 33.18 mmol). The reaction mixture was stirred at 120° C. for 18 h. After cooling to rt, the mixture was poured into water slowly (300 mL) and extracted with EtOAc (3×100 mL). The combined organic layers were washed with brine, dried over Na₂SO₄, filtered, and concentrated under reduced pressure to give a residue which was purified by flash column chromatography on silica gel (eluting with PE:EtOAc=1:1) to afford intermediate 11.2 (900 mg, 15%). LC-MS (ESI+): m/z 178.1 [M+H]⁺. ¹H NMR (400 MHZ, DMSO-d₆) δ 9.20 (d, J=1.3 Hz, 1H), 8.81 (s, 1H), 4.48 (s, 2H), 3.94 (s, 3H).

### Step 1. 6-(4-aminophenyl)-3-(hydroxymethyl)-7-(4-((6-methylpyridin-2-yl)oxy)phenyl)pyrrolo[1,2-a]pyrazine-8-carboxamide

In a similar fashion according to the procedure for intermediate 2.5, intermediate 11.3 was synthesized by replacing 2-(pyrazin-2-yl) acetonitrile with methyl 5-(cyanomethyl) pyrazine-2-carboxylate and replacing 4-((4-methylpyrimidin-2-yl)oxy)benzaldehyde with 4-((6-methylpyridin-2-yl)oxy)benzaldehyde.

To a solution of methyl 6-(4-aminophenyl)-8-carbamoyl-7-(4-((6-methylpyridin-2-yl)oxy)phenyl)pyrrolo[1,2-a]pyrazine-3-carboxylate (150 mg, 0.30 mmol) in DCM (5 mL) and MeOH (5 mL) were added NaBH₄ (57.49 mg, 1.52 mmol) and CaCl₂ (3.37 mg, 0.03 mmol) at 0° C. The mixture was stirred at 25° C. for 5 h. Then the mixture was quenched by water (20 mL) and extracted with DCM (3×20 mL). The combined organic layers were washed with brine, dried over Na₂SO₄, filtered, and concentrated under reduced pressure to give a residue which was purified by flash column chromatography on silica gel (eluting with MeOH:DCM=20:1) to afford the title compound (15 mg, 10%) as a yellow oil. LC-MS (ESI+): m/z 466.2 [M+H]⁺.

### Step 2. 6-(4-acrylamidophenyl)-3-(hydroxymethyl)-7-(4-((6-methylpyridin-2-yl)oxy)phenyl)pyrrolo[1,2-a]pyrazine-8-carboxamide

To a solution of 6-(4-aminophenyl)-3-(hydroxymethyl)-7-(4-((6-methylpyridin-2-yl)oxy)phenyl)pyrrolo[1,2-a]pyrazine-8-carboxamide (15 mg, 0.03 mmol) in acetone (5 mL) were added K₂CO₃ (13 mg, 0.10 mmol, 0.5M in water) and acryloyl chloride (0.1 mL, 0.3 M in acetone) at 0° C. The mixture was stirred at 0° C. for 30 min. Then the mixture was quenched by water (10 mL) and extracted with DCM (3×10 mL). The combined organic layers were washed with brine, dried over Na₂SO₄, filtered, and concentrated under reduced pressure to give a residue which was purified by prep-HPLC (eluted with 35% to 40% MeCN in H₂O containing 0.1% FA) to afford the title compound (1.3 mg, 7%). LC-MS (ESI+): m/z 520.3 [M+H]⁺. ¹H NMR (400 MHZ, DMSO-d₆) δ 10.33 (s, 1H), 9.27 (s, 1H), 7.89 (s, 1H), 7.79-7.71 (m, 3H), 7.40-7.25 (m, 5H), 7.07 (d, J=8.4 Hz, 2H), 7.01 (d, J=7.2 Hz, 1H), 6.76 (d, J=8.0 Hz, 1H), 6.55 (br s, 1H), 6.45 (dd, J₁=16.0, J₂=12.0 Hz, 1H), 6.28 (dd, J₁=16.0, J₂=2.0 Hz, 1H), 5.78 (dd, J₁=10.0, J₂=2.0 Hz, 1H), 5.40 (br s, 1H), 4.53 (s, 2H), 2.34 (s, 3H).

### Example 12:6-(4-acrylamidophenyl)-1-(methylamino)-7-(4-((6-methylpyridin-2-yl)oxy)phenyl)pyrrolo[1,2-a]pyrazine-8-carboxamide

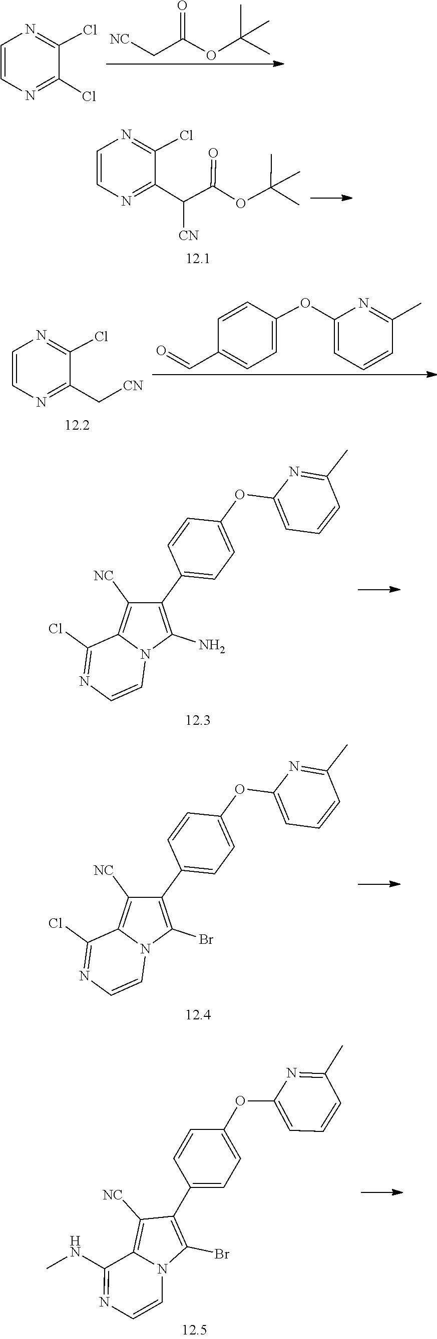

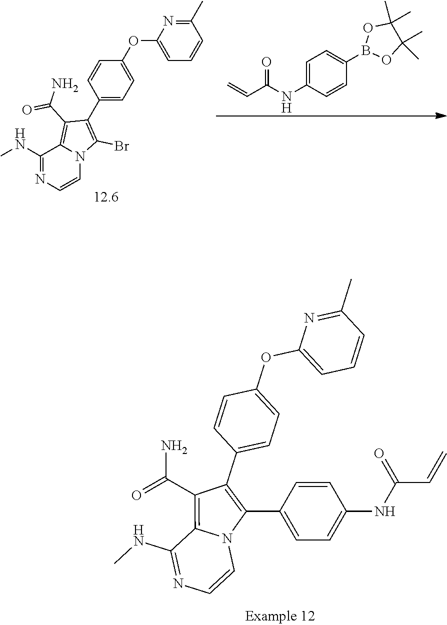

### Step 1. tert-butyl 2-(3-chloropyrazin-2-yl)-2-cyanoacetate

To a solution of 2,3-dichloropyrazine (10.0 g, 67.1 mmol) in DMF (100 mL) was added Cs₂CO₃ (43.7 g, 134.3 mmol). Then tert-butyl 2-cyanoacetate (12.3 g, 87.3 mmol, 12.5 mL) was added. The reaction mixture was stirred at 80° C. for 4 hrs. After cooling to rt, to the mixture was added 300 mL of water. The pH of the mixture was adjusted to 3˜5 by 1N HCl (aq). The mixture was extracted with EtOAc (200 mL*3). The combined organic layers were washed with brine, dried over Na₂SO₄, filtered, and concentrated under reduced pressure to give a residue which was purified by flash column chromatography on silica gel (eluting with 0˜50% ethyl acetate in petroleum ether) to afford the title compound (15.0 g, 88%) as a yellow oil.

### Step 2. 2-(3-chloropyrazin-2-yl) acetonitrile

To a solution of tert-butyl 2-(3-chloropyrazin-2-yl)-2-cyanoacetate (15.0 g, 59.1 mmol) in toluene (170 mL) was added p-TsOH (815 mg, 4.73 mmol). The reaction mixture was stirred at 120° C. for 12 hrs. After cooling to rt, the reaction mixture was concentrated under reduced pressure to remove solvent. The residue was purified by flash column chromatography on silica gel (eluting with 0˜50% ethyl acetate in petroleum ether) to afford the title compound (6.0 g, 66%) as a yellow oil. ¹H NMR (400 MHz, DMSO-d₆) δ 8.70 (d, J=2.5 Hz, 1H), 8.54 (d, J=2.5 Hz, 1H), 4.47 (s, 2H).

### Step 3. 6-amino-1-chloro-7-(4-((6-methylpyridin-2-yl)oxy)phenyl)pyrrolo[1,2-a]pyrazine-8-carbonitrile

To a mixture of 4-((6-methylpyridin-2-yl)oxy)benzaldehyde (694 mg, 3.26 mmol) and 2-(3-chloropyrazin-2-yl) acetonitrile (500 mg, 3.26 mmol) in MeOH (10 mL) were added DBU (1.49 g, 9.77 mmol, 1.47 mL) and acetyl cyanide (225 mg, 3.26 mmol) at 25° C. The reaction mixture was purged with N₂ for 3 times, stirred and heated at 100° C. under microwave irradiation for 20 mins. After cooling to rt, the reaction mixture was concentrated under reduced pressure to give a residue which was purified by flash column chromatography on silica gel (eluting with 0˜80% ethyl acetate in petroleum ether) to afford the title compound (600 mg, 49%). LC-MS (ESI+): m/z 376.0 [M+H]⁺.

### Step 4. 6-bromo-1-chloro-7-(4-((6-methylpyridin-2-yl)oxy)phenyl)pyrrolo[1,2-a]pyrazine-8-carbonitrile

To a solution of 6-amino-1-chloro-7-(4-((6-methylpyridin-2-yl)oxy)phenyl)pyrrolo[1,2-a]pyrazine-8-carbonitrile (300 mg, 798.3 μmol) in HBr/AcOH (10 mL) was added CuBr₂ (232 mg, 1.04 mmol, 48.6 μL) at 0° C. The mixture was stirred for 20 mins. Then a solution of NaNO₂ (275 mg, 3.99 mmol) in H₂O (4 mL) was added. The reaction mixture was stirred at 0° C. for 0.5 hr. Then the pH of the solution was adjusted to 7 by Na₂CO₃ (aq). After extraction with ethyl acetate (20 mL*3), the combined organic layers were washed with brine, dried over Na₂SO₄, filtered, and concentrated under reduced pressure to give a residue which was purified by flash column chromatography on silica gel (eluting with 0˜2% MeOH in DCM) to afford the title compound (300 mg, 86%). LC-MS (ESI+): m/z 438.8 [M+H]⁺.

### Step 5. 6-bromo-1-(methylamino)-7-(4-((6-methylpyridin-2-yl)oxy)phenyl)pyrrolo[1,2-a]pyrazine-8-carbonitrile

To a mixture of 6-bromo-1-chloro-7-(4-((6-methylpyridin-2-yl)oxy)phenyl)pyrrolo[1,2-a]pyrazine-8-carbonitrile (300 mg, 682.3 μmol) and methanamine hydrochloride (138 mg, 2.05 mmol) in THF (3 mL) was added DBU (519 mg, 3.41 mmol) at 25° C. The reaction mixture was purged with N₂ for 3 times, stirred and heated at 120° C. under microwave irradiation for 3 hrs. After cooling to rt, the reaction mixture was concentrated under reduced pressure to give a residue which was purified by flash column chromatography on silica gel (eluting with 0˜30% Ethyl acetate in Petroleum ether) to afford the title compound (210 mg, 71%). LC-MS (ESI+): m/z 433.8 [M+H]⁺.

### Step 6. 6-bromo-1-(methylamino)-7-(4-((6-methylpyridin-2-yl)oxy)phenyl)pyrrolo[1,2-a]pyrazine-8-carboxamide

A mixture of 6-bromo-1-(methylamino)-7-(4-((6-methylpyridin-2-yl)oxy)phenyl)pyrrolo[1,2-a]pyrazine-8-carbonitrile (250 mg, 575.7 μmol) in H₂SO₄ (5.52 g, 56.3 mmol, 3 mL) was stirred at 30° C. for 16 hrs. Then the mixture was poured into water (100 mL) slowly, adjusted to pH˜7 with Na₂CO₃ and extracted with EtOAc (3×100 mL). The combined organic layers were washed with brine, dried over Na₂SO₄, filtered, and concentrated under reduced pressure to give the title compound (260 mg). LC-MS (ESI+): m/z 451.9 [M+H]⁺.

### Step 7. 6-(4-acrylamidophenyl)-1-(methylamino)-7-(4-((6-methylpyridin-2-yl)oxy)phenyl)pyrrolo[1,2-a]pyrazine-8-carboxamide

To a solution of 6-bromo-1-(methylamino)-7-(4-((6-methylpyridin-2-yl)oxy)phenyl)pyrrolo[1,2-a]pyrazine-8-carboxamide (140 mg, 309.5 μmol) and N-(4-(4,4,5,5-tetramethyl-1,3,2-dioxaborolan-2-yl)phenyl) acrylamide (169 mg, 619.1 μmol) in dioxane (3 mL) and H₂O (1 mL) were added K₂CO₃ (128 mg, 928.6 μmol) and Pd-118 (20 mg, 31.0 μmol). The mixture was degassed, purged with N₂ for 3 times and stirred at 90° C. for 2 hours under N₂ atmosphere. After cooling to rt, the reaction mixture was concentrated under reduced pressure to give a residue which was purified by reversed-phase HPLC (column: Boston Prime C₁₈ 150*30 mm*5 um; mobile phase: [water (FA)-ACN]; B %: 20%-40%, 9 min) to afford the title compound (23 mg, 15%). LC-MS (ESI+): m/z 519.2 [M+H]⁺. ¹H NMR (400 MHZ, DMSO-d₆) δ 10.35-10.18 (m, 1H), 9.13-8.82 (m, 1H), 7.78-7.68 (m, 3H), 7.62 (br s, 1H), 7.28-7.15 (m, 6H), 7.11-6.95 (m, 3H), 6.74 (d, J=8.1 Hz, 1H), 6.50-6.22 (m, 3H), 5.82-5.73 (m, 1H), 2.98 (d, J=4.6 Hz, 3H), 2.34 (s, 3H).

### Example 13:6-(4-acrylamidophenyl)-2-methyl-7-(4-((6-methylpyridin-2-yl)oxy)phenyl)-1,2,3,4-tetrahydropyrrolo[1,2-a]pyrazine-8-carboxamide

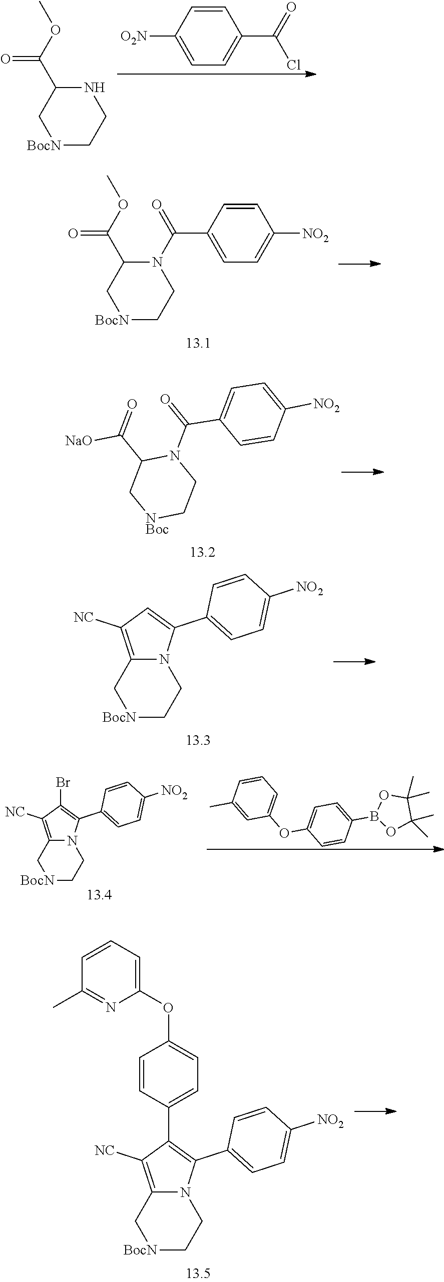

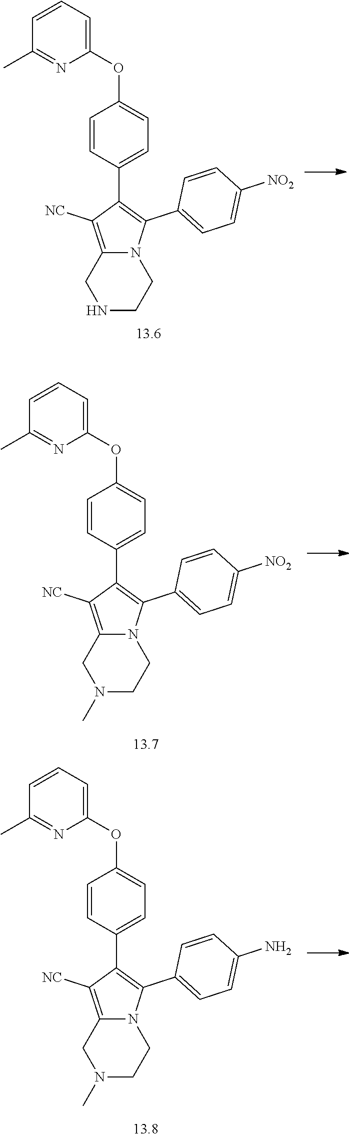 [SMILES: Cc1cccc(Oc2ccc(-c3c(C#N)c4n(c3-c3ccc([N+](=O)[O-])cc3)CCNC4)cc2)n1 ; Cc1cccc(Oc2ccc(-c3c(C#N)c4n(c3-c3ccc([N+](=O)[O-])cc3)CCN(C)C4)cc2)n1 ; Cc1cccc(Oc2ccc(-c3c(C#N)c4n(c3-c3ccc(N)cc3)CCN(C)C4)cc2)n1]

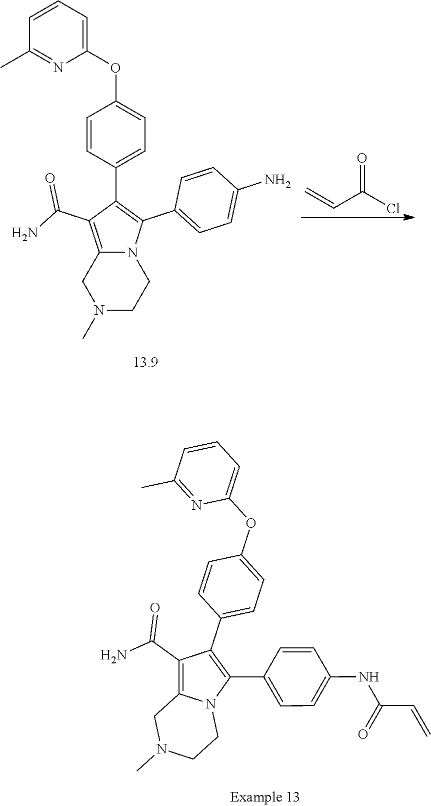

### Step 1. 1-(tert-butyl)₃-methyl 4-(4-nitrobenzoyl) piperazine-1,3-dicarboxylate

To a solution of 1-(tert-butyl)₃-methyl piperazine-1,3-dicarboxylate (17.4 g, 71.23 mmol) in DCM (600 mL) was added TEA (21.8 mL, 156.69 mmol). The mixture was stirred at 0° C. for 30 min. 4-Nitrobenzoyl chloride was added (14.54 g in 85 mL DCM, 78.35 mmol) and the mixture was stirred at rt for 3 h. The mixture was quenched by water (500 mL) and extracted with DCM (3×100 mL). The combined organic layers were dried over Na₂SO₄, filtered, and evaporated to afford the title compound (27 g), which was used directly in the next step. LC-MS (ESI+): m/z 338.2 (M+H−56)+.

### Step 2. sodium 4-(tert-butoxycarbonyl)-1-(4-nitrobenzoyl) piperazine-2-carboxylate

To a solution of 1-(tert-butyl)₃-methyl 4-(4-nitrobenzoyl) piperazine-1,3-dicarboxylate (27 g) in MeOH (300 mL) was added NaOH solution (2N, 41 mL, 82.00 mmol) slowly at 0° C. The mixture was stirred at rt for 18 h. The mixture was then evaporated and dried in vacuum to give the title compound (30 g), which was used directly in the next step. LC-MS (ESI+): m/z 378.1 (M-Na).

### Step 3. tert-butyl 8-cyano-6-(4-nitrophenyl)-3,4-dihydropyrrolo[1,2-a]pyrazine-2 (1H)-carboxylate

To a mixture of sodium 4-(tert-butoxycarbonyl)-1-(4-nitrobenzoyl) piperazine-2-carboxylate (15 g) in DCM (300 mL) was added 4-toluenesulfonyl chloride (7.84 g, 41.11 mmol) at 0° C. After stirring at rt for 1 h, 2-chloroacrylonitrile (3.0 mL, 37.37 mmol) and TEA (11.94 mL, 85.96 mmol) were added successively. The mixture was stirred at rt for 18 h. Then the mixture was quenched by water (400 mL) and extracted with DCM (3×100 mL). The combined organic layers were washed with brine, dried over Na₂SO₄, filtered, and concentrated under reduced pressure to give a residue which was purified by flash column chromatography on silica gel (eluting with PE:EtOAc=1:1) to afford the title compound (3.7 g, total yield 28% for three steps). LC-MS (ESI+): m/z 369.1 [M+H]⁺.

### Step 4. tert-butyl 7-bromo-8-cyano-6-(4-nitrophenyl)-3,4-dihydropyrrolo[1,2-a]pyrazine-2 (1H)-carboxylate

To a solution of tert-butyl 8-cyano-6-(4-nitrophenyl)-3,4-dihydropyrrolo[1,2-a]pyrazine-2 (1H)-carboxylate (3.0 g, 8.14 mmol) in DCM (30 mL) was added NBS (1.59 g, 8.958 mmol) at rt. The mixture was stirred at rt for 2 h. Then the mixture was diluted with DCM (100 mL) and washed with saturated NaHCO₃ solution (2×100 mL). The organic layer was dried over Na₂SO₄, filtered, and evaporated to afford the title compound (3.6 g, 82%). LC-MS (ESI+): m/z 469.0 (M+Na)+.

### Step 5. tert-butyl 8-cyano-7-(4-((6-methylpyridin-2-yl)oxy)phenyl)-6-(4-nitrophenyl)-3,4-dihydropyrrolo[1,2-a]pyrazine-2 (1H)-carboxylate

To a solution of tert-butyl 7-bromo-8-cyano-6-(4-nitrophenyl)-3,4-dihydropyrrolo[1,2-a]pyrazine-2 (1H)-carboxylate (4.27 g, 9.54 mmol) and 2-methyl-6-(4-(4,4,5,5-tetramethyl-1,3,2-dioxaborolan-2-yl) phenoxy)pyridine (3.24 g, 10.42 mmol) in 1,4-dioxane (85 mL) and water (17 mL) were added K₂CO₃ (3.60 g, 26.06 mmol) and XPhos Pd G2 (0.38 g, 0.47 mmol). The mixture was stirred at 100° C. for 1 h under N₂. After cooling to rt, the mixture was poured into water slowly (100 mL) and extracted with EtOAc (3×100 mL). The combined organic layers were washed with brine, dried over Na₂SO₄, filtered, and concentrated under reduced pressure to give a residue which was purified by flash column chromatography on silica gel (eluting with DCM:MeOH=20:1) to afford the title compound (1.4 g, 27%). LC-MS (ESI+): m/z 552.1 [M+H]⁺.

### Step 6. 7-(4-((6-methylpyridin-2-yl)oxy)phenyl)-6-(4-nitrophenyl)-1,2,3,4-tetrahydropyrrolo[1,2-a]pyrazine-8-carbonitrile

To a solution of tert-butyl 8-cyano-7-(4-((6-methylpyridin-2-yl)oxy)phenyl)-6-(4-nitrophenyl)-3,4-dihydropyrrolo[1,2-a]pyrazine-2 (1H)-carboxylate (0.7 g, 1.27 mmol) in DCM (7 mL) was added TFA (3.5 mL) at 0° C. The mixture was stirred at rt for 2 h. The mixture was then poured into saturated NaHCO₃ solution slowly (200 mL) and extracted with EtOAc (3×100 mL). The combined organic layers were dried over Na₂SO₄, filtered, and evaporated to afford the title compound (570 mg, 99%). LC-MS (ESI+): m/z 452.2 [M+H]⁺.

### Step 7. 2-methyl-7-(4-((6-methylpyridin-2-yl)oxy)phenyl)-6-(4-nitrophenyl)-1,2,3,4-tetrahydropyrrolo[1,2-a]pyrazine-8-carbonitrile

To a solution of 7-(4-((6-methylpyridin-2-yl)oxy)phenyl)-6-(4-nitrophenyl)-1,2,3,4-tetrahydropyrrolo[1,2-a]pyrazine-8-carbonitrile (525 mg, 1.16 mmol) in DMF (12 mL) were added K₂CO₃ (193 mg, 1.39 mmol) and Mel (2.3 mL, 1.16 mmol). The mixture was stirred at 30° C. for 1.5 h.

Then the mixture was poured into water (20 mL) and extracted with EtOAc (3×20 mL). The combined organic layers were washed with saturated NH₄Cl solution (3×50 mL), dried over Na₂SO₄, filtered, and concentrated under reduced pressure to give a residue which was purified by flash column chromatography on silica gel (eluting with DCM:MeOH=50:1) to afford the title compound (300 mg, 55%). LC-MS (ESI+): m/z 466.2 [M+H]⁺.

### Step 8. 6-(4-aminophenyl)-2-methyl-7-(4-((6-methylpyridin-2-yl)oxy)phenyl)-1,2,3,4-tetrahydropyrrolo[1,2-a]pyrazine-8-carbonitrile

To a solution of 2-methyl-7-(4-((6-methylpyridin-2-yl)oxy)phenyl)-6-(4-nitrophenyl)-1,2,3,4-tetrahydropyrrolo[1,2-a]pyrazine-8-carbonitrile (200 mg, 0.43 mmol) in EtOH (18 mL) and water (4.5 mL) were added NH₄Cl (115 mg, 2.15 mmol) and iron (120 mg, 2.15 mmol). The mixture was stirred at 70° C. for 5 h. Then the mixture was filtered at 70° C. The filtrate was diluted with water (50 mL) and extracted with DCM (3×50 mL). The combined organic layers were dried over Na₂SO₄, filtered, and evaporated to afford the title compound (120 mg, 63%). LC-MS (ESI+): m/z 436.1 [M+H]⁺.

### Step 9. 6-(4-aminophenyl)-2-methyl-7-(4-((6-methylpyridin-2-yl)oxy)phenyl)-1,2,3,4-tetrahydropyrrolo[1,2-a]pyrazine-8-carboxamide

A mixture of 6-(4-aminophenyl)-2-methyl-7-(4-((6-methylpyridin-2-yl)oxy)phenyl)-1,2,3,4-tetrahydropyrrolo[1,2-a]pyrazine-8-carbonitrile (120 mg, 0.27 mmol) and concentrated H₂SO₄ (10 mL) was stirred at 30° C. for 18 h. Then the mixture was poured into water (100 mL) slowly, adjusted to pH˜8 with 6N NaOH solution and extracted with DCM (3×100 mL). The combined organic layers were washed with brine, dried over Na₂SO₄, filtered, and concentrated under reduced pressure to give a residue which was purified by prep-TLC (DCM:MeOH=20:1) to afford the title compound (70 mg, 56%). LC-MS (ESI+): m/z 454.2 [M+H]⁺.

### Step 10. 6-(4-acrylamidophenyl)-2-methyl-7-(4-((6-methylpyridin-2-yl)oxy)phenyl)-1,2,3,4-tetrahydropyrrolo[1,2-a]pyrazine-8-carboxamide

To a solution of 6-(4-aminophenyl)-2-methyl-7-(4-((6-methylpyridin-2-yl)oxy)phenyl)-1,2,3,4-tetrahydropyrrolo[1,2-a]pyrazine-8-carboxamide (70 mg, 0.154 mmol) in acetone (5 mL) were added K₂CO₃ (0.92 mL, 0.46 mmol, 0.5 mol/L in water) and acryloyl chloride (0.37 mL, 0.18 mmol, 0.5 mol/L in acetone) at 0° C. The mixture was stirred at rt for 2 h. The mixture was quenched by water (50 mL) and extracted with EtOAc (3×20 mL). The combined organic layers were washed with brine, dried over Na₂SO₄, filtered and concentrated under reduced pressure to give a residue which was purified by prep-HPLC (eluted with 20% to 25% MeCN in H₂O containing 0.1% FA) to afford the title compound (22 mg, 28%). LC-MS (ESI+): m/z 508.2 [M+H]⁺. ¹H NMR (400 MHZ, CDCl₃) δ 7.57 (t, J=8.0 Hz, 1H), 7.55-7.37 (m, 3H), 7.19 (d, J=8.0 Hz, 2H), 7.09 (d, J=8.0 Hz, 2H), 7.01 (d, J=8.0 Hz, 2H), 6.89 (d, J=8.0 Hz, 1H), 6.61 (d, J=8.0 Hz, 1H), 6.42 (d, J=16.0 Hz, 1H), 6.25 (dd, J=16.0, 8.0 Hz, 1H), 5.77 (d, J=8.0 Hz, 1H), 5.26 (br s, 2H), 4.20 (br s, 2H), 3.94 (br s, 2H), 2.89 (br s, 2H), 2.60 (s, 3H), 2.44 (s, 3H).

### Example 14:6-(1-acryloylindolin-5-yl)-7-(4-((6-methylpyridin-2-yl)oxy)phenyl)pyrrolo[1,2-a]pyrazine-8-carboxamide

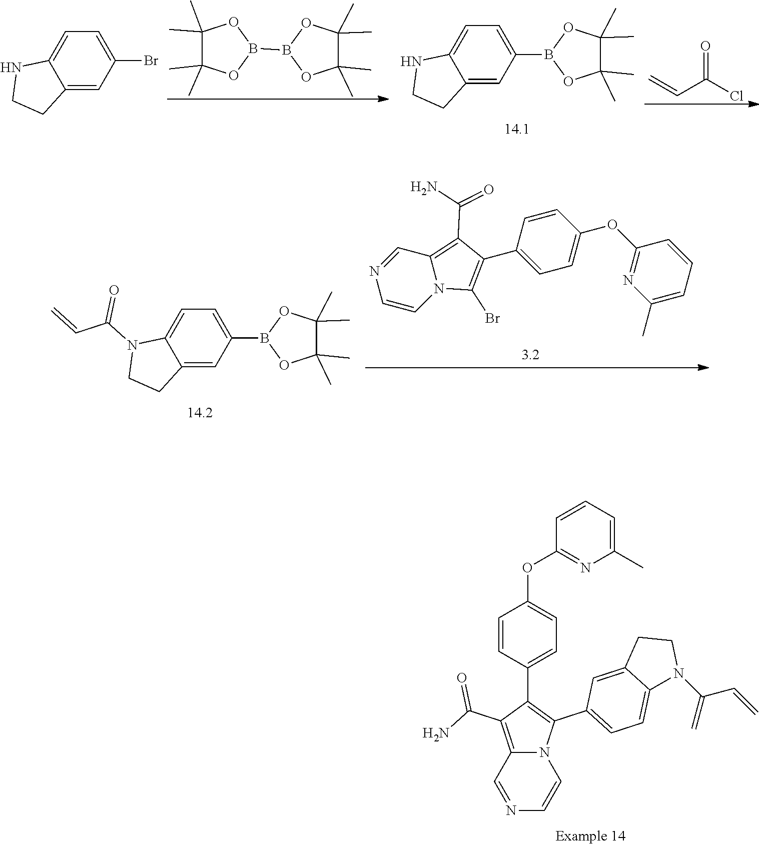

### Step 1. 5-(4,4,5,5-tetramethyl-1,3,2-dioxaborolan-2-yl) indoline

To a solution of 5-bromoindoline (2.0 g, 10.1 mmol) and 4,4,5,5-tetramethyl-2-(4,4,5,5-tetramethyl-1,3,2-dioxaborolan-2-yl)-1,3,2-dioxaborolane (3.85 g, 15.1 mmol) in dioxane (20 mL) were added KOAc (2.97 g, 30.3 mmol) and Pd(dppf)Cl₂ (0.74 g, 1.01 mmol) successively. The reaction mixture was stirred at 80° C. for 16 hours under N₂ atmosphere. After cooling to rt, the mixture was diluted with H₂O (20 mL) and extracted with ethyl acetate (20 mL*3). The combined organic layers were concentrated under reduced pressure to give a residue which was purified by flash column chromatography on silica gel (eluting with 0˜30% ethyl acetate in petroleum ether) to afford the title compound (2.0 g, 81%). LC-MS (ESI+): m/z 245.8 [M+H]⁺.

### Step 2. 1-(5-(4,4,5,5-tetramethyl-1,3,2-dioxaborolan-2-yl) indolin-1-yl) prop-2-en-1-one

To a solution of 5-(4,4,5,5-tetramethyl-1,3,2-dioxaborolan-2-yl) indoline (1.9 g, 7.75 mmol) in THF (20 mL) was added TEA (2.16 mL, 15.5 mmol). A solution of prop-2-enoyl chloride (0.84 g, 9.30 mmol) in THF (5 mL) was added dropwise to the mixture. Then the solution was stirred at 0° C. for 0.5 hr. The mixture was diluted with H₂O (20 mL) and extracted with ethyl acetate (20 mL*3). The combined organic layers were concentrated under reduced pressure to give a residue which was purified by flash column chromatography on silica gel (eluting with 0˜30% ethyl acetate in petroleum ether) to afford the title compound (1.0 g, 43%). LC-MS (ESI+): m/z 300.2 [M+H]⁺.

### Step 3. 6-(1-acryloylindolin-5-yl)-7-(4-((6-methylpyridin-2-yl)oxy)phenyl)pyrrolo[1,2-a]pyrazine-8-carboxamide

To a solution of 6-bromo-7-[4-[(6-methyl-2-pyridyl)oxy]phenyl]pyrrolo[1,2-a]pyrazine-8-carboxamide (150 mg, 354.4 μmol) and 1-[5-(4,4,5,5-tetramethyl-1,3,2-dioxaborolan-2-yl) indolin-1-yl]prop-2-en-1-one (159.0 mg, 531.6 μmol) in dioxane (6 mL) and H₂O (2 mL) were added K₂CO₃ (123 mg, 886.0 μmol) and Pd-118 (23 mg, 35.4 μmol) successively. The reaction mixture was stirred at 90° C. for 2 hours under N₂ atmosphere. After cooling to rt, the reaction mixture was concentrated under reduced pressure to give a residue which was purified by prep-HPLC (column: YMC-Triart Prep C₁₈ 150*40 mm*7 um; mobile phase: [water (FA)-ACN]; B %: 13%-53%, 9 min) to afford the title compound (44 mg, 24%). LC-MS (ESI+): m/z 516.1 [M+H]⁺. ¹H NMR (400 MHZ, DMSO-d₆) δ 9.31 (s, 1H), 8.18 (d, J=8.3 Hz, 1H), 8.06-7.99 (m, 1H), 7.80-7.70 (m, 1H), 7.67 (d, J=4.9 Hz, 1H), 7.40 (br s, 1H), 7.31 (d, J=8.6 Hz, 2H), 7.26 (s, 1H), 7.13 (dd, J=1.5, 8.4 Hz, 1H), 7.08 (d, J=8.6 Hz, 2H), 7.02 (d, J=7.4 Hz, 1H), 6.85-6.70 (m, 2H), 6.61 (br s, 1H), 6.32 (dd, J=1.6, 16.7 Hz, 1H), 5.89-5.79 (m, 1H), 4.25 (t, J=8.3 Hz, 2H), 3.17 (t, J=8.4 Hz, 2H), 2.34 (s, 3H).

### Example 15:7-(4-acrylamidophenyl)-6-(4-((6-methylpyridin-2-yl)oxy)phenyl)pyrrolo[1,2-c]pyrimidine-5-carboxamide

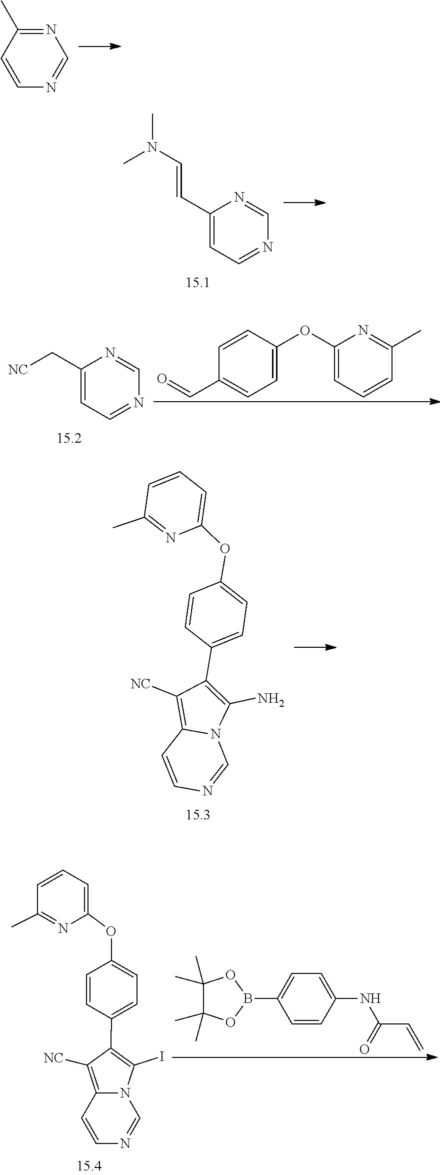

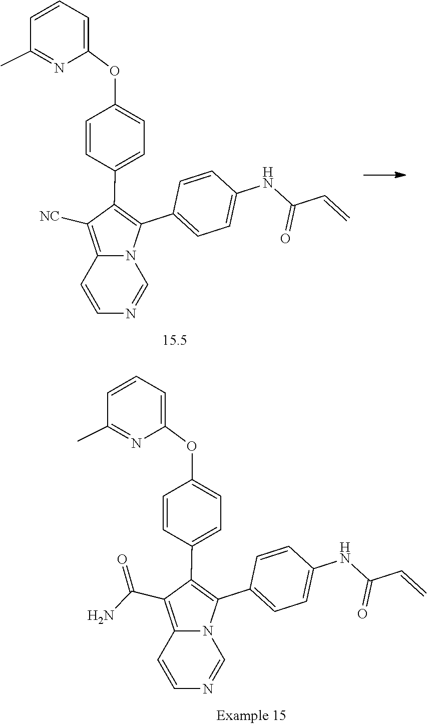

### Step 1. N,N-dimethyl-2-(pyrimidin-4-yl) ethen-1-amine

To a solution of 4-methylpyrimidine (8.0 g, 85.0 mmol) in DMF (39.2 mL) was added DMF-DMA (30.4 g, 255.0 mmol, 33.9 mL). The reaction mixture was stirred at 140° C. for 12 hrs. Then the mixture was concentrated in vacuum to give the title compound (15.0 g) as a brown liquid.

### Step 2. 2-(pyrimidin-4-yl) acetonitrile

To a solution of N,N-dimethyl-2-(pyrimidin-4-yl) ethen-1-amine (8.0 g, 53.6 mmol) in H₂O (80 mL) was added hydroxylamine-O-sulfonic acid (15.2 g, 134.1 mmol). The reaction mixture was stirred at 50° C. for 0.5 hr. After cooling to rt, the pH of the reaction solution was adjusted to 7 by aqueous solution of sodium carbonate. After extraction with ethyl acetate (200 ml*3), the combined organic layers were washed with brine (200 mL), dried over Na₂SO₄, filtered, and concentrated under reduced pressure to give a residue which was purified by flash column chromatography on silica gel (eluting with 0˜30% ethyl acetate in petroleum ether) to afford the title compound (2.0 g, 31%). ¹H NMR (400 MHZ, DMSO-d₆) δ 9.19 (d, J=0.8 Hz, 1H), 8.83 (d, J=5.0 Hz, 1H), 7.61-7.49 (m, 1H), 4.40-4.28 (m, 2H).

### Step 3. 7-amino-6-(4-((6-methylpyridin-2-yl)oxy)phenyl)pyrrolo[1,2-c]pyrimidine-5-carbonitrile

To a mixture of 2-(pyrimidin-4-yl) acetonitrile (400 mg, 3.36 mmol) and 4-((6-methylpyridin-2-yl)oxy)benzaldehyde (716.0 mg, 3.36 mmol) in MeOH (12 mL) were added DBU (1.53 g, 10.1 mmol) and acetyl cyanide (231.9 mg, 3.36 mmol) at 25° C. The reaction mixture was purged with N₂ for 3 times, stirred and heated at 100° C.° C. under microwave irradiation for 20 mins. After cooling to rt, to the reaction mixture was added water (50 mL). After extraction with ethyl acetate (50 mL×3), the combined organic layers were washed with brine (60 mL), dried over Na₂SO₄, filtered, and concentrated under reduced pressure to give a residue which was purified by flash column chromatography on silica gel (eluting with 0˜3% MeOH in DCM) to afford the title compound (410 mg, 36%). LC-MS (ESI+): m/z 342.1 [M+H]⁺.

### Step 4. 7-iodo-6-(4-((6-methylpyridin-2-yl)oxy)phenyl)pyrrolo[1,2-c]pyrimidine-5-carbonitrile

To a solution of 7-amino-6-(4-((6-methylpyridin-2-yl)oxy)phenyl)pyrrolo[1,2-c]pyrimidine-5-carbonitrile (280 mg, 820.2 μmol) and CH₂I₂ (3.36 g, 12.6 mmol, 1.01 mL) in CHCl₃ (5 mL) stirred at 70° C. was added isopentyl nitrite (384 mg, 3.28 mmol) in portions over 30 minutes while maintaining the inner temperature at 70° C. The reaction mixture was stirred at 70° C. for 4 hrs. After cooling to rt, the mixture was concentrated under reduced pressure to give a residue which was purified by flash column chromatography on silica gel (eluting with 0˜20% ethyl acetate in petroleum ether) to afford the title compound (200 mg, 54%). LC-MS (ESI+): m/z 453.0 [M+H]⁺.

### Step 5. N-(4-(5-cyano-6-(4-((6-methylpyridin-2-yl)oxy)phenyl)pyrrolo[1,2-c]pyrimidin-7-yl)phenyl) acrylamide

To a mixture of N-(4-(4,4,5,5-tetramethyl-1,3,2-dioxaborolan-2-yl)phenyl) acrylamide (181 mg, 663.4 μmol) and 7-iodo-6-(4-((6-methylpyridin-2-yl)oxy)phenyl)pyrrolo[1,2-c]pyrimidine-5-carbonitrile (200 mg, 442.2 μmol) in dioxane (3 mL) and H₂O (1 mL) were added K₂CO₃ (183 mg, 1.33 mmol) and Pd-118 (29 mg, 44.2 μmol) successively under N₂. The reaction mixture was stirred at 90° C. for 2 hrs under N₂. After cooling to rt, the mixture was concentrated under reduced pressure to give a residue which was purified by flash column chromatography on silica gel (eluting with 0˜20% ethyl acetate in petroleum ether) to afford the title compound (90 mg, 43%). LC-MS (ESI+): m/z 472.1 [M+H]⁺.

### Step 6. 7-(4-acrylamidophenyl)-6-(4-((6-methylpyridin-2-yl)oxy)phenyl)pyrrolo[1,2-c]pyrimidine-5-carboxamide

A mixture of N-(4-(5-cyano-6-(4-((6-methylpyridin-2-yl)oxy)phenyl)pyrrolo[1,2-c]pyrimidin-7-yl)phenyl) acrylamide (90 mg, 190.9 μmol) in concentrated H₂SO₄ (3 mL) was stirred at 30° C. for 16 hrs. Then the pH of the reaction mixture was adjusted to 7 by aqueous solution of sodium carbonate. After extraction with ethyl acetate (20 ml*3), the combined organic layers were washed with brine (30 mL), dried over Na₂SO₄, filtered, and concentrated under reduced pressure to give a residue which was purified by prep-HPLC (column: Boston Green ODS 150*30 mm*5 um; mobile phase: [water (NH₃H₂O+NH₄HCO₃)-ACN]; B %: 25%-55%, 10 min) to afford the title compound (21 mg, 22%). LC-MS (ESI+): m/z 490.2 [M+H]⁺. ¹H NMR (400 MHZ, DMSO-d₆) δ 10.29 (s, 1H), 8.87 (d, J=1.3 Hz, 1H), 7.94 (d, J=6.4 Hz, 1H), 7.78-7.65 (m, 4H), 7.38-7.16 (m, 5H), 7.11-6.99 (m, 3H), 6.77 (d, J=8.1 Hz, 1H), 6.50-6.40 (m, 1H), 6.32-6.12 (m, 2H), 5.82-5.71 (m, 1H), 2.34 (s, 3H).

### Example 16:6-(4-acrylamidophenyl)-7-(4-(((1-fluorocyclobutyl)methyl) carbamoyl) phenyl)pyrrolo[1,2-a]pyrazine-8-carboxamide

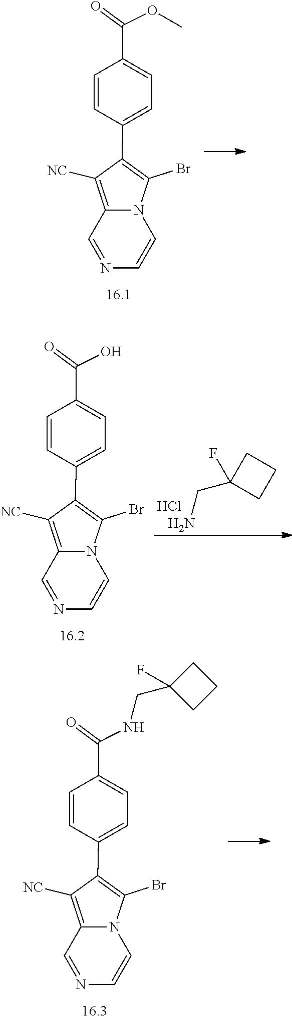 [SMILES: COC(=O)c1ccc(-c2c(C#N)c3cnccn3c2Br)cc1 ; N#Cc1c(-c2ccc(C(=O)O)cc2)c(Br)n2ccncc12 ; Cl ; NCC1(F)CCC1 ; N#Cc1c(-c2ccc(C(=O)NCC3(F)CCC3)cc2)c(Br)n2ccncc12]

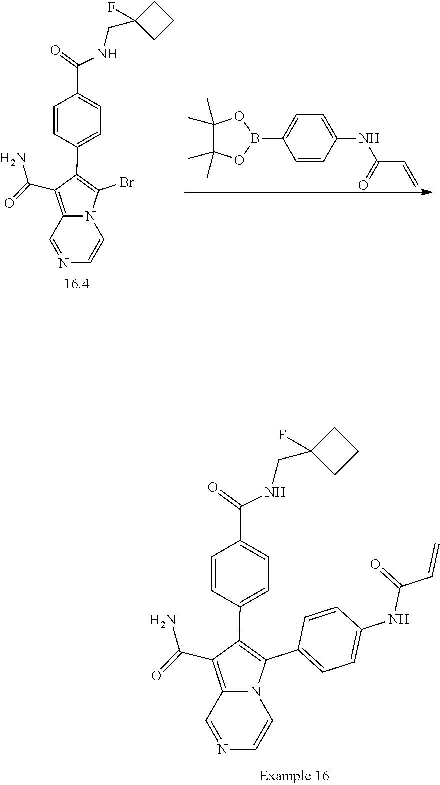 [SMILES: NC(=O)c1c(-c2ccc(C(=O)NCC3(F)CCC3)cc2)c(Br)n2ccncc12 ; C=CC(=O)Nc1ccc(B2OC(C)(C)C(C)(C)O2)cc1 ; C=CC(=O)Nc1ccc(-c2c(-c3ccc(C(=O)NCC4(F)CCC4)cc3)c(C(N)=O)c3cnccn23)cc1]

### Step 1. 4-(6-bromo-8-cyanopyrrolo[1,2-a]pyrazin-7-yl)benzoic acid

In a similar fashion according to the procedure for intermediate 2.2, intermediate 16.1 was synthesized by replacing 4-((4-methylpyrimidin-2-yl)oxy)benzaldehyde with methyl 4-formylbenzoate.

To a solution of methyl 4-(6-bromo-8-cyanopyrrolo[1,2-a]pyrazin-7-yl)benzoate (intermediate 16.1) (480 mg, 1.35 mmol) in THF (10.0 mL) was added LiOH·H₂O (2 M in water, 3.37 mL) at 25° C. The reaction mixture was stirred at 25° C. for 12 hrs. Then the mixture was extracted with DCM (20.0 mL). The pH of the aqueous phase was adjusted to 7 by 1N HCl. After extraction with DCM (20.0 mL*3), the combined organic layers were dried over anhydrous Na₂SO₄, filtered and concentrated in vacuum to afford the title compound (300 mg, 43%). LC-MS (ESI+): m/z 341.8 [M+H]⁺.

### Step 2. 4-(6-bromo-8-cyanopyrrolo[1,2-a]pyrazin-7-yl)-N-((1-fluorocyclobutyl)methyl)benzamide

To a solution of 4-(6-bromo-8-cyanopyrrolo[1,2-a]pyrazin-7-yl)benzoic acid (370 mg, 1.08 mmol) in DCM (10 mL) were added DIPEA (698 mg, 5.41 mmol), (1-fluorocyclobutyl) methanamine hydrochloride (166 mg, 1.19 mmol) and HATU (616 mg, 1.62 mmol) at 25° C. The reaction mixture was stirred at 25° C.° C. for 12 hrs. Then the mixture was diluted with H₂O (50 mL) and extracted with DCM (10 mL*3). The combined organic layers were washed with brine (60 mL), dried over Na₂SO₄, filtered, and concentrated under reduced pressure to give a residue which was purified by flash column chromatography on silica gel (eluting with 1˜50% EtOAc in petroleum ether) to afford the title compound (500 mg). LC-MS (ESI+): m/z 426.8 [M+H]⁺.

### Step 3. 6-bromo-7-(4-(((1-fluorocyclobutyl)methyl) carbamoyl)phenyl)pyrrolo[1,2-a]pyrazine-8-carboxamide

To a solution of 4-(6-bromo-8-cyanopyrrolo[1,2-a]pyrazin-7-yl)-N-((1-fluorocyclobutyl)methyl)benzamide (100 mg, 234.0 μmol) in DMSO (25.0 mL) was added K₂CO₃ (113 mg, 819.0 μmol). H₂O₂ (1.09 mL, 11.3 mmol, 30% purity) was added dropwise at 0° C. The reaction mixture was stirred at 25° C. for 5 hrs. Then the mixture was quenched by addition of aq. Na₂SO₃ (40.0 mL). After extraction with DCM (30.0 mL×3), the combined organic layers were dried over Na₂SO₄, filtered, and concentrated under reduced pressure to give the title compound (100 mg) as a yellow oil. LC-MS (ESI+): m/z 444.9 [M+H]⁺.

### Step 4. 6-(4-acrylamidophenyl)-7-(4-(((1-fluorocyclobutyl)methyl) carbamoyl)phenyl)pyrrolo[1,2-a]pyrazine-8-carboxamide

To a mixture of 6-bromo-7-(4-(((1-fluorocyclobutyl)methyl) carbamoyl)phenyl)pyrrolo[1,2-a]pyrazine-8-carboxamide (100 mg, 224.0 μmol) in dioxane (4.0 mL) and H₂O (1.0 mL) were added N-(4-(4,4,5,5-tetramethyl-1,3,2-dioxaborolan-2-yl)phenyl) acrylamide (122 mg, 449 μmol), Na₂CO₃ (71 mg, 673.0 μmol) and Pd(PPh₃)₄ (26 mg, 22.4 μmol) at 25° C. under N₂. The reaction mixture was stirred at 70° C. for 12 hrs under N₂ atmosphere. After cooling to rt, the reaction mixture was filtered and concentrated under reduced pressure to give a residue which was purified by prep-HPLC (column: Phenomenex luna C₁₈ 150*25 mm*10 um; mobile phase: [water (HCl)-MeOH]; B %: 16%-46%, 10 min) to afford the title compound (20 mg, 17%). LC-MS (ESI+): m/z 512.3 [M+H]⁺. ¹H NMR (400 MHZ, DMSO-d₆) δ 10.30 (s, 1H), 9.26 (d, J=1.3 Hz, 1H), 8.71 (m, 1H), 8.04-7.97 (m, 1H), 7.85-7.62 (m, 5H), 7.41-7.25 (m, 5H), 6.82-6.71 (m, 1H), 6.48-6.39 (m, 1H), 6.31-6.23 (m, 1H), 5.81-5.73 (m, 1H), 3.68-3.56 (m, 2H), 2.23-2.10 (m, 4H), 1.81-1.70 (m, 1H), 1.58-1.42 (m, 1H).

### Preparation of 6-iodo-3-methoxy-7-(4-((6-methylpyridin-2-yl)oxy)phenyl)pyrrolo[1,2-a]pyrazine-8-carbonitrile (intermediate 17.6)

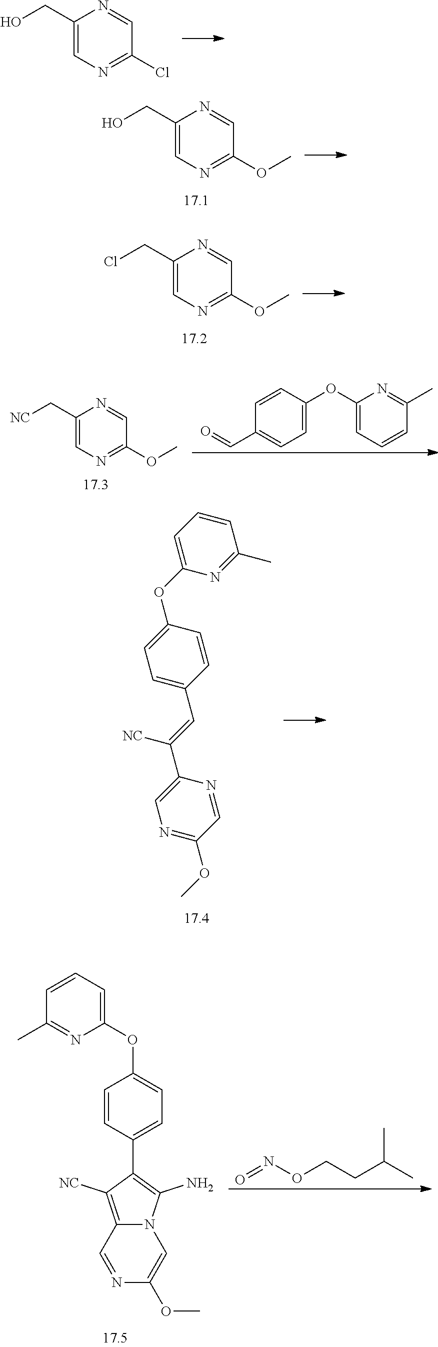

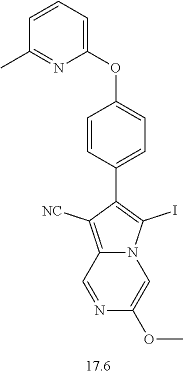

### Step 1. (5-methoxypyrazin-2-yl) methanol

To a solution of (5-chloropyrazin-2-yl) methanol (4.0 g, 27.67 mmol) in methanol (120 mL) was added sodium methanolate (14.95 g, 276.70 mmol) in portions over 10 min at 25° C. The reaction mixture was stirred at 25° C. for 18 hr. Then to the reaction mixture was added water (50 mL). After extraction with ethyl acetate (50 mL×3), the combined organic layers were dried over Na₂SO₄, filtered, and concentrated under reduced pressure to give a residue which was purified by flash column chromatography on silica gel (eluting with 0% to 25% ethyl acetate in petroleum ether) to afford the title compound (2.6 g, 67%). LC-MS (ESI+): m/z 141.0 [M+H]⁺.

### Step 2. 2-(chloromethyl)-5-methoxypyrazine

To a solution of (5-methoxypyrazin-2-yl) methanol (2.6 g, 18.55 mmol) in dichloromethane (52 mL) was added sulfinyl dichloride (6.73 mL, 92.76 mmol) slowly at 25° C. The reaction mixture was stirred at 25° C. for 18 hr. Then the mixture was concentrated under reduced pressure to afford the title compound (2.7 g, 92%) as a yellow oil which was used for next step directly. LC-MS (ESI+): m/z 159.0 [M+H]⁺.

### Step 3. 2-(5-methoxypyrazin-2-yl) acetonitrile

To a solution of 2-(chloromethyl)-5-methoxypyrazine (2.7 g, 17.02 mmol) in ethanol (150 mL) and H₂O (50 mL) were added potassium iodide (0.85 g, 5.11 mmol), K₂CO₃ (2.35 g, 17.03 mmol) and KCN (1.22 g, 18.73 mmol). The reaction mixture was stirred at 80° C. for 18 hr. After cooling to rt, saturated Na₂CO₃ solution (100 mL) was added to the mixture. After extraction with ethyl acetate (100 mL×3), the combined organic layers were dried over Na₂SO₄, filtered, and concentrated under reduced pressure to give a residue which was purified by column chromatography on silica gel (eluting with 0% to 25% ethyl acetate in petroleum ether) to afford the title compound (1.7 g, 67%). LC-MS (ESI+): m/z 150.0 [M+H]⁺.

### Step 4. 2-(5-methoxypyrazin-2-yl)-3-(4-((6-methylpyridin-2-yl)oxy)phenyl) acrylonitrile

To a solution of 2-(5-methoxypyrazin-2-yl) acetonitrile (1.80 g, 12.07 mmol) in methanol (20 mL) were added 4-((4-methylpyrimidin-2-yl)oxy)benzaldehyde (3.09 g, 14.48 mmol) and DBU (5.51 g, 36.20 mmol). The reaction mixture was stirred at 80° C. for 3 hr. After cooling to rt, the precipitate was filtered. The solid was washed with water (20 mL) and dried under reduced pressure to give the title compound (3 g, 72%). LC-MS (ESI+): m/z 345.0 [M+H]⁺.

### Step 5. 6-amino-3-methoxy-7-(4-((6-methylpyridin-2-yl)oxy)phenyl)pyrrolo[1,2-a]pyrazine-8-carbonitrile

To a mixture of 2-(5-methoxypyrazin-2-yl)-3-(4-((6-methylpyridin-2-yl)oxy)phenyl) acrylonitrile (3 g, 8.71 mmol) in methanol (90 mL) were added DBU (4 g, 26.14 mmol) and acetyl cyanide (0.72 g, 10.45 mmol). The reaction mixture was stirred at 120° C. for 20 min under microwave irradiation. After cooling to rt, water (200 mL) was added to the reaction. After extraction with ethyl acetate (200 mL×3), the combined organic layers were dried over Na₂SO₄, filtered, and concentrated under reduced pressure to give a residue which was purified by column chromatography on silica gel (eluting with 0% to 5% methanol in dichloromethane) to afford the title compound (740 mg, 23%). LC-MS (ESI+): m/z 372.3 [M+H]⁺.

### Step 6. 6-iodo-3-methoxy-7-(4-((6-methylpyridin-2-yl)oxy)phenyl)pyrrolo[1,2-a]pyrazine-8-carbonitrile

To a solution of 6-amino-3-methoxy-7-(4-((6-methylpyridin-2-yl)oxy)phenyl)pyrrolo[1,2-a]pyrazine-8-carbonitrile (560 mg, 1.51 mmol) in chloroform (50 mL) were added 3-methyl-1-(nitrosooxy) butane (530 mg, 4.5 mmol) and diiodomethane (606 mg, 2.26 mmol). The reaction mixture was stirred at 70° C. for 1 hr. After cooling to rt, water (50 mL) was added to the reaction. After extraction with DCM (50 mL×3), the combined organic layers were dried over Na₂SO₄, filtered, and concentrated under reduced pressure to give a residue which was purified by column chromatography on silica gel (eluting with 0% to 50% ethyl acetate in petroleum ether) to afford the title compound (50 mg, 7%). LC-MS (ESI+): m/z 482.8 [M+H]⁺.

### Example 17:6-(4-acrylamidophenyl)-3-methoxy-7-(4-((6-methylpyridin-2-yl)oxy)phenyl)pyrrolo[1,2-a]pyrazine-8-carboxamide

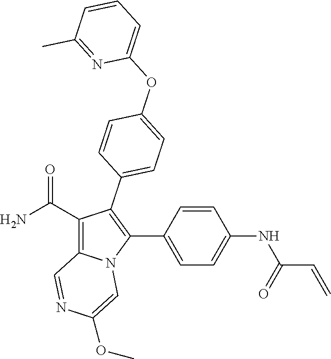 [SMILES: C=CC(=O)Nc1ccc(-c2c(-c3ccc(Oc4cccc(C)n4)cc3)c(C(N)=O)c3cnc(OC)cn23)cc1]

### Example 17

In a similar fashion according to the procedure for Example 1, Example 17 was synthesized by replacing 6-bromo-7-(4-((6-methylpyridin-2-yl)oxy)phenyl)pyrrolo[1,2-a]pyrazine-8-carbonitrile with 6-iodo-3-methoxy-7-(4-((6-methylpyridin-2-yl)oxy)phenyl)pyrrolo[1,2-a]pyrazine-8-carbonitrile. The crude product was purified by prep-HPLC (eluted with 45% to 55% MeCN in H₂O containing 0.1% FA) to give the title compound (6 mg, 20%). LC-MS (ESI+): m/z 520.3 [M+H]⁺. ¹H NMR (400 MHZ, DMSO-d₆) δ 10.33 (s, 1H), 9.14 (d, J=1.0 Hz, 1H), 7.77-7.70 (m, 3H), 7.45 (s, 1H), 7.31-7.27 (m, 5H), 7.07 (d, J=8.0 Hz, 2H), 7.015 (d, J=4.0 Hz, 1H), 6.76 (d, J=8.0 Hz, 1H), 6.50-6.38 (m, 2H), 6.27 (dd, J₁=16.0, J₂=2.0 Hz, 1H), 5.78 (dd, J₁=12.0, J₂=2.0 Hz, 1H), 3.82 (s, 3H), 2.34 (s, 3H).

### Preparation of Intermediate 18.5

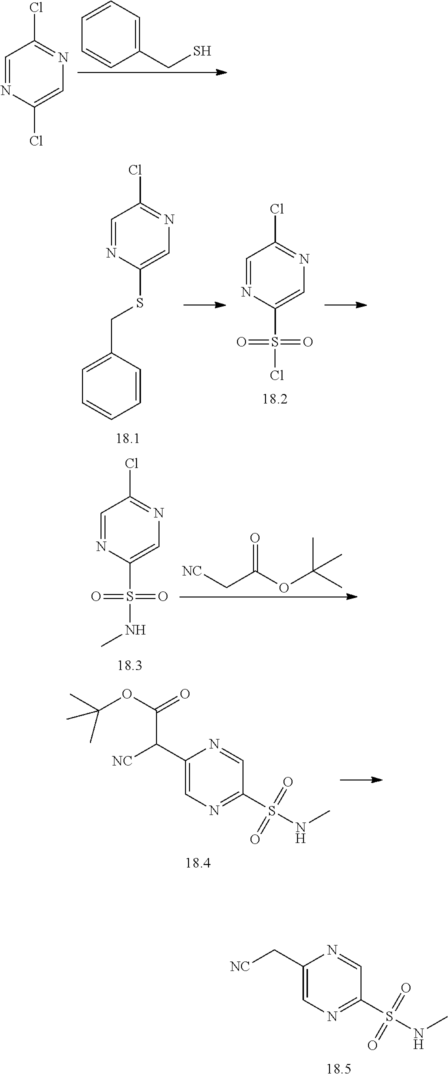

### Step 1. 2-(benzylthio)-5-chloropyrazine

To a solution of 2,5-dichloropyrazine (7.85 g, 52.70 mmol) in 1,4-dioxane (160 mL) were added phenylmethanethiol (6.54 g, 52.70 mmol), Xantphos (6.10 g, 10.54 mmol), Pd₂(dba)₃ (4.83 g, 5.27 mmol) and DIPEA (26.13 mL, 158.08 mmol). The reaction mixture was stirred at 100° C. for 2 hrs under N₂. After cooling to rt, the mixture was diluted with brine (100 mL) and extracted with DCM (100 mL×3). The combined organic layers were dried over Na₂SO₄, filtered, and concentrated under reduced pressure to give a residue which was purified by column chromatography on silica gel eluting with petroleum ether to give the title compound (3.77 g, 30%) as a yellow oil. LC-MS (ESI+): m/z 237.2 [M+H].

### Step 2. 5-chloropyrazine-2-sulfonyl chloride

To a solution of 2-(benzylthio)-5-chloropyrazine (3.1 g, 13.09 mmol) in DCM (60 mL) and H₂O (6 mL) was added sulfuryl chloride (11.84 g, 87.74 mmol) at 0˜10° C. The reaction mixture was stirred at 0° C. for 2 hrs under N₂. Water (100 mL) was added to the reaction mixture. After extraction with DCM (100 mL×3), the combined organic layers were dried over Na₂SO₄, filtered, and concentrated under reduced pressure to give a residue which was purified by column chromatography on silica gel eluting with ethyl acetate (from 0% to 10%) in petroleum ether to afford the title compound (1.99 g, 71%). ¹H NMR (400 MHZ, CDCl₃) δ 9.095 (d, J=4.0 Hz, 1H), 8.775 (d, J=4.0 Hz, 1H).

### Step 3. 5-chloro-N-methylpyrazine-2-sulfonamide

To a suspension of methylamine hydrochloride (0.60 g, 8.92 mmol) in DCM (10 mL) were added triethylamine (1.24 mL, 8.92 mmol) and 5-chloropyrazine-2-sulfonyl chloride (1.9 g, 8.92 mmol) at 0° C. The reaction mixture was stirred at 25° C. for 2 hr. Brine (100 mL) was added to the reaction mixture. After extraction with DCM (100 mL×3), the combined organic layers were dried over Na₂SO₄, filtered, and concentrated under reduced pressure to give a residue which was purified by column chromatography on silica gel eluting with ethyl acetate (from 0% to 50%) in petroleum ether to afford the title compound (1.46 g, 79%) as an oil. ¹H NMR (400 MHZ, CDCl₃) δ 8.985 (d, J=4.0 Hz, 1H), 8.66 (d, J=1.3 Hz, 1H), 4.82 (br s, 1H), 2.835 (d, J=4.0 Hz, 3H).

### Step 4. tert-butyl 2-cyano-2-(5-(N-methylsulfamoyl) pyrazin-2-yl)acetate

To a solution of 5-chloro-N-methylpyrazine-2-sulfonamide (650 mg, 3.13 mmol) in THF (20 mL) were added tert-butyl 2-cyanoacetate (884 mg, 6.26 mmol) and Cs₂CO₃ (2.55 g, 7.82 mmol). The reaction mixture was stirred at 80° C. for 1 hr. After cooling to rt, water (100 mL) was added to the mixture. After extraction with ethyl acetate (50 mL×3), the pH of aqueous layer was adjusted to 3 by diluted HCl solution (IN). After extraction with DCM (100 mL×3), the combined organic layers were dried over Na₂SO₄, filtered, and concentrated under reduced pressure to give a residue which was purified by column chromatography on silica gel eluting with DCM to give the title compound (850 mg, 87%). LC-MS (ESI+): m/z 313.2 [M+H]⁺.

### Step 5. 5-(cyanomethyl)-N-methylpyrazine-2-sulfonamide

To a solution of tert-butyl 2-cyano-2-(5-(N-methylsulfamoyl) pyrazin-2-yl)acetate (840 mg, 2.69 mmol) in toluene (25 mL) was added 4-methylbenzenesulfonic acid (463 mg, 2.69 mmol). The reaction mixture was stirred at 120° C. for 1 hr under N₂. After cooling to rt, to the mixture was added water (100 mL). After extraction with DCM (100 mL×3), the combined organic layers were dried over Na₂SO₄, filtered, and concentrated under reduced pressure to give a residue which was purified by column chromatography on silica gel eluting with dichloromethane to give the title compound (260 mg, 46%) as a yellow oil. ¹H NMR (400 MHZ, DMSO-d₆) δ 9.10 (s, 1H), 8.83 (s, 1H), 8.02 (d, J=8.0 Hz, 1H), 4.49 (s, 2H), 2.585 (d, J=4.0 Hz, 3H).

### Example 18:6-(4-acrylamidophenyl)-7-(4-((6-methylpyridin-2-yl)oxy)phenyl)-3-(N-methylsulfamoyl)pyrrolo[1,2-a]pyrazine-8-carboxamide

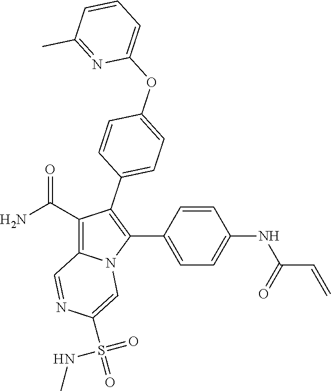 [SMILES: C=CC(=O)Nc1ccc(-c2c(-c3ccc(Oc4cccc(C)n4)cc3)c(C(N)=O)c3cnc(S(=O)(=O)NC)cn23)cc1]

### Example 18

In a similar fashion according to the procedure for Example 1, Example 18 was synthesized by replacing 2-(pyrazin-2-yl) acetonitrile with 5-(cyanomethyl)-N-methylpyrazine-2-sulfonamide (intermediate 18.5). The crude product was purified by pre-HPLC (eluted with 40% to 50% MeCN in H₂O containing 0.1% FA) to give the title compound (8 mg, 16%). LC-MS (ESI+): m/z 583.2 [M+H]⁺. ¹H NMR (400 MHZ, CDCl₃) δ 9.84 (d, J=1.2 Hz, 1H), 8.59 (d, J=1.2 Hz, 1H), 7.69-7.62 (m, 3H), 7.37 (s, 1H), 7.29 (d, J=8.0 Hz, 2H), 7.23 (d, J=8.0 Hz, 2H), 7.13 (d, J=8.0 Hz, 2H), 6.95 (d, J=8.0 Hz, 1H), 6.71 (d, J=8.0 Hz, 1H), 6.46 (d, J=20.0 Hz, 1H), 6.35-6.20 (m, 1H), 5.82 (d, J=12.0 Hz, 1H), 5.67-5.35 (m, 2H), 5.02-4.92 (m, 1H), 2.71 (d, J=4.0 Hz, 3H), 2.47 (s, 3H).

### Example 19:6-(4-acrylamidophenyl)-7-(4-((4-methylthiazol-2-yl)oxy)phenyl)pyrrolo[1,2-a]pyrazine-8-carboxamide

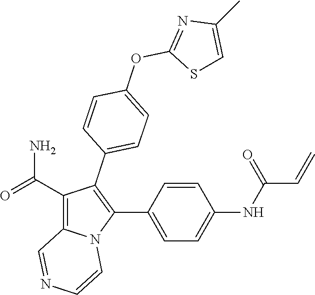 [SMILES: C=CC(=O)Nc1ccc(-c2c(-c3ccc(Oc4nc(C)cs4)cc3)c(C(N)=O)c3cnccn23)cc1]

### Example 19

### Preparation of Intermediate 19.1

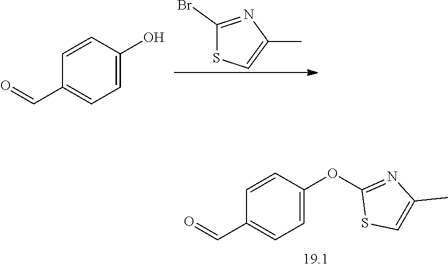

To a solution of 4-hydroxybenzaldehyde (10.0 g, 81.9 mmol) in DMF (100 mL) were added 2-bromo-4-methylthiazole (16.0 g, 90.0 mmol) and CsF (37.3 g, 245.0 mmol) at 25° C. The reaction mixture was stirred at 120° C. for 12 hrs. Then the mixture was diluted with H₂O (100 mL) and extracted with ethyl acetate (50.0 mL*3). The combined organic layers were washed with brine (50.0 mL), dried over Na₂SO₄, filtered, and concentrated under reduced pressure to give a residue which was purified by column chromatography on silica gel eluting with EtOAc (from 1% to 50%) in petroleum ether to afford the title compound (2.50 g, 12%) as a yellow oil. ¹H NMR (400 MHZ, DMSO-d₆) δ 9.99 (s, 1H), 8.05-7.96 (m, 2H), 7.57-7.47 (m, 2H), 6.90 (s, 1H), 2.22 (s, 3H).

In a similar fashion according to the procedure for Example 15, Example 19 was synthesized by replacing 4-((6-methylpyridin-2-yl)oxy)benzaldehyde with 4-((4-methylthiazol-2-yl)oxy)benzaldehyde, and replacing 2-(pyrimidin-4-yl) acetonitrile with 2-(pyrazin-2-yl) acetonitrile. The crude product was purified by prep-HPLC (column: Waters Xbridge 150*25 mm*5 um; mobile phase: [water (NH₄HCO₃)-ACN]; B %: 25%-55%, 8 min) to afford the title compound (25 mg, 12%). LC-MS (ESI+): m/z 496.3 [M+H]⁺. ¹H NMR (400 MHZ, DMSO-d₆) δ 10.31 (s, 1H), 9.26 (d, J=1.3 Hz, 1H), 8.00 (dd, J=1.4, 4.9 Hz, 1H), 7.75 (d, J=8.6 Hz, 2H), 7.65 (d, J=4.9 Hz, 1H), 7.40-7.27 (m, 7H), 6.84-6.68 (m, 2H), 6.49-6.39 (m, 1H), 6.32-6.24 (m, 1H), 5.82-5.74 (m, 1H), 2.21 (d, J=1.0 Hz, 3H).

### Example 20:7-(4-acrylamidophenyl)-6-(4-((6-methylpyridin-2-yl)oxy)phenyl)pyrrolo[1,2-b]pyridazine-5-carboxamide

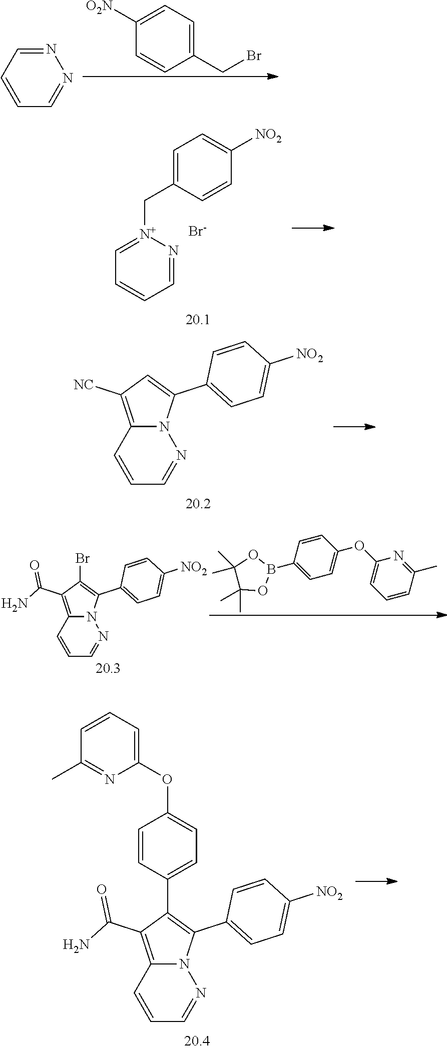

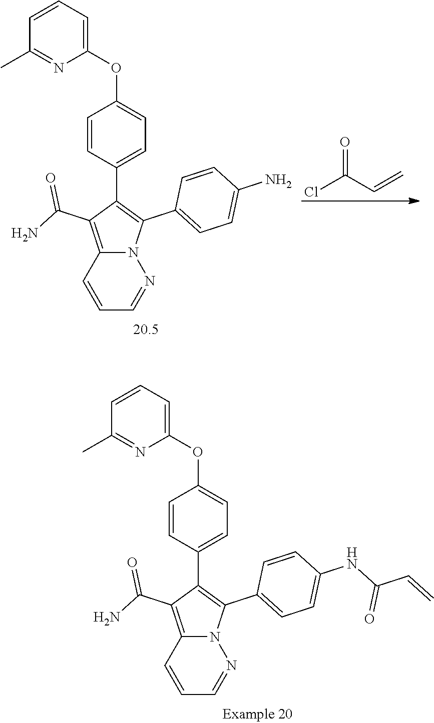

### Step 1. 1-(4-nitrobenzyl)pyridazin-1-ium bromide

To a mixture of pyridazine (5.87 g, 73.2 mmol) in EtOAc (30.0 mL) was added 1-(bromomethyl)-4-nitro-benzene (19.0 g, 87.9 mmol) in one portion at 25° C. under N₂. The mixture was stirred at 80° C. for 12 hours. Then the mixture was cooled to rt and filtered. The filter cake was dried under reduced pressure to give the title compound (15.0 g). LC-MS (ESI+): m/z 216.0 (M+).

### Step 2. 7-(4-nitrophenyl)pyrrolo[1,2-b]pyridazine-5-carbonitrile

To a solution of 1-(4-nitrobenzyl)pyridazin-1-ium bromide (10.0 g, 33.9 mmol) in DME (100 mL) was added prop-2-enenitrile (9.31 g, 175 mmol). MnO₂ (15.2 g, 175 mmol) and TEA (10.7 g, 106 mmol, 14.8 mL) were then added to the solution at rt. Then the reaction mixture was stirred at 80° C. for 7 hrs. After cooling to rt, the mixture was filtered and the filtrate was concentrated under reduced pressure to give a residue which was purified by column chromatography on silica gel eluting with EtOAc (from 1% to 10%) in petroleum ether to afford the title compound (6.40 g, 72%). ¹H NMR (400 MHZ, DMSO-d₆) δ 8.72-8.68 (m, 1H), 8.45-8.39 (m, 3H), 8.39-8.34 (m, 2H), 8.15 (s, 1H), 7.34-7.29 (m, 1H).

### Step 3. 6-bromo-7-(4-nitrophenyl)pyrrolo[1,2-b]pyridazine-5-carboxamide

To a solution of 7-(4-nitrophenyl)pyrrolo[1,2-b]pyridazine-5-carbonitrile (2.00 g, 7.57 mmol) in H₂SO₄ (15.0 mL) was added NBS (2.69 g, 15.1 mmol). The reaction mixture was stirred at 25° C. for 4 hrs. The mixture was poured into ice and the pH was adjusted to 7 by addition of saturation Na₂CO₃ aqueous solution dropwise. After extraction with ethyl acetate (50 mL*3), the combined organic layers were dried over Na₂SO₄, filtered, and concentrated under reduced pressure to give the title compound (800 mg, 29%).

### Step 4. 6-(4-((6-methylpyridin-2-yl)oxy)phenyl)-7-(4-nitrophenyl)pyrrolo[1,2-b]pyridazine-5-carboxamide

To a solution of 6-bromo-7-(4-nitrophenyl)pyrrolo[1,2-b]pyridazine-5-carboxamide (526 mg, 1.46 mmol) in DMF (16 mL) and H₂O (1 mL) were added 2-methyl-6-(4-(4,4,5,5-tetramethyl-1,3,2-dioxaborolan-2-yl) phenoxy)pyridine (498 mg, 1.60 mmol), Pd(DtBPF)Cl₂ (95 mg, 145 μmol) and K₃PO₄ (927 mg, 4.37 mmol). The reaction mixture was stirred at 95° C. for 4 hrs. After cooling to rt, the mixture was diluted with water (20 mL) and extracted with DCM (20 mL*3). The combined organic layers were washed with brine (20 mL), dried over Na₂SO₄, filtered, and concentrated under reduced pressure to give a residue which was purified by prep-HPLC (column: Phenomenex C₁₈ 250*50 mm*10 um; mobile phase: [water (NH₄HCO₃)-ACN]; B %: 37%-67%, 8 min) to afford the title compound (50 mg, 7%). LC-MS (ESI+): m/z 466.1 [M+H]⁺.

### Step 5. 7-(4-aminophenyl)-6-(4-((6-methylpyridin-2-yl)oxy)phenyl)pyrrolo[1,2-b]pyridazine-5-carboxamide

To a solution of 6-(4-((6-methylpyridin-2-yl)oxy)phenyl)-7-(4-nitrophenyl)pyrrolo[1,2-b]pyridazine-5-carboxamide (35 mg, 75.1 μmol) in EtOH (2 mL) and H₂O (2 mL) was added Fe (27 mg, 488 μmol) and CaCl₂) (25 mg, 225 μmol) at rt. The reaction mixture was stirred at 80° C. for 12 hrs. After cooling to rt, the mixture was filtered and the filtrate was concentrated under reduced pressure to give the title compound (30 mg). LC-MS (ESI+): m/z 436.3 [M+H]⁺.

### Step 6. 7-(4-acrylamidophenyl)-6-(4-((6-methylpyridin-2-yl)oxy)phenyl)pyrrolo[1,2-b]pyridazine-5-carboxamide

To a solution of 7-(4-aminophenyl)-6-(4-((6-methylpyridin-2-yl)oxy)phenyl)pyrrolo[1,2-b]pyridazine-5-carboxamide (30 mg, 68.8 μmol) in acetone (2 mL) and H₂O (0.4 mL) was added K₂CO₃ (19.0 mg, 137.0 μmol) at 0° C. Then prop-2-enoylchloride (7.48 mg, 82.6 μmol) was added to the solution at 0° C. The reaction mixture was stirred at 0° C. for 1 hr. Then the mixture was diluted with water (10 mL) and extracted with ethyl acetate (10 mL×3). The combined organic layers were washed with brine (10 mL), dried over anhydrous Na₂SO₄, filtered and concentrated in vacuum to give a residue which was purified by prep-TLC (SiO₂, petroleum ether:ethyl acetate=0:1) to afford the title compound (10 mg, 29%). LC-MS (ESI+): m/z 490.1 [M+H]⁺. ¹H NMR (400 MHZ, DMSO-d₆) δ 10.25 (d, J=8.1 Hz, 1H), 8.50-8.44 (m, 1H), 8.35-8.29 (m, 1H), 7.74 (t, J=7.8 Hz, 1H), 7.64 (d, J=7.6 Hz, 2H), 7.36-7.28 (m, 4H), 7.25 (br s, 1H), 7.13 (d, J=8.6 Hz, 2H), 7.06-6.94 (m, 2H), 6.77 (d, J=8.1 Hz, 1H), 6.53-6.40 (m, 1H), 6.35-6.15 (m, 2H), 5.81-5.70 (m, 1H), 2.36 (s, 3H).

### Example 21:6-(4-acrylamidophenyl)-3-(1-methylazetidin-3-yl)-7-(4-((6-methylpyridin-2-yl)oxy) phenyl)pyrrolo[1,2-a]pyrazine-8-carboxamide

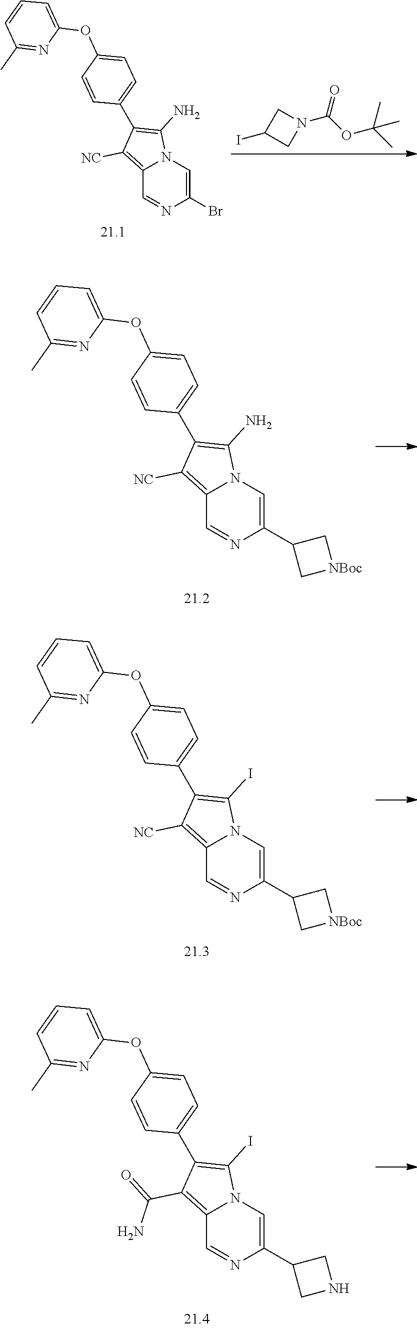 [SMILES: Cc1cccc(Oc2ccc(-c3c(C#N)c4cnc(Br)cn4c3N)cc2)n1 ; CC(C)(C)OC(=O)N1CC(I)C1 ; Cc1cccc(Oc2ccc(-c3c(C#N)c4cnc(C5CN(C(=O)OC(C)(C)C)C5)cn4c3N)cc2)n1 ; Cc1cccc(Oc2ccc(-c3c(C#N)c4cnc(C5CN(C(=O)OC(C)(C)C)C5)cn4c3I)cc2)n1 ; Cc1cccc(Oc2ccc(-c3c(C(N)=O)c4cnc(C5CNC5)cn4c3I)cc2)n1]

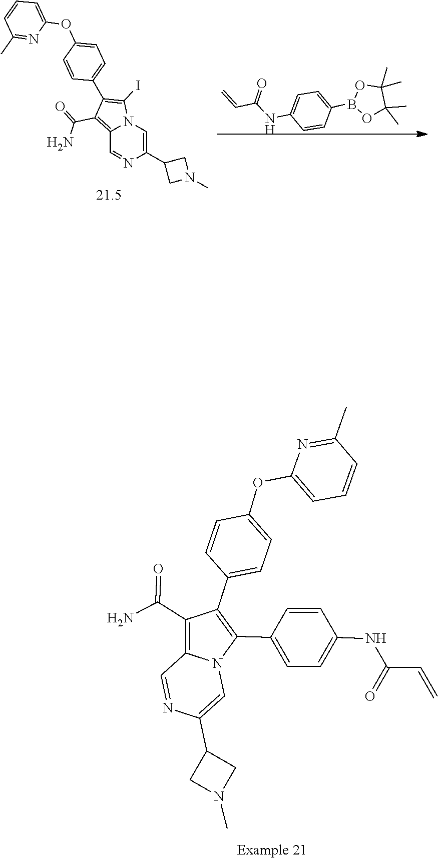

### Step 1. tert-butyl 3-(6-amino-8-cyano-7-(4-((6-methylpyridin-2-yl)oxy)phenyl)pyrrolo[1,2-a]pyrazin-3-yl) azetidine-1-carboxylate

In a similar fashion according to the procedure for intermediate 12.3, intermediate 21.1 was synthesized by replacing 2,3-dichloropyrazine with 2,5-dibromopyrazine.

To a mixture of Zn (498 mg, 7.61 mmol) in THF (10 mL) were added 1,2-dibromoethane (27 mg, 142.77 μmol) and TMSCl (31 mg, 285.54 μmol). The reaction mixture was warmed to 65° C. A solution of tert-butyl 3-iodoazetidine-1-carboxylate (808 mg, 2.86 mmol) in THF (2 mL) was added. Then the mixture was stirred at 65° C. for 1 hour under N₂. 6-Amino-3-bromo-7-[4-[(6-methyl-2-pyridyl)oxy]phenyl]pyrrolo[1,2-a]pyrazine-8-carbonitrile (400 mg, 951.8 μmol), CuI (22.7 mg, 119.0 μmol) and Pd(dppf)Cl₂·CH₂Cl₂ (47 mg, 57.1 μmol) were then added. The reaction mixture was stirred at 80° C. for 1 hr. After cooling to rt, the reaction mixture was diluted with water (100 mL) and extracted with ethyl acetate (50 mL*3). The combined organic layers were washed with brine (100 mL), dried over Na₂SO₄, filtered, and concentrated under reduced pressure to give a residue which was purified by flash column chromatography on silica gel (eluting with 0˜10% MeOH in DCM) to afford the title compound (400 mg, 85%). LC-MS (ESI+): m/z 441.1 (M+H−56)+.

### Step 2. tert-butyl 3-(8-cyano-6-iodo-7-(4-((6-methylpyridin-2-yl)oxy)phenyl)pyrrolo[1,2-a]pyrazin-3-yl) azetidine-1-carboxylate

A mixture of tert-butyl 3-(6-amino-8-cyano-7-(4-((6-methylpyridin-2-yl)oxy)phenyl)pyrrolo[1,2-a]pyrazin-3-yl) azetidine-1-carboxylate (500 mg, 1.01 mmol) in CHCl₃ (5 mL) were added CH₂I₂ (4.13 g, 15.4 mmol) and isopentyl nitrite (472 mg, 4.03 mmol) successively. The reaction mixture was stirred at 70° C. for 1 hour. After cooling to rt, the mixture was concentrated under reduced pressure to give a residue which was purified by flash column chromatography on silica gel (eluting with 0˜35% ethyl acetate in petroleum ether) to afford the title compound (230 mg, 38%). LC-MS (ESI+): m/z 607.8 [M+H]⁺.

### Step 3. 3-(azetidin-3-yl)-6-iodo-7-(4-((6-methylpyridin-2-yl)oxy)phenyl)pyrrolo[1,2-a]pyrazine-8-carboxamide

A mixture of tert-butyl 3-(8-cyano-6-iodo-7-(4-((6-methylpyridin-2-yl)oxy)phenyl)pyrrolo[1,2-a]pyrazin-3-yl) azetidine-1-carboxylate (220 mg, 362.2 μmol) in H₂SO₄ (4.40 mL, 82.5 mmol) was stirred at 30° C. for 12 hrs. After cooling to rt, the mixture was poured into ice water (30 mL) and the pH was adjusted to 7 by aqueous solution of sodium carbonate. After extraction with ethyl acetate (20 mL*3), the combined organic layers were washed with brine (50 mL), dried over Na₂SO₄, filtered, and concentrated under reduced pressure to afford the title compound (150 mg). LC-MS (ESI+): m/z 526.1 [M+H]⁺.

### Step 4. 6-iodo-3-(1-methylazetidin-3-yl)-7-(4-((6-methylpyridin-2-yl)oxy)phenyl)pyrrolo[1,2-a]pyrazine-8-carboxamide

To a solution of 3-(azetidin-3-yl)-6-iodo-7-(4-((6-methylpyridin-2-yl)oxy)phenyl)pyrrolo[1,2-a]pyrazine-8-carboxamide (150 mg, 285.5 μmol) and HCHO (43 mg, 571.1 μmol, 40% purity) in MeOH (5 mL) was added NaBH₃CN (63 mg, 999.4 μmol). The reaction mixture was stirred at 25° C. for 2 hrs. Then the mixture was diluted with water (10 mL) and extracted with ethyl acetate (10 mL*3). The combined organic layers were washed with brine (20 mL), dried over Na₂SO₄, filtered, and concentrated under reduced pressure to afford the title compound (120 mg). LC-MS (ESI+): m/z 540.0 [M+H]⁺.

### Step 5. 6-(4-acrylamidophenyl)-3-(1-methylazetidin-3-yl)-7-(4-((6-methylpyridin-2-yl)oxy) phenyl)pyrrolo[1,2-a]pyrazine-8-carboxamide

To a solution of 6-iodo-3-(1-methylazetidin-3-yl)-7-(4-((6-methylpyridin-2-yl)oxy)phenyl)pyrrolo[1,2-a]pyrazine-8-carboxamide (100 mg, 185.4 μmol) and N-(4-(4,4,5,5-tetramethyl-1,3,2-dioxaborolan-2-yl)phenyl) acrylamide (61 mg, 222.5 μmol) in dioxane (3 mL) and H₂O (1 mL) were added K₂CO₃ (64 mg, 463.5 μmol) and Pd-118 (12 mg, 18.5 μmol) successively. The reaction mixture was stirred at 90° C. for 2 hrs under N₂ atmosphere. After cooling to rt, the mixture was diluted with water (20 mL) and extracted with ethyl acetate (10 mL*3). The combined organic layers were washed with brine (20 mL), dried over Na₂SO₄, filtered, and concentrated under reduced pressure to give a residue which was purified by prep-HPLC (column: Boston Prime C₁₈ 150*30 mm*5 um; mobile phase: [water (ammonia hydroxide v/v)-ACN]; B %: 25%-47%, 22 min) to afford the title compound (1.6 mg, 2%). LC-MS (ESI+): m/z 559.4 [M+H]⁺. ¹H NMR (400 MHZ, CDCl₃) δ 9.88 (s, 1H), 7.77 (s, 1H), 7.70-7.60 (m, 3H), 7.52 (s, 1H), 7.30-7.25 (m, 2H), 7.22 (d, J=8.3 Hz, 2H), 7.12 (d, J=8.7 Hz, 2H), 6.95 (d, J=7.3 Hz, 1H), 6.70 (d, J=7.7 Hz, 1H), 6.52-6.44 (m, 1H), 6.30 (dd, J=10.2, 16.7 Hz, 1H), 5.83 (d, J=10.0 Hz, 1H), 5.60-5.26 (m, 2H), 3.97-3.89 (m, 2H), 3.82-3.73 (m, 1H), 3.63-3.55 (m, 2H), 2.57 (s, 3H), 2.47 (s, 3H).

### Example 22: Preparation of 6-(4-acrylamidophenyl)-7-(4-methoxyphenyl)pyrrolo[1,2-a]pyrazine-8-carboxamide

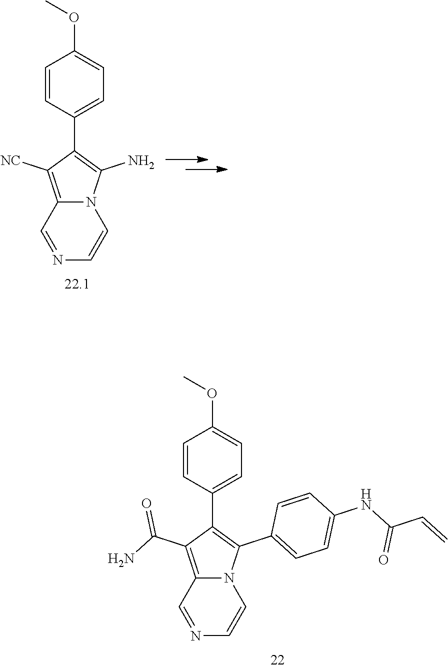 [SMILES: COc1ccc(-c2c(C#N)c3cnccn3c2N)cc1 ; C=CC(=O)Nc1ccc(-c2c(-c3ccc(OC)cc3)c(C(N)=O)c3cnccn23)cc1]

### Step 1: Preparation of Example 22

In a similar fashion according to the procedure for example 15, example 22 was synthesized from compound 22.1. Spectrum data of example 22: LCMS: 413.1 [M+H]⁺; ¹H NMR: (400 MHZ, DMSO-d₆) δ 10.30 (br s, 1H), 9.35 (s, 1H), 8.00 (d, J=4.8 Hz, 1H), 7.73 (d, J=8.3 Hz, 2H), 7.66 (d, J=4.8 Hz, 1H), 7.35-7.25 (m, 3H), 7.21 (d, J=8.8 Hz, 2H), 6.92 (d, J=8.5 Hz, 2H), 6.49-6.38 (m, 1H), 6.35-6.23 (m, 2H), 5.78 (dd, J=10.2, 1.6 Hz, 1H), 3.75 (s, 3H).

### Example 23 Preparation of 5-(4-acrylamidophenyl)-6-(4-((6-methylpyridin-2-yl)oxy)phenyl)pyrrolo[2,1-b]thiazole-7-carboxamide

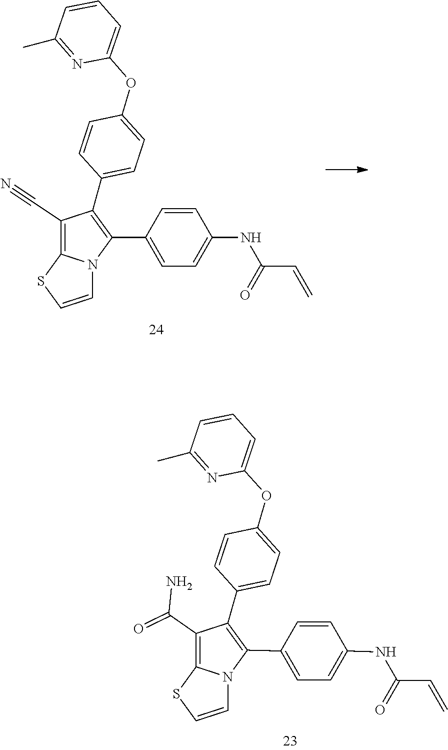 [SMILES: C=CC(=O)Nc1ccc(-c2c(-c3ccc(Oc4cccc(C)n4)cc3)c(C#N)c3sccn23)cc1 ; C=CC(=O)Nc1ccc(-c2c(-c3ccc(Oc4cccc(C)n4)cc3)c(C(N)=O)c3sccn23)cc1]

### Step 1: Preparation of Example 23

A solution of example 24 (45.0 mg, 0.094 mmol) in H₂SO₄ (1 mL) was stirred at 30° C. for 16 h. The reaction solution was poured into the ice and the pH of the reaction solution was adjusted to 7 by aqueous solution of sodium carbonate. After extraction with EA (10 mL×3), the combined organic layers were washed with brine (10 mL), dried over Na₂SO₄, filtered, concentrated, and purified by prep-HPLC to afford example 23. LCMS: m/z 495.1 [M+H]⁺; ¹H NMR: (400 MHZ, DMSO-d₆) δ 10.22 (s, 1H), 7.85 (d, J=4.2 Hz, 1H), 7.75 (t, J=7.8 Hz, 1H), 7.65 (d, J=8.6 Hz, 2H), 7.37 (d, J=8.6 Hz, 2H), 7.32 (d, J=4.2 Hz, 1H), 7.23 (d, J=8.6 Hz, 2H), 7.15 (d, J=8.4 Hz, 2H), 7.03 (d, J=7.3 Hz, 1H), 6.80 (d, J=8.1 Hz, 1H), 6.47-6.37 (m, 1H), 6.30-6.21 (m, 1H), 5.81-5.72 (m, 1H), 2.35 (s, 3H).

### Example 24 Preparation of N-(4-(7-cyano-6-(4-((6-methylpyridin-2-yl)oxy)phenyl)pyrrolo[2,1-b]thiazol-5-yl)phenyl) acrylamide

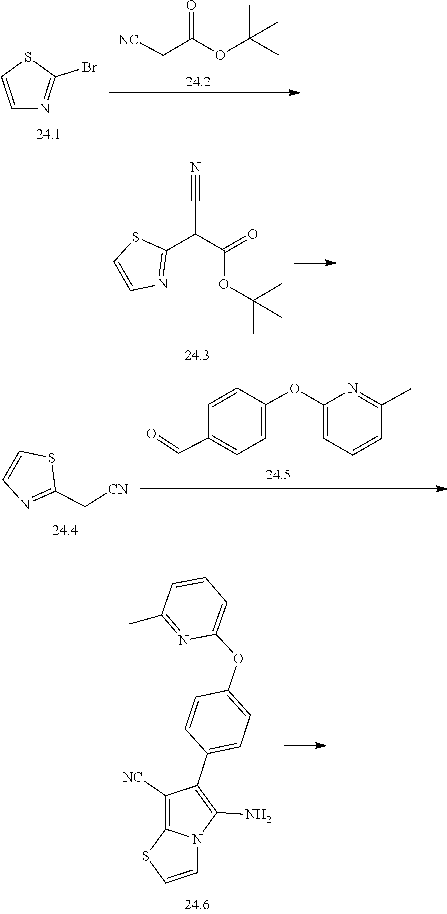

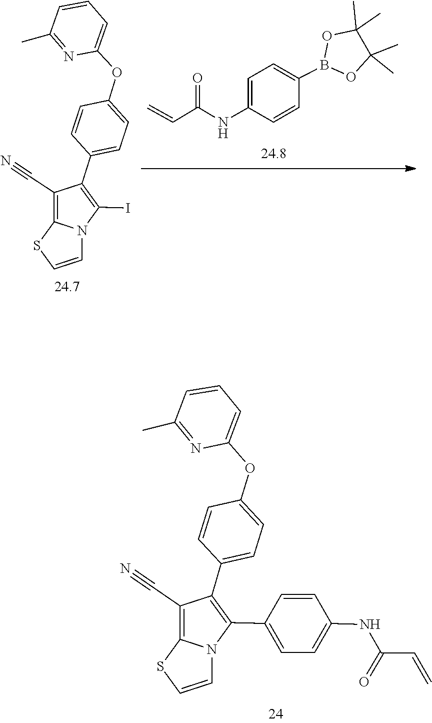

### Step 1: Preparation of Compound 24.3

To a solution of compound 24.1 (10.0 g, 61.0 mmol) in DMF (100 mL) was added Cs₂CO₃ (39.7 g, 121.9 mmol). Then compound 24.2 (11.2 g, 79.3 mmol) was added and the reaction mixture was stirred at 80° C. for 16 h. After cooling to rt, the reaction mixture was filtered and the filtrate was concentrated, purified by flash column chromatography on silica gel to afford compound 24.3 (8.00 g, 58.51%) as a yellow solid. LCMS: 168.8 [M+H−56]⁺.

### Step 2: Preparation of Compound 24.4

To a solution of compound 24.3 (6.00 g, 26.8 mmol) in toluene (90 mL) was added 4-methylbenzenesulfonic acid (0.37 g, 2.14 mmol) and the solution was stirred at 140° C. for 16 h. After cooling to rt, the reaction mixture was concentrated and purified by flash column chromatography on silica gel to afford compound 24.4 (1.50 g, 45.16%) as a yellow solid. LCMS: 124.8 [M+H]⁺.

### Step 3: Preparation of Compound 24.6

To a solution of compound 24.4 (1.50 g, 12.1 mmol) in MeOH (60 mL) was added compound 24.5 (2.58 g, 12.1 mmol) and DBU (5.52 g, 36.2 mmol). Then acetyl cyanide (0.83 g, 12.1 mmol) was dropwise added in the solution under N₂ and the reaction mixture was stirred at 100° C. under microwave for 20 min. After cooling to rt, the reaction mixture was concentrated and purified by flash column chromatography on silica gel to afford compound 24.6. LCMS: 347.2 [M+H]⁺

### Step 4: Preparation of Compound 24.7

To a solution of compound 24.6 (500.0 mg, 1.44 mmol) in chloroform (8 mL) was added diiodomethane (5.80 g, 21.7 mmol). Then 3-methyl-1-(nitrosooxy) butane (676.4 mg, 5.77 mmol) in chloroform (2 mL) was added in the solution at 70° C. and the reaction solution was stirred at 70° C. for 1 h. After cooling to rt, the reaction mixture was concentrated and purified by flash column chromatography on silica gel to afford compound 24.7. LCMS: m/z 457.9 [M+H]⁺

### Step 5: Preparation of Example 24

To a solution of compound 24.7 (100.0 mg, 0.219 mmol) and compound 24.8 (71.7 mg, 0.262 mmol) in dioxane (2 mL) and H₂O (0.4 mL) was added K₂CO₃ (60.4 mg, 0.437 mmol). Then Pd(PPh₃)₄ (25.3 mg, 0.022 mmol) was added in the solution under N₂ and the reaction solution was stirred at 80° C. for 1 h. After cooling to rt, the reaction solution was diluted with water (2 mL) and extracted with EA (2 mL×3). The combined organic layers were washed with brine (2 mL), dried over Na₂SO₄, filtered, concentrated, and purified by prep-HPLC to afford example 24. LCMS: 477.2 [M+H]⁺; ¹H NMR: (400 MHZ, CD₃OD) δ 7.78 (d, J=4.3 Hz, 1H), 7.76-7.69 (m, 3H), 7.38 (dd, J=15.6, 8.5 Hz, 4H), 7.27 (d, J=4.1 Hz, 1H), 7.08 (d, J=8.5 Hz, 2H), 7.03 (d, J=7.4 Hz, 1H), 6.71 (d, J=8.3 Hz, 1H), 6.49-6.37 (m, 2H), 5.81 (dd, J=9.4, 2.1 Hz, 1H), 2.45 (s, 3H).

### Example 25 Preparation of 6-(4-acrylamidophenyl)-7-(3-methoxy-4-((6-methylpyridin-2-yl)oxy)phenyl)pyrrolo[1,2-a]pyrazine-8-carboxamide

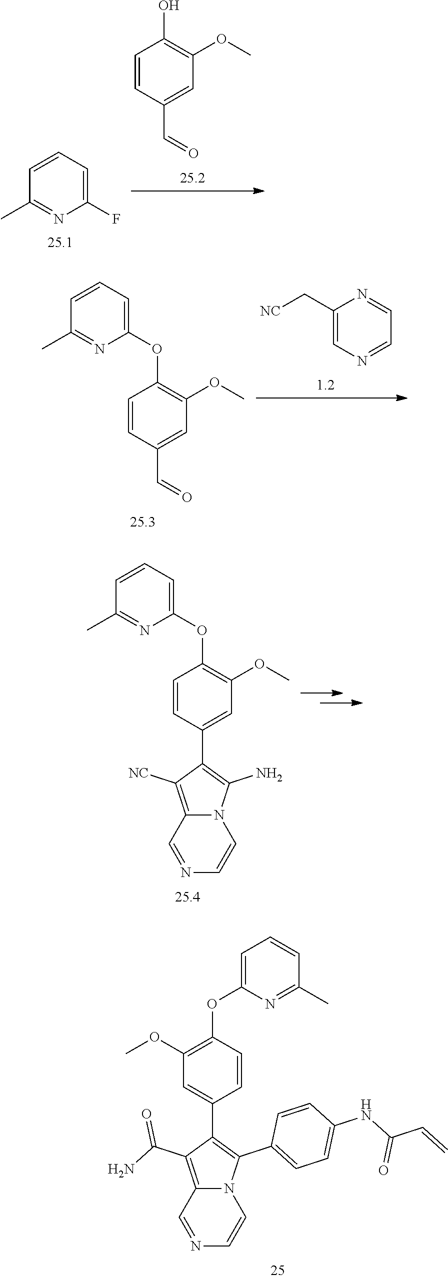

### Step 1: Preparation of Compound 25.3

To a solution of compound 25.1 (11.70 mL, 113.38 mmol) in DMF (110 mL), were added compound 25.2 (34.50 g, 226.75 mmol) and Cs₂CO₃ (110.82 g, 340.13 mmol). The mixture was stirred at 130° C. for 18 h. The reaction mixture was added NaOH (40 mL, 2.00 mmol) and stirred at 25° C. for 0.5 h, extracted with EA (3×100 mL). The organic layers were washed with brine (3×100 mL), dried over Na₂SO₄, filtered, and evaporated to afford a crude product which was purified by flash chromatography, to afford compound 25.3 (1.68 g). ¹H NMR: (400 MHZ, DMSO-d₆) δ 9.97 (s, 1H), 7.72 (t, J=8.0 Hz, 1H), 7.64-7.56 (m, 2H), 7.32 (d, J=8.0 Hz, 1H), 6.97 (d, J=8.0 Hz, 1H), 6.81 (d, J=8.0 Hz, 1H), 3.78 (s, 3H), 2.27 (s, 3H).

### Step 2: Preparation of Compound 25.4

To a solution of compound 1.2 (587.67 mg, 4.93 mmol) in methanol (16 mL) were added compound 25.3 (1.2 g, 4.93 mmol), acetyl cyanide (0.350 mL, 4.93 mmol) and DBU (2.21 mL, 14.80 mmol), and the reaction was stirred at 100° C. for 20 min under microwave irradiation. After cooling to room temperature, the reaction mixture was diluted with water (50 mL), extracted with EA (3×70 mL). The organic layers were washed with brine (3×50 mL), dried over Na₂SO₄, filtered, and evaporated to afford compound 25.4 (1.83 g, 99.0% yield). LCMS: 371.9 [M+H]⁺.

### Step 3: Preparation of Example 25

In a similar fashion according to the procedure for example 15, example 25 was synthesized from compound 25.4. Spectrum data of example 25: LCMS: 520.3 [M+H]⁺; ¹H NMR: (400 MHZ, DMSO-d₆) δ 10.31 (s, 1H), 9.34 (d, J=1.32 Hz, 1H), 8.03 (dd, J=4.8, 1.4 Hz, 1H), 7.77 (d, J=8.6 Hz, 2H), 7.66 (dd, J=6.2, 5.0 Hz, 2H), 7.41 (s, 1H), 7.33 (d, J=8.6 Hz, 2H), 7.05 (dd, J=12.2, 8.1 Hz, 2H), 6.95-6.87 (m, 2H), 6.61 (d, J=8.2 Hz, 2H), 6.45 (dd, J=16.9, 10.0 Hz, 1H), 6.28 (dd, J=17.0, 2.0 Hz, 1H), 5.78 (dd, J=10.0, 1.9 Hz, 1H), 3.52 (s, 3H), 2.29 (s, 3H).

### Example 26 Preparation of 6-(4-acrylamidophenyl)-7-(4-(pyridin-2-yloxy)phenyl)pyrrolo[1,2-a]pyrazine-8-carboxamide

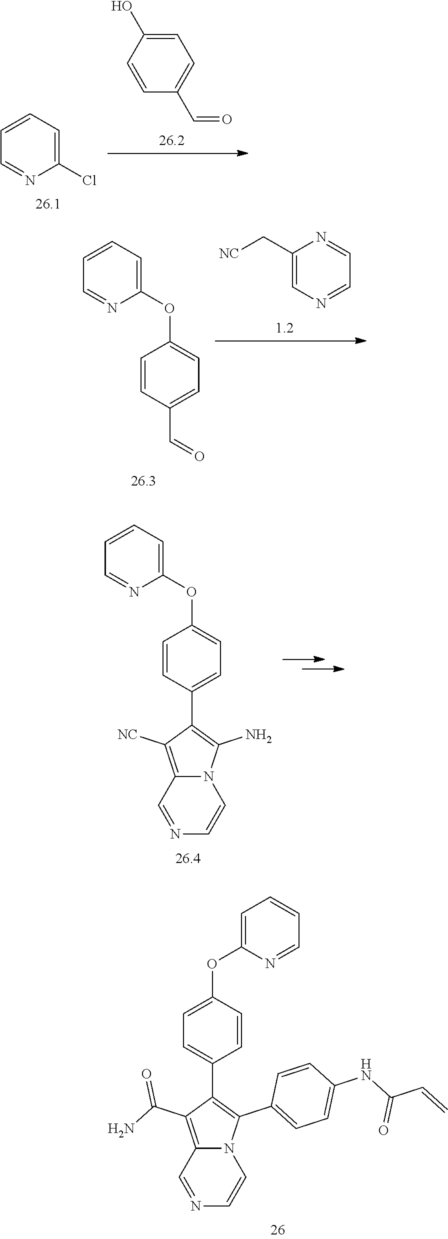 [SMILES: Clc1ccccn1 ; O=Cc1ccc(O)cc1 ; O=Cc1ccc(Oc2ccccn2)cc1 ; N#CCc1cnccn1 ; N#Cc1c(-c2ccc(Oc3ccccn3)cc2)c(N)n2ccncc12 ; C=CC(=O)Nc1ccc(-c2c(-c3ccc(Oc4ccccn4)cc3)c(C(N)=O)c3cnccn23)cc1]

### Step 1: Preparation of Compound 26.3

To a solution of compound 26.1 (4.17 mL, 44.04 mmol) in DMF (50 mL) were added compound 26.2 (8.07 g, 66.06 mmol), potassium carbonate (18.26 g, 132.11 mmol), cuprous oxide (1.28 g, 8.81 mmol). The mixture was stirred at 150° C. for 18 h. After cooling to room temperature, the reaction mixture was poured into water (80 mL), extracted with EA (3×50 mL). The combined organic layers were washed with brine (3×25 mL), dried over Na₂SO₄, filtered, and concentrated under reduced pressure to give a residue, which was purified by column chromatography on silica gel to give compound 26.3 (4.79 g, 54.61% yield). LCMS: 241.0 [M+42]⁺.

### Step 2: Preparation of Compound 26.4

To a solution of compound 26.3 (836.08 mg, 4.20 mmol) in MeOH (15 mL) were added compound 1.2 (500 mg, 4.20 mmol), DBU (1.88 mL, 12.59 mmol), acetyl cyanide (0.30 mL, 4.20 mmol). The mixture was stirred at 120° C. for 20 min. After cooling to room temperature, the reaction mixture was poured into water (80 mL), extracted with EA (3×50 mL). The combined organic layers were dried over Na₂SO₄, filtered, and concentrated under reduced pressure to give a residue which was purified by column chromatography on silica gel to give compound 26.4 (695 mg, 50.48% yield). LCMS: 328.2 [M+H]⁺.

### Step 3: Preparation of Example 26

In a similar fashion according to the procedure for example 15, example 26 was synthesized from compound 26.4. Spectrum data of example 26: LCMS: 476.3 [M+H]⁺. ¹H NMR: (400 MHZ, DMSO-d₆) δ 10.33 (s, 1H), 9.30 (s, 1H), 8.19 (dd, J=5.0, 1.6 Hz, 1H), 7.99 (dd, J=4.8, 0.8 Hz, 1H), 7.91-7.83 (m, 1H), 7.76 (d, J=8.8 Hz, 2H), 7.65 (d, J=4.8 Hz, 1H), 7.38 (s, 1H), 7.31 (d, J=8.4 Hz, 4H), 7.15 (dd, J=4.8, 0.8 Hz, 1H), 7.12-7.07 (dt, J=8.8 Hz, 2.4 Hz, 2H), 7.03 (d, J=8.0 Hz, 1H), 6.63 (s, 1H), 6.45 (dd, J=16.8, 10.0 Hz, 1H), 6.28 (dd, J=17.2, 2.0 Hz, 1H), 5.78 (dd, J=10.0, 2.0 Hz, 1H).

### Example 27 Preparation of 6-(4-acrylamidophenyl)-7-(4-(cyclopropylsulfonyl)phenyl)pyrrolo[1,2-a]pyrazine-8-carboxamide

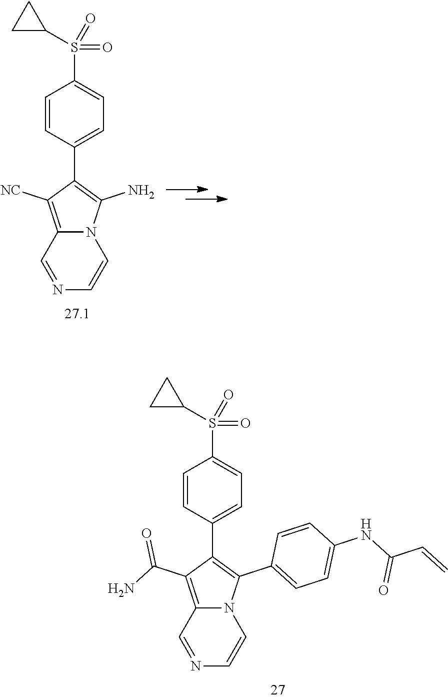 [SMILES: N#Cc1c(-c2ccc(S(=O)(=O)C3CC3)cc2)c(N)n2ccncc12 ; C=CC(=O)Nc1ccc(-c2c(-c3ccc(S(=O)(=O)C4CC4)cc3)c(C(N)=O)c3cnccn23)cc1]

### Step 1: Preparation of Example 27

In a similar fashion according to the procedure for example 12, example 27 was synthesized from compound 27.1. Spectrum data of example 27: LCMS: 487.3 [M+H]⁺; ¹H NMR: (400 MHZ, DMSO-d₆) δ 10.34 (s, 1H), 9.18 (s, 1H), 8.01-7.97 (m, 1H), 7.81 (d, J=8.3 Hz, 2H), 7.76 (d, J=8.5 Hz, 2H), 7.65 (d, J=4.8 Hz, 1H), 7.47 (d, J=8.3 Hz, 2H), 7.42 (br s, 1H), 7.28 (d, J=8.5 Hz, 3H), 6.49-6.39 (m, 1H), 6.32-6.24 (m, 1H), 5.82-5.75 (m, 1H), 2.93-2.83 (m, 1H), 1.15-1.10 (m, 2H), 1.08-1.01 (m, 2H).

### Example 28 Preparation of 6-(4-acrylamidophenyl)-7-(4-(cyclopentyloxy)phenyl)pyrrolo[1,2-a]pyrazine-8-carboxamide

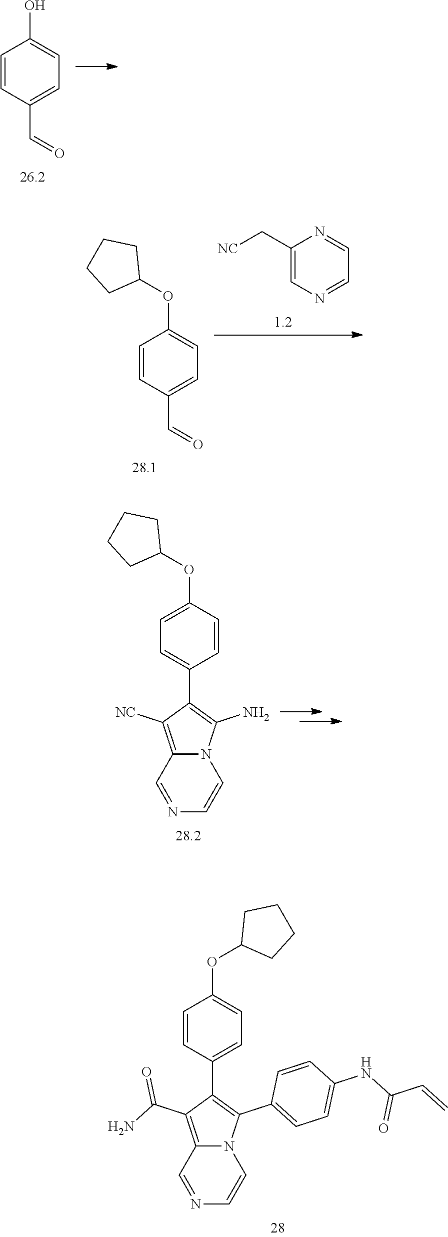 [SMILES: O=Cc1ccc(O)cc1 ; O=Cc1ccc(OC2CCCC2)cc1 ; N#CCc1cnccn1 ; N#Cc1c(-c2ccc(OC3CCCC3)cc2)c(N)n2ccncc12 ; C=CC(=O)Nc1ccc(-c2c(-c3ccc(OC4CCCC4)cc3)c(C(N)=O)c3cnccn23)cc1]

### Step 1: Preparation of Compound 28.1

To a solution of compound 26.2 (5 g, 40.94 mmol) in DMF (60 mL) was added potassium carbonate (11.32 g, 81.88 mmol). The mixture was heated to 65° C., bromocyclopentane (12.20 g, 81.88 mmol) was added. The reaction mixture was stirred at 100° C. for 5 h under N₂. After cooling to room temperature, the reaction was diluted with H₂O (300 mL), extracted with EA (3×300 mL). The combined organic layers were dried over Na₂SO₄, filtered, concentrated under reduced pressure to give compound 28.1 (6.94 g, 89.10% yield). LCMS: 191.2 [M+H]⁺

### Step 2: Preparation of Compound 28.2

To a solution of compound 1.2 (500 mg, 4.19 mmol) in MeOH (15 mL) were added compound 28.1 (798.46 mg, 4.19 mmol), DBU (1916.90 mg, 12.59 mmol) and 2-hydroxy-2-methylpropanenitrile (357.21 mg, 4.19 mmol). The mixture was stirred at 120° C.° C. for 20 min under microwave and N₂. After cooling to room temperature, the mixture was quenched by water (100 mL), extracted with dichloromethane (3×100 mL), the combined organic layers were dried over Na₂SO₄, filtered, concentrated under reduced pressure to give a residue which was purified by column chromatography on silica gel to give compound 28.2 (800 mg, 59.87% yield). LCMS: 319.2 [M+H]⁺.

### Step 3: Preparation of Example 28

In a similar fashion according to the procedure for example 1, example 28 was synthesized from compound 28.2. Spectrum data of example 28: LCMS: 467.2 [M+H]⁺; ¹H NMR: (400 MHZ, DMSO-d₆) δ 10.30 (s, 1H), 9.34 (s, 1H), 7.99 (d, J=4.8 Hz, 1H), 7.72 (d, J=8.4 Hz, 2H), 7.65 (d, J=4.8 Hz, 1H), 7.27 (d, J=8.8 Hz, 3H), 7.18 (d, J=8.8 Hz, 2H), 6.87 (d, J=8.4 Hz, 2H), 6.44 (dd, J=16.8, 10.0 Hz, 1H), 6.27 (d, J=16.8 Hz, 2H), 5.78 (d, J=12 Hz, 1H), 4.80-4.78 (m, 1H), 1.92-1.88 (3, 2H), 1.73-1.67 (m, 4H), 1.65-1.52 (m, 2H).

### Example 29 Preparation of 6-(4-acrylamidophenyl)-3-((dimethylamino)methyl)-7-(4-((6-methylpyridin-2-yl)oxy)phenyl)pyrrolo[1,2-a]pyrazine-8-carboxamide

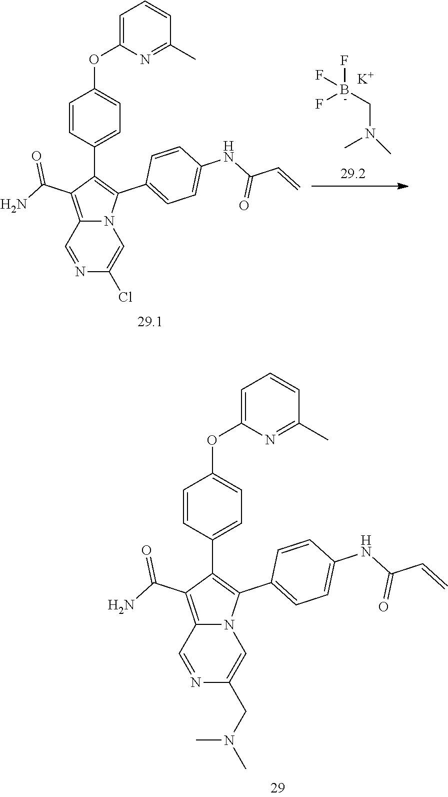

### Step 1: Preparation of Example 29

Compound 29.1 was synthesized in a similar fashion according to the procedure for example 15.

A solution of compound 29.1 (130 mg, 248 μmol), compound 29.2 (81.9 mg, 496 μmol), Cs₂CO₃ (162.0 mg, 496.0 μmol) and Xphos Pd G3 (21.0 mg, 24.8 μmol) in dioxane (2.5 mL) and H₂O (0.5 mL) was stirred at 100° C. for 12 h under N₂. The reaction solution was filtered, and filtrate was concentrated in vacuum. The residue was purified by prep-HPLC to afford example 29. LCMS: 547.4 [M+H]⁺. ¹H NMR: (400 MHZ, DMSO-d₆) δ 10.33 (s, 1H), 9.27 (s, 1H), 7.89 (s, 1H), 7.82-7.69 (m, 3H), 7.38 (br s, 1H), 7.29 (dd, J=8.0, 5.5 Hz, 4H), 7.06 (d, J=8.3 Hz, 2H), 7.01 (d, J=7.3 Hz, 1H), 6.76 (d, J=8.3 Hz, 1H), 6.58 (br s, 1H), 6.49-6.41 (m, 1H), 6.32-6.23 (m, 1H), 5.79 (d, J=11.0 Hz, 1H), 3.54 (s, 2H), 2.33 (s, 3H), 2.23 (s, 6H).

### Example 30 Preparation of 7-(4-(2-azaspiro[3.3]heptane-2-carbonyl)phenyl)-6-(4-acrylamidophenyl)pyrrolo[1,2-a]pyrazine-8-carboxamide

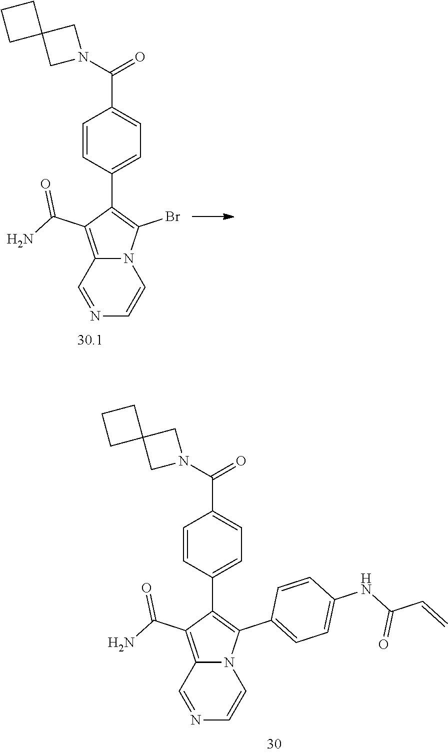 [SMILES: NC(=O)c1c(-c2ccc(C(=O)N3CC4(CCC4)C3)cc2)c(Br)n2ccncc12 ; C=CC(=O)Nc1ccc(-c2c(-c3ccc(C(=O)N4CC5(CCC5)C4)cc3)c(C(N)=O)c3cnccn23)cc1]

### Step 1: Preparation of Example 30

In a similar fashion according to the procedure for example 12, example 30 was synthesized from compound 30.1. Spectrum data of example 30: LCMS: 506.3 [M+H]⁺; ¹H NMR: (400 MHZ, DMSO-d₆) δ 10.32 (s, 1H), 9.24 (s, 1H), 7.99 (d, J=5.0 Hz, 1H), 7.74 (d, J=8.3 Hz, 2H), 7.65 (d, J=5.0 Hz, 1H), 7.56 (d, J=8.0 Hz, 2H), 7.37 (br s, 1H), 7.31 (d, J=8.0 Hz, 2H), 7.26 (d, J=8.3 Hz, 2H), 6.84 (br s, 1H), 6.50-6.38 (m, 1H), 6.35-6.19 (m, 1H), 5.78 (d, J=10.3 Hz, 1H), 4.26 (s, 2H), 3.99 (s, 2H), 2.13 (t, J=7.7 Hz, 4H), 1.84-1.64 (m, 2H).

### Example 32 Preparation of 6-(4-acrylamidophenyl)-7-(3-fluoro-4-((6-methylpyridin-2-yl)oxy)phenyl)pyrrolo[1,2-a]pyrazine-8-carboxamide

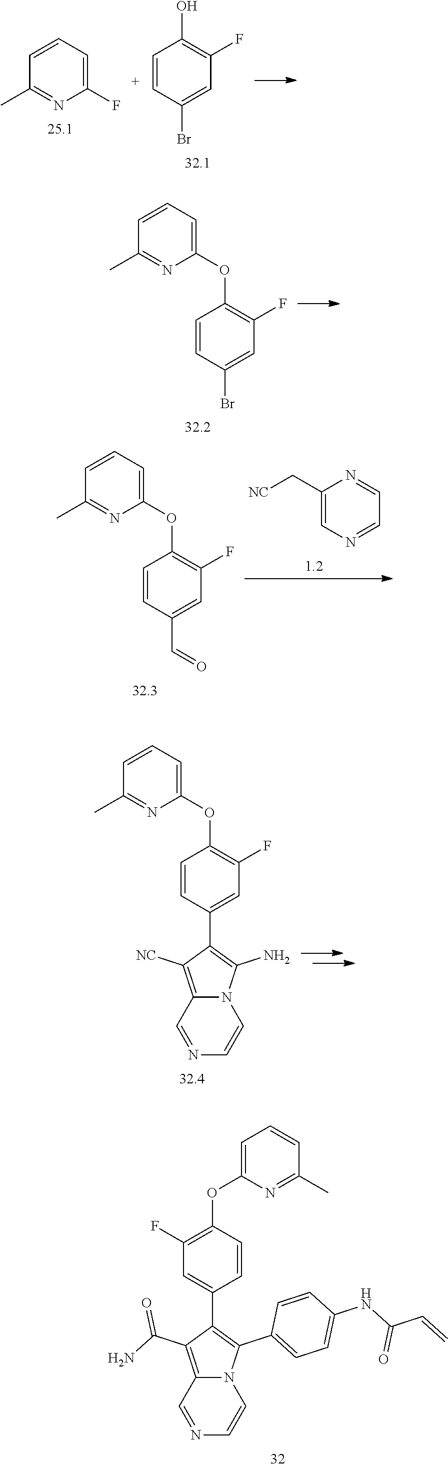

### Step 1: Preparation of Compound 32.2

To a suspension of compound 25.1 (6 g, 54.00 mmol) in NMP (200 mL) were added compound 32.1 (10.31 g, 53.60 mmol) and Cs₂CO₃ (52.78 g, 161.99 mmol). The reaction mixture was stirred at 150° C. for 18 h. After cooling to room temperature, the reaction mixture was diluted with water (200 mL) and extracted with EA (3×200 mL). The combined organic layer layers were washed with saturated brine (200 mL), dried over Na₂SO₄, filtered, concentrated under reduced pressure to give a residue which was purified by column chromatography on silica gel to give compound 32.2 (7.7 g, 48.16% yield). LCMS: 281.8 [M+H]⁺.

### Step 2: Preparation of Compound 32.3

To a solution of compound 32.2 (3 g, 10.63 mmol) in THF (150 mL) was added a solution of BuLi (7.31 mL, 11.70 mmol) in THF at −78° C. The reaction mixture was stirred at −78° C. for 30 min, then the mixture was added DMF (0.98 mL, 12.76 mmol) and the reaction mixture was stirred at −78° C. for 30 min. After warmed to room temperature, the reaction mixture was diluted with water (200 mL) and extracted with EA (3×200 mL). The combined organic layer layers were washed with saturated brine (200 mL), dried over Na₂SO₄, filtered, concentrated under reduced pressure to give a residue which was purified by column chromatography on silica gel to give compound 32.3 (460 mg, 18.71% yield). LCMS: 232.0 [M+H]⁺.

### Step 3: Preparation of Compound 32.4

To a solution of compound 1.2 (400 mg, 3.36 mmol) and compound 32.3 (776 mg, 3.36 mmol) in MeOH (15 mL) were added DBU (1533 mg, 10.07 mmol), 2-oxopropanenitrile (255 mg, 3.69 mmol). The reaction mixture was stirred at 100° C. for 20 min under microwave and N₂. After cooling to room temperature, the reaction mixture was diluted with water (50 mL) and extracted with EA (3×50 mL). The combined organic layer layers were washed with saturated brine (50 mL), dried over Na₂SO₄, filtered, concentrated under reduced pressure to give a residue which was purified by column chromatography on silica gel to give compound 32.4 (900 mg, 74.59% yield). LCMS: 360.0 [M+H]⁺.

### Step 4: Preparation of Example 32

In a similar fashion according to the procedure for example 15, example 32 was synthesized from compound 32.4. Spectrum data of example 32: LCMS: 508.3 [M+H]⁺. ¹H NMR: (400 MHZ, DMSO-d₆) δ 10.36 (s, 1H), 9.26 (d, J=1.2 Hz, 1H), 8.41 (s, 1H), 7.83-7.76 (m, 2H), 7.73 (d, J=8.0 Hz, 1H), 7.66 (d, J=4.8 Hz, 1H), 7.44 (s, 1H), 7.32 (d, J=8.4 Hz, 2H), 7.28-7.17 (m, 2H), 7.09 (d, J=9.2 Hz, 1H), 7.00 (d, J=7.2 Hz, 1H), 6.93 (s, 1H), 6.84 (d, J=8.0 Hz, 1H), 6.46 (dd, J=16.0, 10.1 Hz, 1H), 6.28 (dd, J=16.0, 1.9 Hz, 1H), 5.78 (dd, J=10.0, 1.9 Hz, 1H), 2.29 (s, 3H).

### Example 33 Preparation of 6-(4-acrylamidophenyl)-7-(3-fluoro-4-((4-methylpyrimidin-2-yl)oxy)phenyl)pyrrolo[1,2-a]pyrazine-8-carboxamide

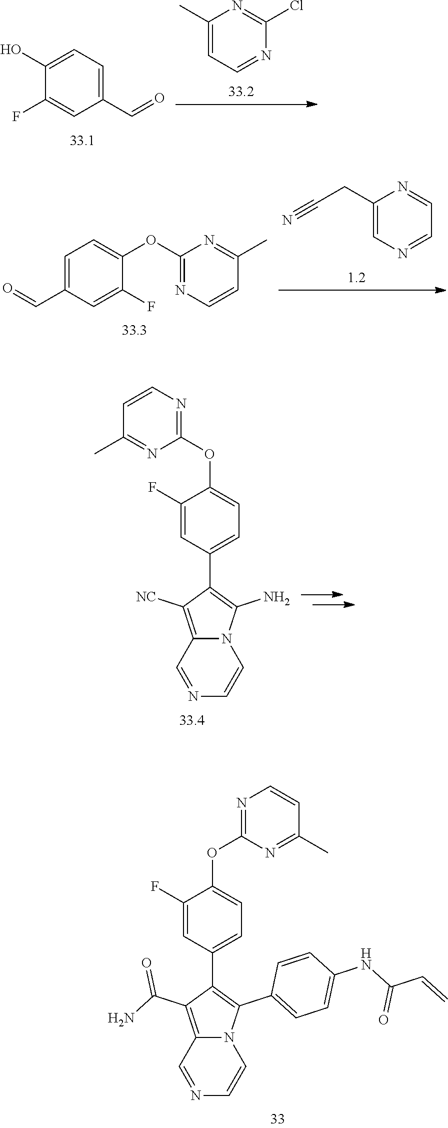

### Step 1: Preparation of Compound 33.3

To a solution of compound 33.1 (15.0 g, 107.1 mmol) and compound 33.2 (16.5 g, 128.5 mmol) in DMF (250 mL) was added CsF (32.5 g, 214.1 mmol). The reaction mixture was stirred at 120° C. for 12 h. After cooling to room temperature, the reaction mixture was filtered and the filtrate was concentrated and purified by flash column chromatography on silica gel to afford compound 33.3 (6.00 g, 24.14%) as a yellow solid. LCMS: 232.9 [M+H]⁺.

### Step 2: Preparation of Compound 33.4

To a mixture of compound 33.3 (974.7 mg, 4.20 mmol) and compound 1.2 (500.0 mg, 4.20 mmol) in n-BuOH (10 mL) was added DBU (1.28 g, 8.39 mmol, 1.27 mL). Then acetyl cyanide (322.1 mg, 4.20 mmol, 90% purity) was added in the solution at 25° C. and purged with N₂ for 3 times. After the addition, the reaction mixture was heated to 100° C. under microwave for 20 min. The reaction solution was cooled to room temperature and diluted with water (100 mL). After extraction with EA (50 mL×3), the combined organic layers were washed with brine (100 mL), dried over Na₂SO₄, filtered, concentrated, and purified by flash column chromatography on silica gel to afford compound 33.4 (400.0 mg, 26.45% yield) as a yellow solid. LCMS: 361.0 [M+H]⁺.

### Step 3: Preparation of Example 33

In a similar fashion according to the procedure for example 15, example 33 was synthesized from compound 33.4. Spectrum data of example 33: LCMS: 509.1 [M+H]⁺; ¹H NMR: (400 MHZ, DMSO-d₆) δ 10.36 (s, 1H), 9.23 (s, 1H), 8.48 (d, J=4.8 Hz, 1H), 7.99 (d, J=4.3 Hz, 1H), 7.79 (d, J=8.3 Hz, 2H), 7.66 (d, J=4.8 Hz, 1H), 7.48 (br s, 1H), 7.38-7.29 (m, 3H), 7.26 (d, J=11.8 Hz, 1H), 7.19 (d, J=5.0 Hz, 1H), 7.11 (d, J=8.5 Hz, 2H), 6.52-6.38 (m, 1H), 6.35-6.21 (m, 1H), 5.85-5.73 (m, 1H), 2.42 (s, 3H).

### Example 34 Preparation of 7-(3-fluoro-4-((4-methylpyrimidin-2-yl)oxy)phenyl)-6-(4-methacrylamidophenyl)pyrrolo[1,2-a]pyrazine-8-carboxamide

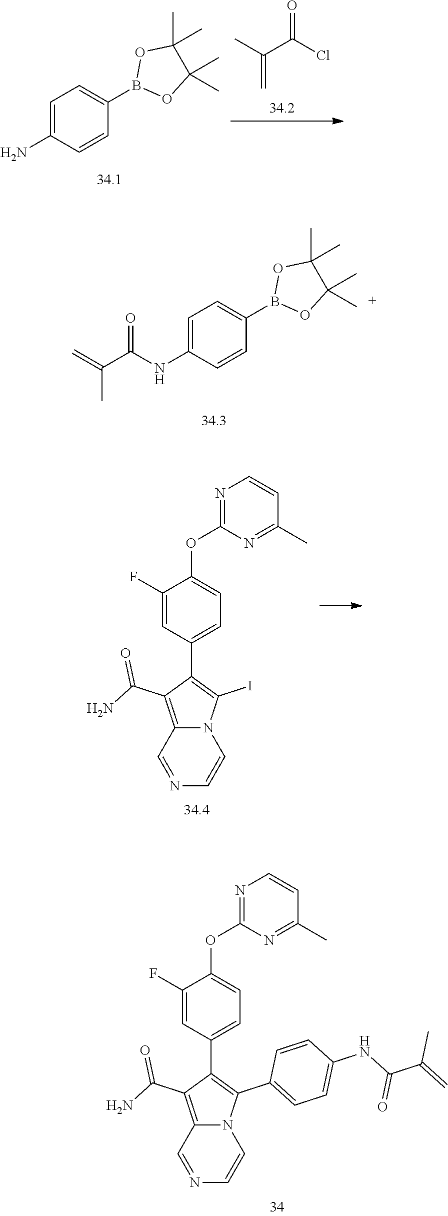

### Step 1: Preparation of Compound 34.3

To a mixture of compound 34.1 (2.00 g, 9.13 mmol) in DCM (20 mL) was added TEA (2.77 g, 27.4 mmol). Then compound 34.2 (1.05 g, 10.0 mmol) dissolved in DCM (10 mL) was added in the solution at 0° C. The reaction was stirred at 0° C. for 1 h. The reaction solution was diluted with water (50 mL) and extracted with DCM (30 mL×3). The combined organic layers were washed with brine (100 mL), dried over Na₂SO₄, filtered, concentrated, and purified by flash column chromatography on silica gel to afford compound 34.3 (1.20 g, 45.78% yield) as a white solid. LCMS: 288.2 [M+H]⁺

### Step 2: Preparation of Example 34

In a similar fashion according to the procedures for example 15, compound 34.4 was synthesized from compound 33.4.

To a solution of compound 34.4 (240.0 mg, 490.6 μmol) and compound 34.3 (211.3 mg, 735.8 μmol) in dioxane (6 mL) and H₂O (3 mL) was added Na₂CO₃ (104.0 mg, 981.1 μmol). Then Pd(PPh₃)₄ (56.7 mg, 49.1 μmol) was added in the mixture and the reaction was stirred at 80° C. for 2 h under N₂. After cooling to rt, the reaction solution was diluted with water (20 mL) and extracted with EA (20 mL×3). The combined organic layers were washed with brine (20 mL), dried over Na₂SO₄, filtered, concentrated, and purified by prep-HPLC to afford example 34. LCMS: 523.4 [M+H]⁺; ¹H NMR: (400 MHz, DMSO-d₆) δ 9.98 (s, 1H), 9.23 (s, 1H), 8.48 (d, J=5.0 Hz, 1H), 7.98 (dd, J=4.9, 1.2 Hz, 1H), 7.82 (d, J=8.6 Hz, 2H), 7.66 (d, J=4.9 Hz, 1H), 7.59-7.37 (m, 1H), 7.36-7.29 (m, 3H), 7.26 (dd, J=11.6, 1.8 Hz, 1H). 7.19 (d, J=5.0 Hz, 1H), 7.15-7.01 (m, 2H), 5.82 (s, 1H), 5.55 (s, 1H), 2.43 (s, 3H), 1.96 (s, 3H).

### Example 35 Preparation of 6-(4-acrylamidophenyl)-7-(3-fluoro-4-((4-methylpyrimidin-2-yl)oxy)phenyl)-3-methylpyrrolo[1,2-a]pyrazine-8-carboxamide

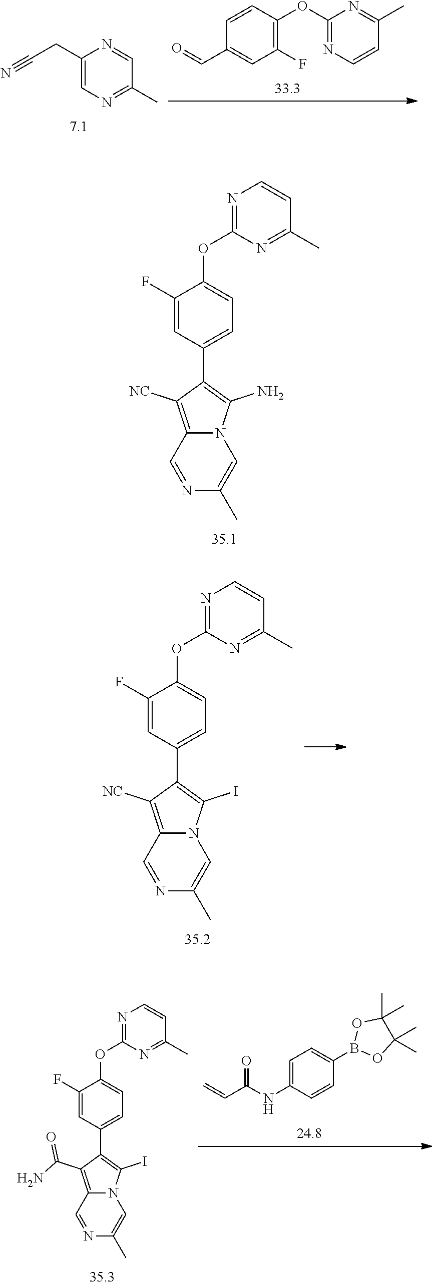

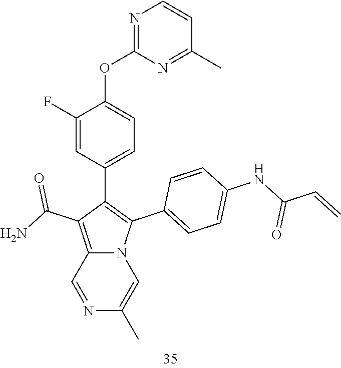 [SMILES: C=CC(=O)Nc1ccc(-c2c(-c3ccc(Oc4nccc(C)n4)c(F)c3)c(C(N)=O)c3cnc(C)cn23)cc1]

### Step 1: Preparation of Compound 35.1

To a solution of compound 33.3 (872.0 mg, 3.76 mmol) and compound 7.1 (500.0 mg, 3.76 mmol) in n-BuOH (10 mL) was added DBU (1.14 g, 7.51 mmol, 1.13 mL). Then acetyl cyanide (288.2 mg, 3.76 mmol, 90% purity) was added in the solution at 25° C. and purged with N₂ for 3 times. After the addition, the reaction mixture was heated to 100° C. under microwave for 20 min. The reaction solution was cooled to room temperature and diluted with water (100 mL). After extraction with EA (50 mL×3), the combined organic layers were washed with brine (100 mL), dried over Na₂SO₄, filtered, concentrated, and purified by flash column chromatography on silica gel to afford compound 35.1 (300.0 mg, 21.34% yield) as a yellow solid. LCMS: 374.8 [M+H]⁺.

### Step 2: Preparation of Compound 35.2

A mixture of compound 35.1 (280.0 mg, 747.9 μmol) in CH₃CN (5 mL) was added CH₂I₂ (3.06 g, 11.44 mmol). Then isopentyl nitrite (350.5 mg, 2.99 mmol) was added in the mixture and the reaction was stirred at 40° C. for 1 h. After cooling to room temperature, the reaction solution was concentrated and purified by flash column chromatography on silica gel to afford compound 35.2. LCMS: 485.9 [M+H]⁺.

### Step 3: Preparation of Compound 35.3

A solution of compound 35.2 (140.0 mg, 288.5 μmol) in H₂SO₄ (9.20 g, 93.8 mmol, 5 mL) was stirred at 30° C. for 16 h. After cooling to room temperature, the reaction solution was diluted with ice water (30 mL) and the pH of the reaction solution was adjusted to 7 by NaHCO₃ (sat. aq). After extraction with EA (20 mL×3), the combined organic layers were washed with brine (50 mL), dried over Na₂SO₄, filtered, and concentrated to afford compound 35.3. LCMS: 504.0 [M+H]⁺.

### Step 4: Preparation of Example 35

To a solution of compound 35.3 (90.0 mg, 178.8 μmol) and compound 24.8 (63.5 mg, 232.5 μmol) in dioxane (3 mL) and H₂O (1 mL) was added Na₂CO₃ (37.9 mg, 357.7 μmol). Then Pd(PPh₃)₄ (20.7 mg, 17.9 μmol) was added in the mixture under N₂ and the mixture was stirred at 80° C. for 2 h. After cooling to room temperature, the reaction solution was diluted with water (20 mL) and extracted with EA (20 mL×3). The combined organic layers were washed with brine (20 mL), dried over Na₂SO₄, filtered, concentrated, and purified by prep-HPLC to afford example 35. LCMS: 523.1 [M+H]⁺. ¹H NMR: (400 MHZ, DMSO-d₆) δ 10.35 (s, 1H), 9.18 (s, 1H), 8.47 (d, J=5.0 Hz, 1H), 7.84-7.76 (m, 3H), 7.50-7.38 (m, 1H), 7.34-7.28 (m, 3H), 7.27-7.21 (m, 1H), 7.19 (d, J=5.0 Hz, 1H), 7.10 (d, J=8.4 Hz, 1H), 7.03-6.90 (m, 1H), 6.50-6.41 (m, 1H), 6.33-6.25 (m, 1H), 5.83-5.77 (m, 1H), 2.42 (s, 3H), 2.35 (s, 3H).

Examples 36-55 were prepared using similar procedures as described in Example 35.

| | | LCMS | |
|---|---|---|---|
| Example | Structure | [M + H]⁺ | ¹H NMR |
| 36 | 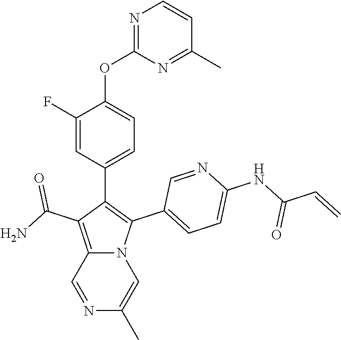 [SMILES: C=CC(=O)Nc1ccc(-c2c(-c3ccc(Oc4nccc(C)n4)c(F)c3)c(C(N)=O)c3cnc(C)cn23)cn1] | 524.1 | (400 MHz, DMSO-d₆) δ 10.92 (s, 1H), 9.22 (s, 1H), 8.48 (d, J = 5.0 Hz, 1H), 8.32 (d, J = 8.8 Hz, 1H), 8.25 (d, J = 2.0 Hz, 1H), 7.96-7.86 (m, 2H), 7.50-7.38 (m, 1H), 7.36- 7.28 (m, 2H), 7.19 (d, J = 5.3 Hz, 1H), 7.11 (d, J = 7.3 Hz, 1H), 7.02- 6.83 (m, 1H), 6.63 (dd, J = 10.3, 17.1 Hz, 1H), 6.35 (dd, J = 1.5, 17.1 Hz, 1H), 5.94-5.74 (m, 1H), 2.42 (s, 3H), 2.36 (s, 3H). |
| | 6-(6-acrylamidopyridin-3-yl)-7- | | |
| | (3-fluoro-4-((4-methylpyrimidin- | | |
| | 2-yl)oxy)phenyl)-3- | | |
| | methylpyrrolo[1,2-a]pyrazine-8- | | |
| | carboxamide | | |
| 37 | 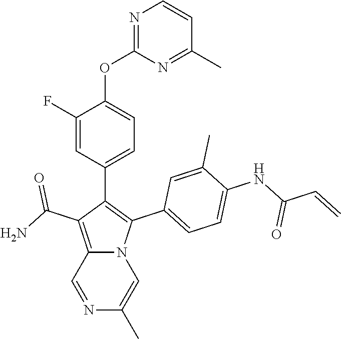 [SMILES: C=CC(=O)Nc1ccc(-c2c(-c3ccc(Oc4nccc(C)n4)c(F)c3)c(C(N)=O)c3cnc(C)cn23)cc1C] | 537.1 | (400 MHz, DMSO-d₆) δ 9.52 (s, 1H), 9.19 (s, 1H), 8.48 (d, J = 5.0 Hz, 1H), 7.83 (s, 1H), 7.73 (d, J = 8.0 Hz, 1H), 7.43 (d, J = 3.3 Hz, 1H), 7.31 (t, J = 8.4 Hz, 1H), 7.27-7.21 (m, 2H), 7.21-7.16 (m, 2H), 7.10 (d, J = 8.8 Hz, 1H), 6.94 (br s, 1H), 6.59 (dd, J = 17.1, 10.3 Hz, 1H), 6.28 (dd, J = 17.1, 1.8 Hz, 1H), 5.85-5.72 (m, 1H), 2.42 (s, 3H), 2.36 (s, 3H), 2.24 (s, 3H) |
| | 6-(4-acrylamido-3- | | |
| | methylphenyl)-7-(3-fluoro-4-((4- | | |
| | methylpyrimidin-2- | | |
| | yl)oxy)phenyl)-3- | | |
| | methylpyrrolo[1,2-a]pyrazine-8- | | |
| | carboxamide | | |
| 38 | 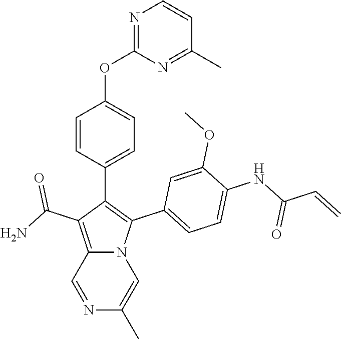 [SMILES: C=CC(=O)Nc1ccc(-c2c(-c3ccc(Oc4nccc(C)n4)cc3)c(C(N)=O)c3cnc(C)cn23)cc1OC] | 535.1 | (400 MHz, DMSO-d₆) δ 9.47 (s, 1 H), 9.28 (s, 1 H), 8.45 (d, J = 4.8 Hz, 1 H), 8.21 (d, J = 7.8 Hz, 1 H), 7.99 (br s, 1 H), 7.34 (d, J = 8.1 Hz, 3 H), 7.23-7.12 (m, 3 H), 7.03 (d, J = 7.8 Hz, 1 H), 6.85 (s, 1 H), 6.73 (dd, J = 16.8, 10.0 Hz, 1 H), 6.58-6.42 (m, 1 H), 6.25 (d, J = 16.9 Hz, 1 H), 5.74 (d, J = 10.0 Hz, 1 H), 3.70 (s, 3 H), 2.41 (s, 3 H), 2.37 (s, 3 H) |
| | 6-(4-acrylamido-3- | | |
| | methoxyphenyl)-3-methyl-7-(4- | | |
| | ((4-methylpyrimidin-2- | | |
| | yl)oxy)phenyl)pyrrolo[1,2- | | |
| | a]pyrazine-8-carboxamide | | |
| 39 | 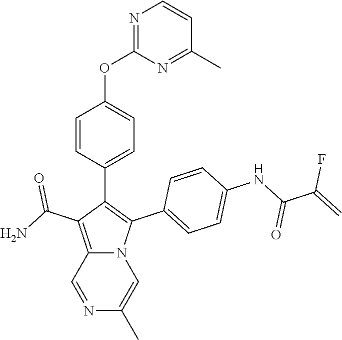 [SMILES: C=C(F)C(=O)Nc1ccc(-c2c(-c3ccc(Oc4nccc(C)n4)cc3)c(C(N)=O)c3cnc(C)cn23)cc1] | 523.5 | (400 MHz, DMSO-d₆) δ 10.45 (s, 1 H), 9.26 (s, 1 H), 8.46 (d, J = 5.0 Hz, 1 H), 7.92-7.75 (m, 3 H), 7.32 (dd, J = 8.4, 5.4 Hz, 5 H), 7.20-7.07 (m, 3 H), 6.56 (br s, 1 H), 5.86-5.63 (m, 1 H), 5.46 (dd, J = 15.6, 3.7 Hz, 1 H), 2.41 (s, 3 H), 2.35 (s, 3 H). |
| | 6-(4-(2- | | |
| | fluoroacrylamido)phenyl)-3- | | |
| | methyl-7-(4-((4-methylpyrimidin- | | |
| | 2-yl)oxy)phenyl)pyrrolo[1,2- | | |
| | a]pyrazine-8-carboxamide | | |
| 40 | 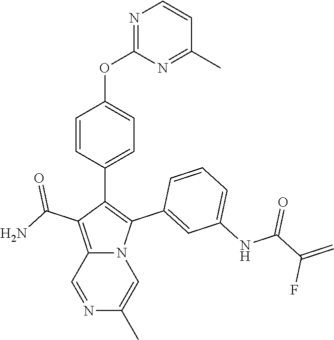 [SMILES: C=C(F)C(=O)Nc1cccc(-c2c(-c3ccc(Oc4nccc(C)n4)cc3)c(C(N)=O)c3cnc(C)cn23)c1] | 523.1 | (400 MHz, DMSO-d₆) δ 10.43 (s, 1H), 9.25 (s, 1H), 8.46 (d, J = 4.8 Hz, 1H), 7.88-7.73 (m, 3H), 7.49- 7.36 (m, 2H), 7.32 (d, J = 8.6 Hz, 2H), 7.18-7.11 (m, 3H), 7.01 (d, J = 7.7 Hz, 1H), 6.74-6.56 (m, 1H), 5.84-5.61 (m, 1H), 5.45 (dd, J = 15.7, 3.6 Hz, 1H), 2.40 (s, 3H), 2.37- 2.33 (m, 3H). |
| | 6-(3-(2- | | |
| | fluoroacrylamido)phenyl)-3- | | |
| | methyl-7-(4-((4-methylpyrimidin- | | |
| | 2-yl)oxy)phenyl)pyrrolo[1,2- | | |
| | a]pyrazine-8-carboxamide | | |
| 41 | 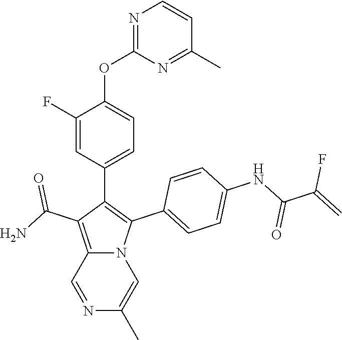 [SMILES: C=C(F)C(=O)Nc1ccc(-c2c(-c3ccc(Oc4nccc(C)n4)c(F)c3)c(C(N)=O)c3cnc(C)cn23)cc1] | 541.1 | (400 MHz, DMSO-d₆) δ 10.48 (s, 1 H), 9.19 (s, 1H), 8.48-8.47 (m, 1H), 7.87-7.81 (m, 3H), 7.36-7.34 (m, 1H), 7.31-7.29 (m, 2H), 7.25-7.23 (m, 1H), 7.19-7.18 (m, 2H), 7.12- 7.10 (m, 1H), 6.95 (brs, 1H), 5.75 (dd, J = 48.0, 3.6 Hz, 1H), 5.47 (dd, J = 15.6, 3.2 Hz, 1H), 2.42 (s, 3H), 2.35 (s, 3H). |
| | 7-(3-fluoro-4-((4- | | |
| | methylpyrimidin-2- | | |
| | yl)oxy)phenyl)-6-(4-(2- | | |
| | fluoroacrylamido)phenyl)-3- | | |
| | methylpyrrolo[1,2-a]pyrazine-8- | | |
| | carboxamide | | |
| 42 | 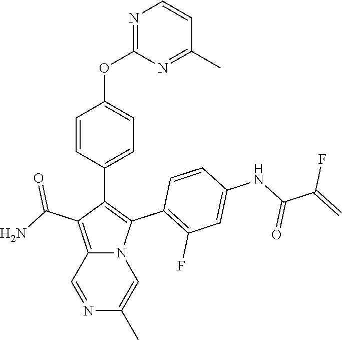 [SMILES: C=C(F)C(=O)Nc1ccc(-c2c(-c3ccc(Oc4nccc(C)n4)cc3)c(C(N)=O)c3cnc(C)cn23)c(F)c1] | 541.2 | (400 MHz, DMSO-d₆) δ 10.66 (s, 1H), 9.24 (d, J = 1.3 Hz, 1H), 8.46 (d, J = 5.0 Hz, 1H), 7.87 (dd, J = 12.3, 2.0 Hz, 1H), 7.74-7.54 (m, 2H), 7.40 (br s, 1H), 7.33-7.25 (m, 3H), 7.21-7.11 (m, 3H), 6.76 (br s, 1H), 5.86-5.69 (m, 1H), 5.51 (dd, J = 15.6, 3.7 Hz, 1H), 2.41 (s, 3H), 2.34 (s, 3H). |
| | 6-(2-fluoro-4-(2- | | |
| | fluoroacrylamido)phenyl)-3- | | |
| | methyl-7-(4-((4-methylpyrimidin- | | |
| | 2-yl)oxy)phenyl)pyrrolo[1,2- | | |
| | a]pyrazine-8-carboxamide | | |
| 43 | 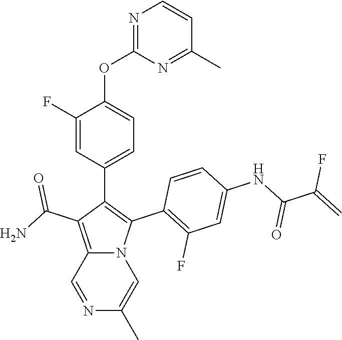 [SMILES: C=C(F)C(=O)Nc1ccc(-c2c(-c3ccc(Oc4nccc(C)n4)c(F)c3)c(C(N)=O)c3cnc(C)cn23)c(F)c1] | 559.2 | (400 MHz, DMSO-d₆) δ 10.68 (s, 1H), 9.18 (s, 1H), 8.48 (d, J = 5.1 Hz, 1H), 7.89 (dd, J = 12.3, 2.0 Hz, 1H), 7.70 (s, 1H), 7.66 (dd, J = 8.6, 2.0 Hz, 1H), 7.51-7.40 (m, 1H), 7.36-7.31 (m, 2H), 7.25 (dd, J = 11.6, 1.9 Hz, 1H), 7.19 (d, J = 5.1 Hz, 1H), 7.16- 7.10 (m, 1H), 7.09-7.05 (m, 1H), 5.89-5.68 (m, 1H), 5.51 (dd, J = 15.6, 3.7 Hz, 1H), 2.42 (s, 3H), 2.36 (s, 3H). |
| | 7-(3-fluoro-4-((4- | | |
| | methylpyrimidin-2- | | |
| | yl)oxy)phenyl)-6-(2-fluoro-4-(2- | | |
| | fluoroacrylamido)phenyl)-3- | | |
| | methylpyrrolo[1,2-a]pyrazine-8- | | |
| | carboxamide | | |
| 44 | 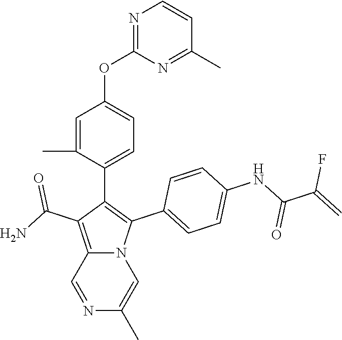 [SMILES: C=C(F)C(=O)Nc1ccc(-c2c(-c3ccc(Oc4nccc(C)n4)cc3C)c(C(N)=O)c3cnc(C)cn23)cc1] | 537.2 | (400 MHz, DMSO-d₆) δ 10.42 (s, 1H), 9.46 (d, J = 0.9 Hz, 1H), 8.46 (d, J = 5.1 Hz, 1H), 7.92 (s, 1H), 7.79 (d, J = 8.6 Hz, 2H), 7.33-7.23 (m, 4H), 7.14 (d, J = 5.1 Hz, 1H), 7.09 (d, J = 2.2 Hz, 1H), 7.04 (dd, J = 8.1, 2.4 Hz, 1H), 5.79-5.65 (m, 2H), 5.45 (dd, J = 15.6, 3.5 Hz, 1H), 2.40 (s, 3H), 2.37 (s, 3H), 1.97 (s, 3H). |
| | fluoroacrylamido)phenyl)-3- | | |
| | methyl-7-(2-methyl-4-((4- | | |
| | methylpyrimidin-2- | | |
| | yl)oxy)phenyl)pyrrolo[1,2- | | |
| | a]pyrazine-8-carboxamide | | |
| 45 | 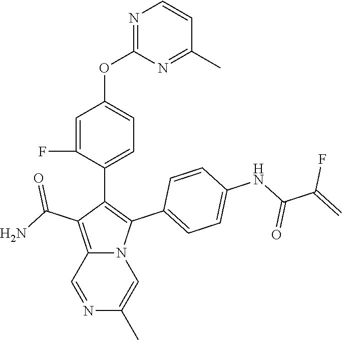 [SMILES: C=C(F)C(=O)Nc1ccc(-c2c(-c3ccc(Oc4nccc(C)n4)cc3F)c(C(N)=O)c3cnc(C)cn23)cc1] | 541.1 | (400 MHz, DMSO-d₆) δ 10.46 (s, 1 H), 9.22 (d, J = 1.1 Hz, 1H), 8.49 (d, J = 5.1 Hz, 1H), 7.90-7.80 (m, 3H), 7.39-7.23 (m, 4H), 7.20-7.13 (m, 2H), 7.00 (dd, J = 8.4, 2.2 Hz, 1H), 6.85 (s, 1H), 5.82-5.65 (m, 1H), 5.46 (dd, J = 15.6, 3.7 Hz, 1H), 2.42 (s, 3H), 2.35 (s, 3H). |
| | 7-(2-fluoro-4-((4- | | |
| | methylpyrimidin-2- | | |
| | yl)oxy)phenyl)-6-(4-(2- | | |
| | fluoroacrylamido)phenyl)-3- | | |
| | methylpyrrolo[1,2-a]pyrazine-8- | | |
| | carboxamide | | |
| 46 | 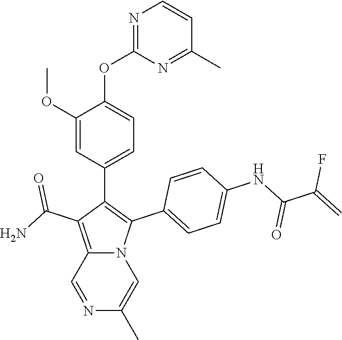 [SMILES: C=C(F)C(=O)Nc1ccc(-c2c(-c3ccc(Oc4nccc(C)n4)c(OC)c3)c(C(N)=O)c3cnc(C)cn23)cc1] | 553.3 | (400 MHz, DMSO-d₆) δ 10.46 (br s, 1H) 9.27 (s, 1H) 8.40 (d, J = 4.9 Hz, 1H), 7.88-7.76 (m, 3 H), 7.51-7.22 (m, 3H), 7.15-7.05 (m, 2H), 7.03 (d, J = 1.5 Hz, 1H), 6.89 (dd, J = 8.1, 1.7 Hz, 1H), 6.59 (br s, 1H), 5.88-5.59 (m, 1H), 5.46 (dd, J = 15.6, 3.7 Hz, 1H), 3.51 (s, 3H), 2.50 (s, 3H), 2.39 (s, 3H). |
| | 6-(4-(2- | | |
| | fluoroacrylamido)phenyl)-7-(3- | | |
| | methoxy-4-((4-methylpyrimidin- | | |
| | 2-yl)oxy)phenyl)-3- | | |
| | methylpyrrolo[1,2-a]pyrazine-8- | | |
| | carboxamide | | |
| 47 | 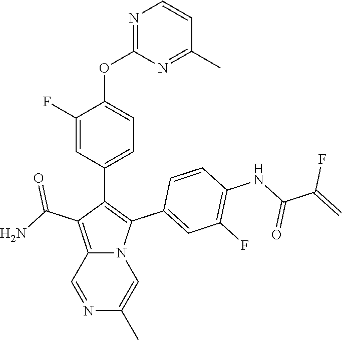 [SMILES: C=C(F)C(=O)Nc1ccc(-c2c(-c3ccc(Oc4nccc(C)n4)c(F)c3)c(C(N)=O)c3cnc(C)cn23)cc1F] | 559.2 | (400 MHz, DMSO-d₆) δ 10.26 (s, 1H), 9.22 (s, 1H), 8.48 (d, J = 5.1 Hz, 1H), 7.92 (s, 1H), 7.66 (t, J = 8.1 Hz, 1H), 7.48-7.15 (m, 6H), 7.11 (dd, J = 8.3, 1.0 Hz, 1H), 6.92 (br s, 1H) 5.83-5.66 (m, 1H), 5.49 (dd, J = 15.6, 3.8 Hz, 1H), 2.42 (s, 3H), 2.38 (s, 3H). |
| | 7-(3-fluoro-4-((4- | | |
| | methylpyrimidin-2- | | |
| | yl)oxy)phenyl)-6-(3-fluoro-4-(2- | | |
| | fluoroacrylamido)phenyl)-3- | | |
| | methylpyrrolo[1,2-a]pyrazine-8- | | |
| | carboxamide | | |
| 48 | 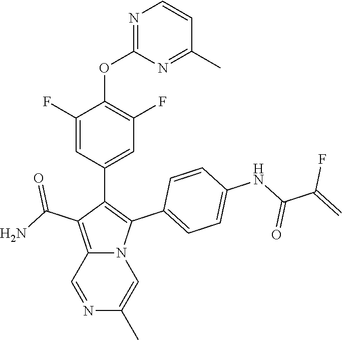 [SMILES: C=C(F)C(=O)Nc1ccc(-c2c(-c3cc(F)c(Oc4nccc(C)n4)c(F)c3)c(C(N)=O)c3cnc(C)cn23)cc1] | 559.2 | (400 MHz, DMSO-d₆) δ 10.50 (s, 1H), 9.17 (s, 1H), 8.51 (d, J = 5.0 Hz, 1H), 7.94-7.78 (m, 3H), 7.49- 7.32 (m, 3H), 7.29-7.03 (m, 4H), 5.86-5.65 (m, 1H), 5.48 (dd, J = 15.6, 3.6 Hz, 1H), 2.44 (s, 3H), 2.35 (s, 3 H). |
| | 7-(3,5-difluoro-4-((4- | | |
| | methylpyrimidin-2- | | |
| | yl)oxy)phenyl)-6-(4-(2- | | |
| | fluoroacrylamido)phenyl)-3- | | |
| | methylpyrrolo[1,2-a]pyrazine-8- | | |
| | carboxamide | | |
| 49 | 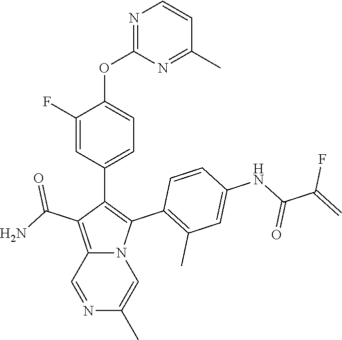 [SMILES: C=C(F)C(=O)Nc1ccc(-c2c(-c3ccc(Oc4nccc(C)n4)c(F)c3)c(C(N)=O)c3cnc(C)cn23)c(C)c1] | 555.1 | (400 MHz, DMSO-d₆) δ 10.40 (s, 1H), 9.18 (d, J = 1.3 Hz, 1H), 8.46 (d, J = 4.8 Hz, 1H), 7.79 (d, J = 1.8 Hz, 1H), 7.71-7.66 (m, 1H), 7.40 (s, 2H), 7.31-7.24 (m, 2H), 7.22-7.16 (m, 2H), 7.05 (dd, J = 8.1, 1.3 Hz, 2H), 5.81-5.66 (m, 1H), 5.46 (dd, J = 15.6, 3.5 Hz, 1H), 2.40 (s, 3H), 2.33 (s, 3H), 1.89 (s, 3H). |
| | 7-(3-fluoro-4-((4- | | |
| | methylpyrimidin-2- | | |
| | yl)oxy)phenyl)-6-(4-(2- | | |
| | fluoroacrylamido)-2- | | |
| | methylphenyl)-3- | | |
| | methylpyrrolo[1,2-a]pyrazine-8- | | |
| | carboxamide | | |
| 51 | 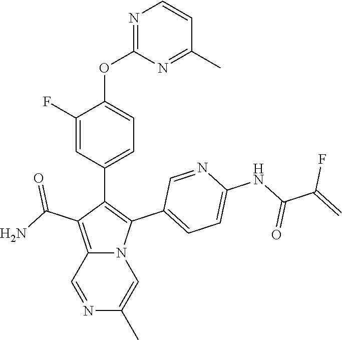 [SMILES: C=C(F)C(=O)Nc1ccc(-c2c(-c3ccc(Oc4nccc(C)n4)c(F)c3)c(C(N)=O)c3cnc(C)cn23)cn1] | 542.2 | (400 MHz, DMSO-d₆) δ 10.95 (br s, 1H), 9.23 (d, J = 1.4 Hz, 1H), 8.48 (d, J = 5.0 Hz, 1H), 8.29 (dd, J = 2.4, 0.8 Hz, 1H), 8.18 (dd, J = 8.6, 0.6 Hz, 1H), 8.08-7.81 (m, 2H), 7.44 (br s, 1H), 7.36-7.28 (m, 2H), 7.20 (d, J = 5.1 Hz, 1H), 7.11 (dd, J = 8.3, 1.4 Hz, 1H), 7.02-6.84 (m, 1H), 5.85-5.74 (m, 1H) 5.52 (dd, J = 15.6, 4.0 Hz, 1H), 2.42 (s, 3H), 2.37 (s, 3H). |
| | 7-(3-fluoro-4-((4- | | |
| | methylpyrimidin-2- | | |
| | yl)oxy)phenyl)-6-(6-(2- | | |
| | fluoroacrylamido)pyridin-3-yl)- | | |
| | 3-methylpyrrolo[1,2-a]pyrazine- | | |
| | 8-carboxamide | | |
| 52 | 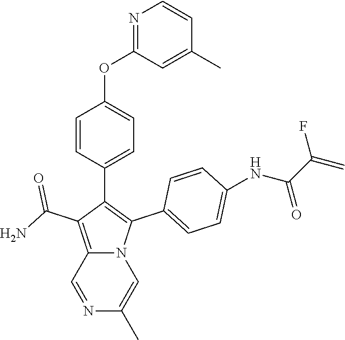 [SMILES: C=C(F)C(=O)Nc1ccc(-c2c(-c3ccc(Oc4cc(C)ccn4)cc3)c(C(N)=O)c3cnc(C)cn23)cc1] | 544.4 [M + Na]⁺ | (400 MHz, DMSO-d₆) δ 10.45 (s, 1H), 9.26 (d, J = 0.9 Hz, 1H), 8.04 (d, J = 5.1 Hz, 1H), 7.87-7.79 (m, 3H), 7.40-7.24 (m, 5 H,) 7.06 (d, J = 8.8 Hz, 2H), 6.99 (d, J = 4.8 Hz, 1H), 6.85 (s, 1H), 6.49 (br s, 1 H), 5.82-5.64 (m, 1H), 5.45 (dd, J = 15.6, 3.5 Hz, 1H), 2.34 (s, 3H), 2.33 (s, 3H). |
| | 6-(4-(2- | | |
| | fluoroacrylamido)phenyl)-3- | | |
| | methyl-7-(4-((4-methylpyridin-2- | | |
| | yl)oxy)phenyl)pyrrolo[1,2- | | |
| | alpyrazine-8-carboxamide | | |
| 53 | 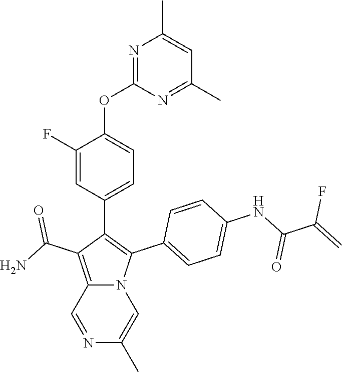 [SMILES: C=C(F)C(=O)Nc1ccc(-c2c(-c3ccc(Oc4nc(C)cc(C)n4)c(F)c3)c(C(N)=O)c3cnc(C)cn23)cc1] | 555.3 | (400 MHz, DMSO-d₆) δ 10.47 (s, 1H), 9.22 (s, 1H), 7.89-7.81 (m, 3H), 7.41 (d, J = 4.0 Hz, 1H), 7.36-7.20 (m, 5 H), 7.13-7.02 (m, 2H), 6.84 (br s, 1 H) 5.83-5.66 (m, 1H), 5.47 (dd, J = 15.6, 3.6 Hz, 1H), 2.36 (s, 3 H), 2.34 (s, 6 H). |
| | 7-(4-((4,6-dimethylpyrimidin-2- | | |
| | yl)oxy)-3-fluorophenyl)-6-(4-(2- | | |
| | fluoroacrylamido)phenyl)-3- | | |
| | methylpyrrolo[1,2-a]pyrazine-8- | | |
| | carboxamide | | |
| 54 | 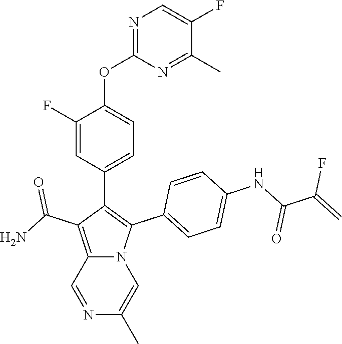 [SMILES: C=C(F)C(=O)Nc1ccc(-c2c(-c3ccc(Oc4ncc(F)c(C)n4)c(F)c3)c(C(N)=O)c3cnc(C)cn23)cc1] | 559.1 | (400 MHz, DMSO-d₆) δ 10.48 (s, 1H), 9.19 (d, J = 1.1 Hz, 1H), 8.57 (d, J = 1.1 Hz, 1H), 7.93-7.77 (m, 3H), 7.43 (br s, 1H), 7.37-7.29 (m, 3H), 7.26 (dd, J = 11.5, 1.9 Hz, 1H), 7.10 (dd, J = 8.3, 1.2 Hz, 1H), 6.97 (br s, 1H), 5.84-5.65 (m, 1H), 5.47 (dd, J= 15.6, 3.8 Hz, 1H), 2.42 (d, J = 2.4 Hz, 3H), 2.35 (s, 3H). |
| | 7-(3-fluoro-4-((5-fluoro-4- | | |
| | methylpyrimidin-2- | | |
| | yl)oxy)phenyl)-6-(4-(2- | | |
| | fluoroacrylamido)phenyl)-3- | | |
| | methylpyrrolo[1,2-a]pyrazine-8- | | |
| | carboxamide | | |
| 55 | 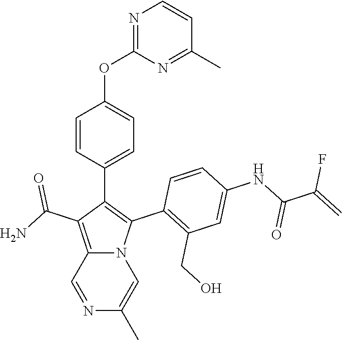 [SMILES: C=C(F)C(=O)Nc1ccc(-c2c(-c3ccc(Oc4nccc(C)n4)cc3)c(C(N)=O)c3cnc(C)cn23)c(CO)c1] | 553.2 | (400 MHz, DMSO-d₆) δ 10.47 (s, 1H), 9.22 (d, J = 1.1 Hz, 1H), 8.44 (d, J = 4.8 Hz, 1H), 8.03 (d, J = 1.8 Hz, 1H), 7.76 (dd, J = 8.4, 2.0 Hz, 1H), 7.41-7.23 (m, 5 H), 7.15-7.09 (m, 3 H), 6.73 (br s, 1H), 5.82-5.67 (m, 1H), 5.45 (dd, J = 15.6, 3.7 Hz, 1H), 5.18 (br s, 1H), 4.08-3.92 (m, 2H), 2.39 (s, 3H), 2.31 (s, 3H). |
| | 6-(4-(2-fluoroacrylamido)-2- | | |
| | (hydroxymethyl)phenyl)-3- | | |
| | methyl-7-(4-((4-methylpyrimidin- | | |
| | 2-yl)oxy)phenyl)pyrrolo[1,2- | | |
| | a]pyrazine-8-carboxamide | | |
| 56 | 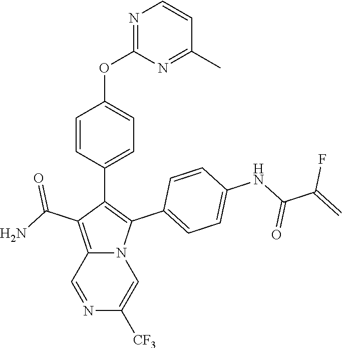 [SMILES: C=C(F)C(=O)Nc1ccc(-c2c(-c3ccc(Oc4nccc(C)n4)cc3)c(C(N)=O)c3cnc(C(F)(F)F)cn23)cc1] | 577.2 | (400 MHz, DMSO-d₆) δ 10.49 (s, 1H), 9.29 (s, 1H), 8.46 (d, J = 5.1 Hz, 1H), 8.28 (s, 1H), 7.87 (d, J = 8.6 Hz, 2H), 7.62 (br s, 1H), 7.41 (d, J = 8.6 Hz, 2H), 7.31 (d, J = 8.6 Hz, 2H), 7.19-7.13 (m, 4H), 5.81-5.67 (m, 1H), 5.46 (dd, J = 15.5, 3.6 Hz, 1H), 2.41 (s, 3H). |
| | 6-(4-(2- | | |
| | fluoroacrylamido)phenyl)-7-(4- | | |
| | ((4-methylpyrimidin-2- | | |
| | yl)oxy)phenyl)-3- | | |
| | (trifluoromethyl)pyrrolo[1,2- | | |
| | a]pyrazine-8-carboxamide | | |

### Example 50 Preparation of 6-(5-(2-fluoroacrylamido)pyridin-2-yl)-3-methyl-7-(4-((4-methylpyrimidin-2-yl)oxy)phenyl)pyrrolo[1,2-a]pyrazine-8-carboxamide

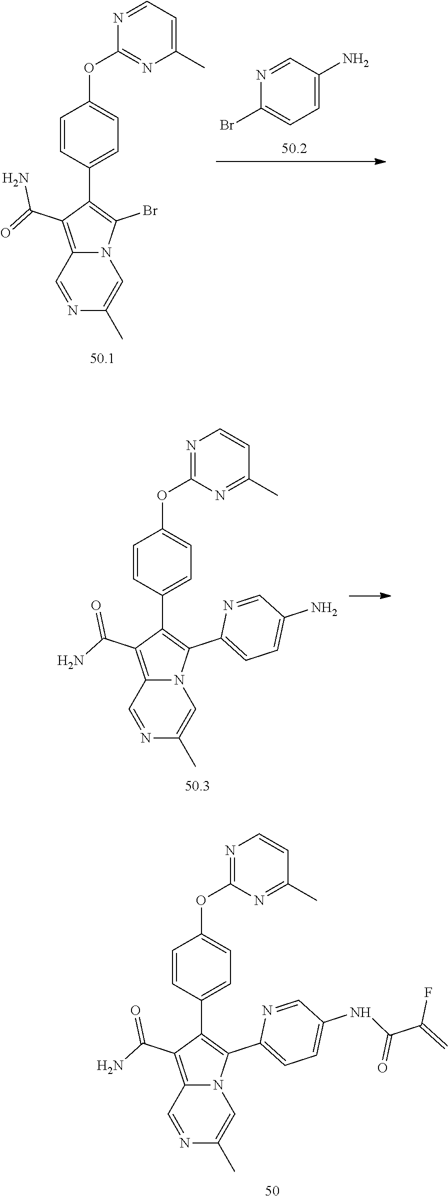 [SMILES: Cc1cn2c(Br)c(-c3ccc(Oc4nccc(C)n4)cc3)c(C(N)=O)c2cn1 ; Nc1ccc(Br)nc1 ; Cc1cn2c(-c3ccc(N)cn3)c(-c3ccc(Oc4nccc(C)n4)cc3)c(C(N)=O)c2cn1 ; C=C(F)C(=O)Nc1ccc(-c2c(-c3ccc(Oc4nccc(C)n4)cc3)c(C(N)=O)c3cnc(C)cn23)nc1]

### Step 1: Preparation of Compound 50.3

Compound 50.1 was synthesized in a similar fashion according to the procedures for example 15 and example 35.

A solution of compound 50.1 (150.0 mg, 0.342 mmol), compound 50.2 (88.8 mg, 0.513 mmol), 4,4,5,5-tetramethyl-2-(4,4,5,5-tetramethyl-1,3,2-dioxaborolan-2-yl)-1,3,2-dioxaborolane (156 mg, 0.616 mmol), K₂CO₃ (94.6 mg, 0.684 mmol) and Pd(dppf)Cl₂ (37.6 mg, 0.051 mmol) in dioxane (3 mL) was stirred at 100° C. for 16 h under N₂. After cooling to rt, the reaction mixture was diluted with EA (50 mL), washed with brine (15 mL), dried over Na₂SO₄, filtered, and purified by flash column chromatography on silica gel to give compound 50.3. LCMS: 451.0 [M+H]⁺.

### Step 2: Preparation of Example 50

To a solution of compound 50.3 (100 mg, 0.221 mmol) and 2-fluoroprop-2-enoic acid (39.9 mg, 0.443 mmol) in pyridine (3 mL) was added EDCI (127 mg, 0.664 mmol). The mixture was stirred at 50° C. for 2 h. The mixture was diluted with DCM (20 mL), washed with water (10 mL×2) and brine (20 mL), dried over Na₂SO₄, filtered, concentrated, and purified by prep-HPLC to afford example 50. LCMS: 524.2 [M+H]⁺. ¹H NMR: (400 MHZ, DMSO-d₆) δ 10.67 (s, 1H), 9.31 (s, 1H), 9.11 (d, J=2.3 Hz, 1H), 8.63 (s, 1H), 8.49 (d, J=4.8 Hz, 1H), 8.03 (dd, J=8.9, 2.4 Hz, 1H), 7.40-7.29 (m, 3H), 7.23 (d, J=8.5 Hz, 2H), 7.18 (d, J=5.0 Hz, 1H), 6.95 (d, J=8.8 Hz, 1H), 6.66-6.48 (m, 1H), 5.85-5.69 (m, 1H), 5.54-5.46 (m, 1H), 2.43 (s, 3H), 2.41 (s, 3H).

### Example 57 Preparation of (E)-6-(4-(4-(dimethylamino) but-2-enamido)phenyl)-3-methyl-7-(4-((4-methylpyrimidin-2-yl)oxy)phenyl)pyrrolo[1,2-a]pyrazine-8-carboxamide

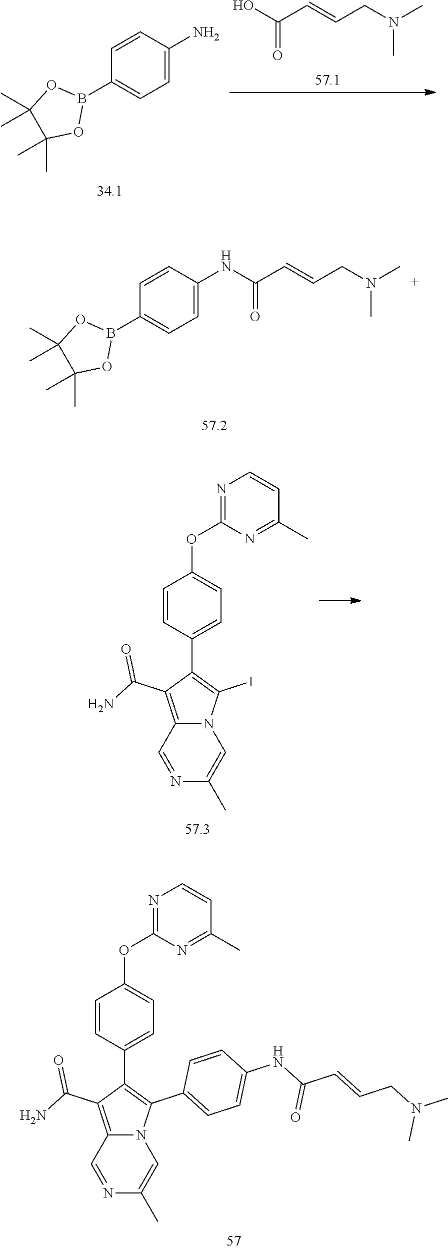

### Step 1: Preparation of Compound 57.2

To a solution of compound 34.1 (600.0 mg, 2.74 mmol) and compound 57.1 (540.0 mg, 3.29 mmol) in DCM (10 mL) was added DIEA (1.06 g, 8.22 mmol). Then HATU (1.09 g, 2.88 mmol) was added in the solution and the solution was stirred at 25° C. for 3 h. The reaction solution was diluted with water (30 mL) and extracted with DCM (20 mL×3). The combined organic layers were washed with brine (30 mL), dried over Na₂SO₄, filtered, concentrated, and purified by flash column chromatography on silica gel to afford compound 57.2 (300.0 mg, 33.17%) as a white solid. LCMS: 331.3 [M+H]⁺.

### Step 2: Preparation of Example 57

Compound 57.3 was synthesized in a similar fashion according to the procedures for compound 35.3.

To a solution of compound 57.3 (120.0 mg, 0.247 mmol) and compound 57.2 (163.3 mg, 0.495 mmol) in dioxane (3 mL) and H₂O (1.5 mL) was added K₂CO₃ (102.5 mg, 0.742 mmol). Then Pd(PPh₃)₄ (28.5 mg, 0.025 mmol) was added in the mixture and the mixture was stirred at 90° C. for 2 h under N₂. After cooling to rt, the reaction mixture was diluted with H₂O (20 mL) and extracted with EA (30 mL×3). The combined organic layers were concentrated and purified by prep-HPLC to afford example 57. LCMS: 562.3 [M+H]⁺. ¹H NMR: (400 MHZ, DMSO-d₆) δ 10.22 (s, 1H), 9.24 (s, 1H), 8.46 (d, J=4.8 Hz, 1H), 7.84-7.70 (m, 3H), 7.33-7.26 (m, 5H), 7.18-7.12 (m, 3H), 6.78-6.71 (m, 1H), 6.32-6.29 (m, 1H), 3.07 (d, J=4.5 Hz, 2H), 2.41 (s, 3H), 2.35 (s, 3H), 2.19 (s, 6H).

### Example 58 Preparation of 6-(4-(2-(hydroxymethyl) acrylamido)phenyl)-3-methyl-7-(4-((4-methylpyrimidin-2-yl)oxy)phenyl)pyrrolo[1,2-a]pyrazine-8-carboxamide

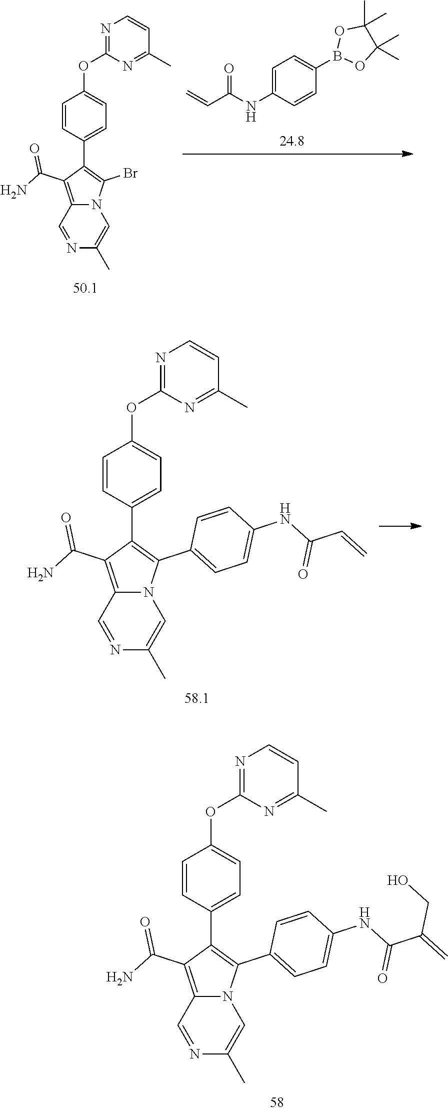

### Step 1: Preparation of Compound 58.1

To a solution of compound 50.1 (250.0 mg, 0.497 mmol) and compound 24.8 (187.0 mg, 0.684 mmol) in dioxane (5 mL) and H₂O (1 mL) was added K₂CO₃ (189.2 mg, 1.37 mmol). Then Pd-118 (30.0 mg, 0.046 mmol) was added in the mixture and the mixture was stirred at 90° C. for 2 h under N₂. After cooling to rt, the reaction solution was diluted with water (10 mL) and extracted with EA (15 mL×3). The combined organic layers were washed with brine (10 mL), dried over Na₂SO₄, filtered, and concentrated under reduced pressure to give a residue which was purified by flash column chromatography on silica gel to afford compound 58.1. LCMS: 505.1 [M+H]⁺.

### Step 2: Preparation of Example 58

To a solution of compound 58.1 (70.0 mg, 0.139 mmol) and formaldehyde (56.3 mg, 0.694 mmol, 37%) in dioxane (1 mL) and H₂O (0.3 mL) was added DABCO (46.7 mg, 0.416 mmol). The mixture was stirred at 80° C. for 8 h. After cooling to rt, the reaction mixture was diluted with H₂O (5 mL) and extracted with EA (10 mL×3). The combined organic layers were concentrated and purified by prep-HPLC to afford example 58. LCMS: 535.1 [M+H]⁺; ¹H NMR: (400 MHZ, DMSO-d₆) δ 10.33 (s, 1H), 9.19 (s, 1H), 8.46 (d, J=5.0 Hz, 1H), 8.11 (t, J=6.3 Hz, 1H), 7.84-7.74 (m, 3H), 7.33-7.26 (m, 4H), 7.17-7.10 (m, 3H), 6.50-6.41 (m, 1H), 6.32-6.25 (m, 1H), 5.82-5.77 (m, 1H), 5.73-5.64 (m, 1H), 4.65 (t, J=6.2 Hz, 2H), 2.41 (s, 3H), 2.35 (s, 3H).

### Example 59 Preparation of 6-(4-(2-((dimethylamino)methyl) acrylamido)phenyl)-3-methyl-7-(4-((4-methylpyrimidin-2-yl)oxy)phenyl)pyrrolo[1,2-a]pyrazine-8-carboxamide

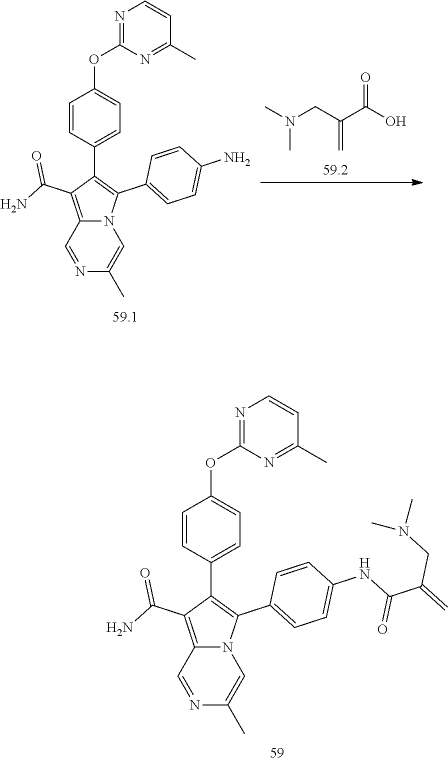

### Step 1: Preparation of Example 59

Compound 59.1 was synthesized in a similar fashion according to the procedures for example 1 and example 35.

In a similar fashion according to the procedures for example 50, example 59 was synthesized from compound 59.1. LCMS: 562.3 [M+H]⁺. ¹H NMR: (400 MHZ, DMSO-d₆) δ 11.27 (s, 1H), 9.25 (s, 1H), 8.47 (d, J=5.0 Hz, 1H), 7.81 (s, 1H), 7.71 (d, J=8.5 Hz, 2H), 7.33-7.29 (m, 5H), 7.20-7.13 (m, 3H), 6.58 (br s, 1H), 6.02 (s, 1H), 5.59 (s, 1H), 3.23 (s, 2H), 2.41 (s, 3H), 2.35 (s, 3H), 2.25 (s, 6H).

### Example 60 Preparation of 6-(4-(2-(methoxymethyl) acrylamido)phenyl)-3-methyl-7-(4-((4-methylpyrimidin-2-yl)oxy)phenyl)pyrrolo[1,2-a]pyrazine-8-carboxamide

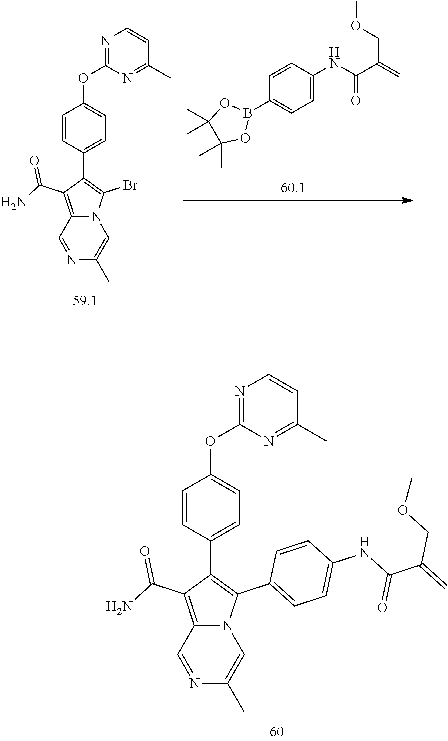 [SMILES: Cc1cn2c(Br)c(-c3ccc(Oc4nccc(C)n4)cc3)c(C(N)=O)c2cn1 ; C=C(COC)C(=O)Nc1ccc(B2OC(C)(C)C(C)(C)O2)cc1 ; C=C(COC)C(=O)Nc1ccc(-c2c(-c3ccc(Oc4nccc(C)n4)cc3)c(C(N)=O)c3cnc(C)cn23)cc1]

### Step 1: Preparation of Example 60

In a similar fashion according to the procedures for example 35, example 60 was synthesized from compound 50.1. LCMS: 549.3 [M+H]⁺. ¹H NMR: (400 MHZ, DMSO-d₆) δ 10.09 (s, 1H), 9.25 (s, 1H), 8.47 (d, J=5.1 Hz, 1H), 7.84-7.74 (m, 3H), 7.41-7.25 (m, 5H), 7.18-7.13 (m, 3H), 6.56 (br s, 1H), 5.95 (s, 1H), 5.71 (s, 1H), 4.18-4.14 (m, 2H), 3.30 (s, 3H), 2.41 (s, 3H), 2.35 (s, 3H).

### Example 61 Preparation of (E)-3-methyl-7-(4-((4-methylpyrimidin-2-yl)oxy)phenyl)-6-(4-(3-(1-methylpyrrolidin-2-yl) acrylamido)phenyl)pyrrolo[1,2-a]pyrazine-8-carboxamide

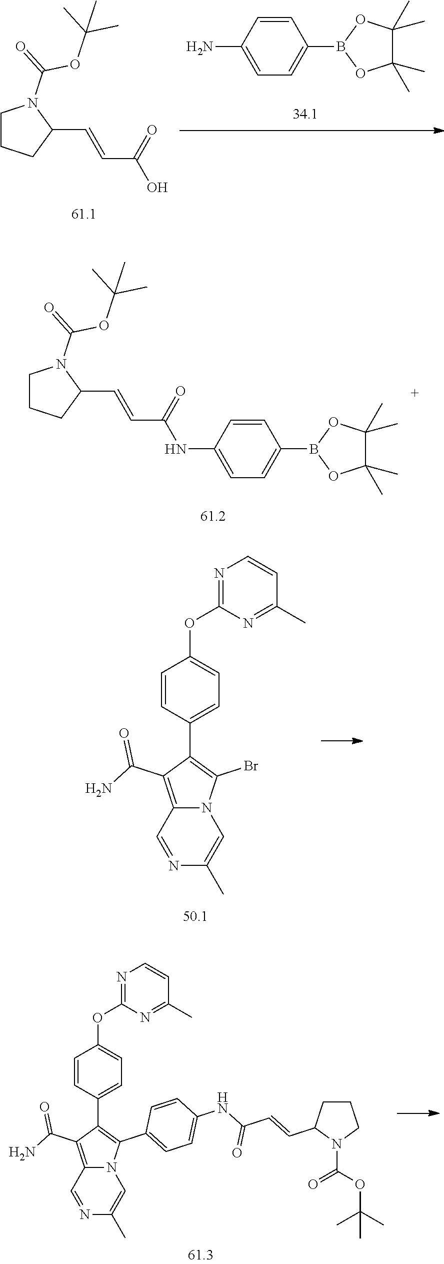 [SMILES: CC(C)(C)OC(=O)N1CCCC1/C=C/C(=O)O ; CC1(C)OB(c2ccc(N)cc2)OC1(C)C ; CC(C)(C)OC(=O)N1CCCC1/C=C/C(=O)Nc1ccc(B2OC(C)(C)C(C)(C)O2)cc1 ; Cc1cn2c(Br)c(-c3ccc(Oc4nccc(C)n4)cc3)c(C(N)=O)c2cn1 ; Cc1cn2c(-c3ccc(NC(=O)/C=C/C4CCCN4C(=O)OC(C)(C)C)cc3)c(-c3ccc(Oc4nccc(C)n4)cc3)c(C(N)=O)c2cn1]

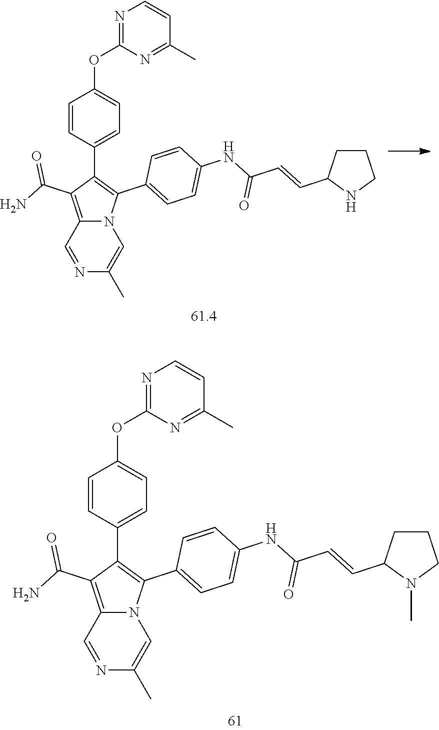 [SMILES: Cc1cn2c(-c3ccc(NC(=O)/C=C/C4CCCN4)cc3)c(-c3ccc(Oc4nccc(C)n4)cc3)c(C(N)=O)c2cn1 ; Cc1cn2c(-c3ccc(NC(=O)/C=C/C4CCCN4C)cc3)c(-c3ccc(Oc4nccc(C)n4)cc3)c(C(N)=O)c2cn1]

### Step 1: Preparation of Compound 61.2

To a solution of compound 61.1 (500.0 mg, 2.07 mmol) in DCM (5 mL) were added compound 34.1 (454.00 mg, 2.07 mmol), DIEA (1.03 mL, 6.22 mmol), and HATU (866.7 mg, 2.28 mmol). The reaction mixture was stirred at 25° C. for 12 h. The reaction mixture was poured into H₂O (10 mL) and extracted with EA (20 mL×3). The combined organic layers were washed with brine (50 mL), concentrated, and purified by flash column chromatography on silica gel to afford compound 61.2 (690.0 mg, 75.3%) as a white solid. LCMS: 443.2 [M+H]⁺.

### Step 2: Preparation of Compound 61.3

A solution of compound 50.1 (200.0 mg, 0.456 mmol) in dioxane (5 mL) and H₂O (1 mL) was added compound 61.2 (242.2 mg, 0.548 mmol). Then Pd-118 (29.7 mg, 0.046 mmol) and K₂CO₃ (189.2 mg, 1.37 mmol) were added in the solution under N₂. The solution was stirred at 90° C. for 2 h. After cooled to rt, the reaction mixture was concentrated and purified by flash column chromatography on silica gel to afford compound 61.3. LCMS: 674.3 [M+H]⁺.

### Step 3: Preparation of Compound 61.4

A solution of compound 61.3 (160.0 mg, 0.24 mmol) in DCM (2 mL) was added TFA (0.5 mL, 0.007 mmol) and the solution was stirred at 25° C. for 1 h. After the reaction was completed, the solution was concentrated under reduced pressure to afford compound 61.4. LCMS: 574.4 [M+H]⁺.

### Step 4: Preparation of Example 61

To a solution of compound 61.4 (160.0 mg, 0.279 mmol) and HCHO (45.3 mg, 0.558 mmol) in MeOH (2 mL) were added NaBH₃CN (61.3 mg, 0.976 mmol) and DIEA (0.138 mL, 0.837 mmol). The mixture was stirred at 25° C.° C. for 2 h. After the reaction was completed, the mixture was diluted with water (10 mL) and extracted with EA (8 mL×3). The combined organic layers were washed with brine (20 mL), dried over Na₂SO₄, filtered, concentrated, and purified by prep-HPLC to afford example 61. LCMS: 588.3 [M+H]⁺; ¹H NMR: (400 MHz, DMSO-d₆) δ 10.21 (s, 1H), 9.24 (s, 1H), 8.46 (d, J=5.1 Hz, 1H), 7.81 (s, 1H), 7.73 (d, J=8.6 Hz, 2H), 7.36 (br s, 1H), 7.31-7.25 (m, 4H), 7.17-7.11 (m, 3H), 6.65 (dd, J=15.2, 7.5 Hz, 1H), 6.58 (br s, 1H), 6.26 (d, J=15.2 Hz, 1H), 3.06-2.99 (m, 1H), 2.77-2.73 (m, 1H), 2.40 (s, 3H), 2.34 (s, 3H), 2.26-2.13 (m, 4H), 2.06-1.95 (m, 1H), 1.79-1.68 (m, 2H), 1.61-1.49 (m, 1H).

### Example 62 Preparation of 6-(4-(but-2-ynamido)phenyl)-3-methyl-7-(4-((4-methylpyrimidin-2-yl)oxy)phenyl)pyrrolo[1,2-a]pyrazine-8-carboxamide

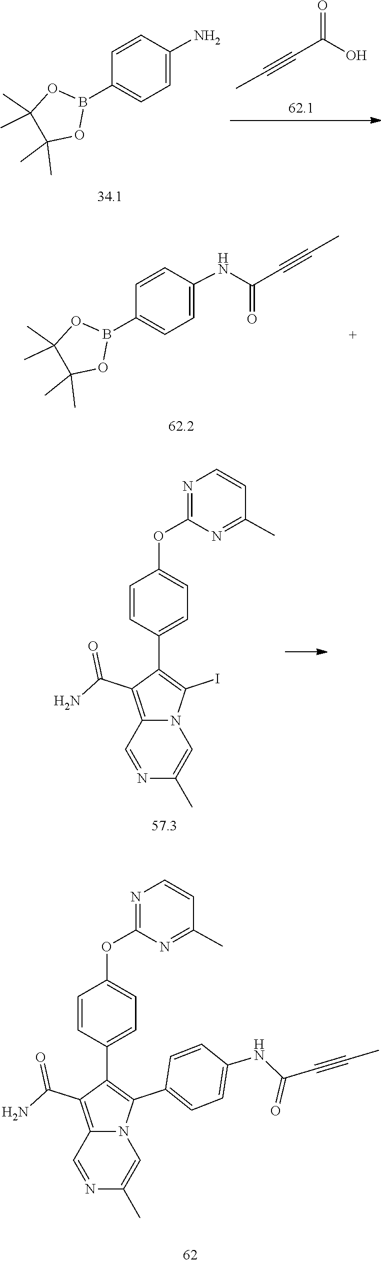

### Step 1: Preparation of Compound 62.2

To a solution of compound 34.1 (700.0 mg, 3.65 mmol) and compound 62.1 (340.0 mg, 4.02 mmol) in DCM (10 mL) was added DIEA (1.42 g, 10.9 mmol). Then HATU (1.46 g, 3.83 mmol) added in the solution and the solution was stirred at 25° C. for 3 h. The reaction solution was diluted with water (30 mL) and extracted with DCM (20 mL×3). The combined organic layers were washed with brine (30 mL), dried over Na₂SO₄, filtered, concentrated, and purified by flash column chromatography on silica gel to afford compound 62.2 (650.0 mg, 62.43%) as a white solid. LCMS: 286.2 [M+H]⁺.

### Step 2: Preparation of Example 62

To a solution of compound 57.3 (120.0 mg, 0.247 mmol) and compound 62.2 (105.8 mg, 0.371 mmol) in dioxane (3 mL) and H₂O (1.5 mL) was added K₂CO₃ (102.5 mg, 0.742 mmol). Then Pd(PPh₃)₄ (28.5 mg, 0.025 mmol) was added in the mixture and the mixture was stirred at 90° C. for 2 h under N₂. After cooling to rt, the reaction mixture was diluted with H₂O (20 mL) and extracted with EA (10 mL×3). The combined organic layers were concentrated and purified by prep-HPLC to afford example 62. LCMS: 517.1 [M+H]⁺. ¹H NMR: (400 MHZ, DMSO-d₆) δ 10.78 (s, 1H), 9.25 (d, J=1.0 Hz, 1H), 8.46 (d, J=5.0 Hz, 1H), 7.81 (s, 1H), 7.67 (d, J=8.4 Hz, 2H), 7.40-7.33 (m, 1H), 7.31-7.26 (m, 4H), 7.18-7.12 (m, 3H), 6.69-6.40 (m, 1H), 2.41 (s, 3H), 2.34 (s, 3H), 2.06 (s, 3H).

### Example 63 Preparation of 6-(4-(2-fluoroacrylamido)phenyl)-7-(4-(((3-fluorooxetan-3-yl)methyl) carbamoyl)phenyl)-3-methylpyrrolo[1,2-a]pyrazine-8-carboxamide

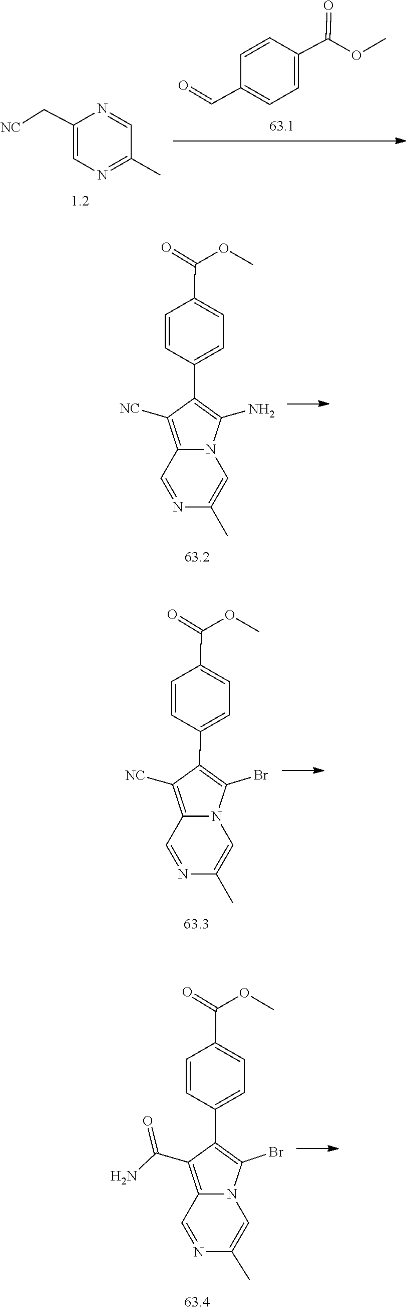

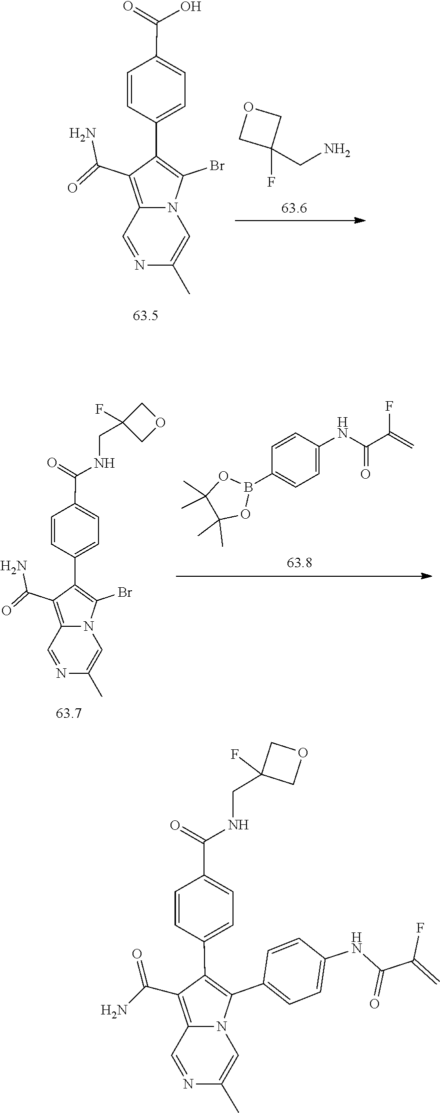

### Step 1: Preparation of Compound 63.2

To a solution of compound 1.2 (500.0 mg, 3.75 mmol) in MeOH (8 mL) were added compound 63.1 (616.4 mg, 3.75 mmol), DBU (114.3 mg, 0.75 mmol), and acetyl cyanide (259.4 mg, 3.75 mmol), the solution was stirred at 100° C. for 30 min. After cooling to rt, the reaction mixture was concentrated and purified by flash column chromatography on silica gel to afford compound 63.2 (690.0 mg, 60.0%) as a yellow solid. LCMS: 307.1 [M+H]⁺.

### Step 2: Preparation of Compound 63.3

To a solution of compound 63.2 (690.0 mg, 2.25 mmol) in HBr/AcOH (5 mL) were added CuBr₂ (654.1 mg, 2.93 mmol), and sodium nitrite (777.2 mg, 11.3 mmol) in H₂O (5 mL) was added at 0° C. The mixture was stirred at 0° C. for 1 h. After cooling to rt, the reaction solution was dissolved in 20 mL water. The pH of the solution was adjusted to 8 by Na₂CO₃ (aq). The reaction solution was extracted with EA (30 mL×3). The combined organic phase was washed with brine (50 mL), dried with anhydrous Na₂SO₄, filtered, concentrated in vacuum, and purified by flash column chromatography on silica gel to afford compound 63.3 (160.0 mg, 19.2%) as a yellow solid. LCMS: 370.0 [M+H]⁺.

### Step 3: Preparation of Compound 63.4

A solution of compound 63.3 (160.0 mg, 0.432 mmol) in H₂SO₄ (1.5 mL) was stirred at 25° C. for 12 h. After cooling to rt, the reaction solution was dissolved in water (20 mL). The pH of the solution was adjusted to 8 by Na₂CO₃ (aq). The reaction solution was extracted with EA (20 mL×3). The combined organic phase was washed with brine (50 mL), dried with anhydrous Na₂SO₄, filtered, and concentrated in vacuum to afford compound 63.4 (100 mg, crude) as a brown solid. LCMS: 390.0 [M+H]⁺.

### Step 4: Preparation of Compound 63.5

A solution of compound 63.4 (95.0 mg, 0.245 mmol) in THF (2 mL) was added potassium trimethylsilanolate (31.4 mg, 0.245 mmol) and the solution was stirred at 25° C. for 2 h. After the solution was completed, the reaction mixture was concentrated to afford compound 63.5. LCMS: 373.9 [M+H]⁺.

### Step 5: Preparation of Compound 63.7

To a solution of compound 63.5 (91.0 mg, 0.243 mmol) in DCM (1 mL) were added compound 63.6 (25.6 mg, 0.243 mmol), DIEA (0.12 mL, 0.73 mmol) and HATU (97.1 mg, 0.255 mmol). The solution was stirred at 25° C. for 12 h. After the reaction was completed, the solution was concentrated and purified by flash column chromatography on silica gel to afford compound 63.7. LCMS: 461.0 [M+H]⁺.

### Step 6: Preparation of Example 63

To a solution of compound 63.7 (50.0 mg, 0.108 mmol) and compound 63.8 (47.3 mg, 0.163 mmol) in dioxane (1 mL) were added Pd(PPh₃)₄ (12.5 mg, 0.011 mmol) and K₂CO₃ (44.9 mg, 0.325 mmol) under N₂. The solution was stirred at 90° C. for 3 h. The reaction solution was diluted with water (10 mL) and extracted with EA (10 mL×3). The combined organic phase was washed with brine (15 mL), dried with anhydrous Na₂SO₄, filtered, concentrated, and purified by prep-HPLC to afford example 63. LCMS: 546.2 [M+H]⁺; ¹H NMR: (400 MHz, DMSO-d₆) δ 10.43 (s, 1H), 9.22 (s, 1H), 8.80 (t, J=6.1 Hz, 1H), 7.85-7.76 (m, 5H), 7.35-7.26 (m, 5H), 6.66 (br s, 1H), 5.81-5.65 (m, 1H), 5.46 (dd, J=15.6, 3.7 Hz, 1H), 4.72-4.56 (m, 4H), 3.83-3.75 (m, 2H), 2.34 (s, 3H).

### Example 64 Preparation of 6-(4-acrylamidophenyl)-7-(4-(((3-fluorooxetan-3-yl)methyl) carbamoyl)phenyl)-3-methylpyrrolo[1,2-a]pyrazine-8-carboxamide

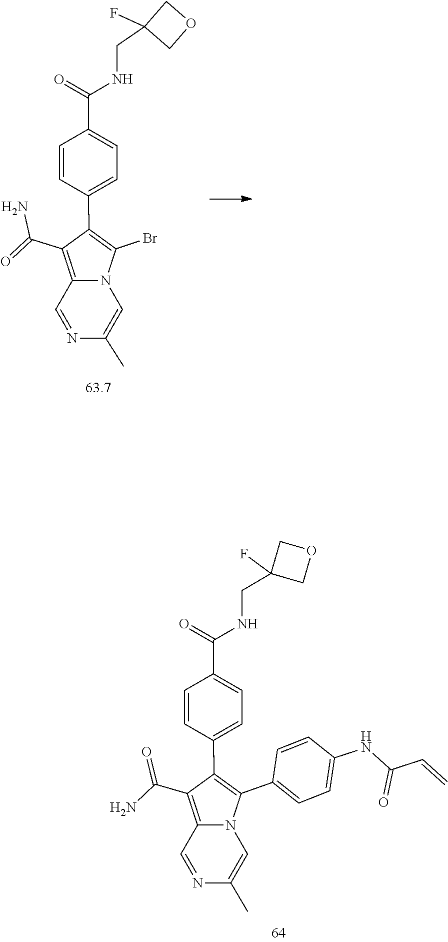 [SMILES: Cc1cn2c(Br)c(-c3ccc(C(=O)NCC4(F)COC4)cc3)c(C(N)=O)c2cn1 ; C=CC(=O)Nc1ccc(-c2c(-c3ccc(C(=O)NCC4(F)COC4)cc3)c(C(N)=O)c3cnc(C)cn23)cc1]

### Step 1: Preparation of Example 64

In a similar fashion according to the procedures for example 63, example 64 was synthesized from compound 63.7. LCMS: 528.1 [M+H]⁺. ¹H NMR: (400 MHZ, DMSO-d₆) δ 10.33 (s, 1H), 9.22 (s, 1H), 8.84-8.76 (m, 1H), 7.84 (s, 1H), 7.80 (d, J=8.1 Hz, 2H), 7.74 (d, J=8.6 Hz, 2H), 7.34 (d, J=8.4 Hz, 2H), 7.28-7.25 (m, 2H), 6.67 (br s, 1H), 6.49-6.39 (m, 1H), 6.33-6.24 (m, 1H), 5.83-5.73 (m, 1H), 4.75-4.54 (m, 5H), 3.80 (dd, J=19.6, 5.9 Hz, 2H), 2.35 (s, 3H).

### Example 65 Preparation of 6-(4-(but-2-ynamido)phenyl)-7-(4-(((3-fluorooxetan-3-yl)methyl) carbamoyl)phenyl)-3-methylpyrrolo[1,2-a]pyrazine-8-carboxamide

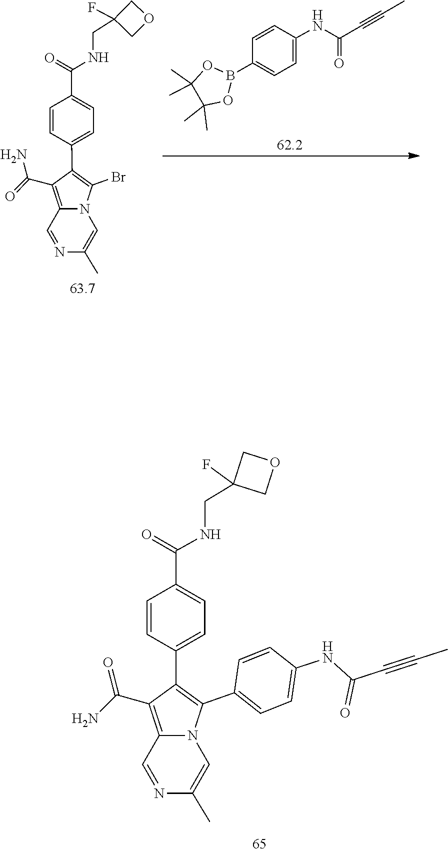 [SMILES: Cc1cn2c(Br)c(-c3ccc(C(=O)NCC4(F)COC4)cc3)c(C(N)=O)c2cn1 ; CC#CC(=O)Nc1ccc(B2OC(C)(C)C(C)(C)O2)cc1 ; CC#CC(=O)Nc1ccc(-c2c(-c3ccc(C(=O)NCC4(F)COC4)cc3)c(C(N)=O)c3cnc(C)cn23)cc1]

### Step 1: Preparation of Example 65

In a similar fashion according to the procedure for example 63, example 65 was synthesized from compound 63.7. Spectrum data of example 65: LCMS: 540.2 [M+H]⁺. ¹H NMR: (400 MHZ, DMSO-d₆) δ 10.77 (s, 1H), 9.23 (s, 1H), 8.80 (t, J=5.6 Hz, 1H), 7.96-7.80 (m, 3H), 7.66-7.64 (m, 2H), 7.34-7.32 (m, 3H), 7.26-7.24 (m, 2H), 6.70-6.61 (m, 1H), 4.71-4.58 (m, 4H), 3.80 (dd, J=20.0, 6.0 Hz, 2H), 2.35 (s, 3H), 2.06 (s, 3H).

### Example 66 Preparation of 6-(2-ethynyl-1-methyl-1H-benzo[d]imidazol-6-yl)-3-methyl-7-(4-((4-methylpyrimidin-2-yl)oxy)phenyl)pyrrolo[1,2-a]pyrazine-8-carboxamide

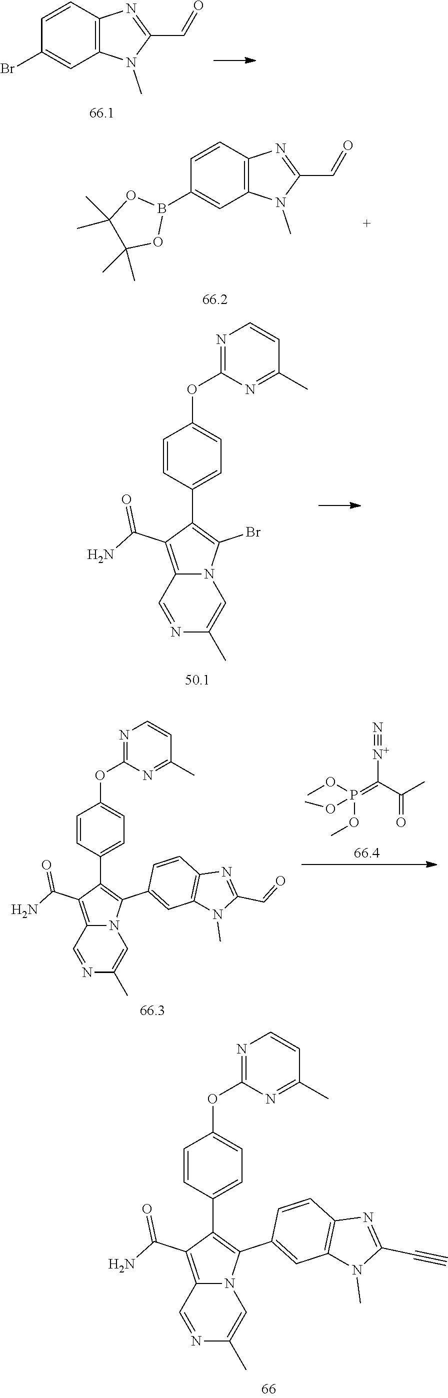 [SMILES: Cn1c(C=O)nc2ccc(Br)cc21 ; Cn1c(C=O)nc2ccc(B3OC(C)(C)C(C)(C)O3)cc21 ; Cc1cn2c(Br)c(-c3ccc(Oc4nccc(C)n4)cc3)c(C(N)=O)c2cn1 ; Cc1cn2c(-c3ccc4nc(C=O)n(C)c4c3)c(-c3ccc(Oc4nccc(C)n4)cc3)c(C(N)=O)c2cn1 ; COP(OC)(OC)=C([N+]#N)C(C)=O ; C#Cc1nc2ccc(-c3c(-c4ccc(Oc5nccc(C)n5)cc4)c(C(N)=O)c4cnc(C)cn34)cc2n1C]

### Step 1: Preparation of Compound 66.2

To a solution of compound 66.1 (1.00 g, 4.18 mmol) and (Bpin)₂ (3.10 g, 8.37 mmol) in dioxane (15 mL) was added AcOK (0.80 g, 8.37 mmol). Then Pd(dppf)Cl₂ (0.30 g, 0.42 mmol) was added in the mixture and the mixture was stirred at 90° C. for 16 h under N₂. After cooling to rt, the reaction solution was diluted with water (20 mL) and extracted with EA (30 mL×3). The combined organic layers were washed with brine (20 mL), dried over Na₂SO₄, filtered, concentrated, and purified by flash column chromatography on silica gel to afford compound 66.2 (450.0 mg, 37.6%) as a white solid. LCMS: 287.1 [M+H]⁺.

### Step 2: Preparation of Compound 66.3

To a solution of compound 66.2 (215.4 mg, 0.753 mmol) and compound 50.1 (300.0 mg, 0.684 mmol) in dioxane (6 mL) and H₂O (1 mL) was added Na₂CO₃ (181.3 mg, 1.71 mmol). Then Pd(PPh₃)₄ (79.1 mg, 0.068 mmol) was added and the mixture was stirred at 90° C. for 16 h under N₂. After cooling to rt, the reaction solution was diluted with water (10 mL) and extracted with EA (10 mL×3). The combined organic layers were washed with brine (10 mL), dried over Na₂SO₄, filtered, concentrated, and purified by flash column chromatography on silica gel to afford compound 66.3. LCMS: 518.3 [M+H]⁺.

### Step 3: Preparation of Example 66

To a solution of compound 66.3 (100.0 mg, 0.193 mmol) in MeOH (5 mL) was added K₂CO₃ (53.4 mg, 0.386 mmol). Then compound 66.4 was added in the solution under N₂ and the mixture was stirred at 25° C. for 2 h. The reaction solution was diluted with water (10 mL) and EA (10 mL×3). The combined organic layers were washed with brine (10 mL), dried over Na₂SO₄, filtered, concentrated, and purified by prep-HPLC to afford example 66. LCMS: 514.2 [M+H]⁺; ¹H NMR: (400 MHz, DMSO-d₆) δ 9.29 (d, J=1.1 Hz, 1H), 8.44 (d, J=5.0 Hz, 1H), 7.89 (s, 1H), 7.73-7.66 (m, 2H), 7.42-7.30 (m, 3H), 7.16-7.10 (m, 4H), 6.69-6.44 (m, 1H), 4.99 (s, 1H), 3.86 (s, 3H), 2.39 (s, 3H), 2.34 (s, 3H).

### Example 67 Preparation of 3-methyl-6-(1-methyl-2-(prop-1-yn-1-yl)-1H-benzo[d]imidazol-6-yl)-7-(4-((4-methylpyrimidin-2-yl)oxy)phenyl)pyrrolo[1,2-a]pyrazine-8-carboxamide

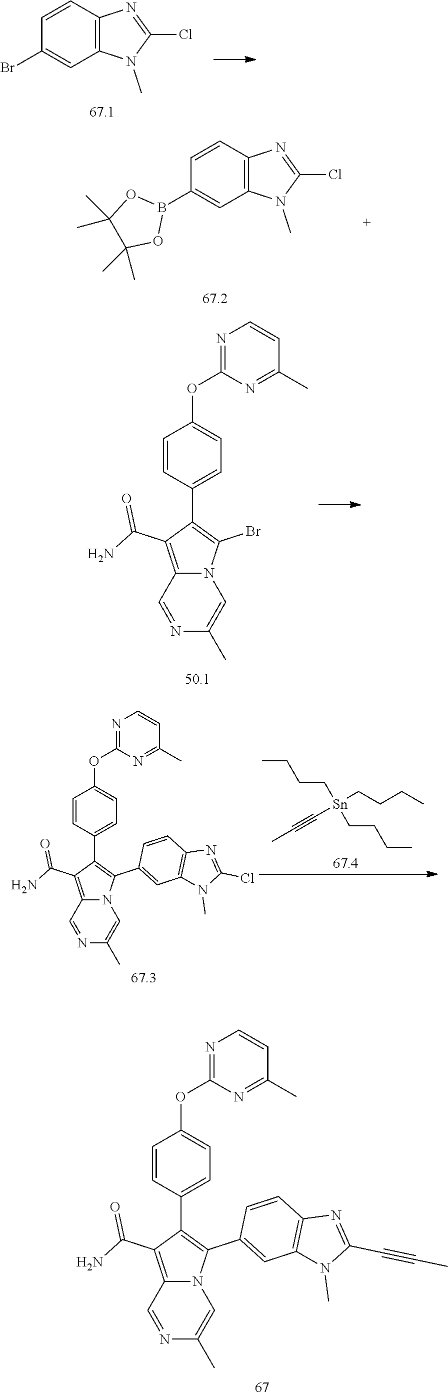

### Step 1: Preparation of Compound 67.2

To a mixture of compound 67.1 (600.0 mg, 2.44 mmol) and (Bpin)₂ (682.6 mg, 2.69 mmol) in dioxane (8 mL) was added K₂CO₃ (844.4 mg, 6.11 mmol). Then Pd(dppf)Cl₂ (178.8 mg, 0.244 mmol) was added in the solution under N₂. The reaction solution was stirred at 90° C. for 18 h under N₂. After cooling to rt, the reaction solution was diluted with water (10 mL) and extracted with EA (15 mL×3). The combined organic layers were washed with brine (10 mL), dried over Na₂SO₄, filtered, concentrated, and purified by flash column chromatography on silica gel to afford compound 67.2 (400.0 mg, 55.9%) as a yellow solid. LCMS: 293.2 [M+H]⁺.

### Step 2: Preparation of Compound 67.3

To a solution of compound 50.1 (240.0 mg, 0.548 mmol) and compound 67.2 (320.4 mg, 1.095 mmol) in dioxane (6 mL) and H₂O (2 mL) was added K₂CO₃ (189.2 mg, 1.37 mmol). Then Pd(PPh₃)₄ (63.3 mg, 0.055 mmol) was added in the solution under N₂. The reaction solution was stirred at 90° C. for 2 h under N₂. After cooling to rt, the reaction solution was diluted with water (5 mL) and extracted with EA (10 mL×3). The combined organic layers were washed with brine (10 mL), dried over Na₂SO₄, filtered, and concentrated under reduced pressure to give a residue which was purified by flash column chromatography on silica gel to afford compound 67.3. LCMS: 524.1 [M+H]⁺.

### Step 5: Preparation of Example 67

To a solution of compound 67.3 (150.0 mg, 0.286 mmol) and compound 67.4 (188.4 mg, 0.573 mmol) in dioxane (5 mL) was added Pd(PPh₃)₄ (33.1 mg, 0.029 mmol). The reaction was stirred at 110° C. for 2 h under N₂. After cooling to rt, the reaction solution was diluted with water (5 mL) and extracted with EA (10 mL×3). The combined organic layers were washed with brine (10 mL), dried over Na₂SO₄, filtered, concentrated, and purified by prep-HPLC to afford the example 67. LCMS: 528.2 [M+H]⁺. ¹H NMR: (400 MHZ, DMSO-d₆) δ 9.28 (d, J=1.3 Hz, 1H), 8.44 (d, J=5.0 Hz, 1H), 7.89 (s, 1H), 7.69-7.58 (m, 2H), 7.45-7.23 (m, 3H), 7.19-7.03 (m, 4H), 6.58 (br s, 1H), 3.82 (s, 3H), 2.39 (s, 3H), 2.33 (s, 3H), 2.24 (s, 3H)

### Example 68 Preparation of 6-(1-(1-acryloylazetidin-3-yl)-1H-pyrazol-4-yl)-3-methyl-7-(4-((4-methylpyrimidin-2-yl)oxy)phenyl)pyrrolo[1,2-a]pyrazine-8-carboxamide

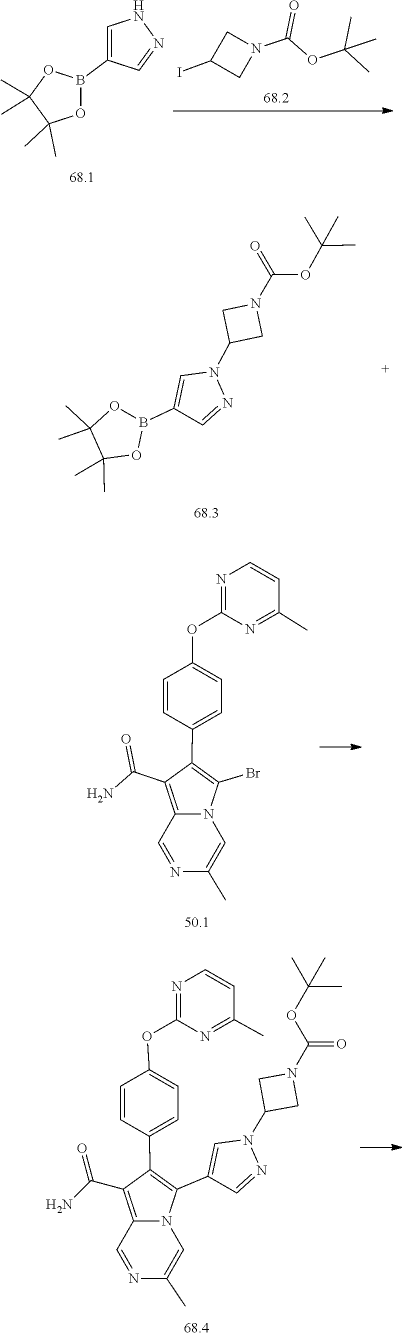

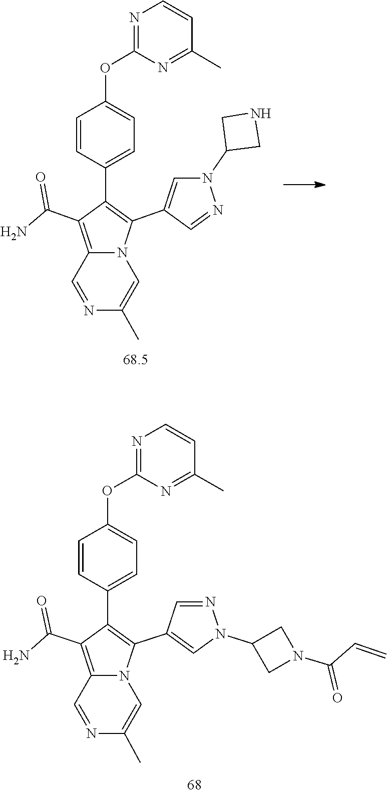 [SMILES: Cc1cn2c(-c3cnn(C4CNC4)c3)c(-c3ccc(Oc4nccc(C)n4)cc3)c(C(N)=O)c2cn1 ; C=CC(=O)N1CC(n2cc(-c3c(-c4ccc(Oc5nccc(C)n5)cc4)c(C(N)=O)c4cnc(C)cn34)cn2)C1]

### Step 1: Preparation of Compound 68.3

To a solution of compound 68.1 (3.00 g, 15.46 mmol) and compound 68.2 (4.40 g, 15.46 mmol) in DMF (30 mL) was added Cs₂CO₃ (10.1 g, 30.93 mmol). Then the mixture was stirred at 80° C. for 12 h. After cooling to rt, the reaction solution was diluted with water (90 mL) and extracted with DCM (30 mL×3). The combined organic layers were washed with brine (30 mL), dried over Na₂SO₄, filtered, concentrated, and purified by flash column chromatography on silica gel to afford compound 68.3 (2.00 g, 37.0%) as a yellow solid. LCMS: 350.2 [M+H]⁺.

### Step 2: Preparation of Compound 68.4

To a solution of compound 68.3 (478.0 mg, 1.369 mmol) and compound 50.1 (300.0 mg, 0.68 mmol) in dioxane (3 mL) and H₂O (1 mL) was added K₂CO₃ (283.8 mg, 2.05 mmol). Then Pd-118 (45.0 mg, 0.068 mmol) was added and the mixture was stirred at 90° C. for 2 h under N₂. After cooling to rt, the reaction solution was diluted with water (10 mL), extracted with (10 mL×3), washed with brine (15 mL), dried over Na₂SO₄, filtered, and concentrated to afford t compound 68.4. LCMS: 581.4 [M+H]⁺.

### Step 3: Preparation of Compound 68.5

To a solution of compound 68.4 (100.0 mg, 0.172 mmol) in DCM (2 mL) was added TFA (0.5 ml, 0.068 mmol) and the mixture was stirred at 25° C. for 1 h. The reaction mixture was concentrated under reduced pressure to afford compound 68.5. LCMS: 481.2 [M+H]⁺.

### Step 4: Preparation of Example 68

To a solution of compound 68.5 (100.0 mg, 0.208 mmol) in acetone (2 mL) and H₂O (0.4 mL) was added K₂CO₃ (28.8 mg, 0.208 mmol). Then acryloyl chloride (37.7 mg, 0.416 mmol) dissolved in acetone was added in the solution slowly. The reaction solution was stirred at 0˜25° C. for 30 min. The reaction solution was diluted with water (10 mL) and extracted with DCM (5 mL×3). The combined organic layers were concentrated and purified by prep-HPLC to afford example 68. LCMS: 535.1 [M+H]⁺; ¹H NMR: (400 MHZ, DMSO-d₆) δ 9.26 (s, 1H), 8.49 (d, J=4.9 Hz, 1H), 8.23 (s, 1H), 7.99 (s, 1H), 7.51 (s, 1H), 7.42-7.28 (m, 3H), 7.22 (d, J=8.5 Hz, 2H), 7.17 (d, J=5.0 Hz, 1H), 6.49-6.27 (m, 2H), 6.15 (dd, J=16.9, 1.9 Hz, 1H), 5.71 (dd, J=10.3, 1.8 Hz, 1H), 5.41-5.26 (m, 1H), 4.70 (br t, J=8.6 Hz, 1H), 4.51 (dd, J=9.1, 5.2 Hz, 1H), 4.41 (br t, J=9.2 Hz, 1H), 4.25 (dd, J=10.1, 5.1 Hz, 1H), 2.43 (s, 3H), 2.40 (s, 3H).

### Example 69 Preparation of 6-(4-(1-acryloylazetidin-3-yl)phenyl)-3-methyl-7-(4-((4-methylpyrimidin-2-yl)oxy)phenyl)pyrrolo[1,2-a]pyrazine-8-carboxamide

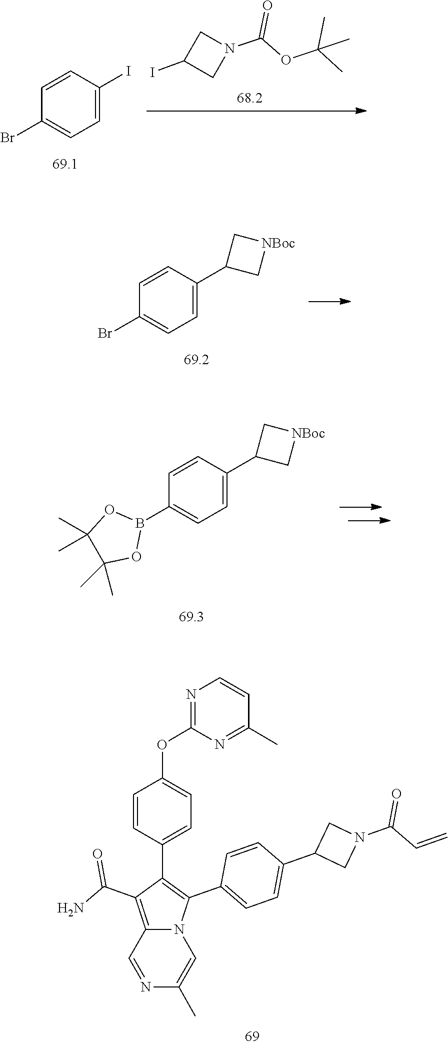

### Step 1: Preparation of Compound 69.2

To a solution of Zn (7.40 g, 113 mmol) in THF (20 mL) was added 1,2-dibromoethane (0.18 mL, 2.12 mmol) and TMSCl (0.54 mL, 4.24 mmol). The mixture was stirred for 30 mins. Then compound 68.2 (12.0 g, 42.4 mmol) in THF (10 mL) was added slowly to the mixture which was immersed in a water bath to keep the temperature below 65° C. The mixture was stirred for 1 h and then degassed by bubbling N₂. Then the solution above was added into a solution of compound 69.1 (4.00 g, 14.139 mmol), Pd(dppf)Cl₂·CH₂Cl₂ (0.70 g, 0.85 mmol), CuI (0.30 g, 1.77 mmol) in THF (20 mL). The resulting mixture was heated at 80° C. for 1 h. After cooling to rt, the reaction solution was filtered. The filtrate was concentrated in vacuum. The residue was purified by flash column chromatography on silica gel to afford compound 69.2 (2.60 g, 58.9%) as a yellow oil. ¹H NMR: (400 MHZ, DMSO-d₆) δ 7.54 (d, J=8.4 Hz, 2H), 7.31 (d, J=8.4 Hz, 2H), 4.23 (br s, 1H), 3.83-3.77 (m, 4H), 1.36 (s, 9H).

### Step 2: Preparation of Compound 69.3

A solution of compound 69.2 (1.00 g, 3.20 mmol), (Bpin)₂ (976 mg, 3.84 mmol), Pd(dppf)Cl₂ (234 mg, 0.32 mmol) and KOAc (628 mg, 6.41 mmol) in dioxane (10 mL) was stirred at 100° C. for 12 h under N₂. The reaction mixture was filtered, and the filtrate was concentrated under reduced pressure. The residue was purified by flash column chromatography on silica gel to afford compound 69.3 (1.08 g, 93.8%) as a colorless oil. LCMS: 304.0 [M−55]⁺.

### Step 3: Preparation of Example 69

In a similar fashion according to the procedure for example 68, example 69 was synthesized from compound 69.3. Spectrum data of example 69: LCMS: 545.3 [M+H]⁺. ¹H NMR: (400 MHZ, DMSO-d₆) δ 9.26 (d, J=1.1 Hz, 1H), 8.45 (d, J=5.1 Hz, 1H), 7.81 (s, 1H), 7.46 (d, J=8.1 Hz, 2H), 7.39-7.28 (m, 5H), 7.14 (dd, J=6.8, 1.8 Hz, 3H), 6.51 (br s, 1H), 6.35 (dd, J=16.9, 10.3 Hz, 1H), 6.12 (dd, J=16.9, 2.2 Hz, 1H), 5.71-5.66 (m, 1H), 4.63 (t, J=8.4 Hz, 1H), 4.38-4.31 (m, 1H), 4.27-4.23 (m, 1H), 3.99-3.90 (m, 2H), 2.40 (s, 3H), 2.34 (s, 3H).

### Example 70 Preparation of 6-(2-acryloyl-2-azaspiro[3.5]non-6-en-7-yl)-3-methyl-7-(4-((4-methylpyrimidin-2-yl)oxy)phenyl)pyrrolo[1,2-a]pyrazine-8-carboxamide

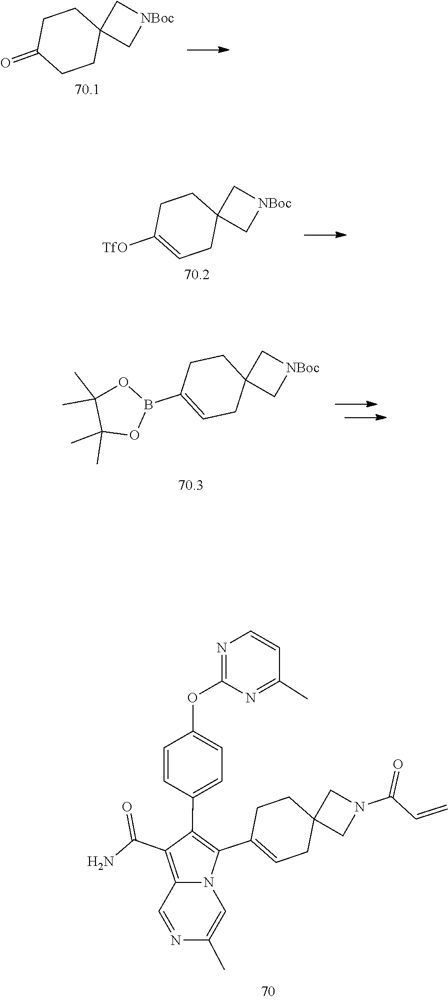 [SMILES: CC(C)(C)OC(=O)N1CC2(CCC(=O)CC2)C1 ; CC(C)(C)OC(=O)N1CC2(CC=C(OS(=O)(=O)C(F)(F)F)CC2)C1 ; CC(C)(C)OC(=O)N1CC2(CC=C(B3OC(C)(C)C(C)(C)O3)CC2)C1 ; C=CC(=O)N1CC2(CC=C(c3c(-c4ccc(Oc5nccc(C)n5)cc4)c(C(N)=O)c4cnc(C)cn34)CC2)C1]

### Step 1: Preparation of Compound 70.2

To a solution of compound 70.1 (1.00 g, 4.18 mmol) in THF (10 mL) was added LiHMDS (3.13 mL, 5.02 mmol) at −78° C. The reaction was stirred at −78° C. for 1 h and then a solution of PhN(Tf)₂ (1.80 g, 5.02 mmol) in THF (10 mL) was added dropwise. The reaction mixture was stirred at 25° C. for 18 h. The reaction was quenched sat. NH₄Cl (30 mL) and extracted with EA (50 mL×3). The combined organic layer was washed with brine (50 mL), dried over Na₂SO₄, and concentrated. The residue was purified by flash column chromatography on silica gel to afford compound 70.2 (1.20 g, 77.3%) as a white solid. ¹H NMR: (400 MHZ, DMSO-d₆) δ 5.82 (br s, 1H), 3.66-3.48 (m, 4H), 2.44-2.36 (m, 4H), 1.90 (t, J=6.2 Hz, 2H), 1.38 (s, 9H).

### Step 2: Preparation of Compound 70.3

A mixture of compound 70.2 (600.0 mg, 1.62 mmol), (Bpin)₂ (615.0 mg, 2.42 mmol), potassium acetate (317.0 mg, 3.23 mmol) and Pd(dppf)Cl₂ (118.0 mg, 0.162 mmol) in dioxane (10 mL) was stirred at 90° C. for 18 h under N₂. After cooling to rt, the reaction mixture was concentrated under vacuum. The residue was purified by flash column chromatography on silica gel to afford compound 70.3 (300.0 mg, 53.2%) as a yellow solid. ¹H NMR: (400 MHz, DMSO-d₆) δ 6.35 (s, 1H), 3.59-3.51 (m, 2H), 3.48 (br s, 2H), 3.35 (s, 2H), 2.27 (d, J=2.9 Hz, 2H), 2.08 (d, J=2.1 Hz, 2H), 1.19 (s, 9H), 1.17 (s, 12H).

### Step 3: Preparation of Example 70

In a similar fashion according to the procedure for example 68, example 70 was synthesized from compound 70.3. Spectrum data of example 70: LCMS: 535.1 [M+H]⁺. ¹H NMR: (400 MHZ, DMSO-d₆) δ 9.16 (s, 1H), 8.49 (d, J=5.1 Hz, 1H), 7.93 (s, 1H), 7.43 (d, J=8.4 Hz, 2H), 7.37-7.28 (m, 1H), 7.25 (d, J=8.1 Hz, 2H), 7.17-7.13 (m, 1H), 6.73-6.60 (m, 1H), 6.32-6.17 (m, 1H), 6.14-6.02 (m, 1H), 5.88 (br s, 1H), 5.60 (d, J=10.8 Hz, 1H), 3.89 (q, J=8.5 Hz, 2H), 3.71-3.60 (m, 2H), 2.40 (s, 8H), 2.07-1.93 (m, 2H), 1.85-1.80 (m, 2H).

### Example 71 Preparation of 6-(1-(2-fluoroacryloyl) indolin-5-yl)-3-methyl-7-(4-((4-methylpyrimidin-2-yl)oxy)phenyl)pyrrolo[1,2-a]pyrazine-8-carboxamide

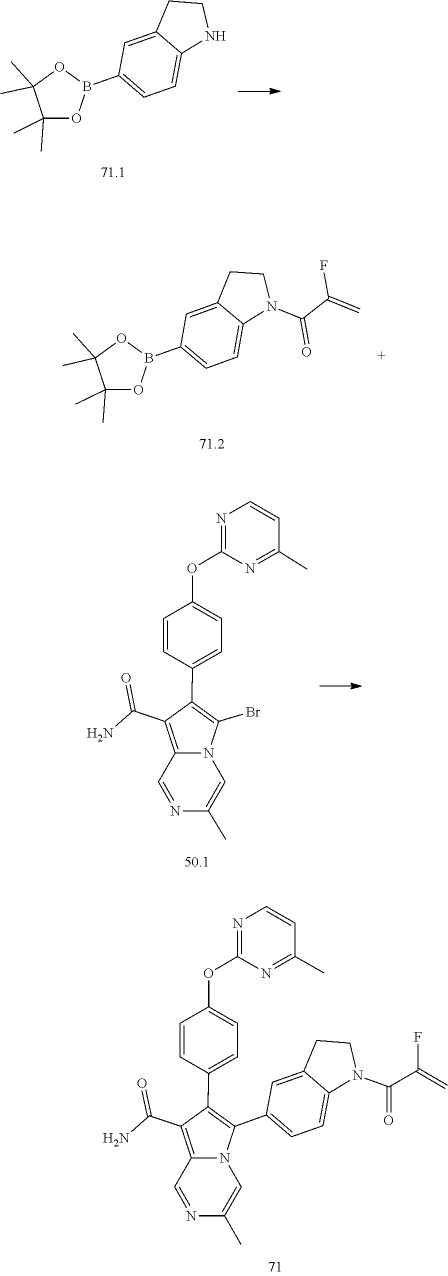 [SMILES: CC1(C)OB(c2ccc3c(c2)CCN3)OC1(C)C ; C=C(F)C(=O)N1CCc2cc(B3OC(C)(C)C(C)(C)O3)ccc21 ; Cc1cn2c(Br)c(-c3ccc(Oc4nccc(C)n4)cc3)c(C(N)=O)c2cn1 ; C=C(F)C(=O)N1CCc2cc(-c3c(-c4ccc(Oc5nccc(C)n5)cc4)c(C(N)=O)c4cnc(C)cn34)ccc21]

### Step 1: Preparation of Compound 71.2

To a solution of compound 71.1 (500.0 mg, 2.04 mmol) and 2-fluoroprop-2-enoic acid (367.6 mg, 4.08 mmol) in pyridine (5 mL) was added EDCI (1.17 g, 6.12 mmol). The mixture was stirred at 50° C. for 3 h. After cooling to rt, the reaction mixture was concentrated and purified by flash column chromatography on silica gel to afford compound 71.2 (400.0 mg, 61.8%) as a yellow solid. LCMS: 317.9 [M+H]⁺.

### Step 2: Preparation of Example 71

To a solution of compound 50.1 (150.0 mg, 0.34 mmol) and compound 71.2 (217.1 mg, 0.68 mmol) in dioxane (3 mL) and H₂O (0.5 mL) were added Pd(PPh₃)₄ (39.5 mg, 0.03 mmol) and K₂CO₃ (118.2 mg, 0.856 mmol). The mixture was stirred at 90° C. for 2 h under N₂. After cooling to rt, the reaction solution was diluted with water (5 mL) and extracted with EA (10 mL×3). The combined organic layers were washed with brine (5 mL), dried over Na₂SO₄, filtered, concentrated, and purified by prep-HPLC to afford example 71. LCMS: 549.3 [M+H]⁺. ¹H NMR: (400 MHZ, DMSO-d₆) δ 9.26 (s, 1H), 8.46 (d, J=4.9 Hz, 1H), 8.00 (br s, 1H), 7.82 (s, 1H) 7.47-7.25 (m, 4H), 7.25-7.02 (m, 4H), 6.55 (br s, 1H), 5.66-5.38 (m, 2H), 4.28 (t, J=6.6 Hz, 2H), 3.17 (t, J=8.1 Hz, 2H), 2.41 (s, 3H), 2.35 (s, 3H).

### Example 72 Preparation of 7-(3-fluoro-4-((4-methylpyrimidin-2-yl)oxy)phenyl)-6-(1-(2-fluoroacryloyl) indolin-5-yl)-3-methylpyrrolo[1,2-a]pyrazine-8-carboxamide

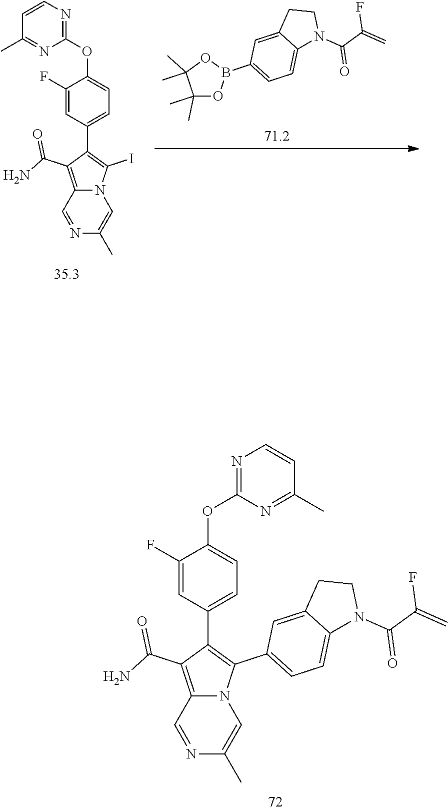 [SMILES: Cc1cn2c(I)c(-c3ccc(Oc4nccc(C)n4)c(F)c3)c(C(N)=O)c2cn1 ; C=C(F)C(=O)N1CCc2cc(B3OC(C)(C)C(C)(C)O3)ccc21 ; C=C(F)C(=O)N1CCc2cc(-c3c(-c4ccc(Oc5nccc(C)n5)c(F)c4)c(C(N)=O)c4cnc(C)cn34)ccc21]

### Step 1: Preparation of Example 72

In a similar fashion according to the procedure for example 71, example 72 was synthesized from compound 35.3. Spectrum data of example 72: LCMS: 567.2 [M+H]⁺. ¹H NMR: (400 MHZ, DMSO-d₆) δ 9.19 (d, J=1.1 Hz, 1H), 8.48 (d, J=5.0 Hz, 1H), 8.03 (br s, 1H), 7.82 (s, 1H), 7.48-7.37 (m, 1H), 7.35-7.29 (m, 2H), 7.26 (dd, J=11.5, 1.9 Hz, 1H), 7.23-7.17 (m, 2H), 7.10 (dd, J=8.3, 1.5 Hz, 1H), 6.93 (br s, 1H), 5.65-5.42 (m, 2H), 4.35-4.25 (m, 2H), 3.18 (t, J=8.1 Hz, 2H), 2.42 (s, 3H), 2.35 (s, 3H).

### Example 73 Preparation of 6-(6-fluoro-1-(2-fluoroacryloyl) indolin-5-yl)-7-(3-fluoro-4-((4-methylpyrimidin-2-yl)oxy)phenyl)-3-methylpyrrolo[1,2-a]pyrazine-8-carboxamide

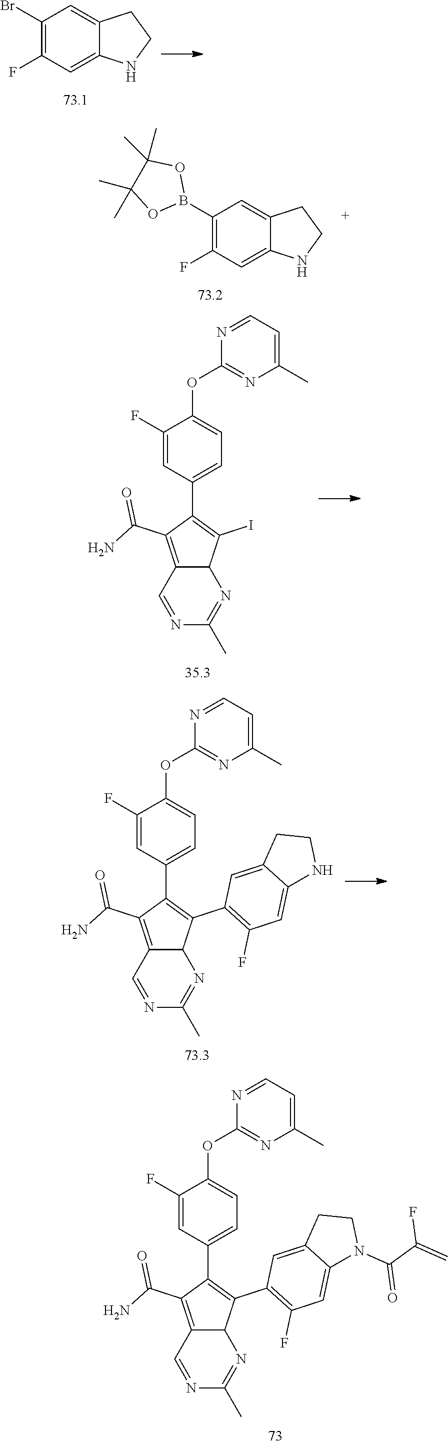

### Step 1: Preparation of Compound 73.2

To a solution of compound 73.1 (500.0 mg, 2.31 mmol) and (Bpin)₂ (587.5 mg, 2.31 mmol) in dioxane (6 mL) were added KOAc (680.9 mg, 6.94 mmol) and Pd(dppf)Cl₂ (169.3 mg, 0.231 mmol) under N₂. The reaction was stirred at 90° C. for 12 h. After cooling to rt, the reaction mixture was concentrated and purified by flash column chromatography on silica gel to afford compound 73.2 (230.0 mg, 37.8%) as a white solid. LCMS: 264.2 [M+H]⁺.

### Step 2: Preparation of Compound 73.3

To a solution of compound 35.3 (150.0 mg, 0.298 mmol) and compound 73.2 (78.4 mg, 0.298 mmol) in dioxane (5 mL) and H₂O (1 mL) was added K₂CO₃ (123.6 mg, 0.894 mmol). Then Pd(PPh₃)₄ (34.4 mg, 0.030 mmol) was added under N₂. The reaction was stirred at 90° C. for 12 h. After cooling to rt, the reaction mixture was concentrated and purified by flash column chromatography on silica gel to afford compound 73.3. LCMS: 513.1 [M+H]⁺.

### Step 3: Preparation of Example 73

To a solution of compound 73.3 (100.0 mg, 0.195 mmol) in pyridine (2 mL) were added 2-fluoroprop-2-enoic acid (26.4 mg, 0.293 mmol) and EDCI (112.2 mg, 0.585 mmol). The reaction solution was stirred at 60° C. for 12 h. After cooling to rt, the reaction solution was concentrated and purified by prep-HPLC to afford example 73. LCMS: 585.1 [M+H]⁺. ¹H NMR: (400 MHZ, DMSO-d₆) δ 9.18 (d, J=1.0 Hz, 1H), 8.47 (d, J=5.0 Hz, 1H), 7.87 (d, J=11.0 Hz, 1H), 7.68 (s, 1H), 7.51-7.39 (m, 1H), 7.34-7.24 (m, 3H), 7.18 (d, J=5.0 Hz, 1H), 7.08 (dd, J=8.3, 1.2 Hz, 2H), 5.66-5.47 (m, 2H), 4.38-4.29 (m, 2H), 3.20-3.07 (m, 2H), 2.42 (s, 3H), 2.35 (s, 3H).

### Example 75 Preparation of 6-(2-fluoro-4-(2-fluoroacrylamido)phenyl)-3-methyl-7-(3-(methylcarbamoyl)phenyl)pyrrolo[1,2-a]pyrazine-8-carboxamide

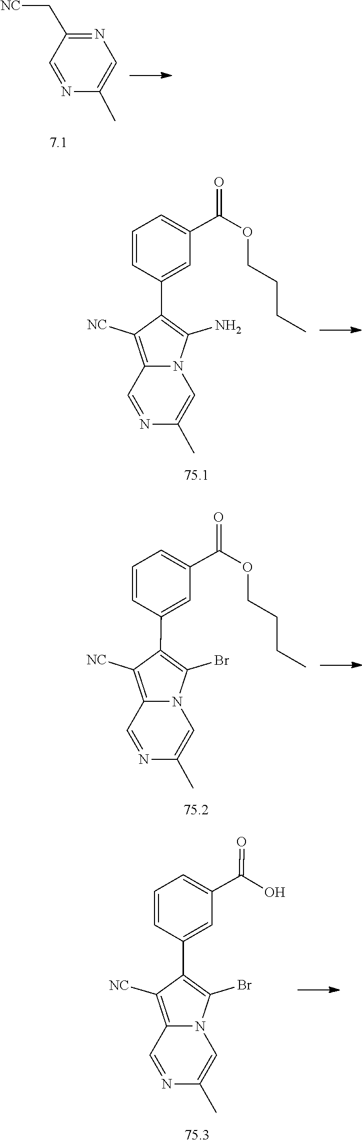

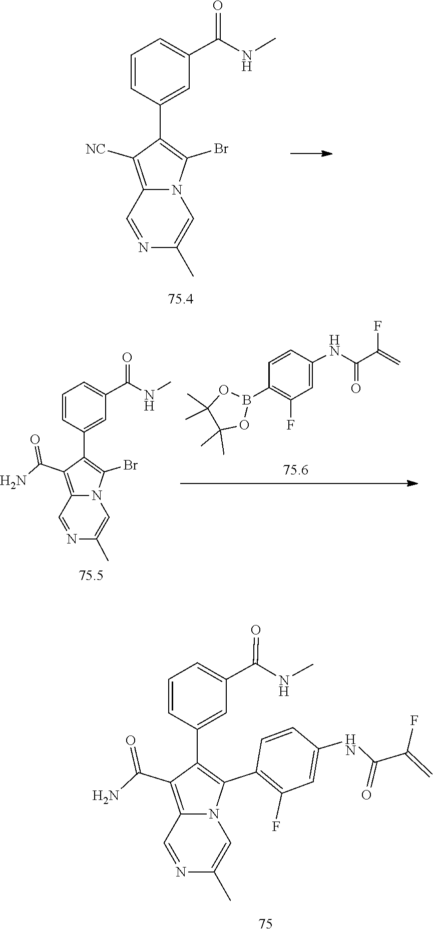

### Step 1: Preparation of Compound 75.1

To a solution of compound 7.1 (1.00 g, 7.51 mmol) and methyl 3-formylbenzoate (1.20 g, 7.51 mmol) in n-BuOH (5 mL) were added DBU (1.10 g, 7.51 mmol) and 2-oxopropanenitrile (0.50 g, 7.51 mmol). Then the mixture was stirred at 100° C. under microwave for 30 min. After cooling to rt, the reaction was concentrated and purified by flash column chromatography on silica gel to afford compound 75.1 (1.30 g, 56.5%) as a yellow solid. LCMS: 349.1 [M+H]⁺.

### Step 2: Preparation of Compound 75.2

A solution of compound 75.1 (1300.0 mg, 3.73 mmol) and CuBr₂ (1.08 g, 4.85 mmol) in HBr/AcOH (30%; 1.80 mL) was cooled to 0° C. Then sodium nitrite (1.29 g, 18.7 mmol) dissolved in H₂O (0.17 mL) was added in the solution and the reaction was stirred at 0° C. for 0.5 h. The reaction solution was diluted with ice water (10 mL) and the pH of the reaction solution was adjusted to 7 by aqueous solution of sodium carbonate. After extraction with EA (20 mL×3), the combined organic layers were washed with brine (10 mL), dried over Na₂SO₄, filtered, concentrated, and purified by flash column chromatography on silica gel to afford compound 75.2 (520.0 mg, 33.8%) as a yellow solid. LCMS: 412.0; 414.0 [M+H]⁺.

### Step 3: Preparation of Compound 75.3

To a solution of compound 75.2 (520.0 mg, 1.26 mmol) in THF (5 mL) and H₂O (1 mL) was added LiOH (264.9 mg, 6.31 mmol). The mixture was stirred at 70° C. for 18 h. The pH of the reaction was adjusted to 7˜8 by 1M HCl (aq) and the reaction solution was extracted with EA (10 mL×3). The combined organic layers were washed with brine (10 mL), dried over Na₂SO₄, filtered, and concentrated to afford compound 75.3 (200.0 mg, crude) as a yellow solid. LCMS: 356.0 [M+H]⁺.

### Step 4: Preparation of Compound 75.4

To a solution of compound 75.3 (200.0 mg, 0.56 mmol) and methylammonium chloride (113.7 mg, 1.68 mmol) in DMF (2 mL) was added DIEA (0.47 mL, 2.81 mmol). Then HATU (234.8 mg, 0.62 mmol) added and the mixture was stirred at 25° C. for 3 h. The reaction mixture was poured into the ice water (10 mL) and extracted with EA (5 mL×3). The combined organic layers were washed with brine (10 mL), dried over Na₂SO₄, filtered, concentrated, and purified by flash column chromatography on silica gel to afford compound 75.4. LCMS: 369.0 [M+H]⁺.

### Step 5: Preparation of Compound 75.5

To a solution of compound 75.4 (100.0 mg, 0.27 mmol) in DMSO (2 mL) was added K₂CO₃ (112.3 mg, 0.81 mmol). Then H₂O₂ (307.0 mg, 2.71 mmol) was added and the mixture was stirred at 25° C. for 18 h. The reaction was quenched with Na₂S₂O₃ (aq. 20 mL) and extracted with DCM (10 mL×3). The combined organic layers were washed with brine (10 mL), dried over Na₂SO₄, filtered, and concentrated to afford compound 75.5. LCMS: 389.0 [M+H]⁺.

### Step 6: Preparation of Example 75

To a solution of compound 75.5 (60.0 mg, 0.16 mmol) and compound 75.6 (71.8 mg, 0.23 mmol) in dioxane (2 mL) and H₂O (0.5 mL) was added K₂CO₃ (53.5 mg, 0.39 mmol). Then Pd(PPh₃)₄ (17.9 mg, 0.02 mmol) was added and the mixture was stirred at 90° C. for 2 h under N₂. After cooling to rt, the reaction solution was diluted with water (5 mL) and extracted with EA (5 mL×3). The combined organic layers were washed with brine (5 mL), dried over Na₂SO₄, filtered, concentrated, and purified by prep-HPLC to afford example 75. LCMS: 490.2 [M+H]⁺. ¹H NMR: (400 MHZ, DMSO-d₆) δ 10.63 (br s, 1H), 9.27 (s, 1H), 8.43 (d, J=4.6 Hz, 1H), 7.83 (dd, J=12.3, 1.8 Hz, 1H), 7.78-7.73 (m, 2H), 7.71 (s, 1H), 7.57 (dd, J=8.5, 1.7 Hz, 1H), 7.43-7.33 (m, 3H), 7.25 (t, J=8.4 Hz, 1H), 6.65-6.49 (m, 1H), 5.84-5.69 (m, 1H), 5.50 (dd, J=15.5, 3.9 Hz, 1H), 2.75 (d, J=4.4 Hz, 3H), 2.37 (s, 3H).

### Example 76 Preparation of 6-(4-acrylamidophenyl)-7-(4-((6-methylpyridin-2-yl)oxy)phenyl)-3-(oxetan-3-yl)pyrrolo[1,2-a]pyrazine-8-carboxamide

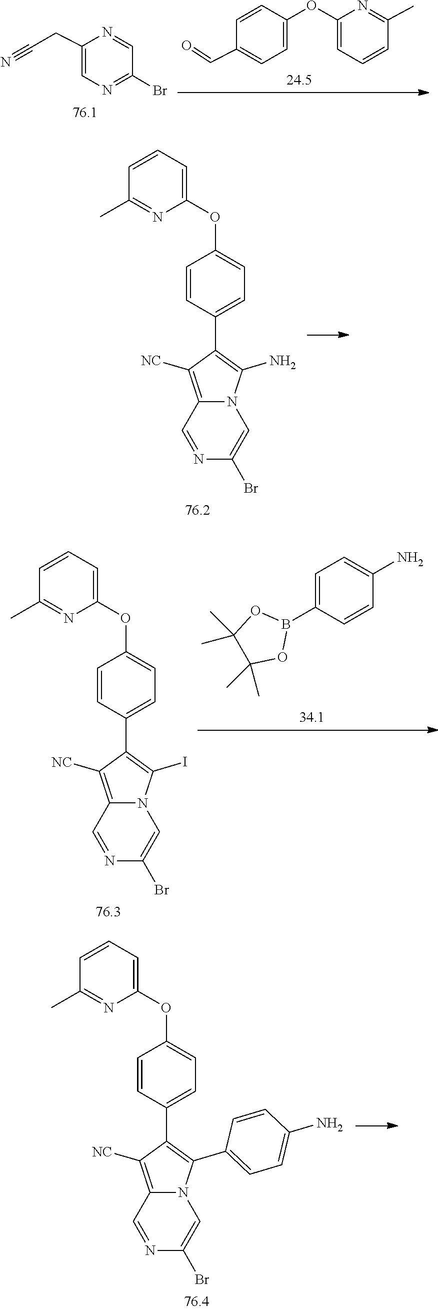 [SMILES: N#CCc1cnc(Br)cn1 ; Cc1cccc(Oc2ccc(C=O)cc2)n1 ; Cc1cccc(Oc2ccc(-c3c(C#N)c4cnc(Br)cn4c3N)cc2)n1 ; Cc1cccc(Oc2ccc(-c3c(C#N)c4cnc(Br)cn4c3I)cc2)n1 ; CC1(C)OB(c2ccc(N)cc2)OC1(C)C ; Cc1cccc(Oc2ccc(-c3c(C#N)c4cnc(Br)cn4c3-c3ccc(N)cc3)cc2)n1]

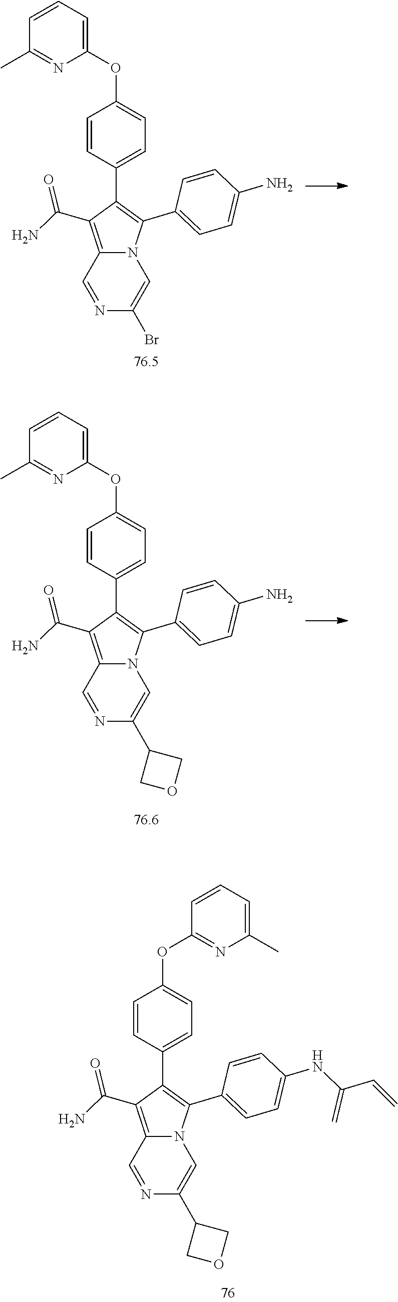 [SMILES: Cc1cccc(Oc2ccc(-c3c(C(N)=O)c4cnc(Br)cn4c3-c3ccc(N)cc3)cc2)n1 ; Cc1cccc(Oc2ccc(-c3c(C(N)=O)c4cnc(C5COC5)cn4c3-c3ccc(N)cc3)cc2)n1 ; C=CC(=C)Nc1ccc(-c2c(-c3ccc(Oc4cccc(C)n4)cc3)c(C(N)=O)c3cnc(C4COC4)cn23)cc1]

### Step 1: Preparation of Compound 76.2

To a mixture of compound 76.1 (500.0 mg, 2.52 mmol) and compound 24.5 (538.4 mg, 2.52 mmol) in n-BuOH (5 mL) were added DBU (1.15 g, 7.57 mmol) and acetyl cyanide (174.4 mg, 2.52 mmol) at 25° C. and purged with N₂ for 3 times. The solution was heated to 100° C. under microwave for 30 min. After cooling to rt, the reaction mixture was concentrated and purified by flash column chromatography on silica gel to afford compound 76.2 (1.00 g, 47.12% yield) as a yellow solid. LCMS: 420.0; 422.0 [M+H]⁺.

### Step 2: Preparation of Compound 76.3

To a solution of compound 76.2 (500.0 mg, 1.19 mmol) in CH₃CN (5 mL) was added diiodomethane (3.19 g, 11.9 mmol) and the reaction was warmed to 40° C. Then isopentyl nitrite (348.4 mg, 2.97 mmol) added and the reaction solution was stirred at 40° C. for 1 h. After cooling to rt, the reaction mixture was concentrated and purified by flash column chromatography on silica gel to afford compound 76.3 (300.0 mg, 47.47%) as a yellow solid. LCMS: 530.9; 532.9 [M+H]⁺.

### Step 3: Preparation of Compound 76.4

To a solution of compound 76.3 (300.0 mg, 0.565 mmol) and compound 34.1 (160.9 mg, 0.734 mmol) in dioxane (3 mL) and H₂O (0.5 mL) was added Na₂CO₃ (119.7 mg, 1.13 mmol). Then Pd(PPh₃)₄ (65.3 mg, 0.056 mmol) added in the solution under N₂. The reaction was stirred at 60° C. for 12 h. After cooling to rt, the reaction solution was diluted with water (10 mL) and extracted with EA (10 mL×3). The combined organic layers were washed with brine (10 mL), dried over Na₂SO₄, filtered, concentrated, and purified by flash column chromatography on silica gel to afford compound 76.4 (100.0 mg, 35.67%) as a yellow gum. LCMS: 496.0; 498.0 [M+H]⁺.

### Step 4: Preparation of Compound 76.5

A solution of compound 76.4 (100.0 mg, 0.201 mmol) in H₂SO₄ (3 mL) was stirred at 30° C. for 12 h. The reaction solution was poured into ice (10 mL) and the pH was adjusted to 7 by Na₂CO₃ (aq). The solution was extracted with EA (10 mL×3), washed with brine (20 mL), dried over Na₂SO₄, filtered, and concentrated to afford compound 76.5. LCMS: 514.0; 516.0 [M+H]⁺.

### Step 5: Preparation of Compound 76.6

To a solution of compound 76.5 (100.0 mg, 0.194 mmol) and 3-bromooxetane (79.9 mg, 0.583 mmol) in DMA (2 mL) were added NaI (7.28 mg, 0.049 mmol) and pyridine-2-carboximidamide hydrochloride (2.22 mg, 0.019 mmol). Then NiCl₂(DME) (4.27 mg, 0.019 mmol), Zn (25.4 mg, 0.389 mmol) and TFA (2.22 mg, 0.019 mmol) were added in the solution under N₂. The reaction solution was stirred at 60° C. for 12 h. After cooling to rt, the reaction solution was filtered, concentrated, and purified by flash column chromatography on silica gel to afford compound 76.6. LCMS: 492.2 [M+H]⁺.

### Step 6: Preparation of Example 76

To a solution of compound 76.6 (30.0 mg, 0.061 mmol) in DCM (2 mL) was added DIEA (0.020 mL, 0.122 mmol). Then acryloyl chloride (8.29 mg, 0.092 mmol) dissolved in DCM was added in the solution. The reaction solution was stirred at 0˜25° C. for 30 min. The reaction solution was diluted with water (20 mL) and extracted with DCM (5 mL×3). The combined organic layers were washed with brine (20 mL), dried over Na₂SO₄, filtered, concentrated, and purified by prep-HPLC to afford example 76. LCMS: 546.1 [M+H]⁺. ¹H NMR: (400 MHZ, DMSO-d₆) δ 10.32 (s, 1H), 9.41 (d, J=1.0 Hz, 1H), 7.87 (s, 1H), 7.78-7.69 (m, 3H), 7.39 (br s, 1H), 7.28 (dd, J=8.6, 1.8 Hz, 4H), 7.06 (d, J=8.7 Hz, 2H), 7.02 (d, J=7.3 Hz, 1H), 6.76 (d, J=8.2 Hz, 1H), 6.63 (br s, 1H), 6.50-6.39 (m, 1H), 6.32-6.23 (m, 1H), 5.79 (dd, J=10.1, 1.9 Hz, 1H), 4.86-4.74 (m, 4H), 4.39-4.35 (m, 1H), 2.34 (s, 3H).

### Example 77 Preparation of 6-(4-acrylamidophenyl)-3-cyano-7-(4-((6-methylpyridin-2-yl)oxy)phenyl)pyrrolo[1,2-a]pyrazine-8-carboxamide

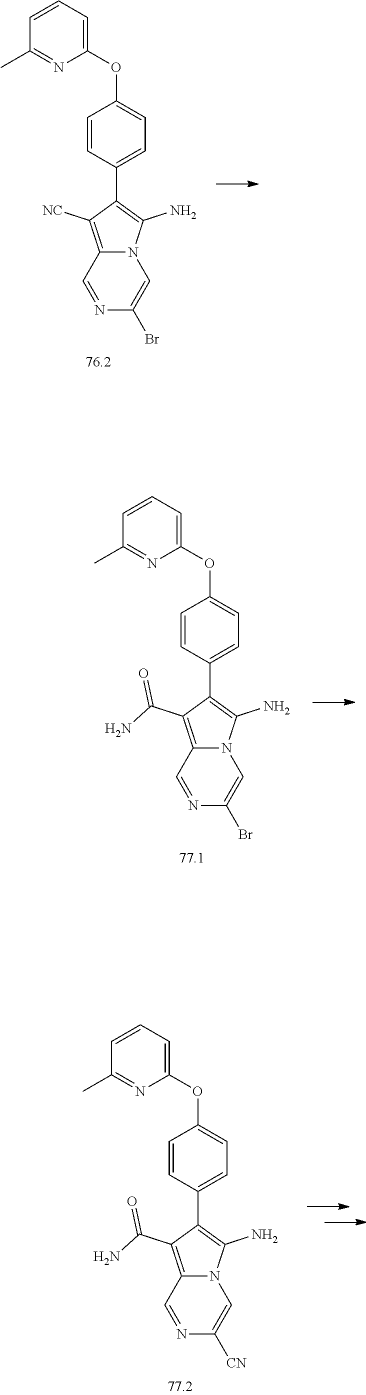 [SMILES: Cc1cccc(Oc2ccc(-c3c(C#N)c4cnc(Br)cn4c3N)cc2)n1 ; Cc1cccc(Oc2ccc(-c3c(C(N)=O)c4cnc(Br)cn4c3N)cc2)n1 ; Cc1cccc(Oc2ccc(-c3c(C(N)=O)c4cnc(C#N)cn4c3N)cc2)n1]

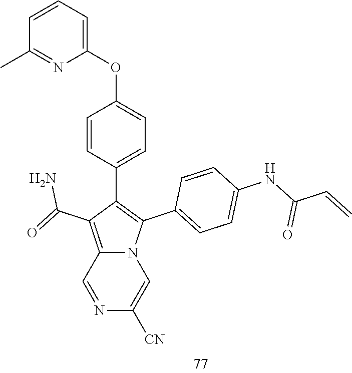 [SMILES: C=CC(=O)Nc1ccc(-c2c(-c3ccc(Oc4cccc(C)n4)cc3)c(C(N)=O)c3cnc(C#N)cn23)cc1]

### Step 1: Preparation of Compound 77.1

A mixture of compound 76.2 (930 mg, 2.21 mmol) in H₂SO₄ (10 mL) was stirred at room temperature for 18 h. The reaction was quenched with NaOH (aq) to pH˜8 and extracted with DCM (3×30 mL). The combined organic layers were dried over Na₂SO₄, filtered, concentrated to give a residue which was purified by silica gel column chromatography to afford compound 77.1 (470 mg, 48.46% yield). LCMS: 439.8 [M+H]⁺.

### Step 2: Preparation of Compound 77.2

To a solution of compound 77.1 (220 mg, 0.50 mmol) in NMP (13 mL) were added CuCN (134.86 mg, 1.51 mmol) and Pd(PPh₃)₄ (12 mg, 0.15 mmol). The reaction mixture was stirred at 190° C. for 1 h under microwave. After cooling to room temperature, the reaction was diluted with water (50 mL) and extracted with EA (50 mL). The organic phase combined and dried over Na₂SO₄, filtered, concentrated to give a residue which was purified by prep-TLC to afford compound 77.2. LCMS: 385.2 [M+H]⁺.

### Step 3: Preparation of Example 77

In a similar fashion according to the procedure for example 35, example 77 was synthesized from compound 77.2. Spectrum data of example 77: LCMS: 515.3 [M+H]⁺; ¹H NMR: (400 MHZ, DMSO-d₆) δ 9.82 (s, 1H), 8.34 (s, 1H), 7.70 (d, J=8.4 Hz, 2H), 7.63 (t, J=7.8 Hz, 1H), 7.44 (s, 1H), 7.28 (d, J=8.6 Hz, 2H), 7.22 (d, J=8.6 Hz, 2H), 7.17-7.09 (m, 2H), 6.94 (d, J=7.4 Hz, 1H), 6.72 (d, J=8.2 Hz, 1H), 6.47 (dd, J=16.8, 1.0 Hz, 1H), 6.27 (dd, J=16.8, 10.2 Hz, 1H), 5.83 (dd, J=10.2, 1.0 Hz, 1H), 5.56-5.49 (m, 2H), 2.45 (s, 3H).

### Example 78 Preparation of 6-(4-acrylamidophenyl)-7-(3-fluoro-4-((4-methylpyrimidin-2-yl)oxy)phenyl)-3-(hydroxymethyl)pyrrolo[1,2-a]pyrazine-8-carboxamide

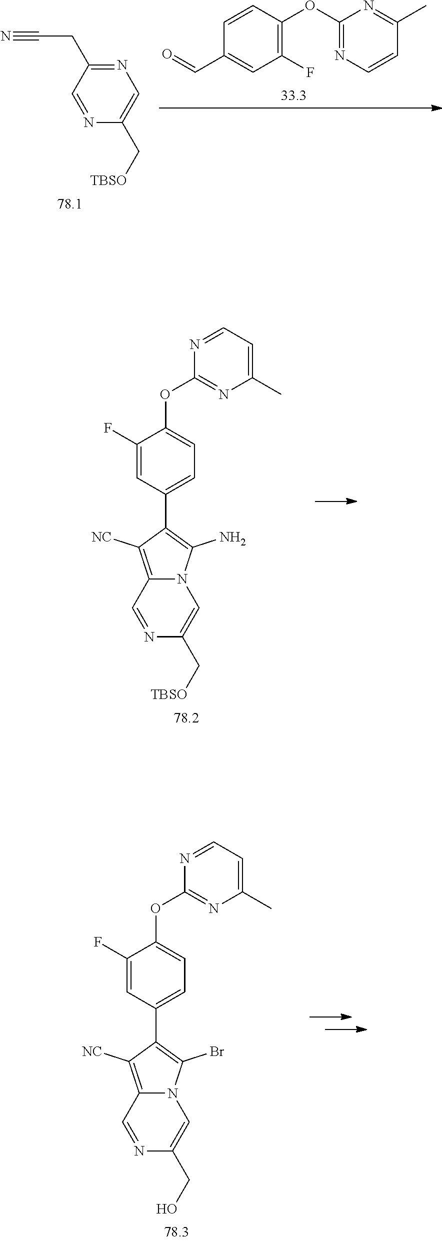

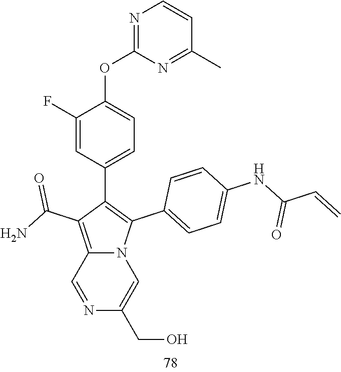 [SMILES: C=CC(=O)Nc1ccc(-c2c(-c3ccc(Oc4nccc(C)n4)c(F)c3)c(C(N)=O)c3cnc(CO)cn23)cc1]

### Step 1: Preparation of Compound 78.2

To a solution of compound 78.1 (440.0 mg, 1.67 mmol) in MeOH (8 mL) were added compound 33.3 (386.2 mg, 1.67 mmol), DBU (50.9 mg, 0.334 mmol) and acetyl cyanide (115.4 mg, 1.67 mmol). The mixture was stirred at 100° C. for 20 min under microwave. The reaction solution was concentrated and purified by flash silica gel chromatography to afford compound 78.2 (440.0 mg, 52.30%) as a yellow solid. LCMS: 505.1 [M+H]⁺.

### Step 2: Preparation of Compound 78.3

To a solution of compound 78.2 (440.0 mg, 0.874 mmol) in HBr/AcOH (5 mL) was added CuBr₂ (253.7 mg, 1.14 mmol). Then sodium nitrite (301.4 mg, 4.37 mmol) dissolved in H₂O (5 mL) was added in the solution. The mixture was stirred at 0° C. for 30 min. The reaction mixture was poured into ice-H₂O (10 mL), adjusted pH to 7˜8 with NaHCO₃(aq) and extracted with EA (20 mL×3). The combined organic layers were washed with brine (50 mL), dried over Na₂SO₄, filtered, concentrated, and purified by flash column chromatography on silica gel to afford compound 78.3. LCMS: 453.9; 455.9 [M+H]⁺.

### Step 3: Preparation of Example 78

In a similar fashion according to the procedure for example 12, example 78 was synthesized from compound 78.3. Spectrum data of example 78: LCMS: 539.2 [M+H]⁺. ¹H NMR: (400 MHZ, DMSO-d₆) δ 10.36 (s, 1H), 9.18 (s, 1H), 8.47 (s, 1H), 7.88-7.77 (m, 3H), 7.44 (br s, 1H), 7.36-7.28 (m, 3H), 7.26 (dd, J=11.7, 2.0 Hz, 1H), 7.18 (d, J=5.1 Hz, 1H), 7.10 (dd, J=8.4, 1.3 Hz, 1H), 7.03 (br s, 1H), 6.50-6.41 (m, 1H), 6.32-6.25 (m, 1H), 5.83-5.78 (m, 1H), 5.41 (t, J=5.7 Hz, 1H), 4.53 (d, J=5.3 Hz, 2H), 2.42 (s, 3H).

### Example 79 Preparation of (E)-6-(4-(4-(dimethylamino) but-2-enamido)phenyl)-3-(hydroxymethyl)-7-(4-((4-methylpyrimidin-2-yl)oxy)phenyl)pyrrolo[1,2-a]pyrazine-8-carboxamide

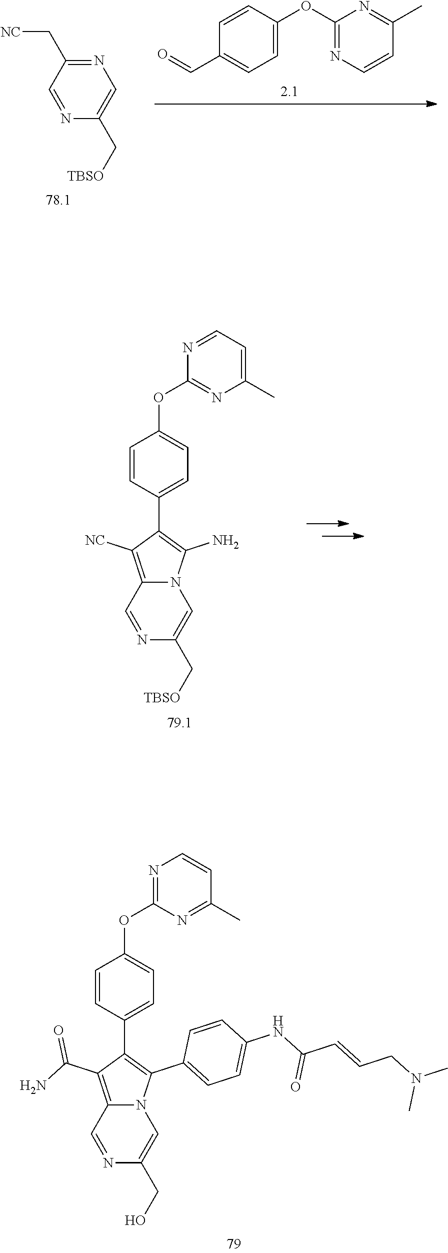 [SMILES: CC(C)(C)[Si](C)(C)OCc1cnc(CC#N)cn1 ; Cc1ccnc(Oc2ccc(C=O)cc2)n1 ; Cc1ccnc(Oc2ccc(-c3c(C#N)c4cnc(CO[Si](C)(C)C(C)(C)C)cn4c3N)cc2)n1 ; Cc1ccnc(Oc2ccc(-c3c(C(N)=O)c4cnc(CO)cn4c3-c3ccc(NC(=O)/C=C/CN(C)C)cc3)cc2)n1]

### Step 1: Preparation of Compound 79.1

To a solution of compound 78.1 (500.0 mg, 1.90 mmol) in MeOH (6.0 mL) were added compound 2.1 (406.6 mg, 1.90 mmol), DBU (57.8 mg, 0.380 mmol) and acetyl cyanide (131.1 mg, 1.90 mmol). Then the solution was stirred at 100° C. under microwave for 20 min. After cooling to rt, the reaction mixture was concentrated and purified by flash column chromatography on silica gel to afford compound 79.1. LCMS: 487.2 [M+H]⁺.

### Step 2: Preparation of Example 79

In a similar fashion according to the procedure for example 12, example 79 was synthesized from compound 79.1. Spectrum data of example 79: LCMS: 578.3 [M+H]⁺; ¹H NMR: (400 MHZ, DMSO-d₆) δ 10.24 (s, 1H), 9.24 (s, 1H), 8.46 (d, J=5.1 Hz, 1H), 7.85 (s, 1H), 7.75 (d, J=8.6 Hz, 2H), 7.39 (br s, 1H), 7.30 (dd, J=10.6, 8.6 Hz, 4H), 7.17-7.12 (m, 3H), 6.78-6.72 (m, 1H), 6.66 (br s, 1H), 6.30-6.26 (m, 1H), 5.41 (t, J=5.7 Hz, 1H), 4.53 (d, J=5.3 Hz, 2H), 3.06 (d, J=5.7 Hz, 2H), 2.40 (s, 3H), 2.17 (s, 6H).

### Example 80 Preparation of 6-(4-(2-fluoroacrylamido)phenyl)-3-(2-hydroxyethyl)-7-(4-((4-methylpyrimidin-2-yl)oxy)phenyl)pyrrolo[1,2-a]pyrazine-8-carboxamide

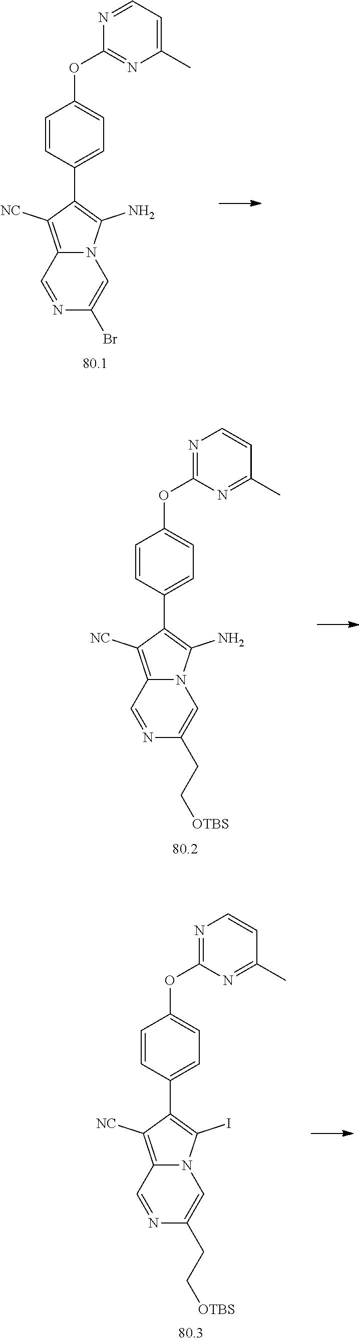 [SMILES: Cc1ccnc(Oc2ccc(-c3c(C#N)c4cnc(Br)cn4c3N)cc2)n1 ; Cc1ccnc(Oc2ccc(-c3c(C#N)c4cnc(CCO[Si](C)(C)C(C)(C)C)cn4c3N)cc2)n1 ; Cc1ccnc(Oc2ccc(-c3c(C#N)c4cnc(CCO[Si](C)(C)C(C)(C)C)cn4c3I)cc2)n1]

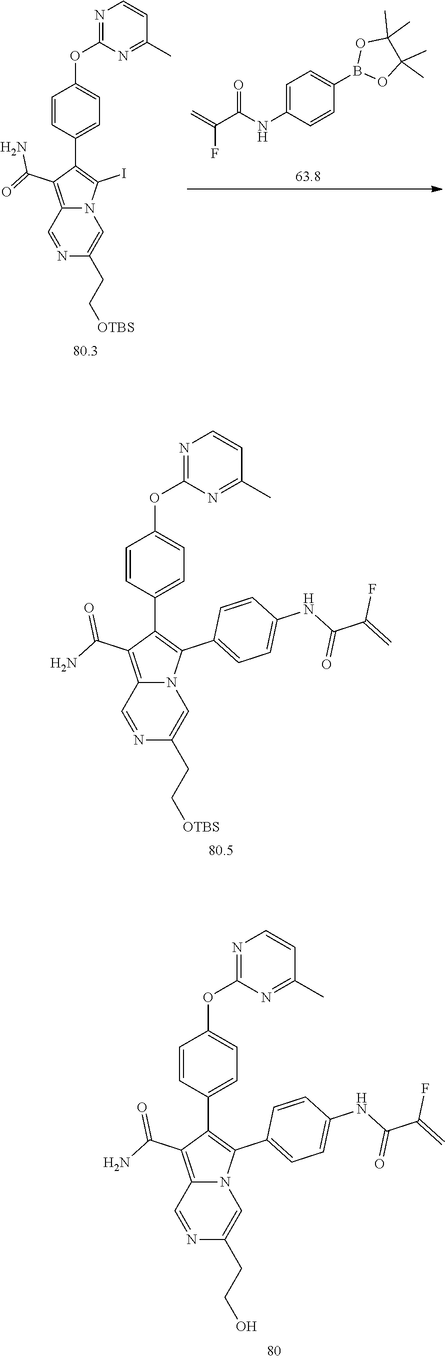 [SMILES: Cc1ccnc(Oc2ccc(-c3c(C(N)=O)c4cnc(CCO[Si](C)(C)C(C)(C)C)cn4c3I)cc2)n1 ; C=C(F)C(=O)Nc1ccc(B2OC(C)(C)C(C)(C)O2)cc1 ; C=C(F)C(=O)Nc1ccc(-c2c(-c3ccc(Oc4nccc(C)n4)cc3)c(C(N)=O)c3cnc(CCO[Si](C)(C)C(C)(C)C)cn23)cc1 ; C=C(F)C(=O)Nc1ccc(-c2c(-c3ccc(Oc4nccc(C)n4)cc3)c(C(N)=O)c3cnc(CCO)cn23)cc1]

### Step 1: Preparation of Compound 80.2

To a solution of compound 80.1 (1.00 g, 2.37 mmol) and (2-bromoethoxy) (tert-butyl)dimethylsilane (1.14 g, 4.75 mmol) in DMA (20 mL) were added pyridine-2-carboximidamide hydrochloride (37.4 mg, 0.237 mmol) and sodium iodide (106.7 mg, 0.712 mmol). Then NiCl₂(DME) (52.1 mg, 0.237 mmol), Zn (310.5 mg, 4.75 mmol) and TFA (27.1 mg, 0.237 mmol) were added in the solution under N₂. The mixture was stirred at 60° C. for 12 h. After cooling to rt, the reaction solution was diluted with water (100 mL) and extracted with EA (100 mL×3). The combined organic layers were washed with brine (100 mL), dried over Na₂SO₄, filtered, concentrated, and purified by flash column chromatography on silica gel to afford compound 80.2 (650.0 mg, 54.7%) as a yellow solid. LCMS: 501.2 [M+H]⁺.

### Step 2: Preparation of Compound 80.3

To a solution of compound 80.2 (600.0 mg, 1.20 mmol) in acetonitrile (6 mL) was added diiodomethane (4.91 g, 18.3 mmol) and 3-methyl-1-(nitrosooxidanyl) butane (701.6 mg, 5.99 mmol). Then the mixture was stirred at 40° C. for 1 h. After cooling to rt, the reaction solution was diluted with water (10 mL) and extracted with EA (10 mL×3). The combined organic layers were washed with brine (10 mL), dried over Na₂SO₄, filtered, and concentrated under reduced pressure to give a residue which was purified by flash column chromatography on silica gel to afford compound 80.3 (260.0 mg, 35.5%) as a yellow solid. LCMS: 612.6 [M+H]⁺.

### Step 3: Preparation of Compound 80.4

To a solution of compound 80.3 (100.0 mg, 0.16 mmol) in DMSO (2 mL) was added K₂CO₃ (67.8 mg, 0.49 mmol) and H₂O₂ (480.0 mg, 4.24 mmol). Then the mixture was stirred at 25° C. for 3 h. The reaction was quenched with Na₂S₂O₃ (aq. 20 mL) and extracted with DCM (10 mL×3). The combined organic layers were washed with brine (10 mL), dried over Na₂SO₄, filtered, and concentrated to afford compound 80.4. LCMS: 630.2 [M+H]⁺.

### Step 4: Preparation of Compound 80.5

To a solution of compound 80.4 (80.0 mg, 0.13 mmol) and compound 63.8 (74.0 mg, 0.25 mmol) in dioxane (3 mL) and H₂O (0.5 mL) were added Pd(PPh₃)₄ (14.7 mg, 0.01 mmol) and K₂CO₃ (43.9 mg, 0.32 mmol). Then the mixture was stirred at 90° C. for 2 h. After cooling to rt, the reaction solution was concentrated and purified by flash column chromatography on silica gel to afford compound 80.5. LCMS: 667.4 [M+H]⁺.

### Step 5: Preparation of Example 80

To a solution of compound 80.5 (40.0 mg, 0.06 mmol) in dioxane (1.0 mL) was added HCl/dioxane (1.0 mL) and the mixture was stirred at 25° C. for 1 h. The solution was concentrated and purified by prep-HPLC to afford example 80. LCMS: 553.6 [M+H]⁺. ¹H NMR: (400 MHZ, DMSO-d₆) δ 10.44 (s, 1H), 9.29 (s, 1H), 8.47 (d, J=4.5 Hz, 1H), 7.90-7.77 (m, 3H), 7.36-7.31 (m, 5H), 7.16 (d, J=8.0 Hz, 3H), 6.73-6.37 (m, 1H), 5.83-5.65 (m, 1H), 5.51-5.40 (m, 1H), 4.54 (s, 1H), 3.71 (d, J=5.3 Hz, 2H), 2.77 (br s, 2H), 2.41 (s, 3H).

### Example 81 Preparation of 6-(2-fluoro-4-(2-fluoroacrylamido)phenyl)-3-(2-hydroxyethyl)-7-(4-((4-methylpyrimidin-2-yl)oxy)phenyl)pyrrolo[1,2-a]pyrazine-8-carboxamide

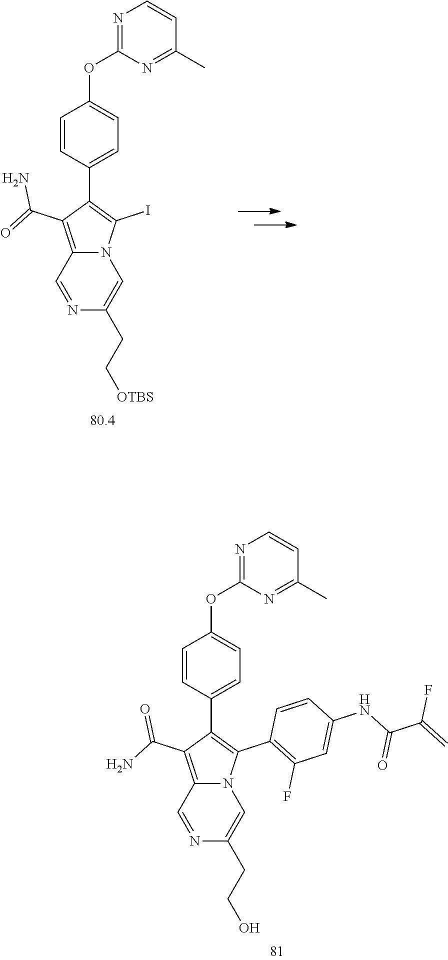 [SMILES: Cc1ccnc(Oc2ccc(-c3c(C(N)=O)c4cnc(CCO[Si](C)(C)C(C)(C)C)cn4c3I)cc2)n1 ; C=C(F)C(=O)Nc1ccc(-c2c(-c3ccc(Oc4nccc(C)n4)cc3)c(C(N)=O)c3cnc(CCO)cn23)c(F)c1]

### Step 1: Preparation of Example 81

In a similar fashion according to the procedure for example 80, example 81 was synthesized from compound 80.4. Spectrum data of example 81: LCMS: 571.1 [M+H]⁺. ¹H NMR: (400 MHZ, DMSO-d₆) δ 10.66 (s, 1H), 9.26 (s, 1H), 8.46 (d, J=4.8 Hz, 1H), 7.87 (d, J=12.4 Hz, 1H), 7.66-7.62 (m, 2H), 7.40 (br s, 1H), 7.33-7.28 (m, 3H), 7.16-7.14 (m, 3H), 6.79 (br s, 1H), 5.84-5.73 (m, 1H), 5.51 (dd, J=15.6, 4.0 Hz, 1H), 4.56 (t, J=5.2 Hz, 1H), 3.73-3.68 (m, 2H), 2.78 (t, J=6.4 Hz, 2H), 2.41 (s, 3H).

### Example 82 Preparation of 7-(3-fluoro-4-((4-methylpyrimidin-2-yl)oxy)phenyl)-6-(2-fluoro-4-(2-fluoroacrylamido)phenyl)-3-(2-hydroxyethyl)pyrrolo[1,2-a]pyrazine-8-carboxamide

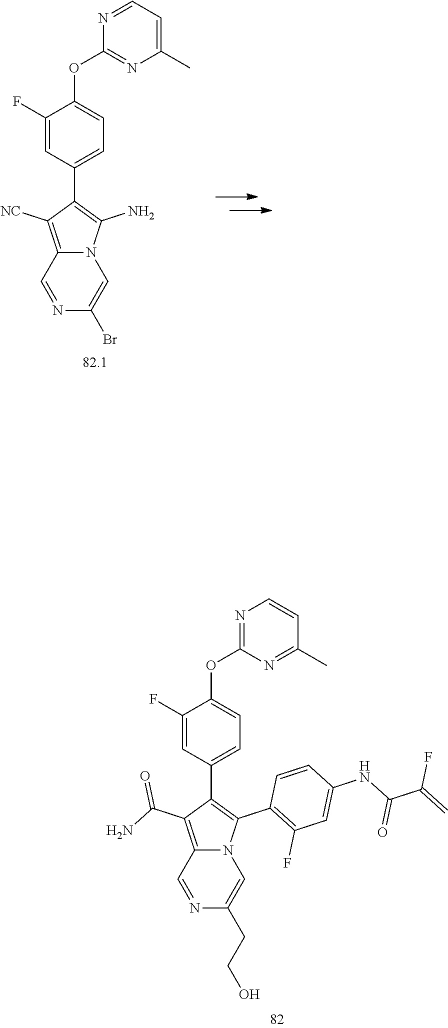 [SMILES: Cc1ccnc(Oc2ccc(-c3c(C#N)c4cnc(Br)cn4c3N)cc2F)n1 ; C=C(F)C(=O)Nc1ccc(-c2c(-c3ccc(Oc4nccc(C)n4)c(F)c3)c(C(N)=O)c3cnc(CCO)cn23)c(F)c1]

### Step 1: Preparation of Example 82

Compound 82.1 was synthesized in a similar fashion according to the procedure for compound 76.2.

In a similar fashion according to the procedure for example 80, example 82 was synthesized from compound 82.1. Spectrum data of example 81: LCMS: 589.1 [M+H]⁺. ¹H NMR: (400 MHZ, DMSO-d₆) δ 10.75 (br s, 1H), 9.29 (s, 1H), 8.56 (d, J=5.2 Hz, 1H), 7.95 (d, J=1.6 Hz, 1H), 7.76-7.74 (m, 2H), 7.73 (br s, 1H), 7.44-7.40 (m, 2H), 7.33-7.28 (m, 3H), 7.16 (d, J=1.6 Hz, 1H), 5.93-5.80 (m, 1H), 5.60 (dd, J=15.6, 4.0 Hz, 1H), 4.65 (t, J=5.2 Hz, 1H), 3.82-3.75 (m, 2H), 2.87 (t, J=6.4 Hz, 2H), 2.50 (s, 3H).

### Example 83 Preparation of 3-acetamido-6-(4-(2-fluoroacrylamido)phenyl)-7-(4-((4-methylpyrimidin-2-yl)oxy)phenyl)pyrrolo[1,2-a]pyrazine-8-carboxamide

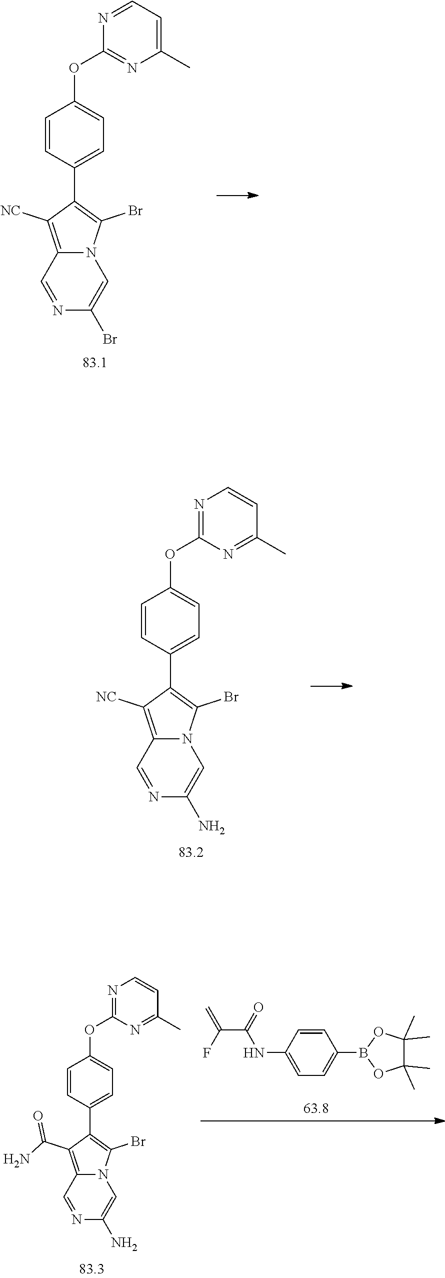

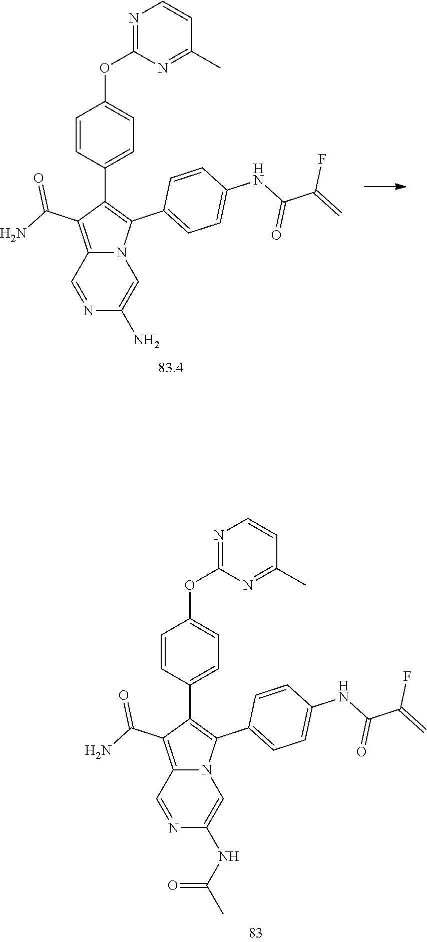 [SMILES: C=C(F)C(=O)Nc1ccc(-c2c(-c3ccc(Oc4nccc(C)n4)cc3)c(C(N)=O)c3cnc(N)cn23)cc1 ; C=C(F)C(=O)Nc1ccc(-c2c(-c3ccc(Oc4nccc(C)n4)cc3)c(C(N)=O)c3cnc(NC(C)=O)cn23)cc1]

### Step 1: Preparation of Compound 83.2

Compound 83.1 was synthesized in a similar fashion according to the procedure for example 76.

A solution of compound 83.1 (600.0 mg, 1.24 mmol) in ammonia (8 mL) was stirred at 100° C. under microwave for 2 h. After cooling to rt, the reaction mixture was concentrated and purified by flash column chromatography on silica gel to afford compound 83.2 (150.0 mg, 28.8%) as a yellow solid. LCMS: 421.1 [M+H]⁺.

### Step 2: Preparation of Compound 83.3

A solution of compound 83.2 (130.0 mg, 0.31 mmol) in H₂SO₄ (2 mL) was stirred at 25° C. for 18 h. The reaction solution was poured into ice (10 mL) and the pH was adjusted to 7 by Na₂CO₃ (aq). The solution was extracted with EA (5 mL×3), washed with brine (5 mL), dried over Na₂SO₄, filtered, and concentrated to afford compound 83.3. LCMS: 437.9; 439.9 [M+H]⁺.

### Step 3: Preparation of Compound 83.4

A solution of compound 83.3 (120.0 mg, 0.27 mmol), compound 63.8 (119.0 mg, 0.41 mmol), K₂CO₃ (75.5 mg, 0.55 mmol) and Pd(PPh₃)₄ (31.6 mg, 0.027 mmol) in dioxane (3 mL) and H₂O (1 mL) was stirred at 90° C. for 2 h. After cooling to rt, the mixture was extracted with EA (10 mL), washed with brine (5 mL), dried over Na₂SO₄, filtered, concentrated and purified by flash column chromatography on silica gel to afford compound 83.4. LCMS: 524.1 [M+H]⁺.

### Step 4: Preparation of Example 83

To a solution of compound 83.4 (50.0 mg, 0.096 mmol) in pyridine (2 mL) was added DMAP (3.50 mg, 0.029 mmol) and acetyl chloride (22.5 mg, 0.287 mmol) at 25° C. The reaction mixture was stirred at 60° C. for 1 h. After cooling to rt, the reaction solution was diluted with DCM (10 mL), washed with brine (5 mL), dried over Na₂SO₄, filtered, concentrated, and purified by prep-HPLC to afford example 83. LCMS: 566.2 [M+H]⁺. ¹H NMR: (400 MHZ, DMSO-d₆) δ 10.55-10.10 (m, 2H), 9.33-9.08 (m, 1H), 8.87 (d, J=2.8 Hz, 1H), 8.54-8.39 (m, 1H), 7.96-7.77 (m, 2H), 7.46-7.26 (m, 5H), 7.16 (d, J=2.1 Hz, 3H), 6.64-6.41 (m, 1H), 5.87-5.62 (m, 1H), 5.56-5.35 (m, 1H), 2.42 (s, 3H), 2.05 (s, 3H).

### Example 84 Preparation of 7-(4-acrylamidophenyl)-1-methyl-6-(4-((6-methylpyridin-2-yl)oxy)phenyl)pyrrolo[1,2-c]pyrimidine-5-carboxamide

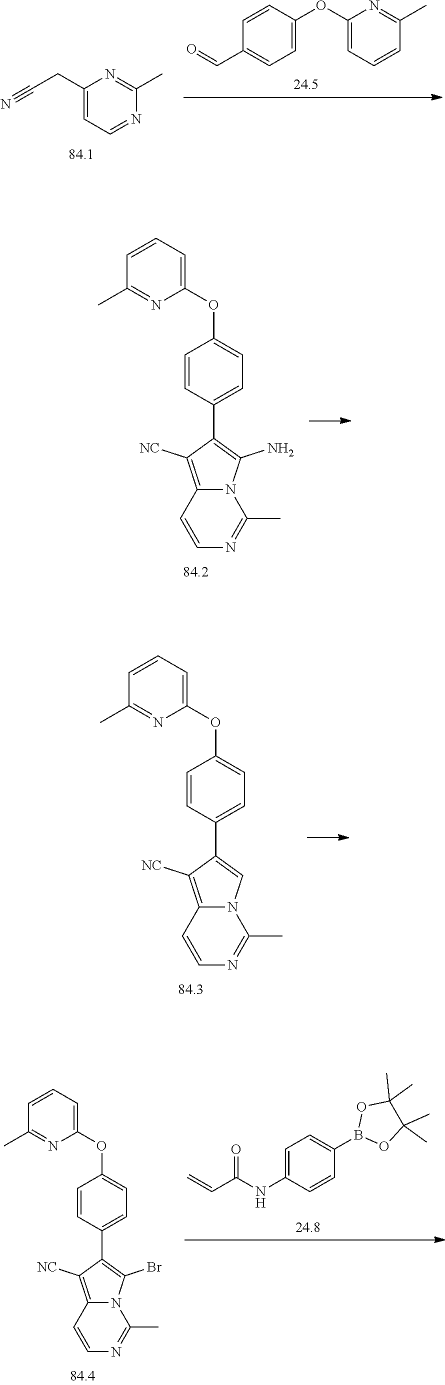

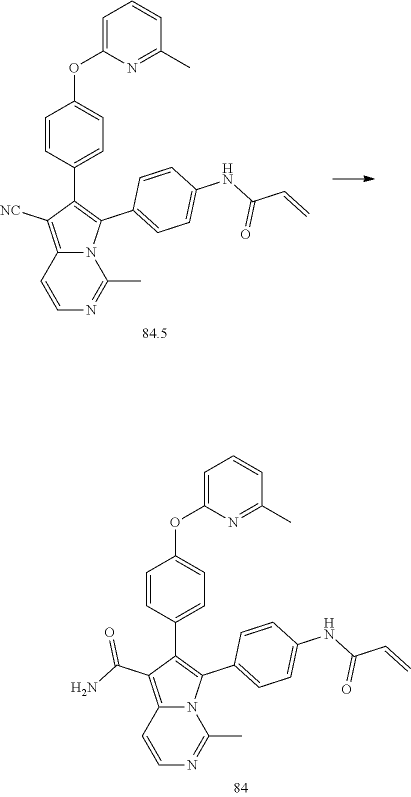 [SMILES: C=CC(=O)Nc1ccc(-c2c(-c3ccc(Oc4cccc(C)n4)cc3)c(C#N)c3ccnc(C)n23)cc1 ; C=CC(=O)Nc1ccc(-c2c(-c3ccc(Oc4cccc(C)n4)cc3)c(C(N)=O)c3ccnc(C)n23)cc1]

### Step 1: Preparation of Compound 84.2

To a solution of compound 84.1 (500.0 mg, 3.76 mmol) in n-BuOH (10 mL) were added compound 24.5 (800.7 mg, 3.76 mmol), DBU (1.14 g, 7.51 mmol) and acetyl cyanide (288.15 mg, 3.76 mmol) at 25° C. Then the reaction mixture was heated to 100° C. under microwave for 20 min. After cooling to rt, the reaction mixture was filtered and the filtrate was concentrated and purified by flash column chromatography on silica gel to afford compound 84.2 (600.0 mg, 44.96% yield) as a yellow solid. LCMS: 356.0 [M+H]⁺.

### Step 2: Preparation of Compound 84.3

To a solution of compound 84.2 (500.0 mg, 1.41 mmol) in THF (5 mL) was added isopentyl nitrite (660.0 mg, 5.13 mmol). The mixture was stirred at 70° C. for 1 h. After cooling to rt, the reaction mixture was filtered and the filtrate was concentrated and purified by flash column chromatography on silica gel to afford compound 84.3 (230.0 mg, 48.03% yield) as a yellow solid. LCMS: 341.1 [M+H]⁺.

### Step 3: Preparation of Compound 84.4

To a solution of compound 84.3 (230.0 mg, 0.674 μmol) in CH₃CN (3 mL) was added NBS (130.0 mg, 0.727 μmol). The mixture was stirred at −78° C. for 0.5 h. After warming to rt, the reaction mixture was poured into H₂O (20 mL) and extracted with EA (20 mL×3). The combined organic layers were washed with brine (15 mL), dried over Na₂SO₄, filtered, concentrated, and purified by flash column chromatography on silica gel to afford compound 84.4. LCMS: 420.8, 422.8 [M+H]⁺.

### Step 4: Preparation of Compound 84.5

To a solution of compound 84.4 (300.0 mg, 0.716 μmol) and compound 24.8 (293.2 mg, 1.07 mmol) in dioxane (6 mL) and H₂O (2 mL) was added K₂CO₃ (197.8 mg, 1.43 mmol). Then Pd-118 (47.0 mg, 0.072 mmol) was added in the mixture and the mixture was stirred at 90° C. for 2 h under N₂. After cooling to rt, the reaction solution was diluted with water (20 ml) and extracted with EA (30 mL×3). The combined organic layers were washed with brine (20 mL), dried over Na₂SO₄, filtered, concentrated, and purified by flash column chromatography on silica gel to afford compound 84.5. LCMS: 486.1 [M+H]⁺.

### Step 5: Preparation of Example 84

A solution of compound 84.5 (150.0 mg, 0.309 mmol) in H₂SO₄ (3 mL, 56.3 mmol) was stirred at 30° C. for 16 h. After cooling to rt, the reaction solution was diluted with ice water (10 mL) and the pH of the reaction solution was adjusted to 7 by aqueous solution of sodium carbonate. After extraction with EA (20 mL×3), the combined organic layers were washed with brine (20 mL), dried over Na₂SO₄, filtered, concentrated, and purified by prep-HPLC to afford example 84. LCMS: 504.3 [M+H]⁺. ¹H NMR: (400 MHZ, DMSO-d₆) δ 10.27 (s, 1H), 7.96 (d, J=6.3 Hz, 1H), 7.78-7.68 (m, 1H), 7.64 (d, J=8.5 Hz, 2H), 7.59 (d, J=6.3 Hz, 1H), 7.36 (d, J=8.5 Hz, 2H), 7.31-7.13 (m, 3H), 7.05-6.97 (m, 3H), 6.72 (d, J=8.3 Hz, 1H), 6.51-6.38 (m, 1H), 6.32-6.22 (m, 1H), 6.02 (br s, 1H), 5.83-5.73 (m, 1H), 2.31 (s, 3H), 2.17 (s, 3H).

### Example 85 Preparation of 6-(3-fluoro-4-((4-methylpyrimidin-2-yl)oxy)phenyl)-7-(4-(2-fluoroacrylamido)phenyl)-3-methylpyrrolo[1,2-c]pyrimidine-5-carboxamide

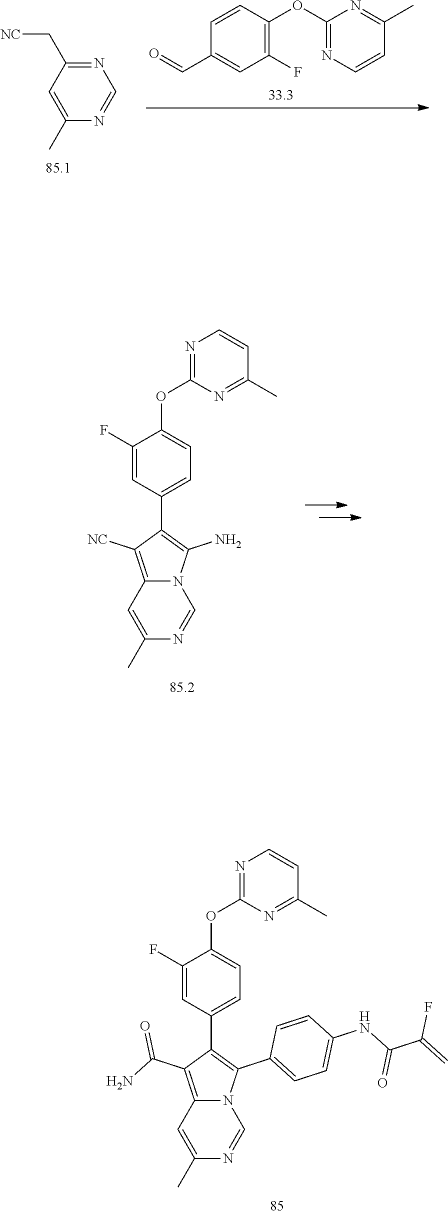

### Step 1: Preparation of Compound 85.2

To a solution of compound 85.1 (600.0 mg, 4.51 mmol) and compound 33.3 (1.05 g, 4.51 mmol) in n-BuOH (3 mL) were added DBU (685.7 mg, 4.51 mmol) and 2-oxopropanenitrile (311.3 mg, 4.51 mmol). Then the mixture heated to 100° C. under microwave for 30 min. After cooling to rt, the reaction mixture was concentrated and purified by flash column chromatography on silica gel to afford compound 85.2 (1.0 g, 59.3% yield) as a white solid. LCMS: 375.2 [M+H]⁺.

### Step 2: Preparation of Example 85

In a similar fashion according to the procedure for example 15, example 85 was synthesized from compound 85.2. Spectrum data of example 85: LCMS: 541.2 [M+H]⁺. ¹H NMR: (400 MHZ, DMSO-d₆) δ 10.44 (s, 1H), 8.77 (s, 1H), 8.48 (d, J=5.0 Hz, 1H), 7.83 (d, J=8.5 Hz, 2H), 7.71 (s, 1H), 7.39 (d, J=8.6 Hz, 2H), 7.35-7.15 (m, 4H), 7.10 (d, J=8.4 Hz, H), 6.63-6.29 (m, 1H) 5.83-5.65 (m, 1H), 5.46 (dd, J=15.6, 3.6 Hz, 1H), 2.43 (s, 3H), 2.42 (s, 3H).

### Example 86 Preparation of 6-(3-fluoro-4-((4-methylpyrimidin-2-yl)oxy)phenyl)-7-(4-(2-fluoroacrylamido)phenyl)-3-(1-methyl-1H-pyrazol-4-yl)pyrrolo[1,2-c]pyrimidine-5-carboxamide

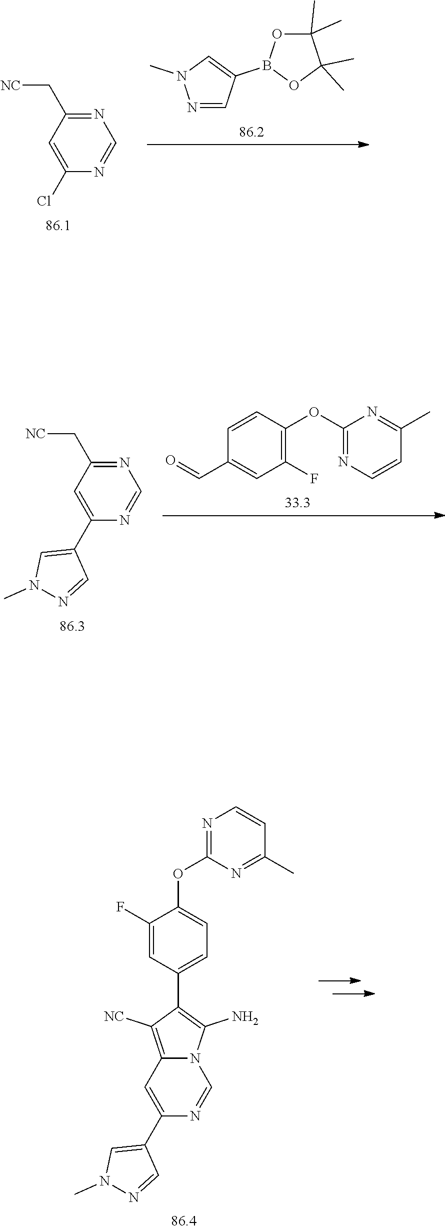

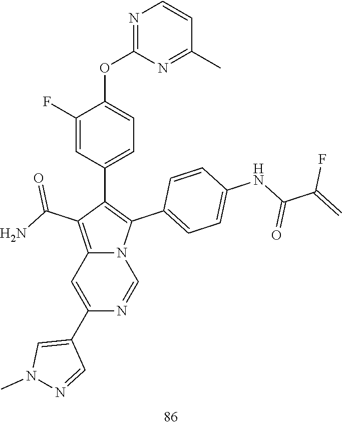 [SMILES: C=C(F)C(=O)Nc1ccc(-c2c(-c3ccc(Oc4nccc(C)n4)c(F)c3)c(C(N)=O)c3cc(-c4cnn(C)c4)ncn23)cc1]

### Step 1: Preparation of Compound 86.3

To a mixture of compound 86.1 (3.00 g, 19.53 mmol) and compound 86.2 (6.10 g, 29.3 mmol) in dioxane (30 mL) and H₂O (10 mL) was added K₂CO₃ (6.70 g, 48.83 mmol). Then Pd-118 (1.30 g, 1.95 mmol) was added in the mixture and the mixture was stirred at 100° C. for 18 h under N₂. After cooling to rt, the reaction solution was diluted with water (50 mL) and extracted with EA (50 mL×3). The combined organic layers were washed with brine (50 mL), dried over Na₂SO₄, filtered, concentrated, and purified by flash column chromatography on silica gel to afford compound 86.3 (1.30 g, 33.4%) as a yellow solid. LCMS: 200.1 [M+H]⁺.

### Step 2: Preparation of Compound 86.4

To a solution of compound 86.3 (700.0 mg, 3.51 mmol) and compound 33.3 (816.0 mg, 3.51 mmol) in n-BuOH (10 mL) was added DBU (534.8 mg, 3.51 mmol). Then 2-oxopropanenitrile (242.8 mg, 3.51 mmol) was added in the solution at 25° C. and purged with N₂ for 3 times. After the addition, the reaction mixture was heated to 100° C. under microwave for 20 min. After cooling to rt, the reaction mixture was concentrated and purified by flash column chromatography on silica gel to afford compound 86.4 (900.0 mg, 58.20%) as a yellow solid. LCMS: 441.1 [M+H]⁺.

### Step 3: Preparation of Example 86

In a similar fashion according to the procedure for example 15, example 86 was synthesized from compound 86.4. Spectrum data of example 86: LCMS: 607.2 [M+H]⁺. ¹H NMR: (400 MHZ, DMSO-d₆) δ 10.46 (s, 1H), 8.85 (d, J=1.0 Hz, 1H), 8.48 (d, J=5.0 Hz, 1H), 8.28 (s, 1H), 7.98 (d, J=2.8 Hz, 2H), 7.84 (d, J=8.5 Hz, 2H), 7.42 (d, J=8.5 Hz, 2H), 7.36-7.22 (m, 3H), 7.19 (d, J=5.0 Hz, 1H), 7.11 (d, J=8.3 Hz, 1H), 6.71-6.51 (m, 1H), 5.88-5.61 (m, 1H), 5.47 (dd, J=15.6, 3.8 Hz, 1H), 3.90 (s, 3H), 2.42 (s, 3H).

### Example 87 Preparation of 6-(3-fluoro-4-((4-methylpyrimidin-2-yl)oxy)phenyl)-7-(4-(2-fluoroacrylamido)phenyl)-3-methylpyrrolo[1,2-b]pyridazine-5-carboxamide

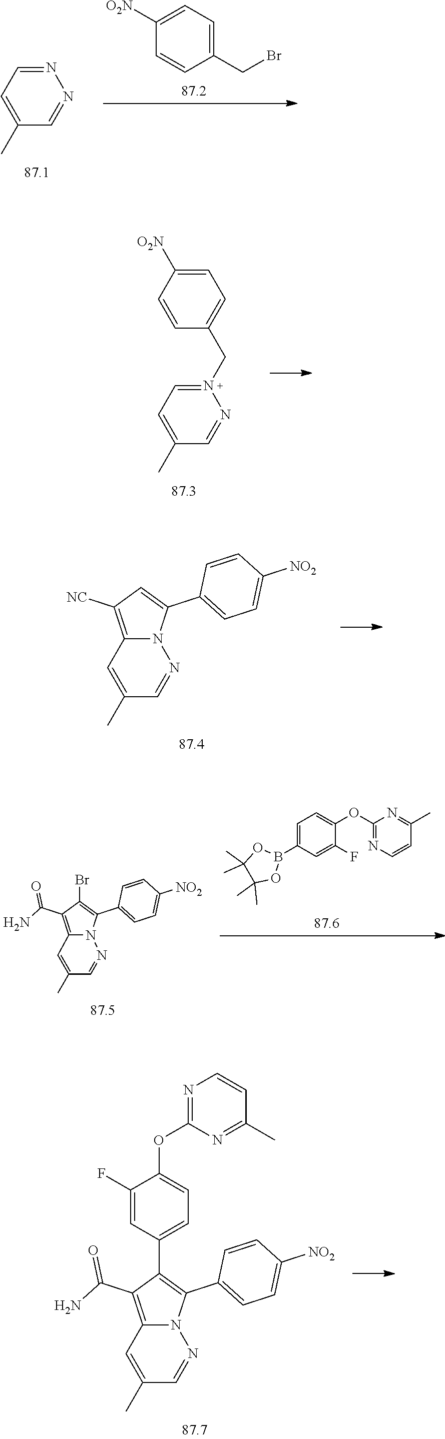

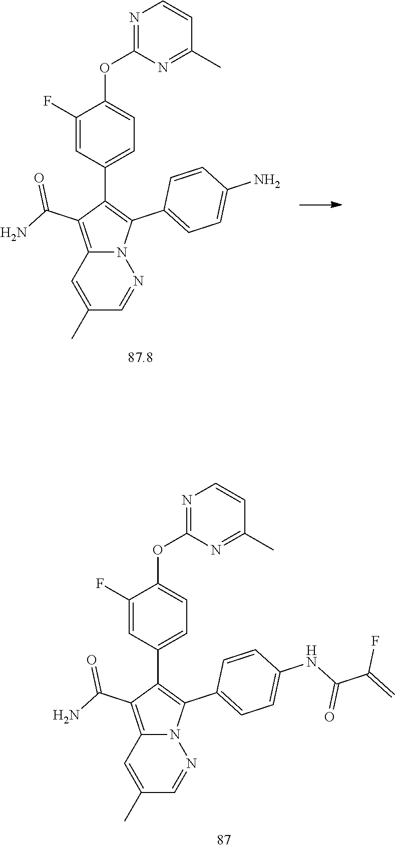 [SMILES: Cc1cnn2c(-c3ccc(N)cc3)c(-c3ccc(Oc4nccc(C)n4)c(F)c3)c(C(N)=O)c2c1 ; C=C(F)C(=O)Nc1ccc(-c2c(-c3ccc(Oc4nccc(C)n4)c(F)c3)c(C(N)=O)c3cc(C)cnn23)cc1]

### Step 1: Preparation of Compound 87.3

A solution of compound 87.1 (3.60 g, 38.3 mmol) and compound 87.2 (9.90 g, 45.9 mmol) in EA (50 mL) was stirred at 80° C. for 12 h under N₂. After cooling to rt, the reaction mixture was filtered and concentrated to afford compound 87.3 (12.0 g, crude) as a brown solid. LCMS: 230.1 [M+H]⁺.

### Step 2: Preparation of Compound 87.4

To a solution of compound 87.3 (7.50 g, 32.6 mmol) in DME (100 mL) was added prop-2-enenitrile (7.07 g, 133.1 mmol). Then MnO₂ (14.2 g, 162.9 mmol) and TEA (13.6 mL, 97.7 mmol) were added and the reaction mixture was stirred at 80° C. for 12 h. After cooling to rt, the reaction mixture was filtered and the filtrate was concentrated and purified by flash column chromatography on silica gel to afford compound 87.4 (1.0 g, 11.0% yield) as a yellow solid. LCMS: 279.3 [M+H]⁺.

### Step 3: Preparation of Compound 87.5

To a solution of compound 87.4 (670.0 mg, 2.41 mmol) in H₂SO₄ (5 mL) was added NBS (857.1 mg, 4.82 mmol). The reaction mixture was stirred at rt for 12 h. The reaction solution was poured into ice (10 mL) and the pH was adjusted to 9 by Na₂CO₃ (aq). The solution was extracted with EA (10 mL×2), washed with brine (20 mL), dried over Na₂SO₄, filtered, concentrated, and purified by flash column chromatography on silica gel to afford compound 87.5 (120.0 mg, 13.3% yield) as a yellow solid. LCMS: 375.0; 377.0 [M+H]⁺.

### Step 4: Preparation of Compound 87.7

To a solution of compound 87.5 (120.0 mg, 0.32 mmol) and compound 87.6 (116.2 mg, 0.352 mmol) in dioxane (1 mL) and H₂O (0.2 mL) were added Cs₂CO₃ (312.6 mg, 0.959 mmol) and Pd(PPh₃)₄ (37.0 mg, 0.032 mmol) under N₂. The reaction was stirred at 90° C. for 12 h. After cooling to rt, the reaction mixture was concentrated and purified by flash column chromatography on silica gel to afford compound 87.7. LCMS: 499.2 [M+H]⁺.

### Step 5: Preparation of Compound 87.8

To a solution of compound 87.7 (110.0 mg, 0.221 mmol) in EtOH (2 mL) and H₂O (0.5 mL) were added Fe (80.0 mg, 1.43 mmol) and CaCl₂) (73.5 mg, 0.662 mmol) at 25° C. Then the mixture was stirred at 80° C. for 12 h. After cooling to rt, the reaction mixture was filtered and the filtrate was concentrated under reduced pressure to afford compound 87.8.

### Step 6: Preparation of Example 87

To a solution of compound 87.8 (100.0 mg, 0.213 mmol) in pyridine (4 mL) were added 2-fluoroprop-2-enoic acid (28.8 mg, 0.32 mmol) and EDCI (122.8 mg, 0.64 mmol). The reaction solution was stirred at 60° C. for 12 h. After cooling to rt, the reaction solution was filtered, concentrated, and purified by prep-HPLC to afford example 87. LCMS: 541.8 [M+H]⁺. ¹H NMR: (400 MHZ, DMSO-d₆) δ 10.38 (s, 1H), 8.48 (d, J=5.0 Hz, 1H), 8.23 (d, J=2.1 Hz, 1H), 8.20 (d, J=1.0 Hz, 1H), 7.73 (d, J=8.8 Hz, 2H), 7.39-7.22 (m, 5H), 7.19 (d, J=5.0 Hz, 1H), 7.12 (dd, J=8.3, 1.3 Hz, 1H), 6.53 (br s, 1H), 5.80-5.64 (m, 1H), 5.44 (dd, J=15.6, 3.6 Hz, 1H), 2.42 (s, 3H), 2.36 (s, 3H).

### Example 91 Preparation of 6-(3-fluoro-4-((4-methylpyrimidin-2-yl)oxy)phenyl)-7-(2-fluoro-4-(2-fluoroacrylamido)phenyl)-3-(1-methyl-1H-pyrazol-4-yl)pyrrolo[1,2-c]pyrimidine-5-carboxamide

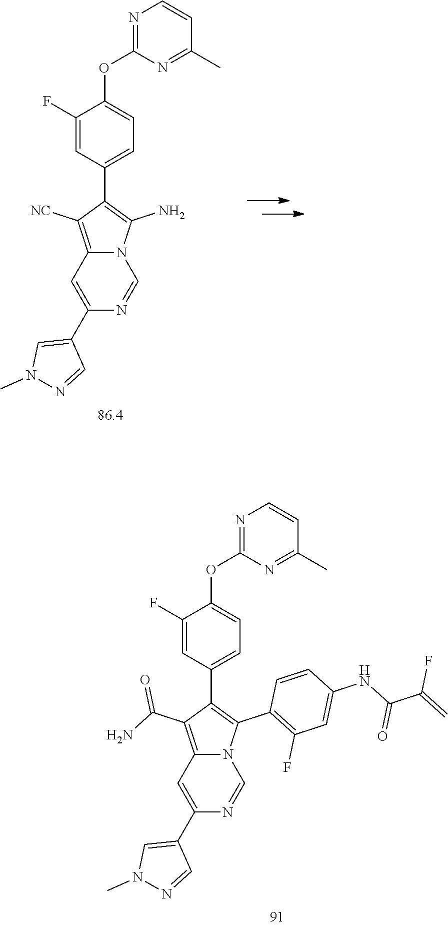

### Step 1: Preparation of Example 91

In a similar fashion according to the procedure for example 15, example 91 was synthesized from compound 86.4. Spectrum data of example 91: LCMS: 625.2 [M+H]⁺. ¹H NMR: (400 MHZ, DMSO-d₆) δ 10.66 (s, 1H), 8.74 (s, 1H), 8.48 (d, J=4.8 Hz, 1H), 8.30 (s, 1H), 7.99 (d, J=7.2 Hz, 2H), 7.83 (d, J=1.6 Hz, 1H), 7.65 (d, J=1.6 Hz, 1H), 7.41-7.23 (m, 5H), 7.20-7.18 (m, 1H), 6.83 (s, 1H), 5.84-5.71 (m, 1H), 5.51 (dd, J=15.6, 3.6 Hz, 1H), 3.91 (s, 3H), 2.42 (s, 3H).

### Example 92 Preparation of 6-(3-fluoro-4-((4-methylpyrimidin-2-yl)oxy)phenyl)-7-(4-(2-fluoroacrylamido)-2-methylphenyl)-3-(1-methyl-1H-pyrazol-4-yl)pyrrolo[1,2-c]pyrimidine-5-carboxamide

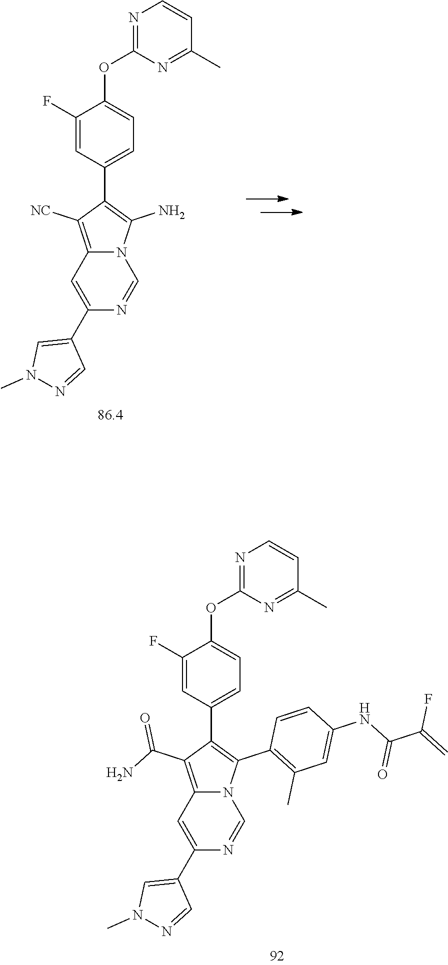

### Step 1: Preparation of Example 92

In a similar fashion according to the procedure for example 15, example 92 was synthesized from compound 86.4. Spectrum data of example 92: LCMS: 621.2 [M+H]⁺. ¹H NMR: (400 MHZ, DMSO-d₆) δ 10.41 (s, 1H), 8.51-8.40 (m, 2H), 8.27 (s, 1H), 7.97 (s, 2H), 7.77 (s, 1H), 7.72-7.67 (m, 1H), 7.40-7.24 (m, 3H), 7.23-7.16 (m, 2H), 7.09-7.04 (m, 1H), 6.91-6.69 (m, 1H), 5.84-5.65 (m, 1H), 5.47 (dd, J=15.6, 3.6 Hz, 1H), 3.90 (s, 3H), 2.41 (s, 3H), 1.93 (s, 3H).

### Example 93 Preparation of 6-(3-fluoro-4-((4-methylpyrimidin-2-yl)oxy)phenyl)-7-(4-(2-fluoroacrylamido)phenyl)-2-methylpyrrolo[1,2-b]pyridazine-5-carboxamide

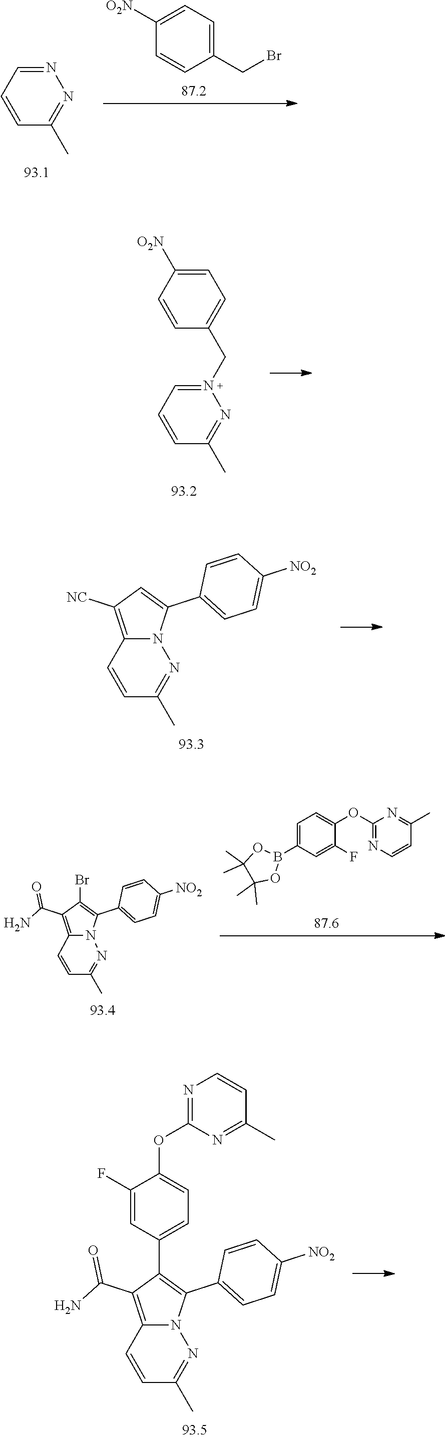

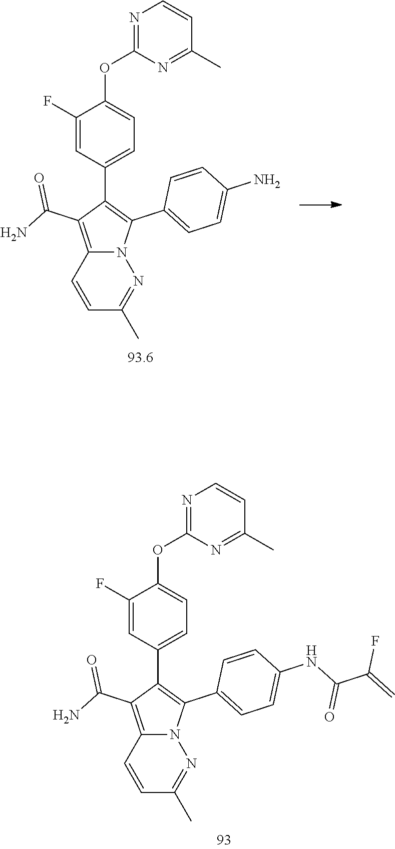 [SMILES: Cc1ccnc(Oc2ccc(-c3c(C(N)=O)c4ccc(C)nn4c3-c3ccc(N)cc3)cc2F)n1 ; C=C(F)C(=O)Nc1ccc(-c2c(-c3ccc(Oc4nccc(C)n4)c(F)c3)c(C(N)=O)c3ccc(C)nn23)cc1]

### Step 1: Preparation of Compound 93.2

A solution of compound 93.1 (6.00 g, 63.8 mmol) and compound 87.2 (16.5 g, 76.5 mmol) in ethyl acetate (100 mL) was stirred at 80° C. for 12 h under N₂. After cooling to rt, the reaction mixture was filtered, and the filtrate was concentrated in vacuum to afford compound 93.2 (16.0 g, crude) as gray solid. ¹H NMR: (400 MHZ, DMSO-d₆) δ 8.69 (dd, J=8.8, 6.0 Hz, 1H), 8.55 (d, J=8.0 Hz, 1H), 8.35-8.19 (m, 2H), 7.82 (d, J=8.8 Hz, 2H), 6.19 (s, 2H), 2.73 (s, 3H).

### Step 2: Preparation of Compound 93.3

To a solution of compound 93.2 (8.0 g, 34.752 mmol) in DME (100 mL) was added prop-2-enenitrile (11.040 g, 207.910 mmol). Then MnO₂ (12.1 g, 139.010 mmol) and TEA (12.04 mL, 86.881 mmol) were added to the solution at 25° C. Finally the reaction mixture was stirred at 80° C. for 7 h. After cooling to rt, the mixture was filtered through a Celite pad, and the filtrate was concentrated to give the crude compound 93.3 (3.41 g, 35.3%) as a yellow solid. LCMS: 279.1 [M+H]⁺.

### Step 3: Preparation of Compound 93.4

To a solution of compound 93.3 (500.0 mg, 1.80 mmol) in H₂SO₄ (5 mL) was added NBS (639.6 mg, 3.59 mmol). Then the reaction was stirred at 25° C. for 12 h. The reaction solution was poured into ice (25 mL) and the pH was adjusted to 7 by Na₂CO₃ (aq). The solution was extracted with DCM (15 mL×3), washed with brine (25 mL), dried over Na₂SO₄, filtered, concentrated, and purified by flash column chromatography on silica gel to afford compound 93.4 (30.0 mg, 4.5%) as a yellow solid. LCMS: 375.3; 377.3 [M+H]⁺.

### Step 4: Preparation of Compound 93.5

To a solution of compound 93.4 (30.0 mg, 0.08 mmol) and compound 87.6 (39.6 mg, 0.12 mmol) in dioxane (1 mL) and H₂O (0.2 mL) was added Cs₂CO₃ (52.1 mg, 0.16 mmol). Then Pd(PPh₃)₄ (9.2 mg, 0.008 mmol) was added in the solution under N₂ and the reaction was stirred at 90° C. for 2 h. After cooling to rt, the reaction mixture was concentrated and purified by flash column chromatography on silica gel to afford compound 93.5. LCMS: 499.1 [M+H]⁺.

### Step 5: Preparation of Compound 93.6

To a solution of compound 93.5 (20.0 mg, 0.040 mmol) in EtOH (2 mL) and H₂O (0.4 mL) was added Fe (11.2 mg, 0.201 mmol) and CaCl₂) (2.2 mg, 0.020 mmol). The reaction was stirred at 80° C. for 3 h. After cooling to rt, the solution was filtered, and the filtrate was concentrated to afford compound 93.6. LCMS: 452.1 [M−16]⁺.

### Step 6: Preparation of Example 93

To a solution of compound 93.6 (20.0 mg, 0.043 mmol) and 2-fluoroprop-2-enoic acid (7.7 mg, 0.085 mmol) in pyridine (2 mL) was added EDCI (8.2 mg, 0.043 mmol). The reaction was stirred at 50° C. for 2 h. After cooling to rt, the reaction solution was concentrated and purified by prep-HPLC to afford example 93. LCMS: 541.2 [M+H]⁺. ¹H NMR: (400 MHZ, DMSO-d₆) δ 10.39 (s, 1H), 8.49 (d, J=4.0 Hz, 1H), 8.29 (d, J=9.2 Hz, 1H), 7.74 (s, 1H), 7.73 (s, 1H), 7.40-7.17 (m, 6H), 7.11 (d, J=8.4 Hz, 1H), 6.92 (d, J=9.6 Hz, 1H), 6.65 (br s, 1H), 5.81-5.64 (m, 1H) 5.45 (dd, J=15.6, 3.6 Hz, 1H), 2.43 (s, 3H), 2.42 (s, 3H).

### Example 94 Preparation of 6-(3-fluoro-4-((4-methylpyrimidin-2-yl)oxy)phenyl)-7-(4-(2-fluoroacrylamido)-2-methylphenyl)-2-methylpyrrolo[1,2-b]pyridazine-5-carboxamide

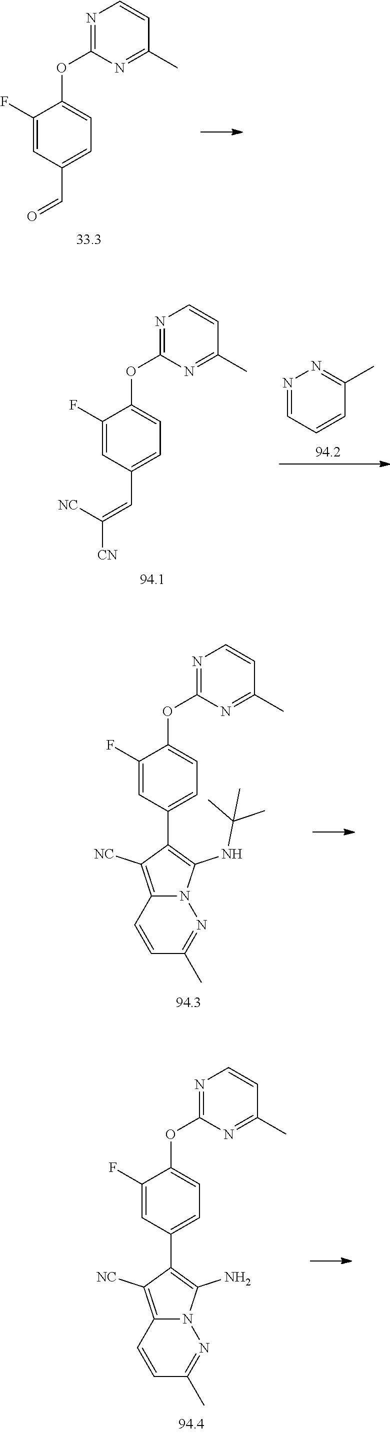

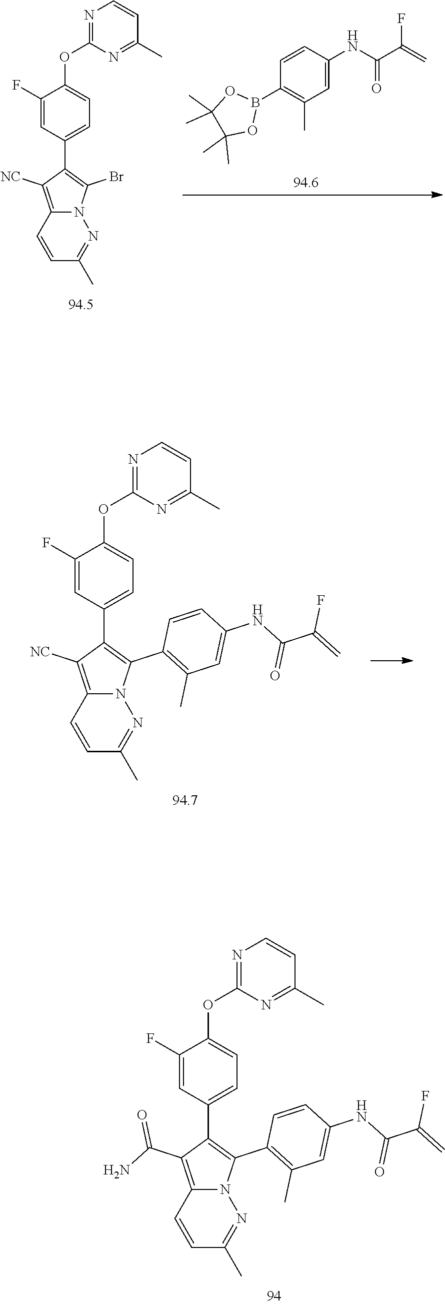 [SMILES: Cc1ccnc(Oc2ccc(-c3c(C#N)c4ccc(C)nn4c3Br)cc2F)n1 ; C=C(F)C(=O)Nc1ccc(B2OC(C)(C)C(C)(C)O2)c(C)c1 ; C=C(F)C(=O)Nc1ccc(-c2c(-c3ccc(Oc4nccc(C)n4)c(F)c3)c(C#N)c3ccc(C)nn23)c(C)c1 ; C=C(F)C(=O)Nc1ccc(-c2c(-c3ccc(Oc4nccc(C)n4)c(F)c3)c(C(N)=O)c3ccc(C)nn23)c(C)c1]

### Step 1: Preparation of Compound 94.1

A solution of compound 33.3 (6.00 g, 25.8 mmol) and propanedinitrile (1.70 g, 25.8 mmol) in EtOH (50 mL) was stirred at 80° C. for 4 h. After cooling to rt, the reaction was filtered, and the filtrate was concentrated to afford compound 94.1 (6.00 g, 82.8%) as a white solid. ¹H NMR: (400 MHZ, DMSO-d₆) δ 8.56 (s, 1H), 8.52 (d, J=4.0 Hz, 1H) 7.98-7.87 (m, 2H), 7.70 (t, J=8.0 Hz, 1H), 7.26 (d, J=4.8 Hz, 1H), 2.44 (s, 3H).

### Step 2: Preparation of Compound 94.3

To a solution of compound 94.2 (700.0 mg, 7.44 mmol), compound 94.1 (2.10 g, 7.44 mmol) and 2-isocyano-2-methylpropane (600.0 mg, 7.44 mmol) in CH₃CN (30.0 mL) was added H₂O (3 mL). The reaction was stirred at 25° C. for 12 h. The reaction was filtered, and the filtrate was concentrated to afford compound 94.3 (1.30 g, 40.6%) as a yellow solid. LCMS: 431.2 [M+H]⁺.

### Step 3: Preparation of Compound 94.4

A solution of compound 94.3 (700.0 mg, 1.63 mmol) in H₂SO₄ (4 mL) was stirred at 0˜25° C. for 3 h. The reaction solution was poured into ice (25 mL) and the pH was adjusted to 7 by Na₂CO₃ (aq). The solution was extracted with DCM (15 mL×3), washed with brine (25 mL), dried over Na₂SO₄, filtered, concentrated to afford compound 94.4 (700.0 mg, crude) as a yellow solid. LCMS: 375.1 [M+H]⁺.

### Step 4: Preparation of Compound 94.5

To a solution of compound 94.4 (600.0 mg, 1.60 mmol) in HBr—AcOH (3 mL) was added copperbromide (464.1 mg, 2.08 mmol). Then sodium nitrate (681.1 mg, 8.01 mmol) dissolved in H₂O (0.4 mL) was added in the solution. The reaction was stirred at 0° C. for 0.5 h. The reaction solution was poured into ice (25 mL) and the pH was adjusted to 7 by Na₂CO₃ (aq). The solution was extracted with DCM (15 mL×3), washed with brine (25 mL), dried over Na₂SO₄, filtered, concentrated, and purified by flash column chromatography on silica gel to afford compound 94.5. LCMS: 437.8; 439.8 [M+H]⁺.

### Step 5: Preparation of Compound 94.7

To a solution of compound 94.5 (250 mg, 0.570 mmol) and compound 94.6 (208.9 mg, 0.684 mmol) in dioxane (3 mL) and H₂O (0.5 mL) was added K₂CO₃ (157.7 mg, 1.141 mmol). Then Pd(PPh₃)₄ (65.9 mg, 0.057 mmol) was added in the solution under N₂. The reaction was stirred at 100° C. for 2 h. After cooling to rt, the reaction mixture was concentrated and purified by flash column chromatography on silica gel to afford compound 94.7. LCMS: 537.2 [M+H]⁺.

### Step 6: Preparation of Example 94

A solution of compound 94.7 (100.0 mg, 0.186 mmol) in H₂SO₄ (1 mL) was stirred at 30° C. for 12 h. The reaction solution was poured into ice (10 mL) and the pH was adjusted to 7 by Na₂CO₃ (aq). The solution was extracted with DCM (10 mL×2), concentrated, and purified by prep-HPLC to afford example 94. LCMS: 555.2 [M+H]⁺. ¹H NMR: (400 MHZ, DMSO-d₆) δ 10.33 (br s, 1H), 8.51-8.42 (m, 1H), 8.28 (d, J=9.2 Hz, 1H), 7.69 (s, 1H), 7.66-7.50 (m, 2H), 7.28 (t, J=8.4 Hz, 1H), 7.22-7.11 (m, 3H), 7.05 (d, J=8.1 Hz, 1H), 6.91 (d, J=9.0 Hz, 1H), 6.79 (br s, 1H), 5.83-5.64 (m, 1H), 5.45 (dd, J=15.6, 3.3 Hz, 1H), 2.41 (s, 3H), 2.38 (s, 3H), 1.96 (s, 3H).

### Example 95 Preparation of 6-(3-fluoro-4-((4-methylpyrimidin-2-yl)oxy)phenyl)-7-(1-(2-fluoroacryloyl) indolin-5-yl)-2-methylpyrrolo[1,2-b]pyridazine-5-carboxamide

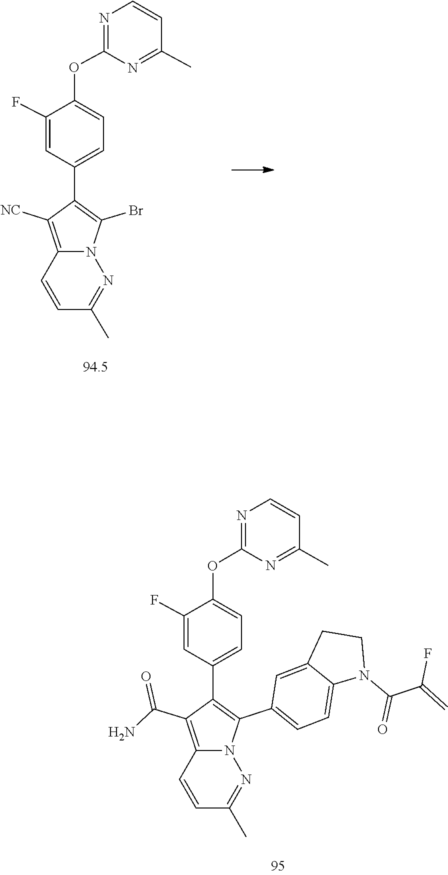 [SMILES: Cc1ccnc(Oc2ccc(-c3c(C#N)c4ccc(C)nn4c3Br)cc2F)n1 ; C=C(F)C(=O)N1CCc2cc(-c3c(-c4ccc(Oc5nccc(C)n5)c(F)c4)c(C(N)=O)c4ccc(C)nn34)ccc21]

### Step 1: Preparation of Example 95

In a similar fashion according to the procedure for example 94, example 95 was synthesized from compound 94.5. Spectrum data of example 95: LCMS: 567.2 [M+H]⁺. ¹H NMR: (400 MHZ, DMSO-d₆) δ 8.49 (d, J=4.8 Hz, 1H), 8.30 (d, J=9.2 Hz, 1H), 7.93 (s, 1H), 7.34-7.10 (m, 7H), 6.92 (d, J=9.2 Hz, 1H), 6.61 (s, 1H), 5.59-5.42 (m, 2H), 4.35-4.15 (m, 2H), 3.16-3.13 (m, 2H), 2.43 (s, 3H), 2.41 (s, 3H).

### Example 96 Preparation of 6-(3-fluoro-4-((4-methylpyrimidin-2-yl)oxy)phenyl)-7-(1-(2-fluoroacryloyl)-6-methylindolin-5-yl)-2-methylpyrrolo[1,2-b]pyridazine-5-carboxamide

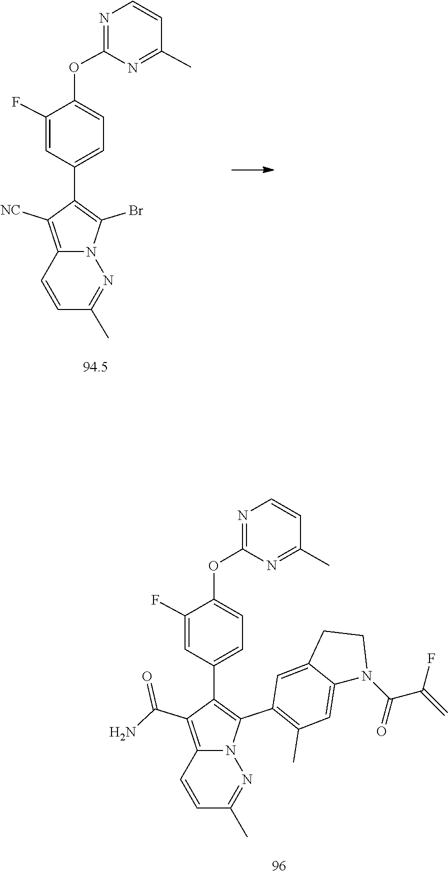 [SMILES: Cc1ccnc(Oc2ccc(-c3c(C#N)c4ccc(C)nn4c3Br)cc2F)n1 ; C=C(F)C(=O)N1CCc2cc(-c3c(-c4ccc(Oc5nccc(C)n5)c(F)c4)c(C(N)=O)c4ccc(C)nn34)c(C)cc21]

### Step 1: Preparation of Example 96

In a similar fashion according to the procedure for example 94, example 96 was synthesized from compound 94.5. Spectrum data of example 96: LCMS: 581.3 [M+H]⁺. ¹H NMR: (400 MHZ, DMSO-d₆) δ 8.48 (d, J=4.8 Hz, 1H), 8.28 (d, J=9.2 Hz, 1H), 7.92 (s, 1H), 7.57-7.54 (m, 1H), 7.31-7.21 (m, 3H), 7.19-7.04 (m, 2H), 6.92-9-6.89 (m, 1H), 6.75 (s, 1H), 5.59-5.42 (m, 2H), 4.27-4.22 (m, 2H), 3.12-3.05 (m, 2H), 2.41 (s, 3H), 2.37 (s, 3H), 1.96 (s, 3H).

### Example 97 Preparation of 7-(3-fluoro-4-((4-methylpyrimidin-2-yl)oxy)phenyl)-6-(1-(2-fluoroacryloyl)-6-methylindolin-5-yl)-3-methylpyrrolo[1,2-a]pyrazine-8-carboxamide

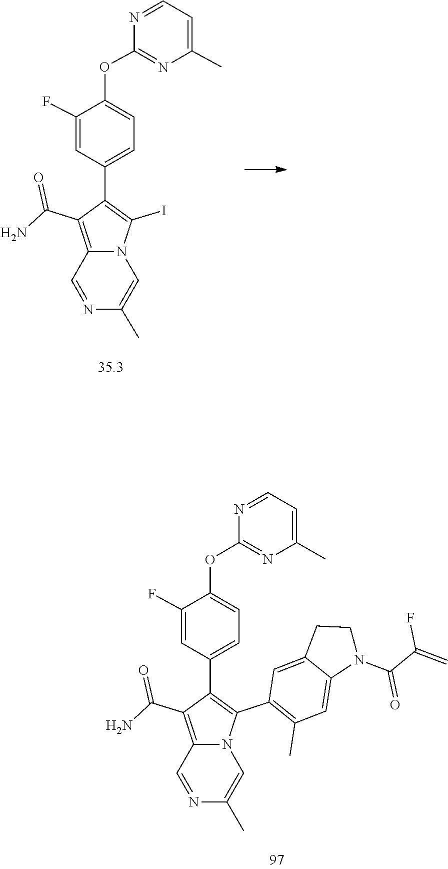 [SMILES: Cc1cn2c(I)c(-c3ccc(Oc4nccc(C)n4)c(F)c3)c(C(N)=O)c2cn1 ; C=C(F)C(=O)N1CCc2cc(-c3c(-c4ccc(Oc5nccc(C)n5)c(F)c4)c(C(N)=O)c4cnc(C)cn34)c(C)cc21]

### Step 1: Preparation of Example 97

In a similar fashion according to the procedure for example 35, example 97 was synthesized from compound 35.3. Spectrum data of example 97: LCMS: 581.2 [M+H]⁺. ¹H NMR: (400 MHZ, DMSO-d₆) δ 9.26 (s, 1H), 8.56 (d, J=4.8 Hz, 1H), 8.15 (br s, 1H), 7.53 (s, 2H), 7.49-7.45 (m, 3H), 7.18-7.15 (m, 3H), 5.79-5.48 (m, 2H), 4.39-4.33 (m, 2H), 3.25-3.15 (m, 2H), 2.43 (s, 3H), 2.36 (s, 3H), 1.95 (s, 3H).

### Example 98 Preparation of 6-(7-fluoro-1-(2-fluoroacryloyl)-1,2,3,4-tetrahydroquinolin-6-yl)-7-(3-fluoro-4-((4-methylpyrimidin-2-yl)oxy)phenyl)-3-methylpyrrolo[1,2-a]pyrazine-8-carboxamide

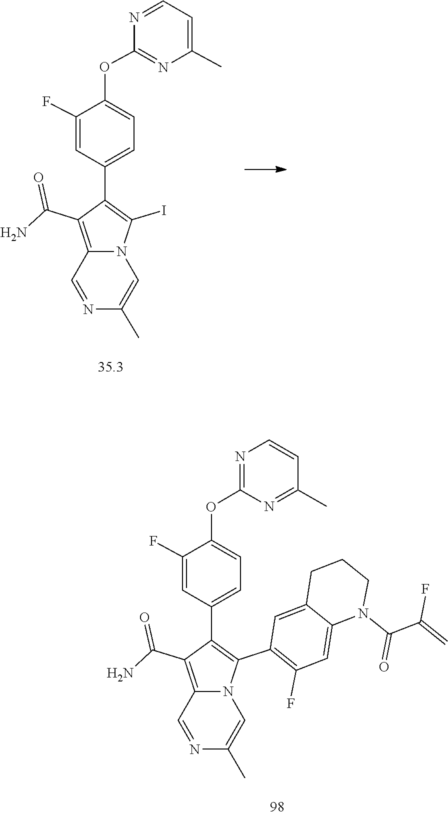 [SMILES: Cc1cn2c(I)c(-c3ccc(Oc4nccc(C)n4)c(F)c3)c(C(N)=O)c2cn1 ; C=C(F)C(=O)N1CCCc2cc(-c3c(-c4ccc(Oc5nccc(C)n5)c(F)c4)c(C(N)=O)c4cnc(C)cn34)c(F)cc21]

### Step 1: Preparation of Example 98

In a similar fashion according to the procedure for example 35, example 98 was synthesized from compound 35.3. Spectrum data of example 98: LCMS: 599.2 [M+H]⁺. ¹H NMR: (400 MHZ, DMSO-d₆) δ 9.19 (s, 1H), 8.47 (d, J=5.2 Hz, 1H), 7.69 (s, 1H), 7.51 (d, J=2.4 Hz, 1H), 7.46 (s, 1H), 7.31 (t, J=8.4 Hz, 1H), 7.24-7.05 (m, 5H), 5.34-5.28 (m, 2H), 3.84-3.70 (m, 2H), 2.72-2.61 (m, 2H), 2.42 (s, 3H), 2.37 (s, 3H), 1.96-1.87 (m, 2H).

### Example 99 Preparation of 6-(4-fluoro-1-(2-fluoroacryloyl) indolin-5-yl)-7-(3-fluoro-4-((4-methylpyrimidin-2-yl)oxy)phenyl)-3-methylpyrrolo[1,2-a]pyrazine-8-carboxamide

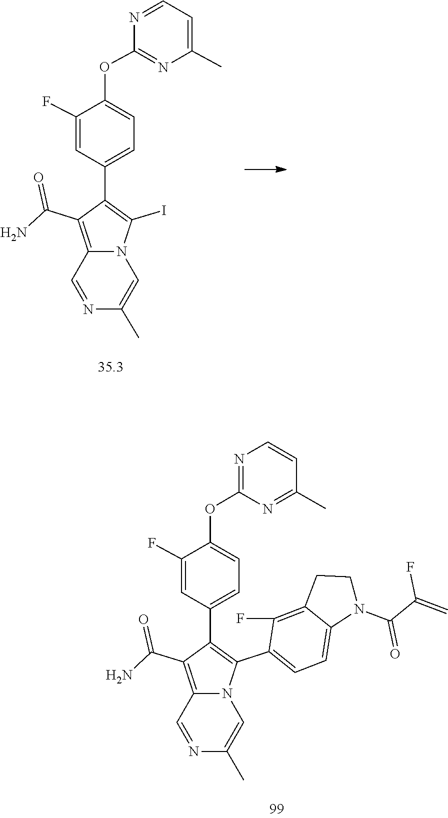 [SMILES: Cc1cn2c(I)c(-c3ccc(Oc4nccc(C)n4)c(F)c3)c(C(N)=O)c2cn1 ; C=C(F)C(=O)N1CCc2c1ccc(-c1c(-c3ccc(Oc4nccc(C)n4)c(F)c3)c(C(N)=O)c3cnc(C)cn13)c2F]

### Step 1: Preparation of Example 99

In a similar fashion according to the procedure for example 15, example 99 was synthesized from compound 35.3. Spectrum data of example 99: LCMS: 585.2 [M+H]⁺. ¹H NMR: (400 MHZ, DMSO-d₆) δ 9.19 (s, 1H), 8.47 (d, J=5.2 Hz, 1H), 7.84-7.82 (m, 1H), 7.70 (s, 1H), 7.55-7.40 (m, 1H), 7.34-7.23 (m, 5H), 7.19-7.10 (m, 1H), 5.66-5.47 (m, 2H), 4.41-4.35 (m, 2H), 3.31-3.21 (m, 2H), 2.42 (s, 3H), 2.33 (s, 3H).

### Example 100 Preparation of 7-(3-fluoro-4-((4-methylpyrimidin-2-yl)oxy)phenyl)-6-(1-(2-fluoroacryloyl) indolin-5-yl)-3-(2-hydroxyethyl)pyrrolo[1,2-a]pyrazine-8-carboxamide

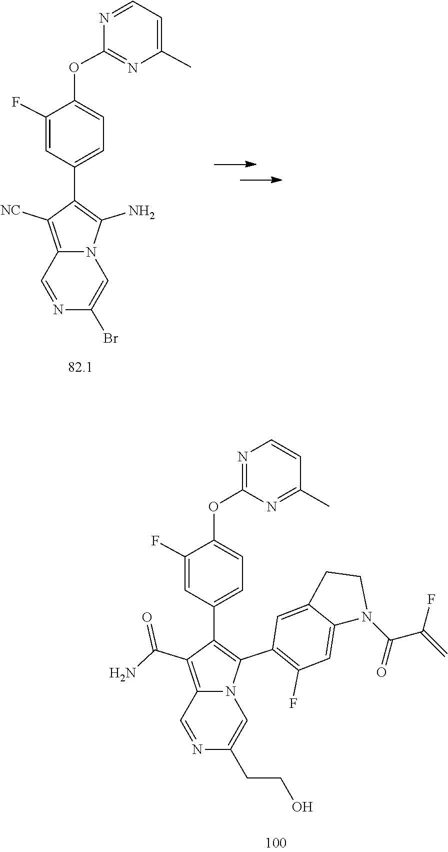 [SMILES: Cc1ccnc(Oc2ccc(-c3c(C#N)c4cnc(Br)cn4c3N)cc2F)n1 ; C=C(F)C(=O)N1CCc2cc(-c3c(-c4ccc(Oc5nccc(C)n5)c(F)c4)c(C(N)=O)c4cnc(CCO)cn34)c(F)cc21]

### Step 1: Preparation of Example 100

In a similar fashion according to the procedure for example 82, example 100 was synthesized from compound 82.1. Spectrum data of example 100: LCMS: 615.3 [M+H]⁺. ¹H NMR: (400 MHZ, DMSO-d₆) δ 9.21 (s, 1H), 8.47 (d, J=4.8 Hz, 1H), 7.89-7.89 (m, 1H), 7.65 (s, 1H), 7.44 (s, 1H), 7.32-7.26 (m, 3H), 7.25-7.23 (m, 1H), 7.18 (s, 1H), 7.11-7.09 (m, 1H), 5.66-5.47 (m, 2H), 4.57 (t, J=5.2 Hz, 1H), 4.36-4.31 (m, 2H), 3.72-3.66 (m, 2H), 3.21-3.07 (m, 2H), 2.79-2.74 (m, 2H), 2.42 (s, 3H).

### Example 101 Preparation of 6-(1-acryloyl-1,2,3,6-tetrahydropyridin-4-yl)-7-(3-fluoro-4-((4-methylpyrimidin-2-yl)oxy)phenyl)-3-methylpyrrolo[1,2-a]pyrazine-8-carboxamide

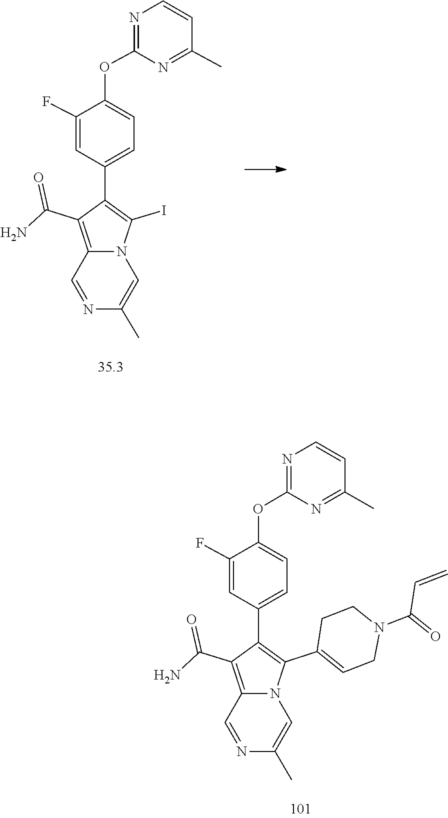 [SMILES: Cc1cn2c(I)c(-c3ccc(Oc4nccc(C)n4)c(F)c3)c(C(N)=O)c2cn1 ; C=CC(=O)N1CC=C(c2c(-c3ccc(Oc4nccc(C)n4)c(F)c3)c(C(N)=O)c3cnc(C)cn23)CC1]

### Step 1: Preparation of Example 101

In a similar fashion according to the procedure for example 15, example 101 was synthesized from compound 35.3. Spectrum data of example 101: LCMS: 513.2 [M+H]⁺. ¹H NMR: (400 MHZ, DMSO-d₆) δ 9.11 (s, 1H), 8.51 (d, J=5.2 Hz, 1H), 8.08 (s, 1H), 7.45-7.35 (m, 3H), 7.27-7.19 (m, 2H), 7.01 (s, 1H), 6.83-6.79 (m, 1H), 6.17-6.10 (m, 2H), 5.73-5.66 (m, 1H), 4.31-4.19 (m, 2H), 3.69-3.65 (m, 2H), 2.43 (s, 3H), 2.39 (s, 3H), 2.11-2.07 (m, 2H).

### Example 102 Preparation of 6-(6′-fluoro-1′-(2-fluoroacryloyl)spiro[cyclopropane-1,3′-indolin]-5′-yl)-7-(3-fluoro-4-((4-methylpyrimidin-2-yl)oxy)phenyl)-3-methylpyrrolo[1,2-a]pyrazine-8-carboxamide

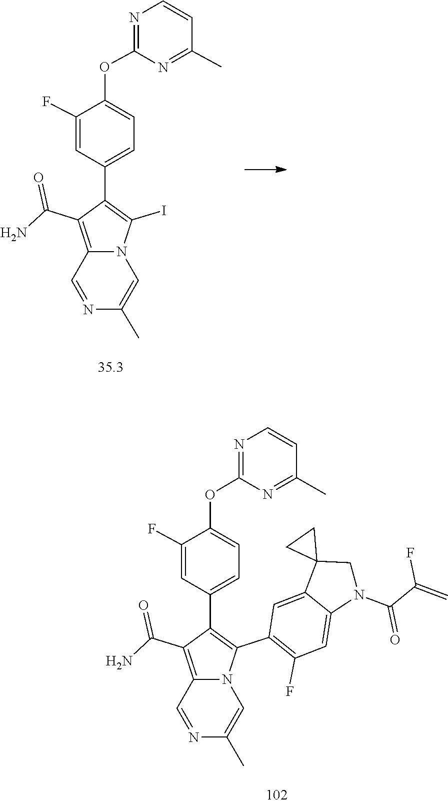 [SMILES: Cc1cn2c(I)c(-c3ccc(Oc4nccc(C)n4)c(F)c3)c(C(N)=O)c2cn1 ; C=C(F)C(=O)N1CC2(CC2)c2cc(-c3c(-c4ccc(Oc5nccc(C)n5)c(F)c4)c(C(N)=O)c4cnc(C)cn34)c(F)cc21]

### Step 1: Preparation of Example 102

In a similar fashion according to the procedure for example 15, example 102 was synthesized from compound 35.3. Spectrum data of example 102: LCMS: 611.1 [M+H]⁺. ¹H NMR: (400 MHZ, DMSO-d₆) δ 9.21 (d, J=1.2 Hz, 1H), 8.47 (d, J=5.2 Hz, 1H), 7.93-7.90 (m, 1H), 7.70 (s, 1H), 7.43 (s, 1H), 7.35-7.30 (m, 2H), 7.29 (d, J=2.0 Hz, 1H), 7.07-7.03 (m, 2H), 6.74 (d, J=7.2 Hz, 1H), 5.69-5.56 (m, 1H), 5.51 (dd, J=15.6, 4.0 Hz, 1H), 4.35-4.32 (m, 2H), 2.43 (s, 3H), 2.37 (s, 3H), 1.14-1.06 (m, 3H), 0.73-0.70 (m, 1H).

### Example 103 Preparation of 7-(4-fluoro-1-(2-fluoroacryloyl) indolin-5-yl)-6-(3-fluoro-4-((4-methylpyrimidin-2-yl)oxy)phenyl)-4-methylpyrrolo[1,2-b]pyridazine-5-carboxamide

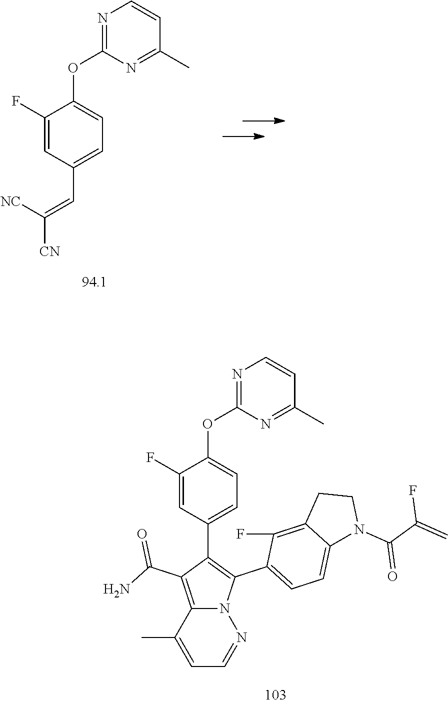 [SMILES: Cc1ccnc(Oc2ccc(C=C(C#N)C#N)cc2F)n1 ; C=C(F)C(=O)N1CCc2c1ccc(-c1c(-c3ccc(Oc4nccc(C)n4)c(F)c3)c(C(N)=O)c3c(C)ccnn13)c2F]

### Step 1: Preparation of Example 103

In a similar fashion according to the procedure for example 94, example 103 was synthesized from compound 94.1. Spectrum data of example 95: LCMS: 585.2 [M+H]⁺. ¹H NMR: (400 MHZ, DMSO-d₆) δ 8.48 (d, J=5.2 Hz, 1H), 8.15 (d, J=4.8 Hz, 1H), 7.98 (s, 1H), 7.78 (s, 1H), 7.65 (s, 1H), 7.32-7.27 (m, 2H), 7.20-7.10 (m, 3H), 6.74 (d, J=4.0 Hz, 1H), 5.64-5.46 (m, 2H), 4.37 (t, J=6.8 Hz, 2H), 3.23 (t, J=8.4 Hz, 2H), 2.57 (s, 3H), 2.43 (s, 3H).

### Example 104 Preparation of 7-(4-fluoro-1-(2-fluoroacryloyl) indolin-5-yl)-6-(3-fluoro-4-((4-methylpyrimidin-2-yl)oxy)phenyl)-4-methylpyrrolo[1,2-b]pyridazine-5-carboxamide

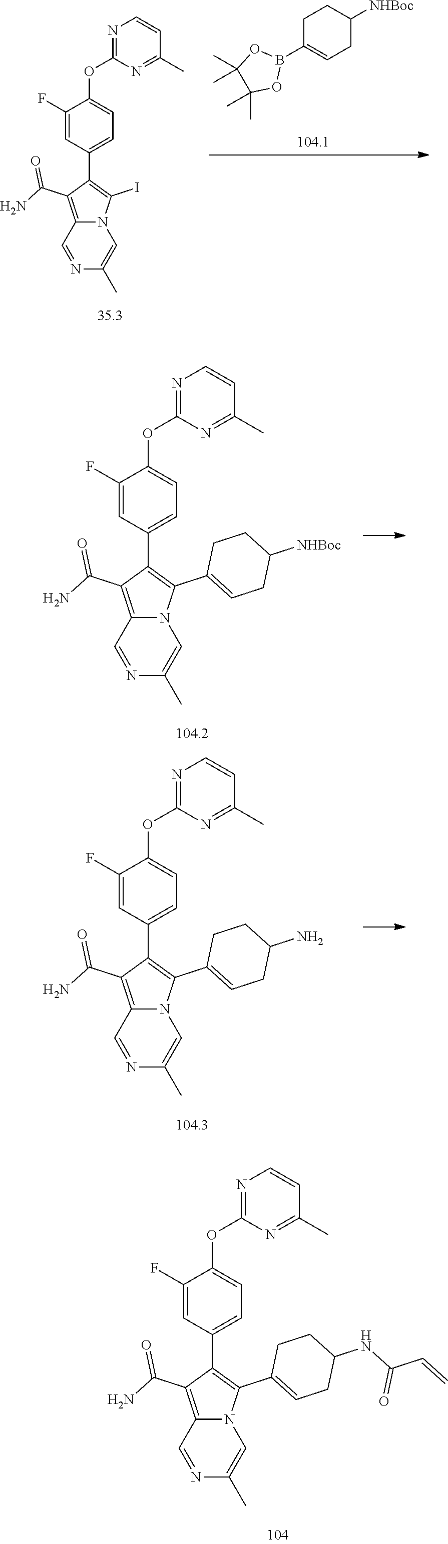

### Step 1: Preparation of Compound 104.2

To a solution of compound 35.3 (150.0 mg, 0.30 mmol), compound 104.1 (144.5 mg, 0.45 mmol) in dioxane (2 mL) and H₂O (0.6 mL) was added K₂CO₃ (103.0 mg, 0.75 mmol). Then Pd (dtbpf) Cl₂ (19.6 mg, 0.03 mmol) was added under N₂ and the reaction mixture was stirred at 90° C. for 2 h. After cooling to rt, the reaction solution was diluted with water (5 mL) and extracted with ethyl acetate (5 mL×3). The combined organic layers were washed with brine (5 mL), dried over Na₂SO₄, filtered, concentrated, and purified by flash column chromatography on silica gel to afford compound 104.2. LCMS: 573.2 [M+H]⁺.

### Step 2: Preparation of Compound 104.3

To a solution of compound 104.2 (70.0 mg, 0.12 mmol) in DCM (3 mL) was added TFA (1 mL). The mixture was stirred at 25° C. for 3 h. The reaction was concentrated to afford compound 104.3. LCMS: 473.2 [M+H]⁺.

### Step 3: Preparation of Example 104

To a solution of compound 104.3 (90.0 mg, 0.19 mmol) in acetone (3 mL) was added NaHCO₃ (32.0 mg, 0.38 mmol). Then prop-2-enoyl chloride (34.5 mg, 0.38 mmol) was added in the solution at 0° C. The reaction was stirred at 0° C. for 0.5 h. The reaction solution was diluted with water (5 mL) and extracted with DCM (5 mL×3). The combined organic layers were washed with brine (5 mL), dried over Na₂SO₄, filtered, concentrated, and purified by prep-HPLC to afford example 104. LCMS: 527.3 [M+H]⁺. ¹H NMR: (400 MHZ, DMSO-d₆) δ 9.17 (s, 1H), 8.59 (d, J=4.8 Hz, 1H), 8.19 (d, J=7.6 Hz, 1H), 8.14 (s, 1H), 7.52-7.44 (m, 3H), 7.36 (d, J=8.0 Hz, 1H), 7.30 (d, J=5.0 Hz, 1H), 7.18-7.05 (m, 1H), 6.37-6.29 (m, 1H), 6.21-6.15 (m, 1H), 6.01 (s, 1H), 5.69-5.65 (m, 1H), 4.15-4.04 (m, 1H), 2.52 (s, 3H), 2.49 (s, 3H), 2.09-2.04 (m, 2H), 1.98-1.84 (m, 2H), 1.74-1.58 (m, 2H).

### Example 105 Preparation of 3-cyclopropyl-6-(3-fluoro-4-((4-methylpyrimidin-2-yl)oxy)phenyl)-7-(4-(2-fluoroacrylamido)phenyl)pyrrolo[1,2-c]pyrimidine-5-carboxamide

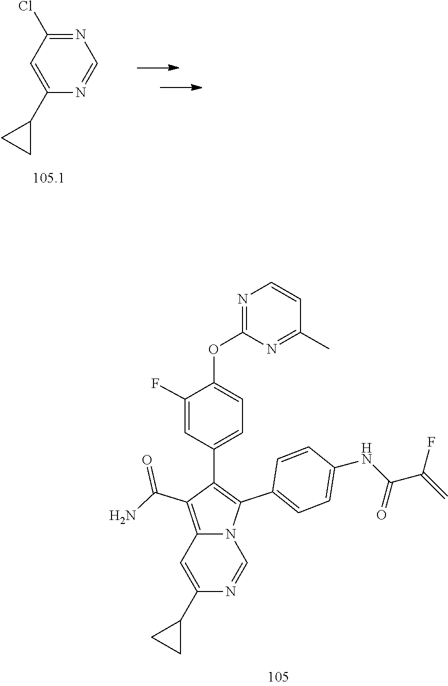 [SMILES: Clc1cc(C2CC2)ncn1 ; C=C(F)C(=O)Nc1ccc(-c2c(-c3ccc(Oc4nccc(C)n4)c(F)c3)c(C(N)=O)c3cc(C4CC4)ncn23)cc1]

### Step 1: Preparation of Example 105

In a similar fashion according to the procedure for example 15, example 105 was synthesized from compound 105.1. Spectrum data of example 105: LCMS: 567.3 [M+H]⁺. ¹H NMR: (400 MHZ, DMSO-d₆) δ 10.47 (s, 1H), 8.79 (s, 1H), 8.48 (d, J=5.2 Hz, 1H), 7.84-7.82 (m, 2H), 7.62 (s, 1H), 7.41-7.38 (m, 2H), 7.33-7.23 (m, 4H), 7.19 (d, J=4.8 Hz, 1H), 6.71-6.54 (m, 1H), 6.52 (d, J=15.2 Hz, 1H), 5.74 (dd, J=48.0, 3.2 Hz, 1H), 5.47 (dd, J=15.2, 3.2 Hz, 1H), 2.42 (s, 1H), 1.92-1.90 (m, 3H), 1.24-1.15 (m, 3H).

### Example 106 Preparation of 3-(1-cyclopropyl-1H-pyrazol-4-yl)-6-(3-fluoro-4-((4-methylpyrimidin-2-yl)oxy)phenyl)-7-(4-(2-fluoroacrylamido)phenyl)pyrrolo[1,2-c]pyrimidine-5-carboxamide

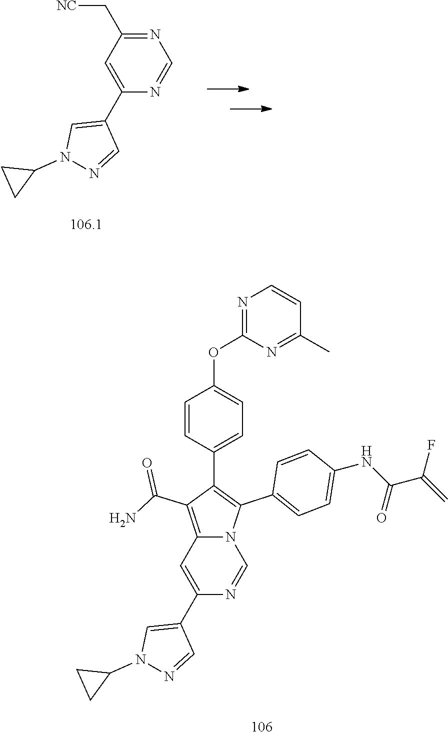 [SMILES: N#CCc1cc(-c2cnn(C3CC3)c2)ncn1 ; C=C(F)C(=O)Nc1ccc(-c2c(-c3ccc(Oc4nccc(C)n4)cc3)c(C(N)=O)c3cc(-c4cnn(C5CC5)c4)ncn23)cc1]

### Step 1: Preparation of Example 106

In a similar fashion according to the procedure for example 15, example 106 was synthesized from compound 106.1. Spectrum data of example 106: LCMS: 615.3 [M+H]⁺. ¹H NMR: (400 MHZ, DMSO-d₆) δ 10.45 (s, 1H), 8.86 (d, J=1.6 Hz, 1H), 8.42 (d, J=1.6 Hz, 1H), 8.37 (s, 1H), 8.07 (d, J=1.2 Hz, 1H), 7.97 (s, 1H), 7.81 (d, J=8.4 Hz, 1H), 7.40 (d, J=8.4 Hz, 1H), 7.32 (d, J=8.4 Hz, 1H), 7.26 (s, 1H), 7.18-7.15 (m, 3H), 6.28 (s, 1H), 5.74 (dd, J=47.6, 3.6 Hz, 1H), 5.46 (dd, J=15.6, 4.0 Hz, 1H), 3.85-3.78 (m, 1H), 2.41 (s, 3H), 1.16-1.11 (m, 2H), 1.01-0.98 (m, 2H).

### Example 107 Preparation of 2-(3-fluoro-4-((4-methylpyrimidin-2-yl)oxy)phenyl)-3-(4-(2-fluoroacrylamido)phenyl) indolizine-1-carboxamide

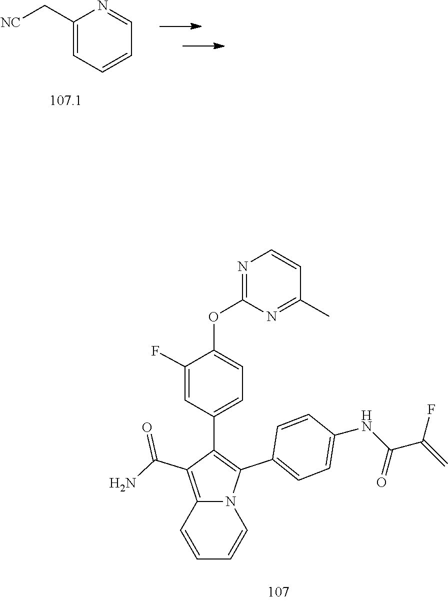 [SMILES: N#CCc1ccccn1 ; C=C(F)C(=O)Nc1ccc(-c2c(-c3ccc(Oc4nccc(C)n4)c(F)c3)c(C(N)=O)c3ccccn23)cc1]

### Step 1: Preparation of Example 107

In a similar fashion according to the procedure for example 15, example 107 was synthesized from compound 107.1. Spectrum data of example 107: LCMS: 526.1 [M+H]⁺. ¹H NMR: (400 MHZ, DMSO-d₆) δ 10.44 (s, 1H), 8.48 (d, J=5.2 Hz, 1H), 8.06 (d, J=9.2 Hz, 1H), 8.01 (d, J=6.8 Hz, 1H), 7.84 (d, J=8.4 Hz, 1H), 7.35-7.27 (m, 3H), 7.19 (d, J=4.8 Hz, 1H), 7.13-7.10 (m, 3H), 6.80-6.77 (m, 1H), 6.35 (s, 1H), 5.74 (dd, J=47.6, 3.6 Hz, 1H), 5.46 (dd, J=15.6, 3.6 Hz, 1H), 2.42 (s, 3H).

### Example 108 Preparation of 6-(3-fluoro-4-((4-methylpyrimidin-2-yl)oxy)phenyl)-7-(4-(2-fluoroacrylamido)phenyl)pyrrolo[2,1-f][1,2,4]triazine-5-carboxamide

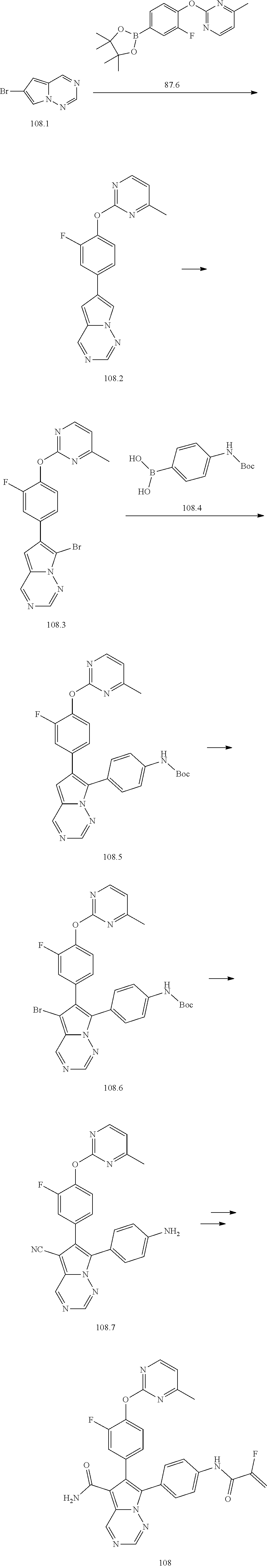

### Step 1: Preparation of Compound 108.2

To a solution of compound 108.1 (1.4 g, 7.07 mmol) and compound 87.6 (3.50 g, 10.6 mmol) in dioxane (12 mL) and H₂O (4 mL) was added K₂CO₃ (2.00 g, 14.1 mmol). Then Pd-118 (500.0 mg, 0.71 mmol) was added in the solution under N₂. The mixture was stirred at 100° C. for 2 h under N₂. After cooling to rt, the reaction mixture was concentrated and purified by flash column chromatography on silica gel to afford compound 108.2 (1.50 g, 66.0%). LCMS: 322.1 [M+H]⁺. ¹H NMR (400 MHZ, DMSO-d₆) δ 9.17 (s, 1H), 8.73 (s, 1H), 8.55 (s, 1H), 8.48 (d, J=4.8 Hz, 1H), 7.93 (dd, J=12.0, 2.0 Hz, 1H), 7.77 (d, J=8.4 Hz, 1H), 7.47-7.40 (m, 2H), 7.20 (d, J=4.8 Hz, 1H), 2.42 (s, 3H).

### Step 2: Preparation of Compound 108.3

To a solution of compound 108.2 (1.30 g, 4.05 mmol) in DMF (20 mL) was added NBS (720.2 mg, 4.05 mmol) at 0° C. The reaction was stirred at 25° C. for 12 h. After completion, the mixture was diluted with H₂O (50 mL) and extracted with ethyl acetate (50 mL×3). The combined organic layers were dried, filtered, concentrated and purified by flash column chromatography on silica gel to afford compound 108.3 (1.27 g, 78.4%). LCMS: 402.1 [M+H]⁺.

### Step 3: Preparation of Compound 108.5

To a solution of compound 108.3 (1.07 g, 2.67 mmol) and compound 108.4 (949 mg, 4.01 mmol) in dioxane (3 mL) and H₂O (1 mL) were added Pd-118 (175.7 mg, 0.267 mmol) and K₂CO₃ (1.11 g, 8.021 mmol). The reaction was stirred at 100° C. for 2 h under N₂. After cooling to rt, the mixture was concentrated in vacuum and purified by flash column chromatography on silica gel to afford compound 108.5 (1.00 g, 73.0%). LCMS: 513.1 [M+H]⁺. ¹H NMR (400 MHz, DMSO-d₆) δ 9.58 (s, 1H), 9.20 (s, 1H), 8.51 (s, 1H), 8.47 (d, J=4.8 Hz, 1H), 7.58 (d, J=8.8 Hz, 2H), 7.40 (d, J=8.4 Hz, 2H), 7.37-7.31 (m, 3H), 7.20-7.16 (m, 2H), 2.42 (s, 3H), 1.49 (s, 9H).

### Step 4: Preparation of Compound 108.6

To a solution of compound 108.5 (1.00 g, 1.95 mmol) in DMF (20 mL) was added NBS (347.3 mg, 1.95 mmol) at 0° C. The reaction was stirred at 25° C. for 1 h. After completion, the reaction mixture was concentrated and purified by flash column chromatography on silica gel to afford compound 108.6. LCMS: 593.2 [M+H]⁺. ¹H NMR (400 MHZ, DMSO-d₆) δ 9.56 (s, 1H), 9.16 (s, 1H), 8.62 (s, 1H), 8.49 (dd, J=5.2, 3.2 Hz, 1H), 7.51 (d, J=8.8 Hz, 2H), 7.41 (t, J=8.4 Hz, 1H), 7.34 (d, J=8.8 Hz, 2H), 7.28 (dd, J=11.4, 2.0 Hz, 1H), 7.20 (d, J=5.2 Hz, 1H), 7.14 (dd, J=8.4, 1.2 Hz, 1H), 2.43 (s, 3H) 1.47 (s, 9H).

### Step 5: Preparation of Compound 108.7

To a solution of compound 108.6 (500.0 mg, 0.85 mmol) in DMF (5 mL) were added Zn(CN)₂ (496.3 mg, 4.23 mmol) and Pd(PPh₃)₄ (293.1 mg, 0.254 mmol). The mixture was stirred at 140° C. for 12 h under N₂. The reaction was quenched by saturated aqueous sodium bicarbonate (5 mL) and extracted with ethyl acetate (10 mL×3). The filtrate was concentrated and purified by flash column chromatography on silica gel to afford compound 108.7. LCMS: 438.0 [M+H]⁺.

### Step 6: Preparation of Example 108

In a similar fashion according to the procedure for example 15, example 108 was synthesized from compound 108.7. Spectrum data of example 108: LCMS: 528.0 [M+H]⁺. ¹H NMR: (400 MHZ, DMSO-d₆) δ 10.45 (s, 1H), 9.39 (s, 1H), 8.68 (s, 1H), 8.48 (d, J=4.8 Hz, 1H), 7.79 (d, J=8.8 Hz, 2H), 7.63 (br s, 1H), 7.41 (d, J=8.8 Hz, 3H), 7.35 (t, J=8.4 Hz, 1H), 7.29 (dd, J=11.6, 2.0 Hz, 1H), 7.19 (d, J=5.2 Hz, 1H), 7.12 (dd, J=8.0, 1.2 Hz, 1H), 5.78 (dd, J=47.6, 3.6 Hz, 1H), 5.45 (dd, J=15.6, 3.6 Hz, 1H), 2.43 (s, 3H).

### Example A: FGFR Biochemical Assay

The following procedure was used to test the compounds described herein:
Dilute the test compounds to 100× of the final desired highest concentration in reaction by 100% DMSO.
Perform a serial dilution in a 384-well source plate by transferring 15 μL to 30 μL of 100% DMSO in the next well and so forth for a total of 10 concentrations.
Dispense 100 nL of compound solution each well from the source plate to a 384-well assay plate in duplicates by Echo (Labcyte).
Prepare a solution of 2×FGFR enzyme (Carna) in kinase buffer (50 mM HEPES, pH 7.5, 10 mM MgCl₂, 4 mM DTT, 0.01% Tween-20 and 0.01% BSA). The final concentration of FGFR1/2/3/4 is 0.01. 0.01, 0.1 and 1 nM, respectively.
Add 5 μL of kinase solution to each well of the compound-containing assay plate, except for low control wells without enzyme (add 5 μL of 1× kinase buffer instead).
Shake the plate and incubate at room temperature for 10 minutes (or 60 min as indicated as * in table 1).
Prepare the substrate solution of Fluorescein-Poly GT (Invitrogen) and ATP (Sigma) in 1× kinase reaction buffer at 2-fold of the final concentration of each reagent desired in the assay (final concentration of ATP is 28, 22, 35 and 238 μM for FGFR1/2/3/4 respectively; Fluorescein-Poly GT is fixed at 20 nM).
Add 5 μL of substrate solution to each well of the assay plate to initiate the kinase reaction.
Shake the plate shortly and incubate at room temperature for 30 minutes.
Prepare 2× detection solution in Antibody Dilution Buffer (LanthaScreen® Tb-PY20 Antibody Kit, Invitrogen).
Add 10 μL of detection solution to each well of the assay plate to stop the reaction.
Mix briefly with centrifuge and incubate at room temperature for 60 minutes before reading on a microplate reader.
Collect data on Envision with excitation at 340 nm and emission at 520 nm and 495 nm.
Calculate Ratio of RFU 520 nm/RFU 495 nm. Convert Ratio values to percent inhibition values (Percent inhibition=(max−sample Ratio)/(max−min)*100) where “min” means the Ratio of no enzyme control and “max” means the Ratio of DMSO control.
Data are analyzed and curve fitting is performed by XLFit Excel add-in version 5.4.0.8. The IC₅₀ calculation equation is: Y=Bottom+(Top-Bottom)/(1+ (IC₅₀/X)\hat{ }HillSlope.

The data from Example A is shown in Table 1.

TABLE 1

| | FGFR1 | FGFR2 | FGFR3 | FGFR4 |
|---|---|---|---|---|
| Example | IC₅₀(nM) | IC₅₀(nM) | IC₅₀(nM) | IC₅₀(nM) |
| 1 | C | A | A | D |
| 2 | C | A | A | D |
| 3 | A | A | A | NT |
| 4 | B | B | B | NT |
| 5 | C | A | B | C |
| 6 | C | A | B | D |
| 7 | C | A | B | C |
| 8 | C | A | B | D |
| 9 | C | A | B | NT |
| 10 | B | A | B | NT |
| 11 | C | A | B | C |
| 12 | D | B | C | NT |
| 13 | C | B | C | D |
| 14 | A | A | A | NT |
| 15 | C | A | B | NT |
| 16 | B | A | A | NT |
| 17 | B | A | B | NT |
| 18 | C | A | B | NT |
| 19 | B | A | B | NT |
| 20 | B | A | B | NT |
| 21 | C | A | B | NT |
| 22 | C | B | C | NT |
| 23 | C | A | B | NT |
| 24 | D | D | D | NT |
| 25 | C | A | B | NT |
| 26 | C | A | B | NT |
| 27 | D | C | C | NT |
| 28 | C | A | A | NT |
| 29 | D | B | C | NT |
| 30 | C | B | C | NT |
| 32 | B | A | A | NT |
| 33 | B | A | B | NT |
| 34 | D | C | C | NT |
| 35 | B | A | A | NT |
| 36 | B | A | A | NT |
| 37 | D | B | C | NT |
| 38 | C | B | B | NT |
| 39* | C | A | B | NT |
| 40 | D | C | C | NT |
| 41* | B | A | B | NT |
| 42* | B | A | A | NT |
| 43* | B | A | A | NT |
| 44* | D | C | D | NT |
| 45* | C | A | B | NT |
| 46* | C | B | C | NT |
| 47* | C | B | B | NT |
| 48* | C | B | B | NT |
| 49* | B | A | A | NT |
| 50* | C | B | C | NT |
| 51 | B | A | B | NT |
| 52 | D | C | D | NT |
| 53* | D | B | C | NT |
| 54* | C | B | B | NT |
| 55* | B | A | B | NT |
| 56* | C | B | C | NT |
| 57 | D | B | B | NT |
| 58* | D | A | B | NT |
| 59 | C | B | B | NT |
| 60 | D | C | C | NT |
| 61 | C | B | B | NT |
| 62 | D | B | B | NT |
| 63* | B | A | B | NT |
| 64 | B | A | A | NT |
| 65 | C | B | C | NT |
| 66 | C | B | B | NT |
| 67 | D | C | D | NT |
| 68 | D | C | C | NT |
| 69 | D | C | C | NT |
| 70 | C | C | C | NT |
| 71* | C | A | B | NT |
| 72* | C | A | B | NT |
| 73* | B | A | A | NT |
| 75 | C | C | C | NT |
| 76 | B | A | A | NT |
| 77 | D | B | C | NT |
| 78 | C | A | A | NT |
| 79 | B | A | B | NT |
| 80* | C | A | B | NT |
| 81* | B | A | A | NT |
| 82* | B | A | A | NT |
| 83* | C | A | B | NT |
| 84 | C | A | B | NT |
| 85* | C | A | B | NT |
| 86* | B | A | B | NT |
| 87* | B | A | B | NT |
| 91* | B | A | A | NT |
| 92* | B | A | A | NT |
| 93* | C | B | B | NT |
| 94* | B | A | B | NT |
| 95* | D | B | C | NT |
| 96* | B | A | A | NT |
| 97* | B | A | A | NT |
| 98* | D | C | C | NT |
| 99* | B | A | A | NT |
| 100* | B | A | A | NT |
| 101* | B | B | B | NT |
| 102* | A | B | B | NT |
| 105* | B | A | B | NT |
| 106* | B | A | B | NT |
| 107* | C | A | B | NT |
| 108* | D | B | C | NT |

A = IC₅₀ > 0 and ≤10 nM;
B = IC₅₀ > 10 nM and ≤100 nM;
C = IC₅₀ > 100 nM and ≤1000 nM;
D = IC₅₀ > 1000 nM;
NT = not tested
*means incubation at room temperature for 60 minutes before addition of substrate solution.

What is claimed is:

1. A compound of Formula (I), or a pharmaceutically acceptable salt or stereoisomer thereof: 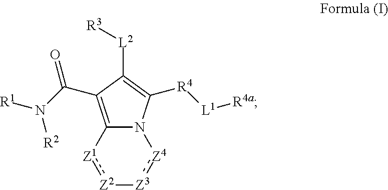
wherein:
Z¹ is absent, C(R⁵), C(R⁵)(R^{5a}), N, or N(R⁹);
Z² is C(R⁶), C(R⁶)(R^{6a}), O, S, N, or N(R⁹);
Z³ is C(R⁷), C(R⁷)(R^{7a}), O, S, N, or N(R⁹);
Z⁴ is C(R⁸), C(R⁸)(R^{8a}), O, S, N, or N(R⁹);
wherein when Z¹ is C(R⁵), C(R⁵)(R^{5a}), N, or N(R⁹), then no more than two of Z¹, Z², Z³, and Z⁴ are N or N(R⁹); and when Z¹ is absent, then no more than one of Z², Z³, and Z⁴ is O, S, N or N(R⁹);
R¹ and R² are independently selected from hydrogen, C₁₋₆alkyl, and C₁₋₆haloalkyl;
R³ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three groups selected from R^{15a},
R⁴ is selected from C₃₋₆cycloalkylene, C₂₋₉heterocycloalkylene, C₆₋₁₀arylene, and C₁₋₉heteroarylene, wherein C₃₋₆cycloalkylene, C₂₋₉heterocycloalkylene, C₆₋₁₀arylene, and C₁₋₉heteroarylene are optionally substituted with one, two, or three groups selected from R^{15b};
L¹ is a bond, —N(R^{9a})—, —N(R^{9a})C(O)—, —C(O)N(R^{9a})—, —N(R^{9a})S(O)₂—, —S(O)₂N(R^{9a})—, —O—, —C(O)—, —OC(O)—, —C(O)O—, —C(S)—, —S—, —S(O)—, —S(O)₂—, —OS(O)—, —OS(O)₂—, C₁₋₆alkylene, C₂₋₆alkenylene, C₂₋₆alkynylene, C₃₋₆cycloalkylene, C₂₋₉heterocycloalkylene, or C₁₋₉heteroarylene, wherein C₁₋₆alkylene, C₂₋₆alkenylene, C₂₋₆alkynylene, C₃₋₆cycloalkylene, C₂₋₉heterocycloalkylene, and C₁₋₉heteroarylene, are optionally substituted with one, two, or three groups selected from R^{15c};
L² is a bond, C₁₋₆alkylene, C₂₋₆alkenylene, or C₂₋₆alkynylene, wherein C₁₋₆alkylene, C₂₋₆alkenylene, or C₂₋₆alkynylene are optionally substituted with one, two, or three groups selected from R^{15c},
R^{4a} is selected from halogen, —CN, 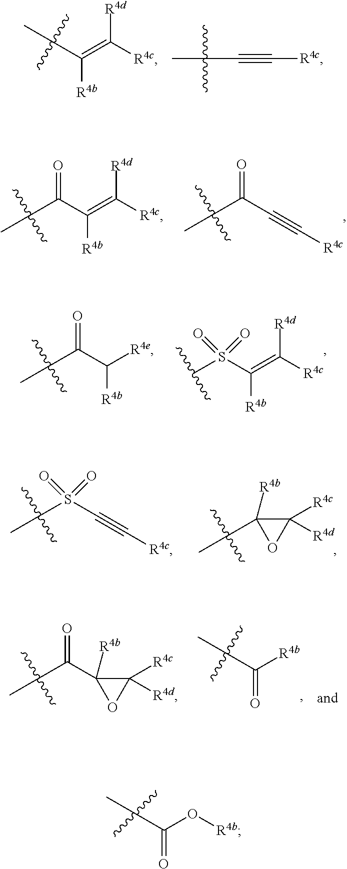
R^{4b}, R^{4c}, and R^{4d} are each independently hydrogen, halogen, —CN, —C(O)R¹³, —C(O)OR¹⁰, —C(O)N(R¹⁰)(R¹¹), —N(R¹⁰)(R¹¹), —C₁₋₆alkyl-N(R¹⁰)(R¹¹), —C(O)N(R¹⁰)OR¹⁰, C₁₋₆alkyl, phenyl, 3 to 7-membered heterocycloalkyl, or 5 or 6-membered heteroaryl, wherein the C₁₋₆alkyl, phenyl, 3 to 7-membered heterocycloalkyl, or 5 or 6-membered heteroaryl is optionally substituted with one, two, or three groups selected from R^{15d}; or
R^{4b} and R^{4c} together with the atoms to which they are attached form a 3 to 14-membered cycloalkyl or 3 to 14-membered heterocycloalkyl; wherein the 3 to 14-membered cycloalkyl and 3 to 14-membered heterocycloalkyl are optionally substituted with 1, 2, 3, or 4 R^{15d}, or R^{4d} and R^{4c} together with the atoms to which they are attached form a 3 to 14-membered cycloalkyl or 3 to 14-membered heterocycloalkyl; wherein the 3 to 14-membered cycloalkyl and 3 to 14-membered heterocycloalkyl are optionally substituted with 1, 2, 3, or 4 R^{15d};
R^{4e} is halogen or —OS(O)₂R¹³;
R⁵, R^{5a}, R⁶, R^{6a}, R⁷, R^{7a}, R⁸, and R^{8a}are independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)(R¹³), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15e};
R⁹ is selected from hydrogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹⁰, —C(O)R¹³, —S(O)R¹³, —C(O)N(R¹⁰)(R¹¹), and —S(O)₂R¹³, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15f};
R^{9a} is selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15g};
each R¹⁰ is independently selected from hydrogen, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, —CH₂—C₆₋₁₀aryl, C₆₋₁₀aryl, C₁₋₉heteroaryl, and —CH₂—C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, —CH₂—C₆₋₁₀aryl, C₆₋₁₀aryl, C₁₋₉heteroaryl, and —CH₂—C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl; or R¹⁰ and R¹¹, together with the nitrogen to which they are attached, form a C₂₋₉heterocycloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R^{15a}, R^{15b}, R^{15c}, R^{15d}, R^{15c}, R^{15f}, and R^{15g} are each independently selected from halogen, oxo, —CN, C₁₋₆alkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)(R¹³), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)(R¹³), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹) and —P(O)(R¹⁰)₂, and [symbol]
indicates a single or double bond such that all valences are satisfied.

2. The compound of claim 1, or a pharmaceutically acceptable salt or stereoisomer thereof, having the structure of Formula (Ia):

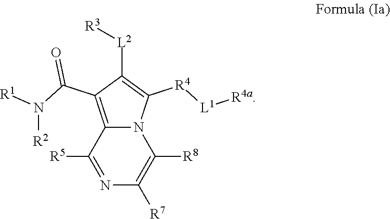

3. The compound of claim 1, or a pharmaceutically acceptable salt or stereoisomer thereof, having the structure of Formula (Ib):

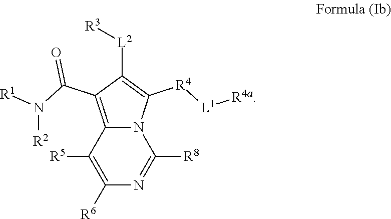

4. The compound of claim 1, or a pharmaceutically acceptable salt or stereoisomer thereof, having the structure of Formula (Ic):

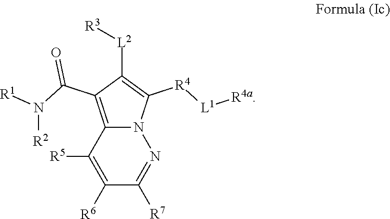

5. The compound of claim 1, or a pharmaceutically acceptable salt or stereoisomer thereof, having the structure of Formula (Id):

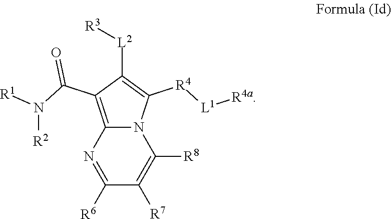

6. The compound of claim 1, or a pharmaceutically acceptable salt or stereoisomer thereof, having the structure of Formula (Ie):

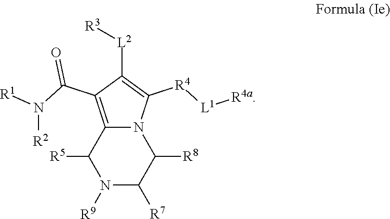

7. The compound of claim 6, or a pharmaceutically acceptable salt or stereoisomer thereof, wherein R⁹ is selected from hydrogen, C₁₋₆alkyl, and C₁₋₆haloalkyl.

8. The compound of claim 7, or a pharmaceutically acceptable salt or stereoisomer thereof, wherein R⁹ is C₁₋₆alkyl.

9. The compound of any one of claims 1-8, or a pharmaceutically acceptable salt or stereoisomer thereof, wherein R⁵, R⁶, R⁷, and R⁸ are independently selected from hydrogen, halogen, —OR¹⁰, and C₁₋₆alkyl, wherein C₁₋₆alkyl is optionally substituted with one, two, or three groups selected from R^{15e}.

10. The compound of any one of claims 1-9, or a pharmaceutically acceptable salt or stereoisomer thereof, wherein R⁵, R⁶, R⁷, and R⁸ are independently selected from hydrogen and unsubstituted C₁₋₆alkyl.

11. The compound of any one of claims 1-10, or a pharmaceutically acceptable salt or stereoisomer thereof, wherein R⁵, R⁶, R⁷, and R⁸ are each hydrogen.

12. The compound of any one of claims 1-11, or a pharmaceutically acceptable salt or stereoisomer thereof, wherein R³ is selected from C₆₋₁₀aryl and C₁₋₉heteroaryl, wherein C₆₋₁₀aryl and C₁₋₉heteroaryl are optionally substituted with one, two, or three groups selected from R^{15a}.

13. The compound of any one of claims 1-12, or a pharmaceutically acceptable salt or stereoisomer thereof, wherein R³ is phenyl optionally substituted with one, two, or three groups selected from R^{15a}.

14. The compound of any one of claims 1-13, or a pharmaceutically acceptable salt or stereoisomer thereof, wherein R³ is phenyl substituted with one, two, or three groups selected from R^{15a}.

15. The compound of claim 13, or a pharmaceutically acceptable salt or stereoisomer thereof, having the structure of Formula (Ia′) or Formula (Ia″): 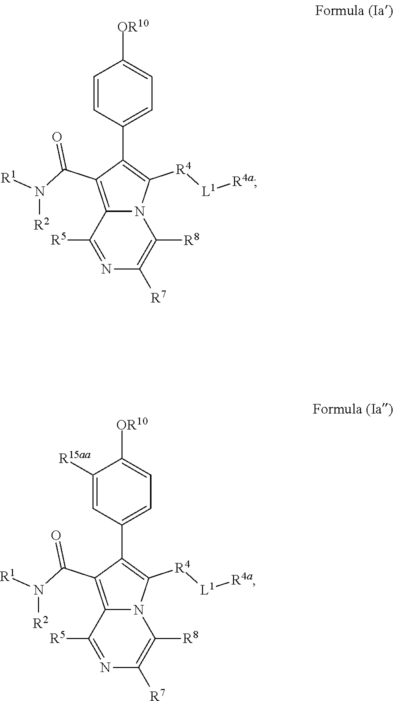
wherein
R¹⁰ is C₁₋₉heteroaryl optionally substituted with one, two, or three groups selected from halogen, —CN, hydroxy, C₁₋₆alkyl, C₁₋₆haloalkyl, and C₁₋₆alkoxy; and
R^{15aa} has the same meaning as R^{15a} in claim 1.

16. The compound of claim 13, or a pharmaceutically acceptable salt or stereoisomer thereof, having the structure of Formula (Ib′) or Formula (Ib″): 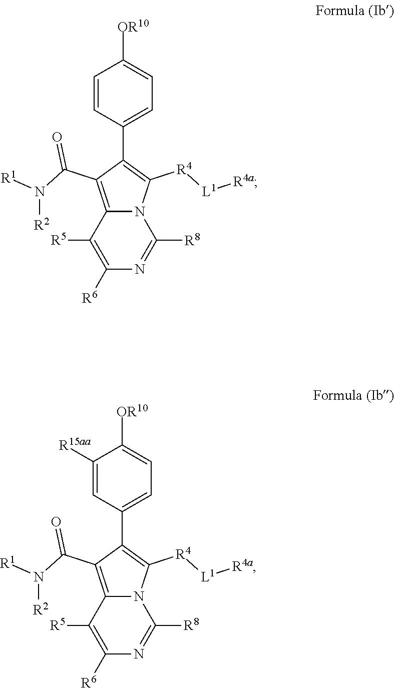
wherein
R¹⁰ is C₁₋₉heteroaryl optionally substituted with one, two, or three groups selected from halogen, —CN, hydroxy, C₁₋₆alkyl, C₁₋₆haloalkyl, and C₁₋₆alkoxy; and
R^{15aa} has the same meaning as R^{15a} in claim 1.

17. The compound of claim 13, or a pharmaceutically acceptable salt or stereoisomer thereof, having the structure of Formula (Ic′) or Formula (Ic″): 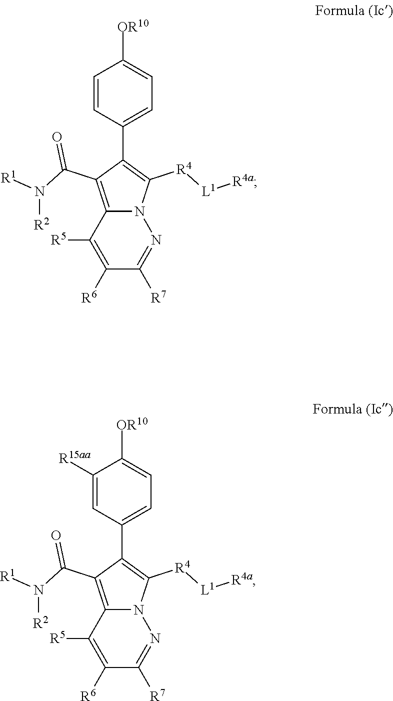
wherein
R¹⁰ is C₁₋₉heteroaryl optionally substituted with one, two, or three groups selected from halogen, —CN, hydroxy, C₁₋₆alkyl, C₁₋₆haloalkyl, and C₁₋₆alkoxy; and
R^{15aa} has the same meaning as R^{15a} in claim 1.

18. The compound of any one of claims 15-17, or a pharmaceutically acceptable salt or stereoisomer thereof, wherein R^{15aa} is selected from halogen, C₁₋₆alkyl, and —OCH₃.

19. The compound of any one of claims 15-18, or a pharmaceutically acceptable salt or stereoisomer thereof, wherein R^{15aa} is halogen.

20. The compound of any one of claims 15-19, or a pharmaceutically acceptable salt or stereoisomer thereof, wherein R¹⁰ is C₁₋₉heteroaryl optionally substituted with one, two, or three groups selected from halogen, —CN, hydroxy, C₁₋₆alkyl, C₁₋₆haloalkyl, and C₁₋₆alkoxy.

21. The compound of any one of claims 15-20, or a pharmaceutically acceptable salt or stereoisomer thereof, wherein R¹⁰ is pyridyl or pyrimidinyl, wherein the pyridyl and pyrimidinyl are optionally substituted with one, two, or three groups selected from halogen, —CN, hydroxy, C₁₋₆alkyl, C₁₋₆haloalkyl, and C₁₋₆alkoxy.

22. The compound of any one of claims 15-21, or a pharmaceutically acceptable salt or stereoisomer thereof, wherein R¹⁰ is pyridyl or pyrimidinyl, wherein the pyridyl and pyrimidinyl are substituted with one, two, or three groups selected from C₁₋₆alkyl.

23. The compound of any one of claims 15-22, or a pharmaceutically acceptable salt or stereoisomer thereof, wherein R¹⁰ is

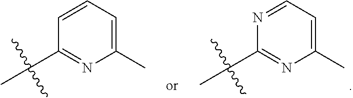

24. The compound of any one of claims 1-23, or a pharmaceutically acceptable salt or stereoisomer thereof, wherein R⁴ is selected from C₂₋₉heterocycloalkylene, C₆₋₁₀aryl and C₁₋₉heteroaryl, wherein C₂₋₉heterocycloalkylene, C₆₋₁₀aryl and C₁₋₉heteroaryl are optionally substituted with one, two, or three groups selected from R^{15b}.

25. The compound of any one of claims 1-24, or a pharmaceutically acceptable salt or stereoisomer thereof, wherein R⁴ is phenyl optionally substituted with one, two, or three groups selected from R^{15b}.

26. The compound of any one of claims 1-25, or a pharmaceutically acceptable salt or stereoisomer thereof, wherein R⁴ is phenyl substituted with one, two, or three groups selected from R^{15b}.

27. The compound of any one of claims 1-26, or a pharmaceutically acceptable salt or stereoisomer thereof, wherein L¹ is a bond, —N(R^{9a})—, —N(R^{9a})C(O)—, —C(O)N(R^{9a})—, —N(R^{9a})S(O)₂—, —S(O)₂N(R^{9a})—, —O—, —C(O)—, —OC(O)—, —C(O)O—, —S—, —S(O)—, —S(O)₂—, or C₁₋₆alkylene optionally substituted with one, two, or three groups selected from R^{15c}.

28. The compound of any one of claims 1-27, or a pharmaceutically acceptable salt or stereoisomer thereof, wherein L¹ is a bond, —N(R^{9a})—, —N(R^{9a})C(O)—, —C(O)N(R^{9a})—, or —C(O)—.

29. The compound of any one of claims 1-28, or a pharmaceutically acceptable salt or stereoisomer thereof, wherein L¹ is a —N(R^{9a})—.

30. The compound of any one of claims 1-29, or a pharmaceutically acceptable salt or stereoisomer thereof, wherein R^{9a} is selected from hydrogen and C₁₋₆alkyl optionally substituted with one, two, or three groups selected from R^{15g}.

31. The compound of any one of claims 1-30, or a pharmaceutically acceptable salt or stereoisomer thereof, wherein R^{9a} is hydrogen.

32. The compound of any one of claims 1-31, or a pharmaceutically acceptable salt or stereoisomer thereof, wherein R^{4a} is selected from

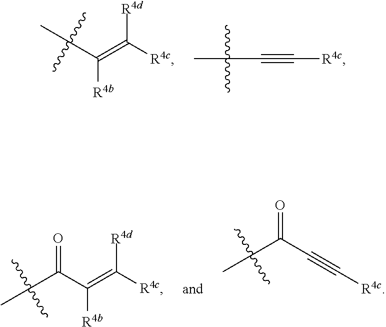

33. The compound of any one of claims 1-32, or a pharmaceutically acceptable salt or stereoisomer thereof, wherein R^{4a} is

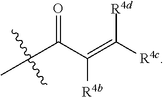

34. The compound of any one of claims 1-33, or a pharmaceutically acceptable salt or stereoisomer thereof, wherein R^{4b}, R⁴° C., and R^{4d} are each independently hydrogen, halogen, C₁₋₆alkyl, or —C₁₋₆alkyl-N(R¹⁰)(R¹¹), wherein the alkyl is optionally substituted with one, two, or three R^{15d}.

35. The compound of any one of claims 1-34, or a pharmaceutically acceptable salt or stereoisomer thereof, wherein R^{4c} and R^{4d} are each independently hydrogen or —C₁₋₆alkyl-N(R¹⁰)(R¹¹).

36. The compound of any one of claims 1-35, or a pharmaceutically acceptable salt or stereoisomer thereof, wherein R^{4c} and R^{4d} are each independently hydrogen or —CH₂—N(CH₃)₂.

37. The compound of any one of claims 1-36, or a pharmaceutically acceptable salt or stereoisomer thereof, wherein R^{4b} is hydrogen, halogen, or C₁₋₆alkyl, wherein the alkyl is optionally substituted with one, two, or three R^{15d}.

38. The compound of any one of claim 1-14 or 18-37, or a pharmaceutically acceptable salt or stereoisomer thereof, wherein L² is a bond.

39. The compound of any one of claims 1-38, or a pharmaceutically acceptable salt or stereoisomer thereof, wherein R¹ and R² are independently selected from hydrogen and C₁₋₆alkyl.

40. The compound of any one of claims 1-39, or a pharmaceutically acceptable salt or stereoisomer thereof, wherein R¹ and R² are hydrogen.

41. A compound, or a pharmaceutically acceptable salt or stereoisomer thereof, selected from:

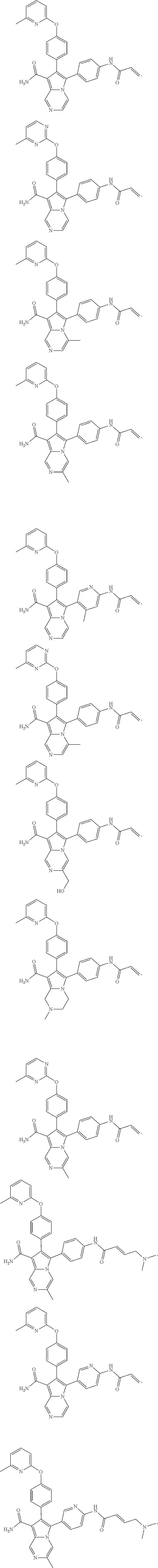

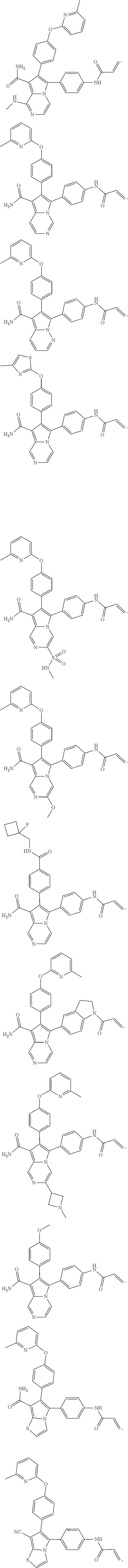

[chemical structure]

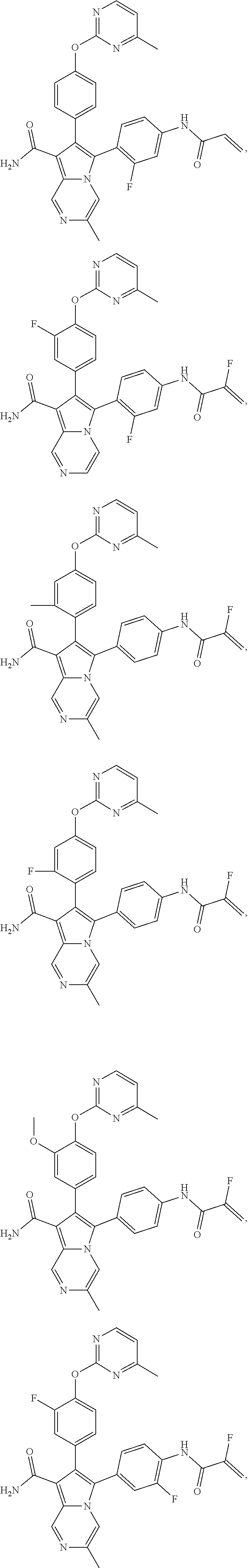

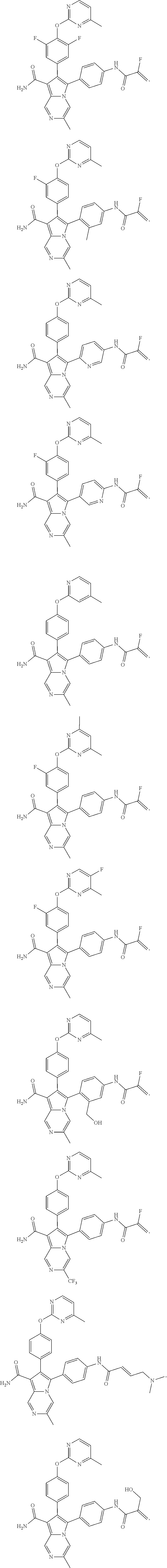

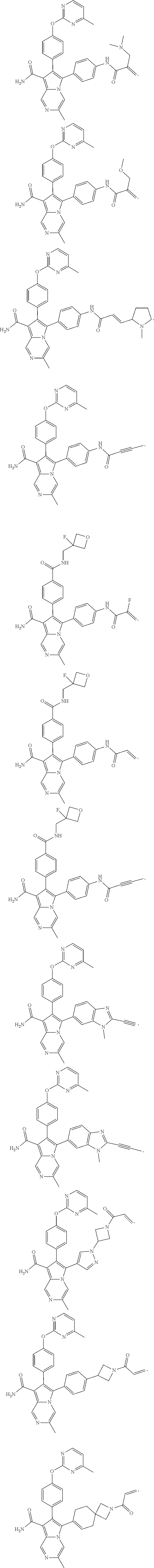

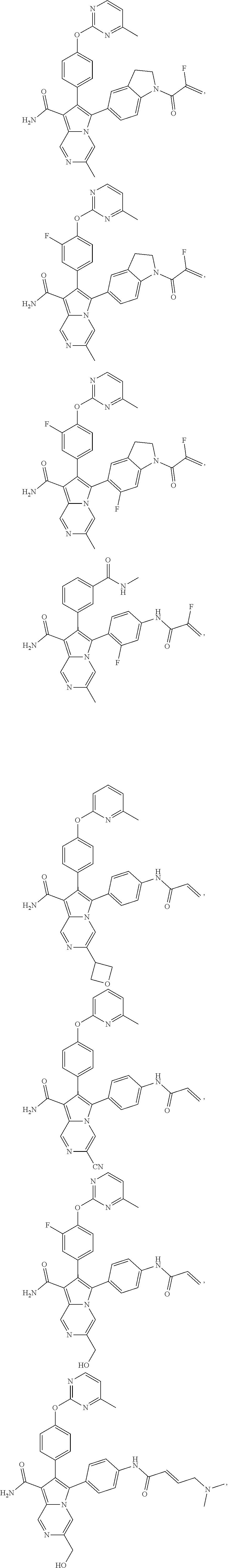

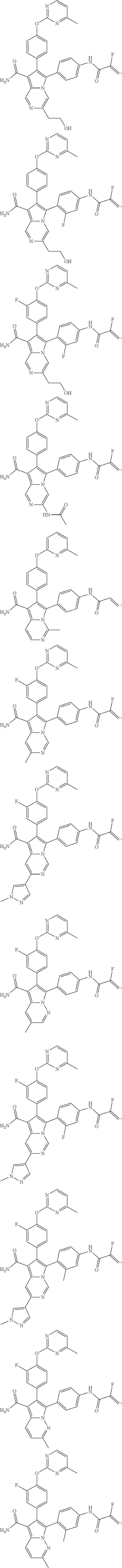

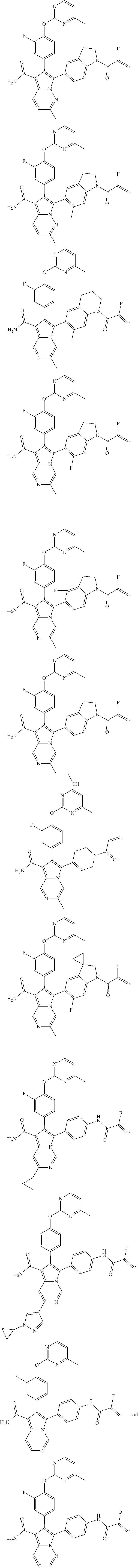

42. A pharmaceutical composition comprising a compound of any one of claims 1-41, or a pharmaceutically acceptable salt or stereoisomer thereof, and at least one pharmaceutically acceptable excipient.

43. A method of treating cancer in a mammal in need thereof, comprising administering to the mammal a compound of any one of claims 1-41, or a pharmaceutically acceptable salt or stereoisomer thereof.

44. The method of claim 43, wherein the cancer is a solid tumor.

45. The method of claim 44, wherein the cancer is intra-hepatic cholangiocarcinoma, urothelial cancer, gastric cancer, bladder cancer, breast cancer, endometrial cancer, kidney cancer, liver cancer, lung cancer, melanoma, pancreatic cancer, prostate cancer, or thyroid cancer.
